(19) United States
(12) Patent Application Publication
(10) Pub. No.: US 2026/0023081 A1
(43) Pub. Date: Jan. 22, 2026

(54) SOLID PHASE LIBRARY PREPARATION FOR POLYPEPTIDE SEQUENCING

(71) Applicant: Quantum-Si Incorporated, Branford, CT (US)

(72) Inventors: Haidong Huang, Madison, CT (US); Matthew T. Richers, Clinton, CT (US); Mandeep Singh, Branford, CT (US)

(73) Assignee: Quantum-Si Incorporated, Branford, CT (US)

(21) Appl. No.: 19/277,269

(22) Filed: Jul. 22, 2025

Related U.S. Application Data
(60) Provisional application No. 63/674,237, filed on Jul. 22, 2024.

(51) Int. Cl. G01N 33/68; C07K 1/04; C12Q 1/37; G01N 33/543

(52) U.S. Cl. CPC G01N 33/6824; C07K 1/047; C12Q 1/37; G01N 33/543; G01N 2333/948

(57) ABSTRACT

Provided herein are compounds of Formulae (I), (II), (III), and (III-d). Also provided herein are methods of preparation of Formulae (I), (II), and (III-d). Further provided herein are methods of functionalizing or sequencing a peptide by reaction of compounds of Formula (III-d) with peptidases.

### CROSS-REFERENCE TO RELATED APPLICATIONS

The present application claims the benefit of priority of U.S. Provisional Application No. 63/674,237, filed Jul. 22, 2024, the entire content of which is incorporated herein by reference.

### REFERENCE TO AN ELECTRONIC SEQUENCE LISTING

The contents of the electronic sequence listing (R070870178US01-SEQ-WLC.xml; Size: 57,498 bytes; and Date of Creation: Jul. 22, 2025) are herein incorporated by reference in their entirety.

### BACKGROUND OF THE INVENTION

Proteomics has emerged as an important and necessary complement to genomics and transcriptomics in the study of biological systems. The proteomic analysis of an individual organism can provide insights into cellular processes and response patterns, which can lead to improved diagnostic and therapeutic strategies. The complexity surrounding protein structure, composition, and modification present challenges in determining large-scale protein sequencing information for a biological sample.

Previous work has led to the development of methods of polypeptide sequencing that involve using a degradation process of a polypeptide with peptidases to produce an amino acid sequence representative of the polypeptide. See, e.g., PCT International Publication No. WO2020/102741A1, filed Nov. 15, 2019, PCT International Publication No. WO2021/236983A2, filed May 20, 2021, and PCT International Publication No. WO2024/086826A2, filed Oct. 20, 2023, each of which is incorporated by reference in its entirety. There is a need for improvements in the sample preparation for such methods, to minimize loss of polypeptide over each step and reduce the total amount of polypeptide needed.

### SUMMARY OF THE INVENTION

The present disclosure provides novel compounds for solid-phase library preparation, and methods for peptide functionalization and sequencing.

In one aspect, the present disclosure provides a compound of Formula (I):

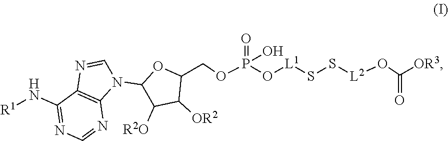

or a salt thereof, wherein R¹, R², R³, L¹, and L² are as defined herein.

In another aspect, the present disclosure provides a compound of Formula (II):

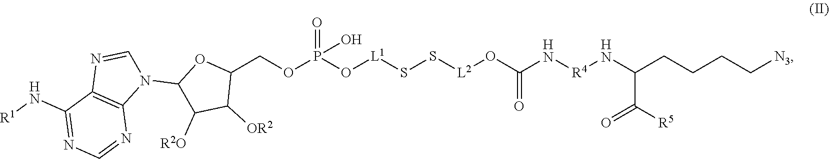

or a salt thereof, wherein R¹, R², R⁴, R⁵, L¹, and L² are as defined herein.

In another aspect, the present disclosure provides a compound of Formula (III):

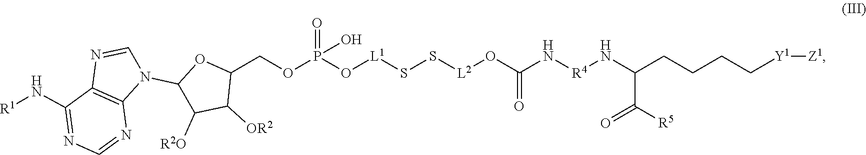

or a salt thereof, wherein R¹, R², R⁴, R⁵, L¹, L², Y¹, and Z¹ are as defined herein.

In another aspect, the present disclosure provides a method of preparing a compound of Formula (I):

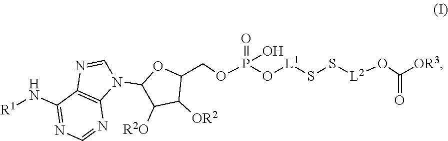

or a salt thereof, comprising coupling a compound of Formula (IV):

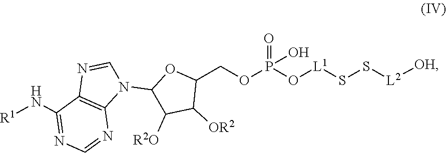

or a salt thereof, with a compound of Formula (V):

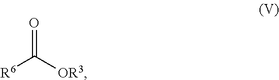

or a salt thereof, under suitable conditions to obtain the compound of Formula (I), or salt thereof, wherein: R¹, R², R³, R⁶, L¹, and L² are as defined herein.

In another aspect, the present disclosure provides a method of preparing a compound of Formula (II):

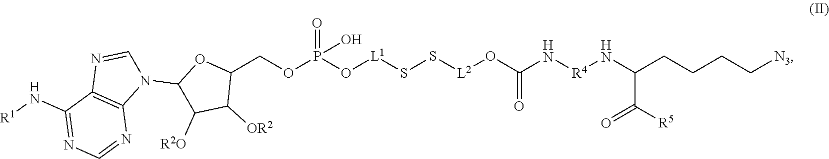

or a salt thereof, comprising coupling a compound of Formula (I):

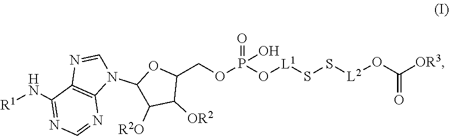

or a salt thereof, with a compound of Formula (XII):

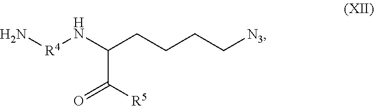

or a salt thereof, under suitable conditions to obtain the compound of Formula (II), or salt thereof, wherein R¹, R², R³, R⁴, R⁵, L¹, and L² are as defined herein.

In another aspect, the present disclosure provides a method of preparing a compound of Formula (III-d):

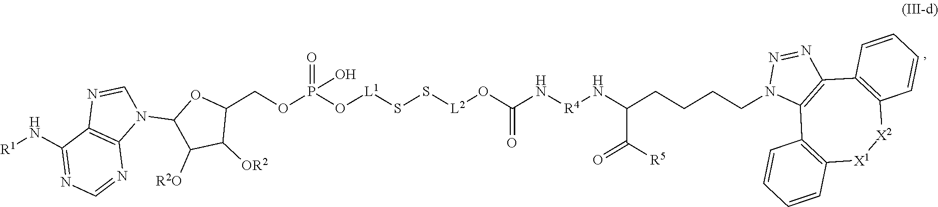

or a salt thereof, comprising coupling a compound of Formula (II):

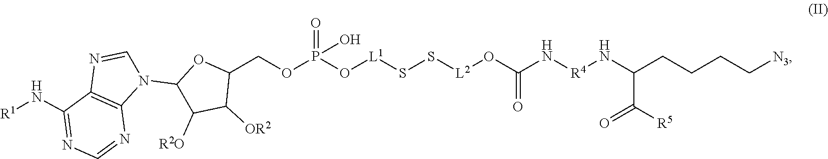

or a salt thereof, with a compound of Formula (XIII):

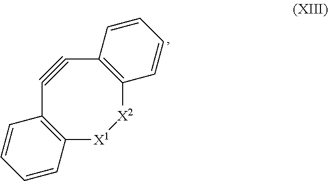

or a salt thereof, under suitable conditions to obtain the compound of Formula (III-d), or salt thereof, wherein one of X¹ and X² is CH and the other is N—Z¹, wherein Z¹ comprises an oligonucleotide, and R¹, R², R³, R⁴, R⁵, L¹, and L² are as defined herein.

In another aspect, the present disclosure provides a method of functionalizing a first terminus of a peptide comprising:
(a) conjugating a second terminus of the peptide to a solid support group; and
(b) conjugating the first terminus of the peptide to a linking group.

In another aspect, the present disclosure provides a method of sequencing a peptide, comprising:
(a) conjugating a second terminus of the peptide to a solid support group;
(b) conjugating the first terminus of the peptide to a linking group;
(c) exposing the peptide to a peptidase in a degradation process;
(d) obtaining data during the degradation process;
(e) analyzing the data to determine portions of the data corresponding to amino acids that are sequentially exposed at the second terminus of the peptide during the degradation process; and
(f) outputting an amino acid sequence representative of the peptide.

In another aspect, the present disclosure provides a method of sequencing a peptide, comprising exposing a compound of Formula (III-d):

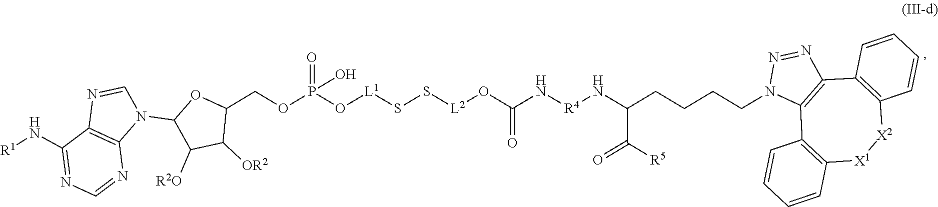

or a salt thereof, to a peptidase in a degradation process;
obtaining data during the degradation process;
analyzing the data to determine portions of the data corresponding to amino acids that are sequentially exposed at a terminus of the peptide during the degradation process; and
outputting an amino acid sequence representative of the peptide;
   wherein R¹, R², R⁴, R⁵, L¹, and L² are as defined herein; and one of X¹ and X² is CH and the other is N—Z¹, wherein Z¹ comprises an oligonucleotide.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

FIG. 1 shows the standard protein library preparation for protein sequencing.

FIGS. 2A-2B show an overview of solid-phase library preparation (SPLP).

FIG. 3 shows the catch and release of sulfo-Cy3-amine on 5 mg PEGA-SS-BME-PNP.

FIG. 4 shows the catch and release of a representative sample peptide on 5 mg PEGA-SS-BME-PFP resin.

FIG. 5 shows the catch and release of 10 nmol sulfo-Cy3-Gly-Gly on 5 mg Glen-SS-BME-PFP resin.

FIG. 6 shows the synthesis of the Glen-SS-BME-PFP resin.

FIGS. 7A-7B show schematics of solid-phase library preparation (SPLP) workflow, for on-chip streptavidinylation (FIG. 7A) and on-resin streptavidinylation (FIG. 7B).

FIG. 8 shows sequencing of CDNF library (0.5 nmol) using SPLP workflow.

FIGS. 9A-9D show comparisons between peptide sequencing preparations. FIG. 9A shows 500 pmol SOP CDNF library vs 5 pmol synthetic CDNF mix (SPLP). SOP Input=500 pmol protein; Alignments=20357; #peptides=6; Alignments/RRL 1+=6.233%. SPLP Input=5 pmol synthetic CDNF; Alignments=10054; #peptides=5; Alignments/RRL 1+=1.591%. FIG. 9B shows 500 pmol SOP CDNF library vs 5 pmol Synthetic CDNF mix SPLP with library prep reagents. SOP Input=500 pmol protein; Alignments=16148; #peptides=6; Alignments/RRL 1+=5.117%. SPLP Input=5 pmol synthetic CDNF; Alignments=5272; #peptides=3; Alignments/RRL 1+=1.096%. FIG. 9C shows 500 pmol SOPK2C8 library vs 5 pmol SOP K2C8 library. SOP Input=500 pmol K2C8; Alignments=3461; #peptides=8; Alignments/RRL 1+=1.036%. 5 pmol K2C8 solution phase; Input=5 pmol K2C8; Alignments=32; #peptides=1; Alignments/RRL 1+=0.039%. FIG. 9D shows 500 pmol SOP (v1) K2C8 library vs 5 pmol SPLP K2C8 library. SOP Input=500 pmol protein; Alignments=6524; #peptides=7; Alignments/RRL 1+=1.428%. SPLP Input=5 pmol K2C8 digest; Alignments=1632; #peptides=3; Alignments/RRL 1+=0.4544%.

FIG. 10 shows a comparison of catching between the BME-PFP and BME-NHS resins, in the absorbance of supernatant in catch of 100 uM sulfo-Cy3-Gly-Gly on 5 mg Glen-SS-BME-PFP vs. Glen-SS-BME-NHS.

FIG. 11 shows a comparison of the metastable species clearance of two resins, BME and BMP. Shown is percentage of metastable intermediate cleaved from sulfo-Cy3-Gly-Gly with 50 mM DTT in RT pH 8.0 sample buffer (HPLC assay).

FIG. 12 shows ratio MS signal catch supernatant/precatch for catch of 30 pmol synthetic CDNF with different resins and conditions.

FIG. 13 shows clearance of metastable peptide-BME adducts at an elevated temperature, through ratio MS abundance release/precatch with 30 min DTT release (t0), followed by 45° C. incubation.

FIG. 14 shows the effect of SV passivation on reads. The “AS” ROIs are associated with SV passivation and negatively impact the alignment of synthetic CDNF control.

FIGS. 15A-15D show that excess SV is tied to the artifact “AS” ROIs. FIG. 15A shows a schematic of three different SPLP workflows. The reads are shown for the V0 (FIG. 15B), V1.0 (FIG. 15C), and V1.1 (FIG. 15D) workflows. 5 pmol CDNF SPLP libraries, topped ranked motifs in different workflows.

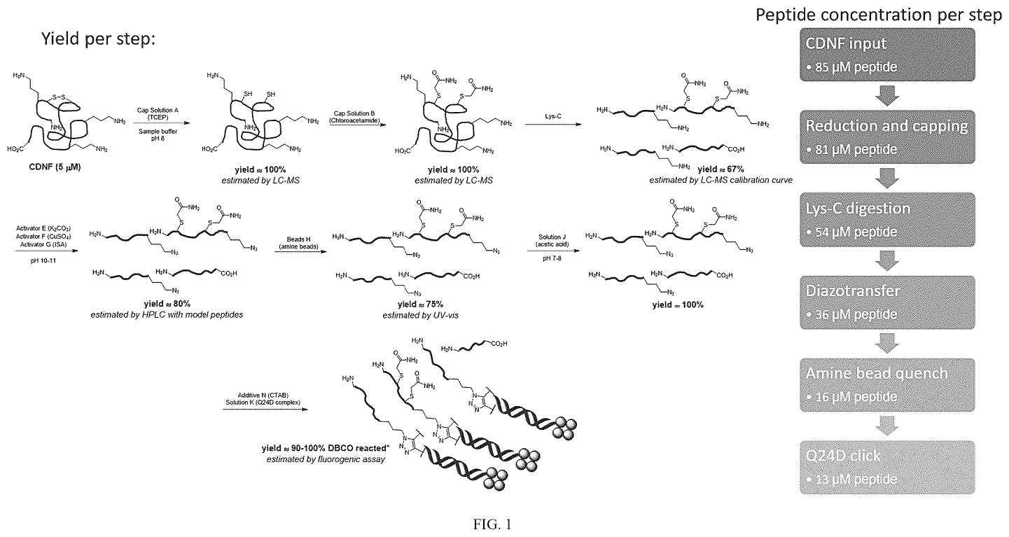

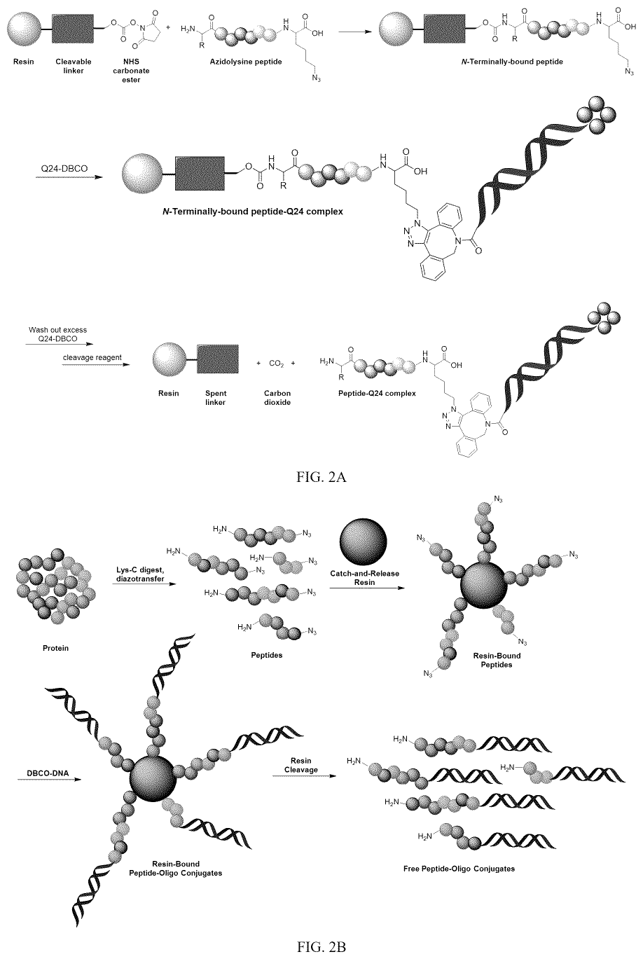

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modem Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The present disclosure additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₃, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and lor 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

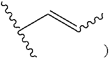

may be in the (E)- or (Z)-configuration.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzo-thienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydro-pyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, SO₂N(R^{bb})₂, —SO₂R^{aa}, SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxy,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, NHCO₂R^{aa}, NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, 4,4′-dimethoxytrityl (4,4′-dimethoxytriphenylmethyl or DMT), a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methanesulfonate, trifluoromethanesulfonate (triflate), p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₄⁻, AsF₄⁻, SbF₄⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. In some embodiments, a leaving group is an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

These and other exemplary substituents are described in more detail in the Detailed Description, Examples, and Claims. The present disclosure is not limited in any manner by the above exemplary listing of substituents.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

As used herein, the term “about X,” or “approximately X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

Amino acid residues may be indicated by their corresponding single letter codes, e.g., R (arginine), H (histidine), K (lysine), D (aspartic acid), E (glutamic acid), S (serine), T (threonine), N (asparagine), Q (glutamine), C (cysteine), G (glycine), P (proline), A (alanine), V (valine), I (isoleucine), L (leucine), M (methionine), F (phenylalanine), Y (tyrosine), W (tryptophan).

A “peptidase,” “protease,” or “proteinase” is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. An exopeptidase in accordance with the application may be an “aminopeptidase” or a “carboxypeptidase,” which cleaves a single amino acid from an amino- or a carboxy-terminus, respectively. A peptidase (e.g., an aminopeptidase) may also be referred to as a “cutter” or a “cleaving reagent.”

A “TET aminopeptidase” is composed of 12 monomers that assemble into a tetrahedral structure with 3 active sites in each corner. To access the active sites for digestion, a polypeptide may pass through a pore that leads into the central chamber of the tetrahedron. Each of the 4 faces of the tetrahedron contain one pore in the center of the face. The pore is narrow and does not permit larger compounds (e.g., double-stranded DNA) to pass through.

The term “avidin protein” refers to a biotin-binding protein, generally having a biotin binding site at each of four subunits of the avidin protein. Avidin proteins include, for example, avidin, streptavidin, traptavidin, tamavidin, bradavidin, xenavidin, and homologs and variants thereof. In some cases, the monomeric, dimeric, or tetrameric form of the avidin protein can be used. In some embodiments, the avidin protein of an avidin protein complex is streptavidin in a tetrameric form (e.g., a homotetramer).

The term “click chemistry” refers to a chemical synthesis technique introduced by K. Barry Sharpless of The Scripps Research Institute, describing chemistry tailored to generate covalent bonds quickly and reliably by joining small units comprising reactive groups together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2]cycloaddition). Exemplary click chemistry reactions include, but are not limited to, azide-alkyne Huisgen cycloaddition; and Diels-Alder reactions (e.g., tetrazine [4+2]cycloaddition). In some embodiments, click chemistry reactions are modular, wide in scope, give high chemical yields, generate inoffensive byproducts, are stereospecific, exhibit a large thermodynamic driving force >84 kJ/mol to favor a reaction with a single reaction product, and/or can be carried out under physiological conditions. In some embodiments, a click chemistry reaction exhibits high atom economy, can be carried out under simple reaction conditions, use readily available starting materials and reagents, uses no toxic solvents or use a solvent that is benign or easily removed (preferably water), and/or provides simple product isolation by non-chromatographic methods (crystallization or distillation).

The term “click chemistry handle,” as used herein, refers to a reactant, or a reactive group, that can partake in a click chemistry reaction. For example, a strained alkyne, e.g., a cyclooctyne, is a click chemistry handle, since it can partake in a strain-promoted cycloaddition (see, e.g., Table 1). In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles suitable for use according to some aspects of this invention are described herein, for example, in Tables 1 and 2. In some embodiments, click chemistry handles are used that can react to form covalent bonds in the presence of a metal catalyst, e.g., copper (II). In some embodiments, click chemistry handles are used that can react to form covalent bonds in the absence of a metal catalyst. Additional suitable click chemistry handles are well known to those of skill in the art, and such click chemistry handles include, but are not limited to, the click chemistry reaction partners, groups, and handles described in Becer, Hoogenboom, and Schubert, Click Chemistry beyond Metal-Catalyzed Cycloaddition, Angewandte Chemie International Edition (2009) 48: 4900-4908 and PCT/US2012/044584 and references therein, which references are incorporated herein by reference for click chemistry handles and methodology.

TABLE 1
Exemplary click chemistry handles and reactions.

| | |
|---|---|
| 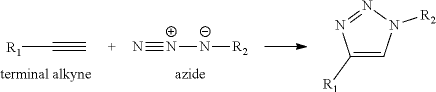 | 1,3-dipolar cycloaddition |
| 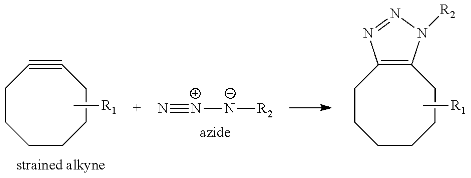 | Strain-promoted cycloaddition |
| 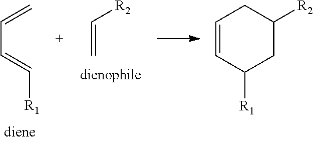 | Diels-Alder reation |
| 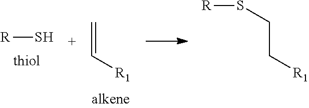 | Thiol-ene reaction |

TABLE 2
Exemplary click chemistry handles and reactions (from Becer,
Hoogenboom, and Schubert, Click Chemistry Beyond Metal-
Catalyzed Cycloaddition, Angewandte Chemie International Edition
(2009) 48: 4900-4908.).

| | Reagent A | Reagent B | Mechanism | Notes on reaction^{[a]} | Reference |
|---|---|---|---|---|---|
| 0 | azide | alkyne | Cu-catalyzed [3 + 2] | 2 h at 60° C. in H₂O | [9] |
| | | | azide-alkyne | | |
| | | | cycloaddition (CuAAC) | | |
| 1 | azide | cyclooctyne | strain-promoted [3 + 2] | 1 h at RT | [6-8, |
| | | | azide-alkyne | | 10, 11] |
| | | | cycloaddition (SPAAC) | | |
| 2 | azide | activated | [3 + 2] Huisgen | 4 h at 50° C. | [12] |
| | | alkyne | cycloaddition | | |
| 3 | azide | electron- | [3 + 2] | 12 h at RT in H₂O | [13] |
| | | deficient | cycloaddition | | |
| | | alkyne | | | |
| 4 | azide | aryne | [3 + 2] | 4 h at RT in THF with crown ether or | [14, 15] |
| | | | cycloaddition | 24 h at RT in CH₃CN | |
| 5 | tetrazine | alkene | Diels-Alder retro-[4 + 2] | 40 min at 25° C. (100% yield) | [36-38] |
| | | | cycloaddition | N₂ is the only by-product | |
| 6 | tetrazole | alkene | 1,3-dipolar cycloaddition | few min UV irradiation and then | [39, 40] |
| | | | (photoclick) | overnight at 4° C. | |
| 7 | dithioester | diene | hetero-Diels-Alder | 10 min at RT | [43] |
| | | | cycloaddition | | |
| 8 | anthracene | maleimide | [4 + 2] Diels-Alder | 2 days at reflux in toluene | [41] |
| | | | reaction | | |
| 9 | thiol | alkene | radical addition | 30 min UV (quantitative conv.) or | [19-23] |
| | | | (thio click) | 24 h UV irradiation (>96%) | |
| 10 | thiol | enone | Michael addition | 24 h at RT in CH₃CN | [27] |
| 11 | thiol | maleimide | Michael addition | 1 h at 40° C. in THF or | [24-26] |
| | | | | 16 h at RT in dioxane | |
| 12 | thiol | para-fluoro | nucleophilic substitution | overnight at RT in DMF or | [32] |
| | | | | 60 min 40° C. in DMF | |
| 13 | amine | para-fluoro | nucleophilic substitution | 20 min MW at 95° C. in NMP as solvent | [30] |

^{[a]}RT = room temperature, DMF = N,N-dimethylformamide, NMP = N-methylpyrolidone, THF = tetrahydrofuran, CH₃CN = acetonitrile.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, the present disclosure provides a compound of Formula (I):

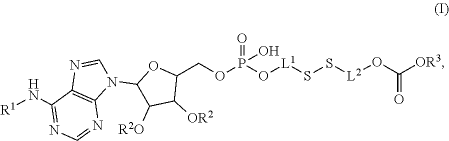

or a salt thereof, wherein:
R¹ is a solid support;
each instance of R² is independently hydrogen or an oxygen protecting group;
R³ is optionally substituted alkyl, optionally substituted heterocyclyl, optionally substituted aryl, or optionally substituted heteroaryl;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof; and
L² is optionally substituted C₂ alkylene.

In some embodiments, the compound of Formula (I) is of Formula (I′):

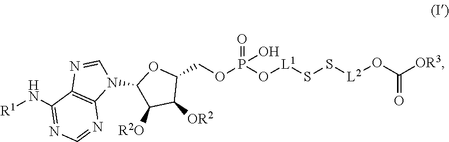

or a salt thereof.

In another aspect, the present disclosure provides a compound of Formula (II):

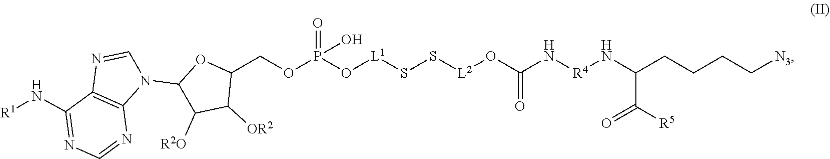

or a salt thereof, wherein:
R¹ is a solid support;
each instance of R² is independently hydrogen or an oxygen protecting group;
R⁴ is a peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof; and
L² is optionally substituted C₂ alkylene.

In some embodiments, the compound of Formula (II) is of Formula (II′):

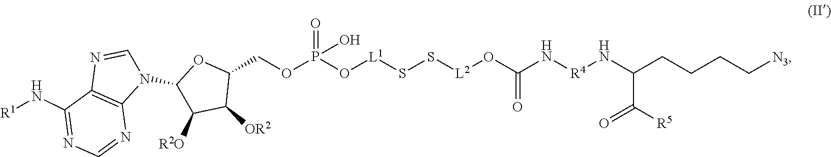

or a salt thereof.

In another aspect, the present disclosure provides a compound of Formula (III):

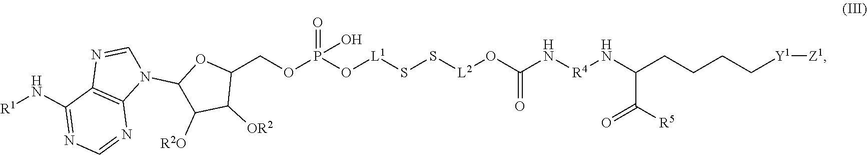

or a salt thereof, wherein:
R¹ is a solid support;
each instance of R² is independently hydrogen or an oxygen protecting group;
R⁴ is a peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof;
L² is optionally substituted C₂ alkylene;
Y¹ comprises a click chemistry adduct; and
Z¹ comprises an oligonucleotide.

In some embodiments, the compound of Formula (III) is of Formula (III′):

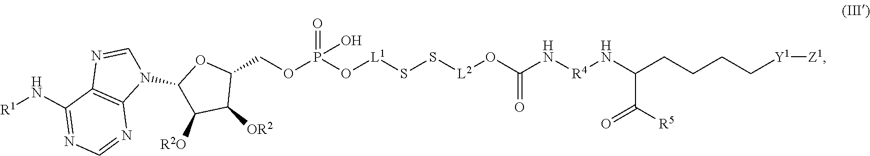

or a salt thereof.

### R¹

As generally described herein, R¹ is a solid support.

In some embodiments, a solid support refers to a material, layer, or other structure having a surface, such as a receiving surface, that is capable of supporting a deposited material.

In some embodiments, the solid support is beads (such as magnetic beads, polystyrene beads, or gold beads); resin; fiber; sheet; biocompatible polymer or material; a nanoparticle; a matrix; a hydrogel; a biomaterial, biocompatible, and/or biodegradable scaffold material; or the like. In some embodiments, the solid support is beads (such as magnetic beads, polystyrene beads, or gold beads). In some embodiments, the solid support is magnetic beads. In some embodiments, the solid support is polystyrene beads. In some embodiments, the solid support is gold beads. In some embodiments, the solid support is a resin. In some embodiments, the solid support is a fiber. In some embodiments, the solid support is a sheet. In some embodiments, the solid support is biocompatible polymer or material. In some embodiments, the solid support is a nanoparticle. In some embodiments, the solid support is a matrix. In some embodiments, the solid support is a biomaterial. In some embodiments, the solid support is a biocompatible and/or biodegradable scaffold material.

In some embodiments, R¹ is a polymeric support. In some embodiments, R¹ is a polymeric support of Oligo-Affinity Support (PS) (5′-Dimethoxytrityl-Adenosine-2′,3′-diacetate-N-Linked-Polymeric Support), available from Glen Research (Catalog Number 26-4001).

### R²

As generally described herein, each instance of R² is independently hydrogen or an oxygen protecting group.

In some embodiments, at least one instance of R² is hydrogen.

In some embodiments, at least one instance of R² is an oxygen protecting group. In some embodiments, at least one instance of R² is —C(═O)R^{2a}, wherein R^{2a} is optionally substituted alkyl or optionally substituted aryl.

In some embodiments, at least one instance of R² is —C(═O)R^{2a}, wherein R^{2a} is optionally substituted alkyl. In some embodiments, at least one instance of R² is —C(═O)R^{2a}, wherein R^{2a} is substituted alkyl. In some embodiments, at least one instance of R² is —C(═O)R^{2a}, wherein R^{2a} is unsubstituted alkyl.

In some embodiments, at least one instance of R² is —C(═O)R^{2a}, wherein R^{2a} is optionally substituted C₁₋₆ alkyl. In some embodiments, at least one instance of R² is —C(═O)R^{2a}, wherein R^{2a} is substituted C₁₋₆ alkyl. In some embodiments, at least one instance of R² is —C(═O)R^{2a}, wherein R^{2a} is unsubstituted C₁₋₆ alkyl.

In some embodiments, at least one instance of R² is —C(═O)R^{2a}, wherein R^{2a} is optionally substituted C₁₋₃ alkyl. In some embodiments, at least one instance of R² is —C(═O)R^{2a}, wherein R^{2a} is substituted C₁₋₃ alkyl. In some embodiments, at least one instance of R² is —C(═O)R^{2a}, wherein R^{2a} is unsubstituted C₁₋₃ alkyl.

In some embodiments, at least one instance of R² is —C(═O)CH₃.

In some embodiments, each instance of R² is independently hydrogen.

In some embodiments, each instance of R² is independently an oxygen protecting group. In some embodiments, each instance of R² is independently —C(═O)R^{2a}, wherein R^{2a} is optionally substituted alkyl or optionally substituted aryl.

In some embodiments, each instance of R² is independently —C(═O)R^{2a}, wherein R^{2a} is optionally substituted alkyl. In some embodiments, each instance of R² is independently —C(═O)R^{2a}, wherein R^{2a} is substituted alkyl. In some embodiments, each instance of R² is independently —C(═O)R^{2a}, wherein R^{2a} is unsubstituted alkyl.

In some embodiments, each instance of R² is independently —C(═O)R^{2a}, wherein R^{2a} is optionally substituted C₁₋₆ alkyl. In some embodiments, each instance of R² is independently —C(═O)R^{2a}, wherein R^{2a} is substituted C₁₋₆ alkyl. In some embodiments, each instance of R² is independently —C(═O)R^{2a}, wherein R^{2a} is unsubstituted C₁₋₆ alkyl.

In some embodiments, each instance of R² is independently —C(═O)R^{2a}, wherein R^{2a} is optionally substituted C₁₋₃ alkyl. In some embodiments, each instance of R² is independently —C(═O)R^{2a}, wherein R^{2a} is substituted C₁₋₃ alkyl. In some embodiments, each instance of R^{Z} is independently —C(═O)R^{2a}, wherein R^{2a} is unsubstituted C₁₋₃ alkyl.

In some embodiments, each instance of R² is independently —C(═O)CH₃.

In some embodiments, at least one instance of R² is —C(═O)R^{2a}, wherein R^{2a} is optionally substituted aryl. In some embodiments, at least one instance of R² is —C(═O)R^{2a}, wherein R^{2a} is substituted aryl. In some embodiments, at least one instance of R² is —C(═O)R^{2a}, wherein R^{2a} is unsubstituted aryl.

In some embodiments, at least one instance of R² is —C(═O)R^{2a}, wherein R^{2a} is optionally substituted phenyl. In some embodiments, at least one instance of R² is —C(═O)R^{2a}, wherein R^{2a} is substituted phenyl. In some embodiments, at least one instance of R² is —C(═O)R^{2a}, wherein R^{2a} is unsubstituted phenyl (i.e., phenyl).

In some embodiments, each instance of R² is independently —C(═O)R^{2a}, wherein R^{2a} is optionally substituted aryl. In some embodiments, each instance of R² is independently —C(═O)R^{2a}, wherein R^{2a} is substituted aryl. In some embodiments, each instance of R² is independently —C(═O)R^{2a}, wherein R^{2a} is unsubstituted aryl.

In some embodiments, each instance of R² is independently —C(═O)R^{2a}, wherein R^{2a} is optionally substituted phenyl. In some embodiments, each instance of R² is independently —C(═O)R^{2a}, wherein R^{2a} is substituted phenyl. In some embodiments, each instance of R² is independently —C(═O)R^{2a}, wherein R^{2a} is unsubstituted phenyl (i.e., phenyl).

### R³

As generally described herein, R³ is optionally substituted alkyl, optionally substituted heterocyclyl, optionally substituted aryl, or optionally substituted heteroaryl.

In some embodiments, R³ is optionally substituted heterocyclyl or optionally substituted aryl. In some embodiments, R³ is substituted heterocyclyl or substituted aryl. In some embodiments, R³ is substituted 5-6 membered heterocyclyl or substituted phenyl. In some embodiments, R³ is

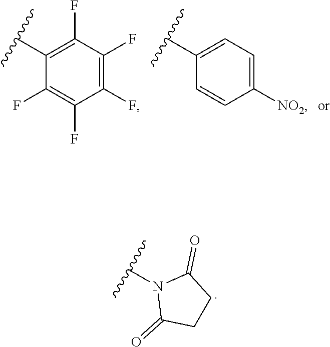

In some embodiments, R³ is optionally substituted alkyl. In some embodiments, R³ is optionally substituted C₁₋₆ alkyl. In some embodiments, R³ is optionally substituted C₁₋₃ alkyl.

In some embodiments, R³ is substituted alkyl. In some embodiments, R³ is substituted C₁₋₆ alkyl. In some embodiments, R³ is substituted C₁₋₃ alkyl. In some embodiments, R³ is alkyl substituted with one or more substituents selected from halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, —B(OR^{A})₂, ═O, and ═S; wherein each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In some embodiments, R³ is alkyl substituted with one or more substituents selected from halogen, —CN, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, and ═O. In some embodiments, R³ is alkyl substituted with one or more halogen. In some embodiments, R³ is alkyl substituted with one or more fluoro.

In some embodiments, R³ is unsubstituted alkyl. In some embodiments, R³ is unsubstituted C₁₋₆ alkyl. In some embodiments, R³ is unsubstituted C₁₋₃ alkyl.

In some embodiments, R³ is optionally substituted heterocyclyl. In some embodiments, R³ is substituted heterocyclyl. In some embodiments, R³ is unsubstituted heterocyclyl.

In some embodiments, R³ is optionally substituted 5-6 membered heterocyclyl. In some embodiments, R³ is substituted 5-6 membered heterocyclyl. In some embodiments, R³ is unsubstituted 5-6 membered heterocyclyl. In some embodiments, R³ is substituted 5-6 membered heterocyclyl containing 1 ring N atom.

In some embodiments, R³ is optionally substituted 5 membered heterocyclyl. In some embodiments, R³ is substituted 5 membered heterocyclyl. In some embodiments, R³ is unsubstituted 5 membered heterocyclyl. In some embodiments, R³ is substituted 5 membered heterocyclyl containing 1 ring N atom.

In some embodiments, R³ is 5-6 membered heterocyclyl substituted with one or more substituents selected from halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, —B(OR^{A})₂, ═O, and ═S; wherein each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In some embodiments, R³ is 5-6 membered heterocyclyl substituted with one or more ═O. In some embodiments, R³ is 5-6 membered heterocyclyl substituted with two ═O. In some embodiments, R³ is 5 membered heterocyclyl substituted with one or more ═O. In some embodiments, R³ is 5 membered heterocyclyl substituted with two ═O. In some embodiments, R³ is 5-6 membered heterocyclyl containing 1 ring N atom, wherein the heterocyclyl is substituted with one or more ═O. In some embodiments, R³ is 5-6 membered heterocyclyl containing 1 ring N atom, wherein the heterocyclyl is substituted with two ═O. In some embodiments, R³ is 5 membered heterocyclyl containing 1 ring N atom, wherein the heterocyclyl is substituted with one or more ═O. In some embodiments, R³ is 5 membered heterocyclyl containing 1 ring N atom, wherein the heterocyclyl is substituted with two ═O. In some embodiments, R³ is

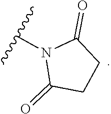 [SMILES: *N1C(=O)CCC1=O]

In some embodiments, R³ is optionally substituted aryl. In some embodiments, R³ is substituted aryl. In some embodiments, R³ is unsubstituted aryl.

In some embodiments, R³ is optionally substituted phenyl. In some embodiments, R³ is substituted phenyl. In some embodiments, R³ is unsubstituted phenyl.

In some embodiments, R³ is phenyl substituted with one or more substituents selected from halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and —B(OR^{A})₂; wherein each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In some embodiments, R³ is phenyl substituted with one or more substituents selected from halogen, optionally substituted alkyl, optionally substituted heteroalkyl, —CN, —OR^{A}, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, and —S(═O)₂N(R^{A})₂. In some embodiments, R³ is phenyl substituted with one or more substituents selected from halogen, optionally substituted alkyl, optionally substituted heteroalkyl, —CN, —OR^{A}, —N(R^{A})₂, and —NO₂. In some embodiments, R³ is phenyl substituted with one or more substituents selected from halogen and —NO₂. In some embodiments, R³ is phenyl substituted with one or more substituents selected from fluoro, chloro, bromo, iodo, and —NO₂. In some embodiments, R³ is phenyl substituted with one or more substituents selected from fluoro, chloro, bromo, and —NO₂. In some embodiments, R³ is phenyl substituted with one or more substituents selected from fluoro, chloro, and —NO₂. In some embodiments, R³ is phenyl substituted with one or more substituents selected from fluoro and —NO₂. In some embodiments, R³ is phenyl substituted with at least one halogen or —NO₂. In some embodiments, R³ is phenyl substituted with at least one fluoro or —NO₂. In some embodiments, R³ is phenyl substituted with at least one fluoro. In some embodiments, R³ is phenyl substituted with at least one —NO₂.

In some embodiments, R³ is

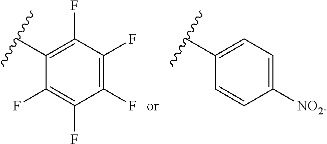 [SMILES: *c1c(F)c(F)c(F)c(F)c1F ; *c1ccc([N+](=O)[O-])cc1]

In some embodiments, R³ is

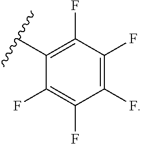 [SMILES: *c1c(F)c(F)c(F)c(F)c1F]

In some embodiments, R³ is

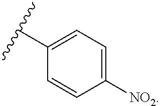 [SMILES: *c1ccc([N+](=O)[O-])cc1]

In some embodiments, R³ is of formula:

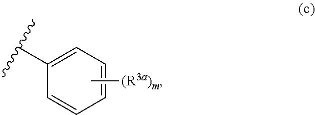

wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
m is 0, 1, 2, 3, 4, or 5.

In some embodiments, at least one instance of R^{3a} is halogen, optionally substituted alkyl, optionally substituted heteroalkyl, —CN, —OR^{A}, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, or —S(═O)₂N(R^{A})₂. In some embodiments, at least one instance of R^{3a} is halogen, optionally substituted alkyl, optionally substituted heteroalkyl, —CN, —OR^{A}, —N(R^{A})₂, or —NO₂. In some embodiments, at least one instance of R^{3a} is halogen or —NO₂. In some embodiments, at least one instance of R^{3a} is fluoro, chloro, bromo, iodo, or —NO₂. In some embodiments, at least one instance of R^{3a} is fluoro, chloro, bromo, or —NO₂. In some embodiments, at least one instance of R^{3a} is fluoro, chloro, or —NO₂. In some embodiments, at least one instance of R^{3a} is fluoro or —NO₂. In some embodiments, at least one instance of R^{3a} is fluoro. In some embodiments, at least one instance of R^{3a} is —NO₂. In some embodiments, each instance of R^{3a} is independently halogen.

In some embodiments, the R³ of formula (c) is of formula:

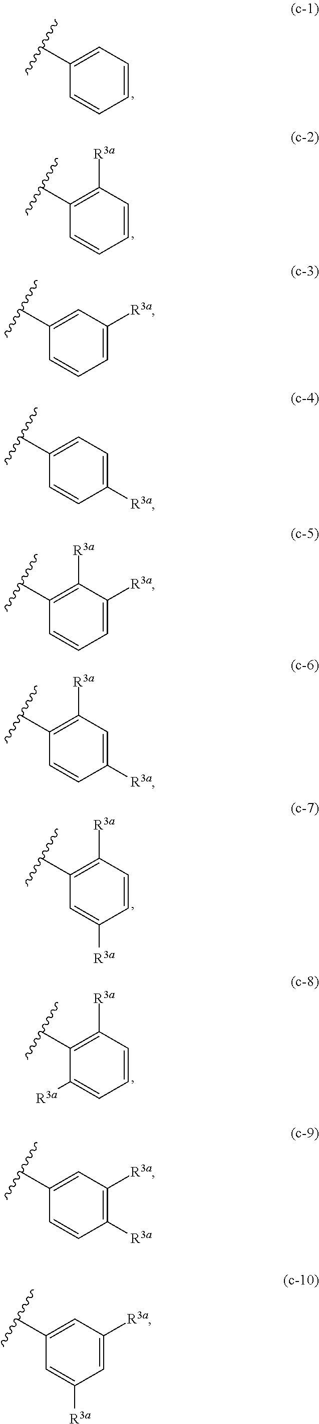

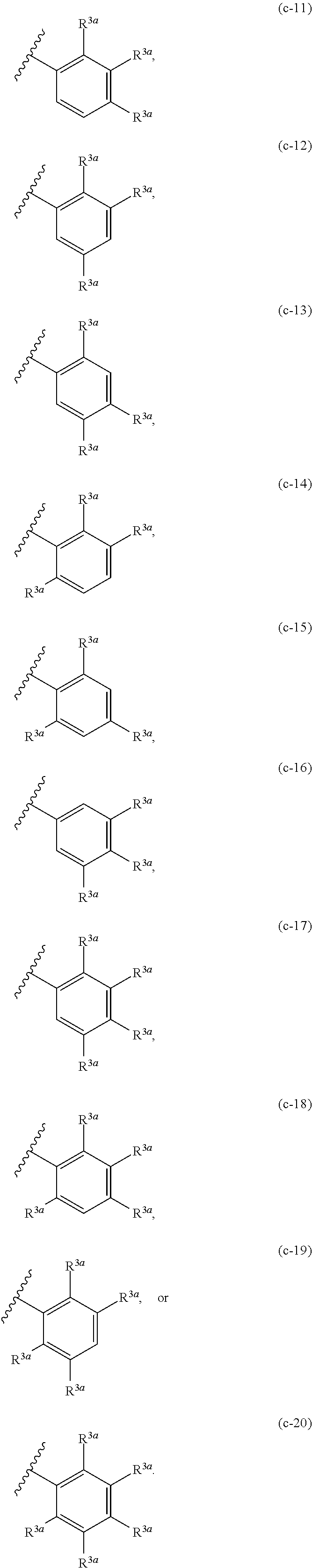

In some embodiments, R³ is of formula: (c-1), (c-2), (c-3), (c-4), (c-5), (c-6), (c-7), (c-8), (c-9), (c-10), (c-11), (c-12), (c-13), (c-14), (c-15), (c-16), (c-17), (c-18), (c-19), or (c-20), and at least one instance of R^{3a} is halogen or —NO₂. In some embodiments, R³ is of formula: (c-1), (c-2), (c-3), (c-4), (c-5), (c-6), (c-7), (c-8), (c-9), (c-10), (c-11), (c-12), (c-13), (c-14), (c-15), (c-16), (c-17), (c-18), (c-19), or (c-20), and each instance of R^{3a} is independently halogen. In some embodiments, R³ is of formula: (c-1), (c-2), (c-3), (c-4), (c-5), (c-6), (c-7), (c-8), (c-9), (c-10), (c-11), (c-12), (c-13), (c-14), (c-15), (c-16), (c-17), (c-18), (c-19), or (c-20), and at least one instance of R^{3a} is fluoro. In some embodiments, R³ is of formula: (c-1), (c-2), (c-3), (c-4), (c-5), (c-6), (c-7), (c-8), (c-9), (c-10), (c-11), (c-12), (c-13), (c-14), (c-15), (c-16), (c-17), (c-18), (c-19), or (c-20), and at least one instance of R^{3a} is —NO₂.

In some embodiments, R³ is optionally substituted heteroaryl. In some embodiments, R³ is substituted heteroaryl. In some embodiments, R³ is unsubstituted heteroaryl.

In some embodiments, R³ is optionally substituted 5-6 membered heteroaryl. In some embodiments, R³ is substituted 5-6 membered heteroaryl. In some embodiments, R³ is unsubstituted 5-6 membered heteroaryl.

In some embodiments, R³ is 5-6 membered heteroaryl substituted with one or more substituents selected from halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(RA)₃, —OSi(R^{A})₂OR^{A}, —OSi(R^{A})(OR^{A})₂, —OSi(OR^{A})₃, and —B(OR^{A})₂; wherein each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In some embodiments, R³ is 5-6 membered heteroaryl substituted with one or more substituents selected from halogen, optionally substituted alkyl, optionally substituted heteroalkyl, —CN, —OR^{A}, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, and —S(═O)₂N(R^{A})₂. In some embodiments, R³ is 5-6 membered heteroaryl substituted with one or more substituents selected from halogen, optionally substituted alkyl, optionally substituted heteroalkyl, —CN, —OR^{A}, —N(R^{A})₂, and —NO₂. In some embodiments, R³ is 5-6 membered heteroaryl substituted with one or more substituents selected from halogen and —NO₂. In some embodiments, R³ is 5-6 membered heteroaryl substituted with one or more substituents selected from fluoro, chloro, bromo, iodo, and —NO₂. In some embodiments, R³ is 5-6 membered heteroaryl substituted with one or more substituents selected from fluoro, chloro, bromo, and —NO₂. In some embodiments, R³ is 5-6 membered heteroaryl substituted with one or more substituents selected from fluoro, chloro, and —NO₂. In some embodiments, R³ is 5-6 membered heteroaryl substituted with one or more substituents selected from fluoro and —NO₂. In some embodiments, R³ is 5-6 membered heteroaryl substituted with at least one halogen or —NO₂. In some embodiments, R³ is 5-6 membered heteroaryl substituted with at least one fluoro or —NO₂. In some embodiments, R³ is 5-6 membered heteroaryl substituted with at least one fluoro. In some embodiments, R³ is 5-6 membered heteroaryl substituted with at least one —NO₂.

As generally described herein, each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.

In some embodiments, at least one occurrence of R^{A} is hydrogen. In some embodiments, at least one occurrence of R^{A} is optionally substituted acyl. In some embodiments, at least one occurrence of R^{A} is optionally substituted alkyl. In some embodiments, at least one occurrence of R^{A} is optionally substituted alkenyl. In some embodiments, at least one occurrence of R^{A} is optionally substituted alkynyl. In some embodiments, at least one occurrence of R^{A} is optionally substituted heteroalkyl. In some embodiments, at least one occurrence of R^{A} is optionally substituted heteroalkenyl. In some embodiments, at least one occurrence of R^{A} is optionally substituted heteroalkynyl. In some embodiments, at least one occurrence of R^{A} is optionally substituted carbocyclyl. In some embodiments, at least one occurrence of R^{A} is optionally substituted heterocyclyl. In some embodiments, at least one occurrence of R^{A} is optionally substituted aryl. In some embodiments, at least one occurrence of R^{A} is optionally substituted heteroaryl. In some embodiments, at least one occurrence of R^{A} is a nitrogen protecting group when attached to a nitrogen atom. In some embodiments, at least one occurrence of R^{A} is an oxygen protecting group when attached to an oxygen atom. In some embodiments, at least one occurrence of R^{A} is a sulfur protecting group when attached to a sulfur atom. In some embodiments, two occurrences of R^{A} are joined together with their intervening atom to form an optionally substituted heterocyclic ring. In some embodiments, two occurrences of R^{A} are joined together with their intervening atom to form an optionally substituted heteroaryl ring.

As generally described herein, m is 0, 1, 2, 3, 4, or 5. In some embodiments, m is 0. In some embodiments, m is 1. In some embodiments, m is 2. In some embodiments, m is 3. In some embodiments, m is 4. In some embodiments, m is 5.

R⁴ and R⁵

As generally described herein, R⁴ is a peptide.

In some embodiments, R⁴ comprises one or more amino acids selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, R⁴ comprises an amino acid comprising a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, R⁴ comprises an amino acid comprising an arginine post-translational modification. For example, as described herein, arginine modifications include symmetric dimethylarginine (SDMA), asymmetric dimethylarginine (ADMA), and citrullinated arginine.

In some embodiments, R⁴ comprises an amino acid comprising a phosphorylated side chain. In some embodiments, R⁴ comprises an amino acid comprising phosphorylated threonine (e.g., phospho-threonine). In some embodiments, R⁴ comprises an amino acid comprising phosphorylated tyrosine (e.g., phospho-tyrosine). In some embodiments, R⁴ comprises an amino acid comprising phosphorylated serine (e.g., phospho-serine).

In some embodiments, R⁴ comprises an amino acid comprising a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, R⁴ comprises an amino acid comprising an oxidative modification. In some embodiments, R⁴ comprises an amino acid comprising a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, R⁴ comprises an amino acid comprising a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

As generally described herein, R⁵ is —OH or —NH₂.

In some embodiments, R⁵ is —OH. In some embodiments, R⁵ is —NH₂.

### L¹

As generally described herein, L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof.

In some embodiments, L¹ comprises optionally substituted alkylene, optionally substituted heteroalkylene, or a combination thereof.

In some embodiments, L¹ comprises optionally substituted alkylene. In some embodiments, L¹ comprises optionally substituted C₁-C₁₀₀ alkylene. In some embodiments, L¹ comprises optionally substituted C₁-C₇₅ alkylene. In some embodiments, L¹ comprises optionally substituted C₁-C₅₀ alkylene. In some embodiments, L¹ comprises optionally substituted C₁-C₂₅ alkylene. In some embodiments, L¹ comprises optionally substituted C₁-C₁₂ alkylene. In some embodiments, L¹ comprises optionally substituted C₁-C₆ alkylene. In some embodiments, L¹ comprises optionally substituted C₁-C₃ alkylene. In some embodiments, L¹ comprises optionally substituted linear alkylene. In some embodiments, L¹ comprises optionally substituted linear C₁-C₁₂ alkylene. In some embodiments, L¹ comprises optionally substituted linear C₁-C₆ alkylene. In some embodiments, L¹ comprises optionally substituted linear C₁-C₃ alkylene.

In some embodiments, L¹ comprises substituted alkylene. In some embodiments, L¹ comprises substituted C₁-C₁₀₀ alkylene. In some embodiments, L¹ comprises substituted C₁-C₇₅ alkylene. In some embodiments, L¹ comprises substituted C₁-C₅₀ alkylene. In some embodiments, L¹ comprises substituted C₁-C₂₅ alkylene. In some embodiments, L¹ comprises substituted C₁-C₁₂ alkylene. In some embodiments, L¹ comprises substituted C₁-C₆ alkylene. In some embodiments, L¹ comprises substituted C₁-C₃ alkylene. In some embodiments, L¹ comprises substituted linear alkylene. In some embodiments, L¹ comprises substituted linear C₁-C₁₂ alkylene. In some embodiments, L¹ comprises substituted linear C₁-C₆ alkylene. In some embodiments, L¹ comprises substituted linear C₁-C₃ alkylene.

In some embodiments, L¹ comprises unsubstituted alkylene. In some embodiments, L¹ comprises unsubstituted C₁-C₁₀₀ alkylene. In some embodiments, L¹ comprises unsubstituted C₁-C₇₅ alkylene. In some embodiments, L¹ comprises unsubstituted C₁-C₅₀ alkylene. In some embodiments, L¹ comprises unsubstituted C₁-C₂₅ alkylene. In some embodiments, L¹ comprises unsubstituted C₁-C₁₂ alkylene. In some embodiments, L¹ comprises unsubstituted C₁-C₆ alkylene. In some embodiments, L¹ comprises unsubstituted C₁-C₃ alkylene. In some embodiments, L¹ comprises unsubstituted linear alkylene. In some embodiments, L¹ comprises unsubstituted linear C₁-C₁₂ alkylene. In some embodiments, L¹ comprises unsubstituted linear C₁-C₆ alkylene. In some embodiments, L¹ comprises unsubstituted linear C₁-C₃ alkylene.

In some embodiments, L¹ comprises methylene, ethylene, n-propylene, n-butylene, n-pentylene, or n-hexylene. In some embodiments, L¹ comprises methylene, ethylene, or n-propylene. In some embodiments, L¹ comprises ethylene.

In some embodiments, L¹ comprises optionally substituted alkenylene. In some embodiments, L¹ comprises optionally substituted C₁-C₁₀₀ alkenylene. In some embodiments, L¹ comprises optionally substituted C₁-C₇₅ alkenylene. In some embodiments, L¹ comprises optionally substituted C₁-C₅₀ alkenylene. In some embodiments, L¹ comprises optionally substituted C₁-C₂₅ alkenylene. In some embodiments, L¹ comprises optionally substituted C₁-C₁₂ alkenylene. In some embodiments, L¹ comprises optionally substituted C₁-C₆ alkenylene. In some embodiments, L¹ comprises optionally substituted C₁-C₃ alkenylene. In some embodiments, L¹ comprises substituted alkenylene. In some embodiments, L¹ comprises substituted C₁-C₁₂ alkenylene. In some embodiments, L¹ comprises substituted C₁-C₆ alkenylene. In some embodiments, L¹ comprises substituted C₁-C₃ alkenylene. In some embodiments, L¹ comprises unsubstituted alkenylene. In some embodiments, L¹ comprises unsubstituted C₁-C₁₂ alkenylene. In some embodiments, L¹ comprises unsubstituted C₁-C₆ alkenylene. In some embodiments, L¹ comprises unsubstituted C₁-C₃ alkenylene.

In some embodiments, L¹ comprises optionally substituted alkynylene. In some embodiments, L¹ comprises optionally substituted C₁-C₁₀₀ alkynylene. In some embodiments, L¹ comprises optionally substituted C₁-C₇₅ alkynylene. In some embodiments, L¹ comprises optionally substituted C₁-C₅₀ alkynylene. In some embodiments, L¹ comprises optionally substituted C₁-C₂₅ alkynylene. In some embodiments, L¹ comprises optionally substituted C₁-C₁₂ alkynylene. In some embodiments, L¹ comprises optionally substituted C₁-C₆ alkynylene. In some embodiments, L¹ comprises optionally substituted C₁-C₃ alkynylene. In some embodiments, L¹ comprises substituted alkynylene. In some embodiments, L¹ comprises substituted C₁-C₁₂ alkynylene. In some embodiments, L¹ comprises substituted C₁-C₆ alkynylene. In some embodiments, L¹ comprises substituted C₁-C₃ alkynylene. In some embodiments, L¹ comprises unsubstituted alkynylene. In some embodiments, L¹ comprises unsubstituted C₁-C₁₂ alkynylene. In some embodiments, L¹ comprises unsubstituted C₁-C₆ alkynylene. In some embodiments, L¹ comprises unsubstituted C₁-C₃ alkynylene.

In some embodiments, L¹ comprises optionally substituted heteroalkylene. In some embodiments, L¹ comprises optionally substituted C₁-C₁₀₀ heteroalkylene. In some embodiments, L¹ comprises optionally substituted C₁-C₇₅ heteroalkylene. In some embodiments, L¹ comprises optionally substituted C₁-C₅₀ heteroalkylene. In some embodiments, L¹ comprises optionally substituted C₁-C₂₅ heteroalkylene. In some embodiments, L¹ comprises optionally substituted C₁-C₁₂ heteroalkylene. In some embodiments, L¹ comprises optionally substituted C₁-C₆ heteroalkylene. In some embodiments, L¹ comprises optionally substituted C₁-C₃ heteroalkylene. In some embodiments, L¹ comprises optionally substituted linear heteroalkylene. In some embodiments, L¹ comprises optionally substituted linear C₁-C₁₂ heteroalkylene. In some embodiments, L¹ comprises optionally substituted linear C₁-C₆ heteroalkylene. In some embodiments, L¹ comprises optionally substituted linear C₁-C₃ heteroalkylene.

In some embodiments, L¹ comprises substituted heteroalkylene. In some embodiments, L¹ comprises substituted C₁-C₁₀₀ heteroalkylene. In some embodiments, L¹ comprises substituted C₁-C₇₅ heteroalkylene. In some embodiments, L¹ comprises substituted C₁-C₅₀ heteroalkylene. In some embodiments, L¹ comprises substituted C₁-C₂₅ heteroalkylene. In some embodiments, L¹ comprises substituted C₁-C₁₂ heteroalkylene. In some embodiments, L¹ comprises substituted C₁-C₆ heteroalkylene. In some embodiments, L¹ comprises substituted C₁-C₃ heteroalkylene. In some embodiments, L¹ comprises substituted linear heteroalkylene. In some embodiments, L¹ comprises substituted linear C₁-C₁₂ heteroalkylene. In some embodiments, L¹ comprises substituted linear C₁-C₆ heteroalkylene. In some embodiments, L¹ comprises substituted linear C₁-C₃ heteroalkylene.

In some embodiments, L¹ comprises unsubstituted heteroalkylene. In some embodiments, L¹ comprises unsubstituted C₁-C₁₀₀ heteroalkylene. In some embodiments, L¹ comprises unsubstituted C₁-C₇₅ heteroalkylene. In some embodiments, L¹ comprises unsubstituted C₁-C₅₀ heteroalkylene. In some embodiments, L¹ comprises unsubstituted C₁-C₂₅ heteroalkylene. In some embodiments, L¹ comprises unsubstituted C₁-C₁₂ heteroalkylene. In some embodiments, L¹ comprises unsubstituted C₁-C₆ heteroalkylene. In some embodiments, L¹ comprises unsubstituted C₁-C₃ heteroalkylene. In some embodiments, L¹ comprises unsubstituted linear heteroalkylene. In some embodiments, L¹ comprises unsubstituted linear C₁-C₁₂ heteroalkylene. In some embodiments, L¹ comprises unsubstituted linear C₁-C₆ heteroalkylene. In some embodiments, L¹ comprises unsubstituted linear C₁-C₃ heteroalkylene.

In some embodiments, L¹ comprises optionally substituted heteroalkenylene. In some embodiments, L¹ comprises optionally substituted C₁-C₁₀₀ heteroalkenylene. In some embodiments, L¹ comprises optionally substituted C₁-C₇₅ heteroalkenylene. In some embodiments, L¹ comprises optionally substituted C₁-C₅₀ heteroalkenylene. In some embodiments, L¹ comprises optionally substituted C₁-C₂₅ heteroalkenylene. In some embodiments, L¹ comprises optionally substituted C₁-C₁₂ heteroalkenylene. In some embodiments, L¹ comprises optionally substituted C₁-C₆ heteroalkenylene. In some embodiments, L¹ comprises optionally substituted C₁-C₃ heteroalkenylene. In some embodiments, L¹ comprises substituted heteroalkenylene. In some embodiments, L¹ comprises substituted C₁-C₁₂ heteroalkenylene. In some embodiments, L¹ comprises substituted C₁-C₆ heteroalkenylene. In some embodiments, L¹ comprises substituted C₁-C₃ heteroalkenylene. In some embodiments, L¹ comprises unsubstituted heteroalkenylene. In some embodiments, L¹ comprises unsubstituted C₁-C₁₂ heteroalkenylene. In some embodiments, L¹ comprises unsubstituted C₁-C₆ heteroalkenylene. In some embodiments, L¹ comprises unsubstituted C₁-C₃ heteroalkenylene.

In some embodiments, L¹ comprises optionally substituted heteroalkynylene. In some embodiments, L¹ comprises optionally substituted C₁-C₁₀₀ heteroalkynylene. In some embodiments, L¹ comprises optionally substituted C₁-C₇₅ heteroalkynylene. In some embodiments, L¹ comprises optionally substituted C₁-C₅₀ heteroalkynylene. In some embodiments, L¹ comprises optionally substituted C₁-C₂₅ heteroalkynylene. In some embodiments, L¹ comprises optionally substituted C₁-C₁₂ heteroalkynylene. In some embodiments, L¹ comprises optionally substituted C₁-C₆ heteroalkynylene. In some embodiments, L¹ comprises optionally substituted C₁-C₃ heteroalkynylene. In some embodiments, L¹ comprises substituted heteroalkynylene. In some embodiments, L¹ comprises substituted C₁-C₁₂ heteroalkynylene. In some embodiments, L¹ comprises substituted C₁-C₆ heteroalkynylene. In some embodiments, L¹ comprises substituted C₁-C₃ heteroalkynylene. In some embodiments, L¹ comprises unsubstituted heteroalkynylene. In some embodiments, L¹ comprises unsubstituted C₁-C₁₂ heteroalkynylene. In some embodiments, L¹ comprises unsubstituted C₁-C₆ heteroalkynylene. In some embodiments, L¹ comprises unsubstituted C₁-C₃ heteroalkynylene.

In some embodiments, L¹ comprises optionally substituted carbocyclylene. In some embodiments, L¹ comprises optionally substituted C₃-C₁₀ carbocyclylene. In some embodiments, L¹ comprises optionally substituted C₃-C₆ carbocyclylene. In some embodiments, L¹ comprises substituted carbocyclylene. In some embodiments, L¹ comprises substituted C₃-C₁₀ carbocyclylene. In some embodiments, L¹ comprises substituted C₃-C₆ carbocyclylene. In some embodiments, L¹ comprises unsubstituted carbocyclylene. In some embodiments, L¹ comprises unsubstituted C₃-C₁₀ carbocyclylene. In some embodiments, L¹ comprises unsubstituted C₃-C₆ carbocyclylene.

In some embodiments, L¹ comprises optionally substituted heterocyclylene. In some embodiments, L¹ comprises optionally substituted 3-10 membered heterocyclylene. In some embodiments, L¹ comprises optionally substituted 3-6 membered heterocyclylene. In some embodiments, L¹ comprises substituted heterocyclylene. In some embodiments, L¹ comprises substituted 3-10 membered heterocyclylene. In some embodiments, L¹ comprises substituted 3-6 membered heterocyclylene. In some embodiments, L¹ comprises unsubstituted heterocyclylene. In some embodiments, L¹ comprises unsubstituted 3-10 membered heterocyclylene. In some embodiments, L¹ comprises unsubstituted 3-6 membered heterocyclylene.

In some embodiments, L¹ comprises optionally substituted arylene. In some embodiments, L¹ comprises optionally substituted phenylene. In some embodiments, L¹ comprises substituted arylene. In some embodiments, L¹ comprises substituted phenylene. In some embodiments, L¹ comprises unsubstituted arylene. In some embodiments, L¹ comprises unsubstituted phenylene.

In some embodiments, L¹ comprises optionally substituted heteroarylene. In some embodiments, L¹ comprises optionally substituted 5-10 membered heteroarylene. In some embodiments, L¹ comprises optionally substituted 5-6 membered monocyclic heteroarylene. In some embodiments, L¹ comprises substituted heteroarylene. In some embodiments, L¹ comprises substituted 5-10 membered heteroarylene. In some embodiments, L¹ comprises substituted 5-6 membered monocyclic heteroarylene. In some embodiments, L¹ comprises unsubstituted heteroarylene. In some embodiments, L¹ comprises unsubstituted 5-10 membered heteroarylene. In some embodiments, L¹ comprises unsubstituted 5-6 membered monocyclic heteroarylene.

In some embodiments, L¹ comprises

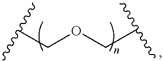

wherein n is an integer between 0 and 30, inclusive. In some embodiments, L¹ comprises —NHC(═O)— or —C(═O)NH—. In some embodiments, L¹ is

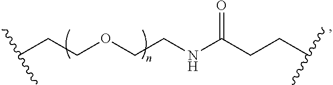

wherein n is an integer between 0 and 30, inclusive.

As generally described herein, n is an integer between 0 and 30.

In some embodiments, n is an integer between 0 and 25, inclusive; between 0 and 20, inclusive; between 0 and 15, inclusive; between 0 and 10, inclusive; or between 0 and 5, inclusive. In some embodiments, n is an integer between 1 and 30, inclusive; between 1 and 25, inclusive; between 1 and 20, inclusive; between 1 and 15, inclusive; between 1 and 10, inclusive; or between 1 and 5, inclusive.

In some embodiments, n is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 1, 2, 3, 4, 5, 6, 7, 8, 9, or 10. In some embodiments, n is 1, 2, 3, 4, 5, 6, 7, 8, or 9. In some embodiments, n is 1, 2, 3, 4, 5, 6, 7, or 8. In some embodiments, n is 1, 2, 3, 4, 5, 6, or 7. In some embodiments, n is 1, 2, 3, 4, 5, or 6. In some embodiments, n is 1, 2, 3, 4, or 5. In some embodiments, n is 1, 2, 3, or 4. In some embodiments, n is 1, 2, or 3. In some embodiments, n is 1 or 2. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, L¹ comprises

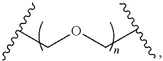

wherein n is an integer between 1 and 30, inclusive. In some embodiments, L¹ comprises

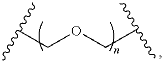

wherein n is an integer between 1 and 10, inclusive. In some embodiments, L¹ comprises

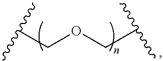

wherein n is an integer between 1 and 5, inclusive.

In some embodiments, L¹ comprises

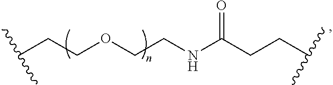

wherein n is an integer between 1 and 30, inclusive. In some embodiments, L¹ comprises

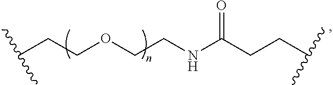

wherein n is an integer between 1 and 10, inclusive. In some embodiments, L¹ comprises

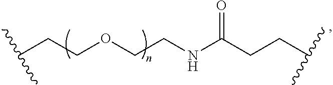

wherein n is an integer between 1 and 5, inclusive.

In some embodiments, L¹ is

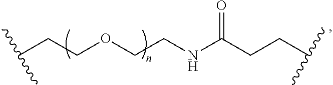

wherein n is an integer between 1 and 30, inclusive. In some embodiments, L¹ is

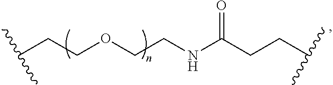

wherein n is an integer between 1 and 10, inclusive. In some embodiments, L¹ is

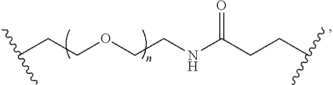

wherein n is an integer between 1 and 5, inclusive.

### L²

As generally described herein, L² is optionally substituted C₂ alkylene.

In some embodiments, L² is substituted C₂ alkylene. In some embodiments, L² is C₂ alkylene substituted with one or more substituents selected from halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, —B(OR^{A})₂, ═O, and ═S; wherein each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.

In some embodiments, L² is C₂ alkylene substituted with one or more substituents selected from halogen, optionally substituted alkyl, optionally substituted heteroalkyl, —OR^{A}, —N(R^{A})₂, and ═O.

In some embodiments, L² is C₂ alkylene substituted with optionally substituted alkyl. In some embodiments, L² is C₂ alkylene substituted with optionally substituted C₁₋₆ alkyl. In some embodiments, L² is C₂ alkylene substituted with optionally substituted C₁₋₃ alkyl. In some embodiments, L² is C₂ alkylene substituted with unsubstituted C₁₋₆ alkyl. In some embodiments, L² is C₂ alkylene substituted with unsubstituted C₁₋₃ alkyl. In some embodiments, L² is C₂ alkylene substituted with methyl, ethyl, n-propyl, or isopropyl. In some embodiments, L² is C₂ alkylene substituted with methyl.

In some embodiments, L² is unsubstituted C₂ alkylene (i.e., ethylene).

In some embodiments, L² is

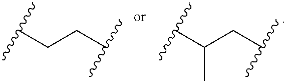

Y¹ and Z¹

As generally described herein, Y¹ comprises a click chemistry adduct.

In some embodiments, Y¹ comprises a click chemistry adduct formed by a click reaction between a strained alkyne-containing moiety and an azide group. In some embodiments, Y¹ comprises a click chemistry adduct formed by a click reaction between a cyclooctyne-containing moiety or an azacyclooctyne-containing moiety; and an azide group. In some embodiments, Y¹ comprises a click chemistry adduct formed by a click reaction between a cyclooctyne-containing moiety and an azide group. In some embodiments, Y¹ comprises a click chemistry adduct formed by a click reaction between an azacyclooctyne-containing moiety and an azide group. In some embodiments, Y¹ comprises a click chemistry adduct formed by a click reaction between dibenzoazacyclooctyne-containing moiety (DIBAC or DBCO), biarylazacyclooctynone-containing moiety (BARAC), dibenzocyclooctyne-containing moiety (DIBO), difluorinated cyclooctyne-containing moiety (DIFO), bicyclononyne-containing moiety (BCN), dimethoxyazacyclooctyne-containing moiety (DIMAC), monofluorinated cyclooctyne-containing moiety (MOFO), cyclooctyne-containing moiety (OCT), and/or aryl-less cyclooctyne-containing moiety (ALO); and an azide group. In some embodiments, Y¹ comprises a click chemistry adduct formed by a click reaction between dibenzoazacyclooctyne-containing moiety (DIBAC or DBCO) and an azide group. In some embodiments, Y¹ comprises a click chemistry adduct formed by a click reaction between biarylazacyclooctynone-containing moiety (BARAC) and an azide group. In some embodiments, Y¹ comprises a click chemistry adduct formed by a click reaction between dibenzocyclooctyne-containing moiety (DIBO) and an azide group. In some embodiments, Y¹ comprises a click chemistry adduct formed by a click reaction between difluorinated cyclooctyne-containing moiety (DIFO) and an azide group. In some embodiments, Y¹ comprises a click chemistry adduct formed by a click reaction between bicyclononyne-containing moiety (BCN) and an azide group. In some embodiments, Y¹ comprises a click chemistry adduct formed by a click reaction between dimethoxyazacyclooctyne-containing moiety (DIMAC) and an azide group. In some embodiments, Y¹ comprises a click chemistry adduct formed by a click reaction between monofluorinated cyclooctyne-containing moiety (MOFO) and an azide group. In some embodiments, Y¹ comprises a click chemistry adduct formed by a click reaction between cyclooctyne-containing moiety (OCT) and an azide group. In some embodiments, Y¹ comprises a click chemistry adduct formed by a click reaction between aryl-less cyclooctyne-containing moiety (ALO) and an azide group.

In some embodiments, Y¹ comprises a click chemistry adduct of formula:

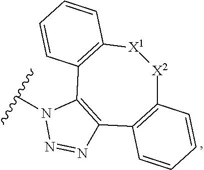

wherein one of X¹ and X² is CH and the other is N*, and wherein * indicates the point of attachment to Z¹. In some embodiments, Y¹ comprises a click chemistry adduct of formula:

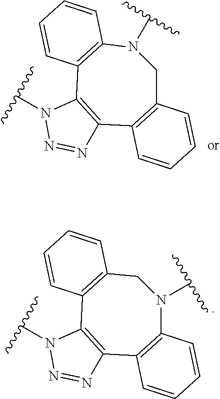 [SMILES: *N1Cc2ccccc2-c2nnn(*)c2-c2ccccc21 ; "or" ; *N1Cc2ccccc2-c2c(nnn2*)-c2ccccc21 ; "."]

In some embodiments, Y¹ comprises a click chemistry adduct of formula:

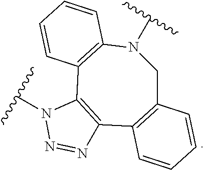 [SMILES: *N1Cc2ccccc2-c2nnn(*)c2-c2ccccc21 ; "."]

In some embodiments, Y¹ comprises a click chemistry adduct of formula:

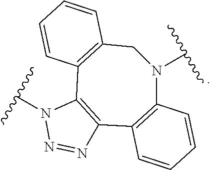 [SMILES: *N1Cc2ccccc2-c2c(nnn2*)-c2ccccc21 ; "."]

In some embodiments, the moiety —Y¹—Z¹ is of formula:

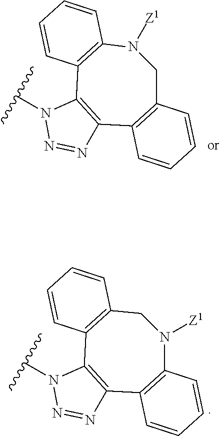

In some embodiments, the moiety —Y¹—Z¹ is of formula:

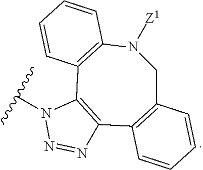

In some embodiments, the moiety —Y¹—Z¹ is of formula:

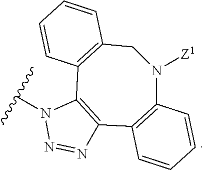

As generally described herein, Z¹ comprises an oligonucleotide. In certain embodiments, the oligonucleotide is a single-stranded oligonucleotide. In certain embodiments, the oligonucleotide is a double-stranded oligonucleotide. In certain embodiments, the oligonucleotide has a length of at least 15, at least 20, at least 25, at least 30, at least 35, at least 40, at least 45, or at least 50 nucleotides. In certain embodiments, the oligonucleotide has a length in a range from 15 to 20, 15 to 25, 15 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, 25 to 50, 30 to 35, 30 to 40, 30 to 45, 30 to 50, 35 to 40, 35 to 45, 35 to 50, 40 to 45, 40 to 50, or 45 to 50 nucleotides. In some embodiments, the oligonucleotide has a length of at least 25 nucleotides.

In certain embodiments, at least one strand of the oligonucleotide has a sequence that is at least 50%, at least 55%, at least 60%, at least 65%, at least 70%, at least 75%, at least 80%, at least 85%, at least 90%, at least 95%, at least 99%, or 100% identical to 5′-CCACGCGTGGAACCCTTGGGATCCA-3′ (SEQ ID NO: 32). In some embodiments, at least one strand of the oligonucleotide has a sequence that is at least 80% identical to 5′-CCACGCGTGGAACCCTTGGGATCCA-3′ (SEQ ID NO: 32). In certain embodiments, at least one strand of the oligonucleotide has a sequence that is at least 50%, at least 55%, at least 60%, at least 65%, at least 70%, at least 75%, at least 80%, at least 85%, at least 90%, at least 95%, at least 99%, or 100% identical to 5′-TGG AGT CAA GGT CCT CTG ATG CCA T-3′ (SEQ ID NO: 33).

In some embodiments, Z¹ further comprises a linking group.

In some embodiments, the linking group comprises a polypeptidyl group. In certain embodiments, the polypeptidyl group comprises at least 5 amino acid residues, at least 10 amino acid residues, at least 15 amino acid residues, or at least 20 amino acid residues. In certain embodiments, the polypeptidyl group comprises between 5 and 10 amino acid residues, between 5 and 15 amino acid residues, between 5 and 20 amino acid residues, between 10 and 15 amino acid residues, between 10 and 20 amino acid residues, or between 15 and 20 amino acid residues. In some embodiments, the polypeptidyl group comprises between 5 and 15 amino acid residues.

In some embodiments, the polypeptidyl group has a length of at least about 20 Å, 25 Å, 30 Å, 35 Å, 40 Å, 45 Å, 50 Å, 55 Å, 60 Å, 65 Å, 70 Å, or 75 Å. In certain embodiments, the polypeptidyl group has a length in a range from 20 Å to 30 Å, 20 Å to 35 Å, 20 Å to 40 Å, 20 Å to 45 Å, 20 Å to 50 Å, 20 Å to 55 Å, 20 Å to 60 Å, 20 Å to 65 Å, 20 Å to 70 Å, 20 Å to 75 Å, 30 Å to 40 Å, 30 Å to 45 Å, 30 Å to 50 Å, 30 Å to 55 Å, 30 Å to 60 Å, 30 Å to 65 Å, 30 Å to 70 Å, 30 Å to 75 Å, 40 Å to 50 Å, 40 Å to 55 Å, 40 Å to 60 Å, 40 Å to 65 Å, 40 Å to 70 Å, 40 Å to 75 Å, 50 Å to 60 Å, 50 Å to 65 Å, 50 Å to 70 Å, 50 Å to 75 Å, 60 Å to 70 Å, or 60 Å to 75 Å.

In some embodiments, the polypeptidyl group comprises at least 1 negatively charged moiety at physiological pH. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 negatively charged moieties at physiological pH. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 negatively charged moieties at physiological pH. In some embodiments, the polypeptidyl group comprises between 1 and 10 negatively charged moieties at physiological pH.

In some embodiments, the polypeptidyl group comprises at least 1 aspartate residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 aspartate residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 aspartate residues. In some embodiments, the polypeptidyl group comprises between 1 and 10 aspartate residues.

In some embodiments, the polypeptidyl group comprises at least 1 phenylalanine residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 phenylalanine residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 phenylalanine residues.

In some embodiments, the polypeptidyl group comprises at least 1 glycine residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 glycine residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 glycine residues.

In some embodiments, the polypeptidyl group comprises at least 1 proline residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 proline residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 proline residues.

In some embodiments, the polypeptidyl group comprises at least 1 DD repeat, GG repeat, FF repeat, DDD repeat, GGG, and/or FFF repeat. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 DD repeats, GG repeats, FF repeats, DDD repeats, GGG, and/or FFF repeats. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 DD repeats, GG repeats, FF repeats, DDD repeats, GGG, and/or FFF repeats.

In some embodiments, the polypeptidyl group comprises a sequence selected from the group consisting of GPPPPPPPPG (SEQ ID NO: 34), isoEGWRW (SEQ ID NO: 35), DDGGGDDDFF (SEQ ID NO: 36), GGSSSGSGNDEEFQ (SEQ ID NO: 37), GGGGGDPDPDFF (SEQ ID NO: 38), GDGDGDGDGDFF (SEQ ID NO: 39), NNGGGNNNFF (SEQ ID NO: 40), and DDGGGCyCyCyFF (SEQ ID NO: 41), or a salt thereof, wherein Cy is a cysteic acid. In some embodiments, the polypeptidyl group comprises DDGGGDDDFF (SEQ ID NO: 36). In some embodiments, the oligonucleotide has a length of at least 25 nucleotides, and the polypeptidyl group comprises DDGGGDDDFF (SEQ ID NO: 36).

In some embodiments, the linking group further comprises at least one of optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof.

In some embodiments, Z¹ further comprises a binding group. In some embodiments, the binding group comprises a biotin moiety. In some embodiments, Z¹ further comprises a biotin moiety. In some embodiments, the biotin moiety is a bis-biotin moiety.

In some embodiments, the binding group comprises at least one tag sequence. In certain embodiments, the at least one tag sequence comprises at least one biotin ligase recognition sequence that permits biotinylation of the compound comprising Z¹ (e.g., incorporation of one or more biotin moieties, including biotin and bis-biotin moieties). In certain embodiments, the at least one tag sequence comprises at least one biotin ligase recognition sequence that permits biotinylation of Z¹. In certain embodiments, the at least one tag sequence comprises two biotin ligase recognition sequences oriented in tandem. In some cases, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem. In certain embodiments, the binding group comprises at least one biotin ligase recognition sequence having a biotin moiety attached thereto or at least two biotin ligase recognition sequences, each having a biotin moiety attached thereto.

In some embodiments, the binding group comprises or is conjugated to an avidin protein. In some embodiments, Z¹ further comprises an avidin protein. In some embodiments, the biotin moiety comprises an avidin protein. In some embodiments, the biotin moiety is conjugated to an avidin protein. The term “avidin protein” refers to a biotin-binding protein, generally having a biotin binding site at each of four subunits of the avidin protein. Non-limiting examples of avidin proteins include avidin, streptavidin, traptavidin, tamavidin, bradavidin, xenavidin, and homologs and variants thereof. In some cases, the avidin protein may have a monomeric, dimeric, or tetrameric form. In certain embodiments, the avidin protein is streptavidin in a tetrameric form (e.g., a homotetramer). In certain embodiments, the streptavidin in a tetrameric form may be bound to one component (e.g., a first component comprising a first mono-biotin moiety or a first bis-biotin moiety), two components (e.g., a first component comprising a first mono-biotin moiety or a first bis-biotin moiety and a second component comprising a second mono-biotin moiety or a second bis-biotin moiety), three components (e.g., a first component comprising a first bis-biotin moiety, a second component comprising a first mono-biotin moiety, and a third component comprising a second mono-biotin moiety), or four components (e.g., four components, each comprising a mono-biotin moiety).

In some embodiments, the compound comprising Z¹ is immobilized to a surface (e.g., a surface of a sample well). In some embodiments, the compound comprising Z¹ is immobilized to a surface of a sample well. In some embodiments, Z¹ is immobilized to a surface (e.g., a surface of a sample well). In some embodiments, Z¹ is immobilized to a surface of a sample well. In some embodiments, the avidin protein is immobilized to a surface (e.g., a surface of a sample well). In some embodiments, the avidin protein is immobilized to a surface of a sample well. As used herein, in some embodiments, a surface refers to a surface of a substrate or solid support. In some embodiments, a solid support refers to a material, layer, or other structure having a surface, such as a receiving surface, that is capable of supporting a deposited material, such as a compound described herein. In some embodiments, a receiving surface of a substrate may optionally have one or more features, including nanoscale or microscale recessed features such as an array of sample wells. In some embodiments, an array is a planar arrangement of elements such as sensors or sample wells. An array may be one or two dimensional. A one dimensional array is an array having one column or row of elements in the first dimension and a plurality of columns or rows in the second dimension. The number of columns or rows in the first and second dimensions may or may not be the same. In some embodiments, the array may include, for example, 10², 10³, 10⁴, 10⁵, 10⁶, or 10⁷ sample wells. In some embodiments, Z¹ is immobilized to a bottom surface or a sidewall surface of a sample well. In some embodiments, surface immobilization of Z¹ allows the compound to be confined to a desired region of a surface for real-time monitoring of a reaction involving the compound. In certain embodiments, the compound is immobilized to a surface through Z¹. In certain embodiments, the compound is immobilized to a surface through Z¹ such that the compound may be monitored without interference from other reaction components in solution. In some embodiments, surface immobilization of Z¹ allows the compound to be confined to a desired region of a surface for real-time monitoring of a reaction involving the compound.

### Additional Subgeneric Embodiments

In some embodiments, the compound of Formula (I) is of Formula (I-a):

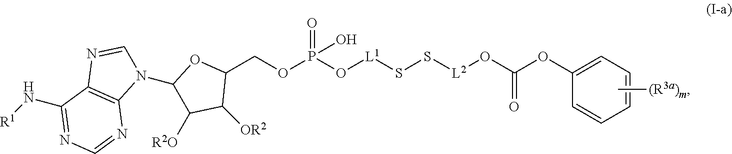

or a salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
m is 0, 1, 2, 3, 4, or 5.

In some embodiments, the compound of Formula (I-a) is of Formulae (I-a-1) or (I-a-2):

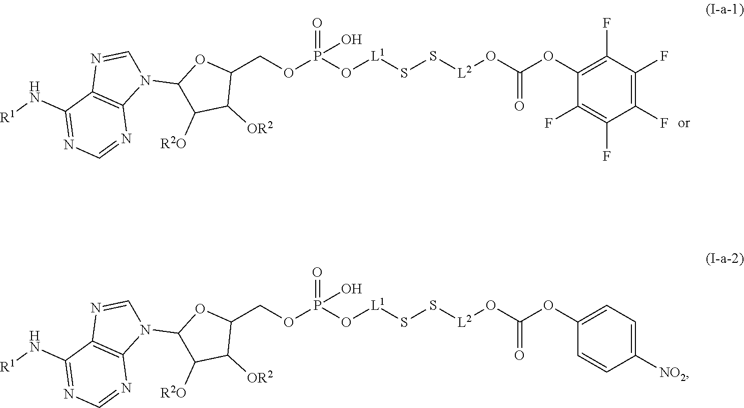

or a salt thereof.

In some embodiments, the compound of Formula (I-a) is of Formula (I-a-1), or a salt thereof. In some embodiments, the compound of Formula (I-a) is of Formula (I-a-2), or a salt thereof.

In some embodiments, the compound of Formula (I-a) is of Formula (I′-a):

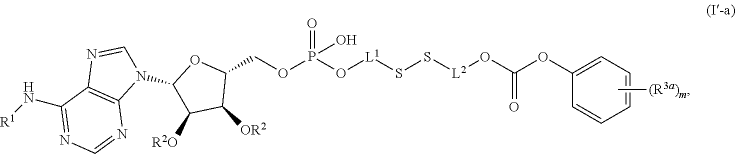

or a salt thereof.

In some embodiments, the compound of Formula (I′-a) is of Formulae (I′-a-1) or (I′-a-2):

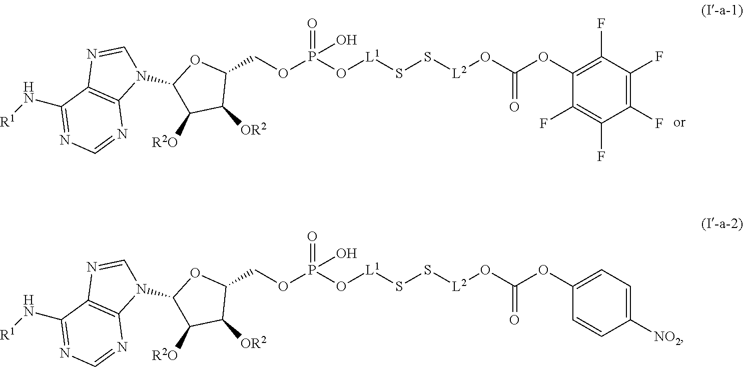

or a salt thereof.

In some embodiments, the compound of Formula (I′-a) is of Formula (I′-a-1), or a salt thereof. In some embodiments, the compound of Formula (I′-a) is of Formula (I′-a-2), or a salt thereof.

In some embodiments of Formulae (I-a) and (I′-a), at least one instance of R^{3a} is halogen or —NO₂. In some embodiments of Formulae (I-a) and (I′-a), each instance of R^{3a} is independently halogen. In some embodiments of Formulae (I-a) and (I′-a), at least one instance of R^{3a} is fluoro. In some embodiments of Formulae (I-a) and (I′-a), at least one instance of R^{3a} is —NO₂.

In some embodiments, the compound of Formula (I) is of Formula (I-b):

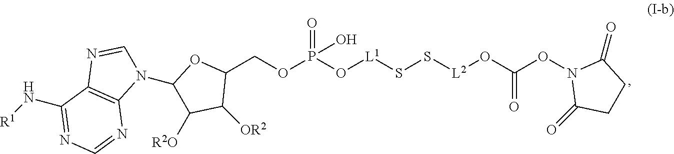

or a salt thereof.

In some embodiments, the compound of Formula (I-b) is of Formula (I′-b):

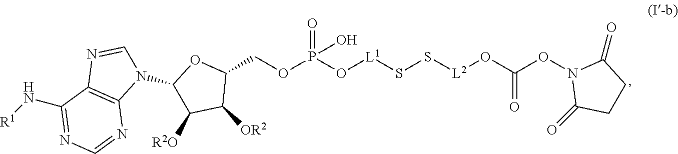

or a salt thereof.

In some embodiments, the compound of Formula (I) is of Formula (I-c) or (I-cc):

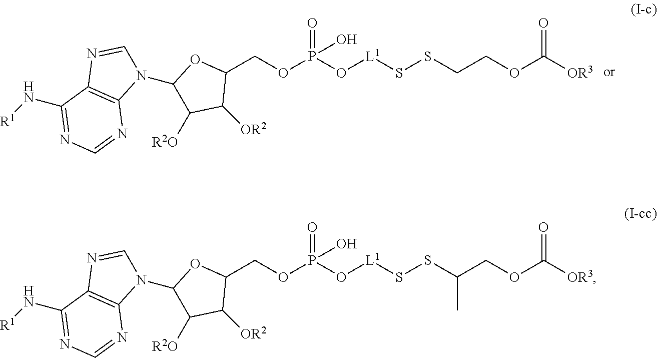

or a salt thereof.

In some embodiments, the compound of Formula (I) is of Formula (I-c), or a salt thereof. In some embodiments, the compound of Formula (I) is of Formula (I-cc), or a salt thereof.

In some embodiments, the compound of Formula (I-c) or (I-cc) is of Formula (I′-c) or (I′-cc):

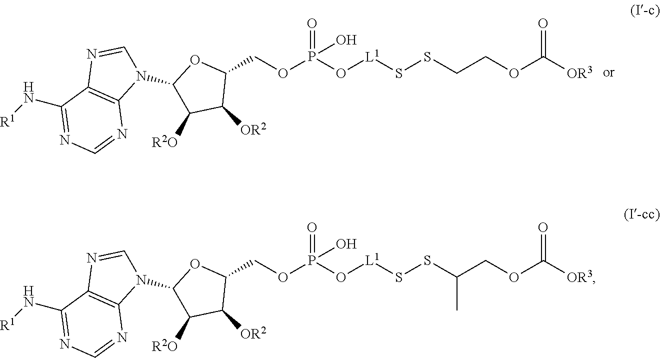

or a salt thereof.

In some embodiments, the compound of Formula (I-c) is of Formula (I′-c), or a salt thereof. In some embodiments, the compound of Formula (I-cc) is of Formula (I′-cc), or a salt thereof.

In some embodiments, the compound of Formula (I) is of Formula (I-d) or (I-dd):

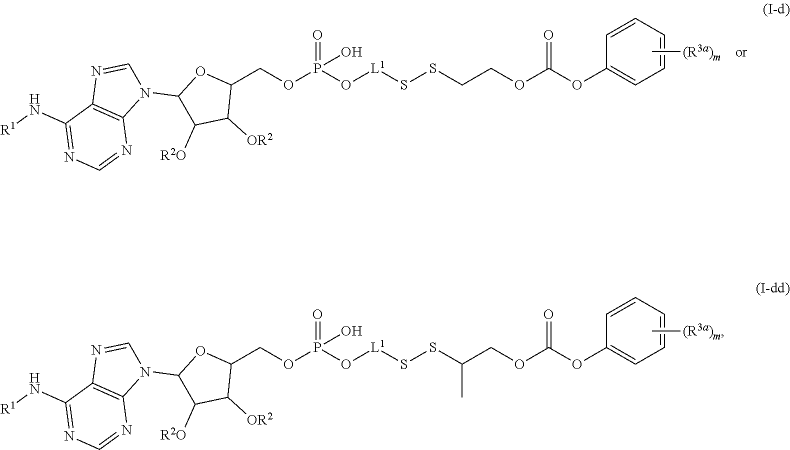

or a salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
m is 0, 1, 2, 3, 4, or 5.

In some embodiments, the compound of Formula (I) is of Formula (I-d), or a salt thereof. In some embodiments, the compound of Formula (I) is of Formula (I-dd), or a salt thereof.

In some embodiments, the compound of Formula (I-d) is of Formulae (I-d-1) or (I-d-2):

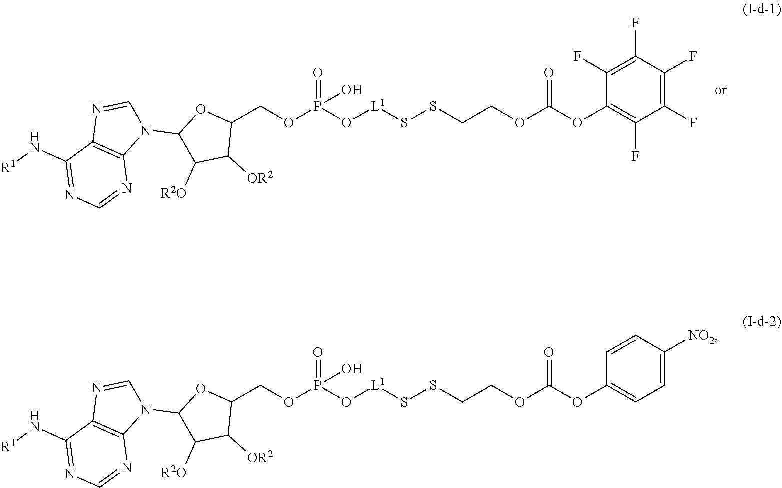

or a salt thereof.

In some embodiments, the compound of Formula (I-d) is of Formula (I-d-1), or a salt thereof. In some embodiments, the compound of Formula (I-d) is of Formula (I-d-2), or a salt thereof.

In some embodiments, the compound of Formula (I-d) is of Formula (I′-d):

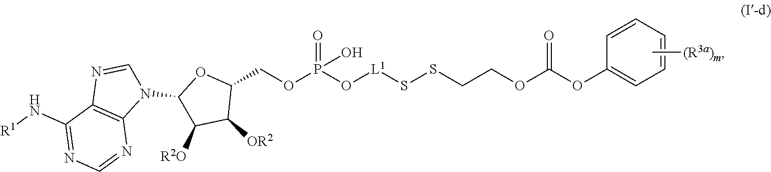

or a salt thereof.

In some embodiments of Formulae (I-d) and (I′-d), at least one instance of R^{3a} is halogen or —NO₂. In some embodiments of Formulae (I-d) and (I′-d), each instance of R^{3a} is independently halogen. In some embodiments of Formulae (I-d) and (I′-d), at least one instance of R^{3a} is fluoro. In some embodiments of Formulae (I-d) and (I′-d), at least one instance of R^{3a} is —NO₂.

In some embodiments, the compound of Formula (I′-d) is of Formulae (I′-d-1) or (I′-d-2):

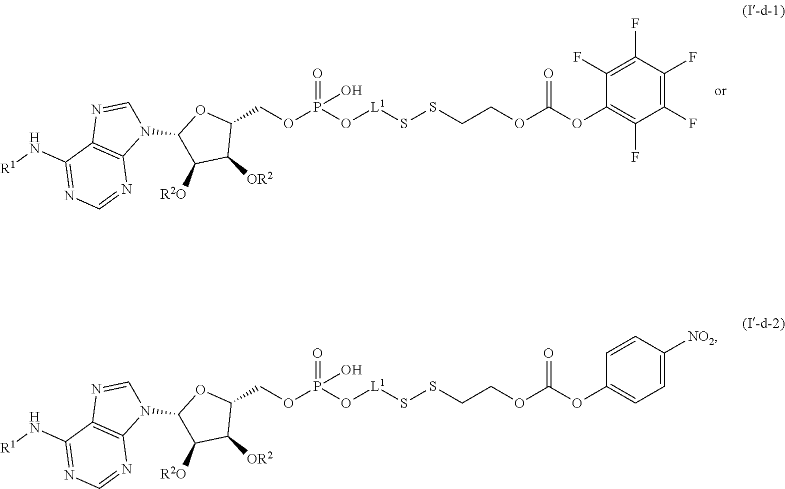

or a salt thereof.

In some embodiments, the compound of Formula (I′-d) is of Formula (I′-d-1), or a salt thereof. In some embodiments, the compound of Formula (I′-d) is of Formula (I′-d-2), or a salt thereof.

In some embodiments, the compound of Formula (I-dd) is of Formulae (I-dd-1) or (I-dd-2):

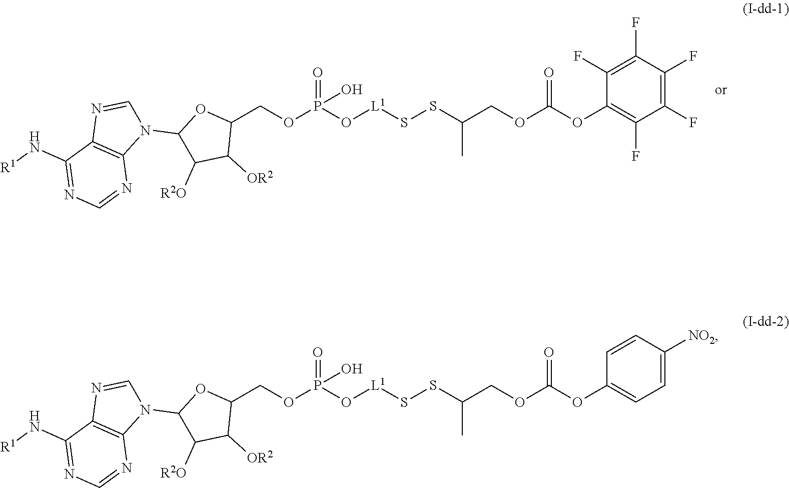

or a salt thereof.

In some embodiments, the compound of Formula (I-dd) is of Formula (I-dd-1), or a salt thereof. In some embodiments, the compound of Formula (I-dd) is of Formula (I-dd-2), or a salt thereof.

In some embodiments, the compound of Formula (I-dd) is of Formula (I′-dd):

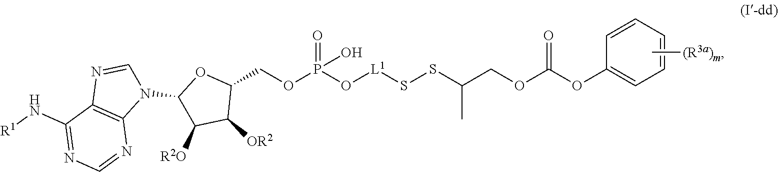

or a salt thereof.

In some embodiments of Formulae (I-dd) and (I′-dd), at least one instance of R^{3a} is halogen or —NO₂. In some embodiments of Formulae (I-dd and (I′-dd), each instance of R^{3a} is independently halogen. In some embodiments of Formulae (I-dd) and (I′-dd), at least one instance of R^{3a} is fluoro. In some embodiments of Formulae (I-dd) and (I′-dd), at least one instance of R^{3a} is —NO₂.

In some embodiments, the compound of Formula (I′-dd) is of Formulae (I′-dd-1) or (I′-dd-1):

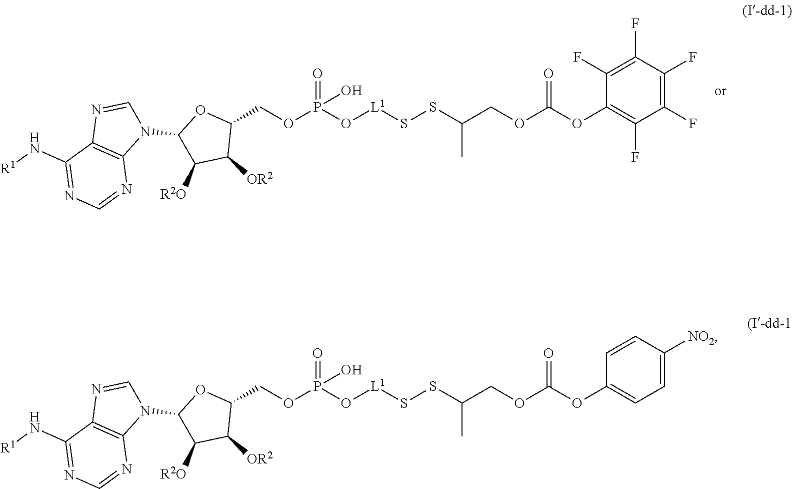

or a salt thereof.

In some embodiments, the compound of Formula (I′-dd) is of Formula (I′-dd-1), or a salt thereof. In some embodiments, the compound of Formula (I′-dd) is of Formula (I′-dd-2), or a salt thereof.

In some embodiments, the compound of Formula (I) is of Formula (I-e) or (I-ee):

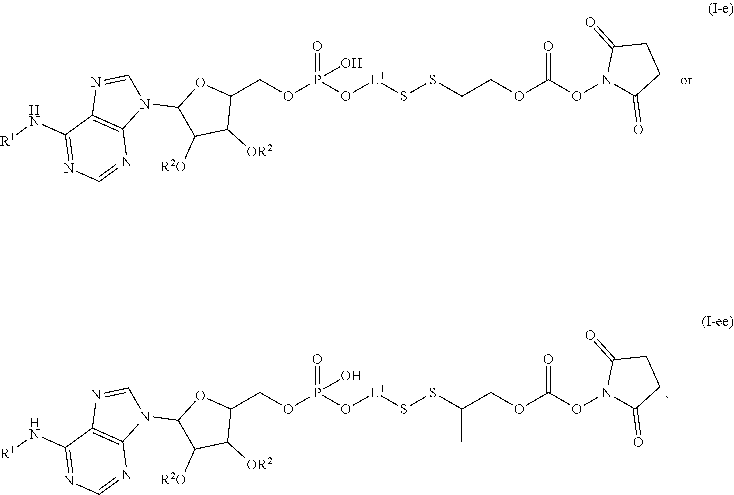

or a salt thereof.

In some embodiments, the compound of Formula (I) is of Formula (I-e), or a salt thereof. In some embodiments, the compound of Formula (I) is of Formula (I-ee), or a salt thereof.

In some embodiments, the compound of Formula (I-e) or (I-ee) is of Formula (I′-e) or (I′-ee):

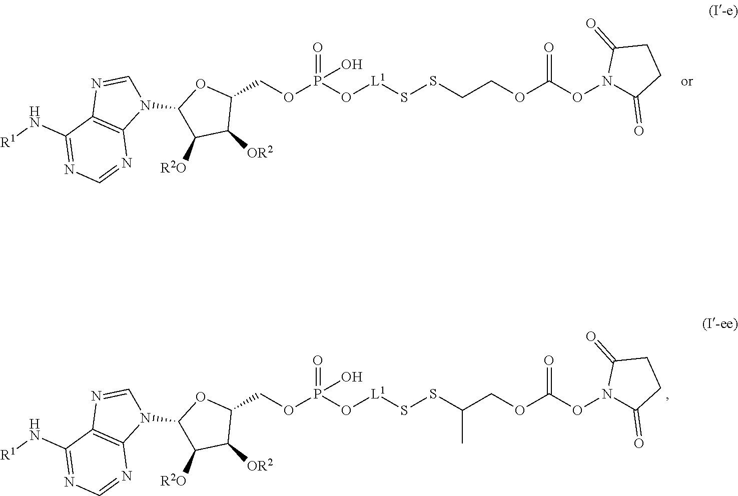

or a salt thereof.

In some embodiments, the compound of Formula (I-e) is of Formula (I′-e), or a salt thereof. In some embodiments, the compound of Formula (I-ee) is of Formula (I′-ee), or a salt thereof.

In some embodiments, the compound of Formula (I) is of Formula (I-f):

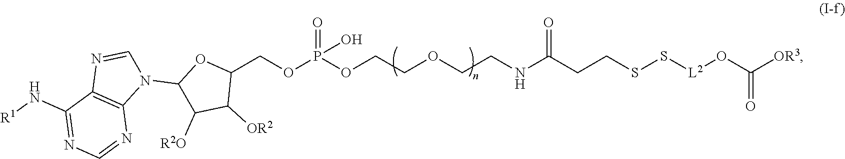

or a salt thereof, wherein n is an integer between 0 and 30, inclusive.

In some embodiments, the compound of Formula (I-f) is of Formula (I′-f):

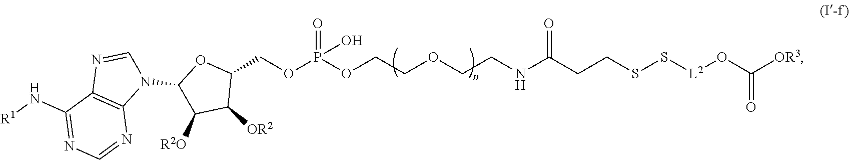

or a salt thereof.

In some embodiments, the compound of Formula (I) is of Formula (I-g):

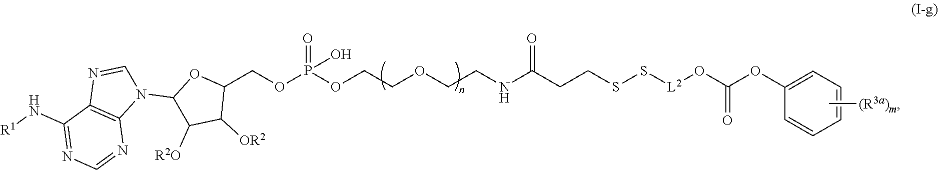

or a salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring;
m is0, 1, 2, 3, 4, or 5; and
n is an integer between 0 and 30, inclusive.

In some embodiments, the compound of Formula (I-g) is of Formulae (I-g-1) or (I-g-2):

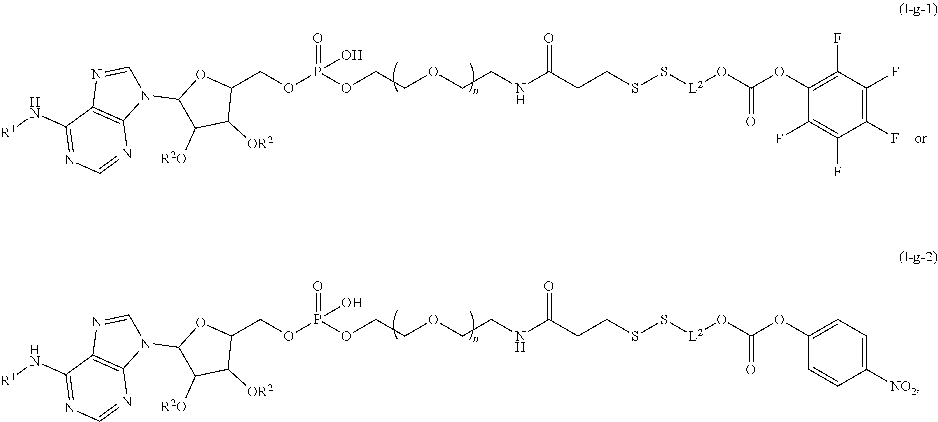

or a salt thereof.

In some embodiments, the compound of Formula (I-g) is of Formula (I-g-1), or a salt thereof. In some embodiments, the compound of Formula (I-g) is of Formula (I-g-2), or a salt thereof.

In some embodiments, the compound of Formula (I-g) is of Formula (I′-g):

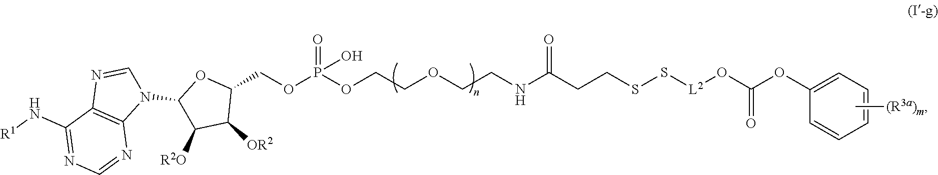

or a salt thereof.

In some embodiments of Formulae (I-g) and (I′-g), at least one instance of R^{3a} is halogen or —NO₂. In some embodiments of Formulae (I-g) and (I′-g), each instance of R^{3a} is independently halogen. In some embodiments of Formulae (I-g) and (I′-g), at least one instance of R^{3a} is fluoro. In some embodiments of Formulae (I-g) and (I′-g), at least one instance of R^{3a} is —NO₂.

In some embodiments, the compound of Formula (I′-g) is of Formulae (I′-g-1) or (I′-g-2):

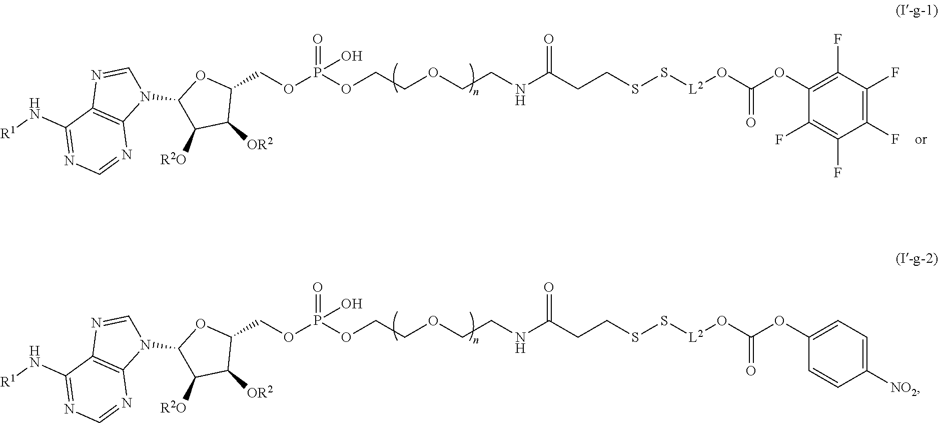

or a salt thereof.

In some embodiments, the compound of Formula (I′-g) is of Formula (I′-g-1), or a salt thereof. In some embodiments, the compound of Formula (I′-g) is of Formula (I′-g-2), or a salt thereof.

In some embodiments, the compound of Formula (I) is of Formula (I-h):

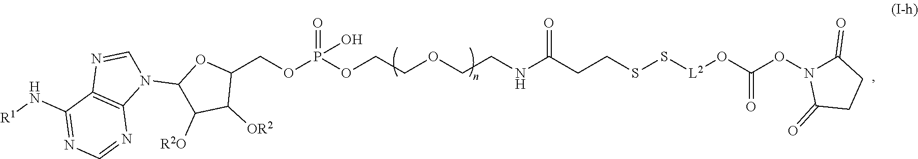

or a salt thereof, wherein n is an integer between 0 and 30, inclusive.

In some embodiments, the compound of Formula (I-h) is of Formula (I′-h):

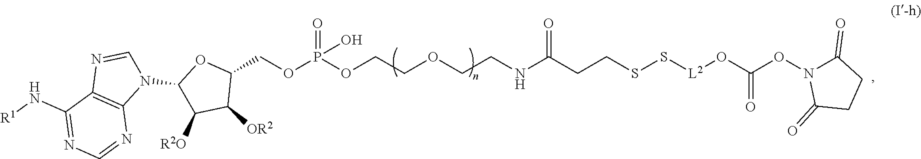

or a salt thereof, wherein n is an integer between 0 and 30, inclusive.

In some embodiments, the compound of Formula (I) is of Formula (I-i) or (I-ii):

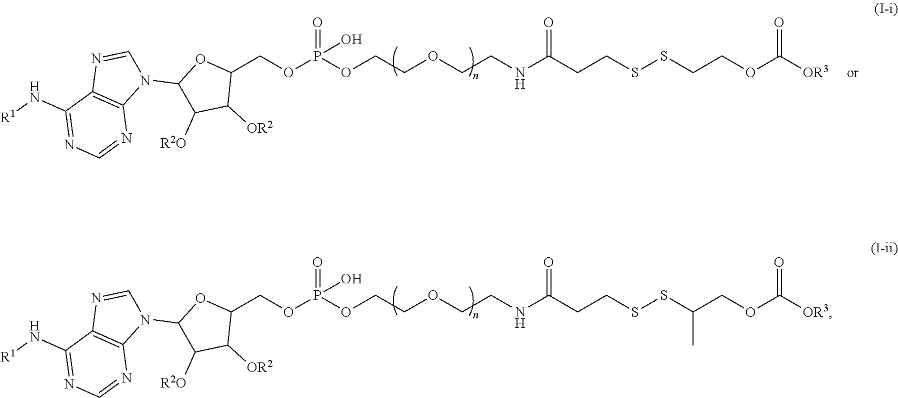

or a salt thereof, wherein n is an integer between 0 and 30, inclusive.

In some embodiments, the compound of Formula (I) is of Formula (I-i), or a salt thereof. In some embodiments, the compound of Formula (I) is of Formula (I-ii), or a salt thereof.

In some embodiments, the compound of Formula (I-i) or (I-ii) is of Formula (I′-i) or (I′-ii):

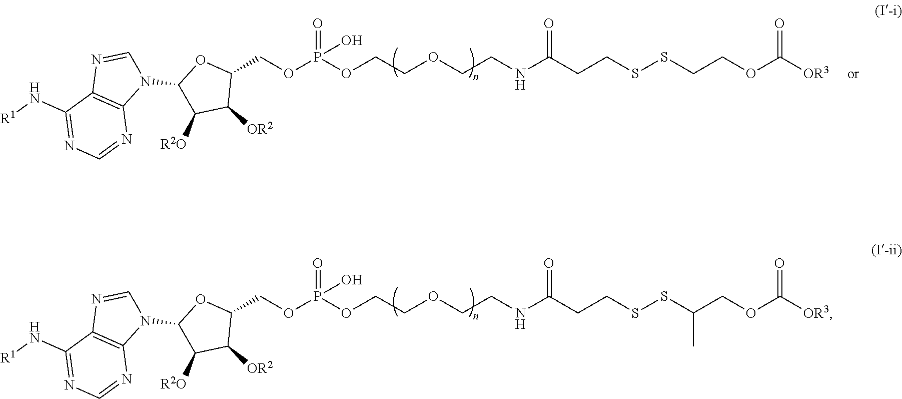

or a salt thereof, wherein n is an integer between 0 and 30, inclusive.

In some embodiments, the compound of Formula (I-i) is of Formula (I′-i), or a salt thereof. In some embodiments, the compound of Formula (I-ii) is of Formula (I′-ii), or a salt thereof.

In some embodiments, the compound of Formula (I) is of Formula (I-j) or (I-jj):

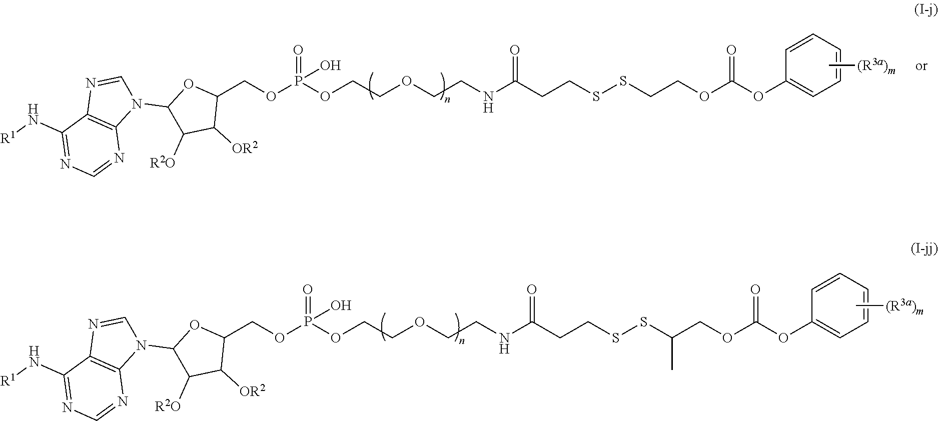

or a salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring;
m is 0, 1, 2, 3, 4, or 5; and
n is an integer between 0 and 30, inclusive.

In some embodiments, the compound of Formula (I) is of Formula (I-j), or a salt thereof. In some embodiments, the compound of Formula (I) is of Formula (I-jj), or a salt thereof.

In some embodiments, the compound of Formula (I-j) is of Formulae (I-j-1) or (I-j-2):

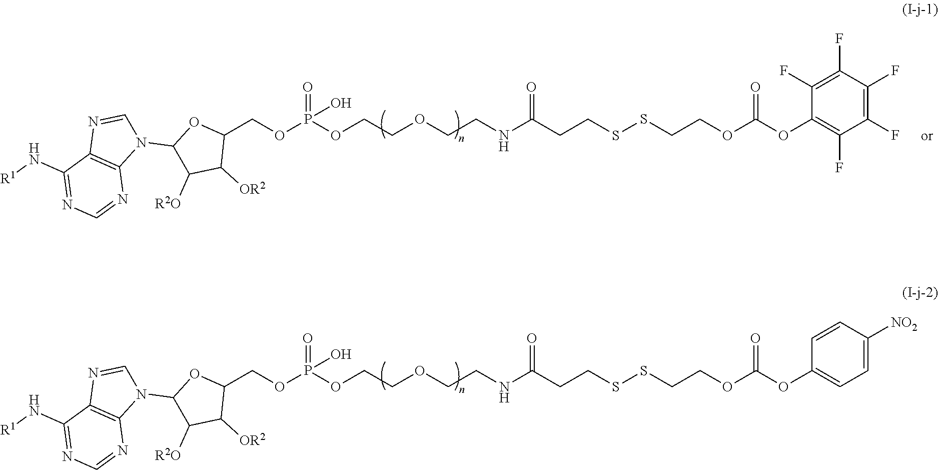

or a salt thereof.

In some embodiments, the compound of Formula (I-j) is of Formula (I-j-1), or a salt thereof. In some embodiments, the compound of Formula (I-j) is of Formula (I-j-2), or a salt thereof.

In some embodiments, the compound of Formula (I-j) is of Formulae (I-j-3) or (I-j-4):

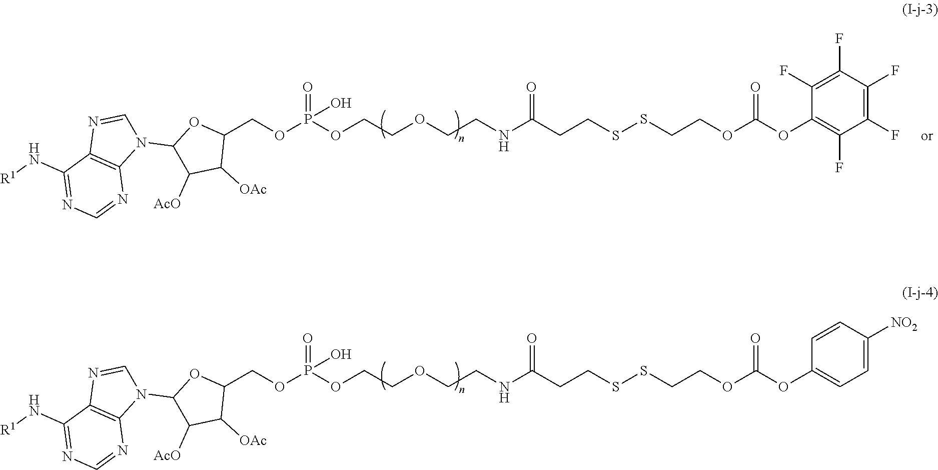

or a salt thereof.

In some embodiments, the compound of Formula (I-j) is of Formula (I-j-3), or a salt thereof. In some embodiments, the compound of Formula (I-j) is of Formula (I-j-4), or a salt thereof.

In some embodiments, the compound of Formula (I) is of Formulae (I-j-5) or (I-j-6):

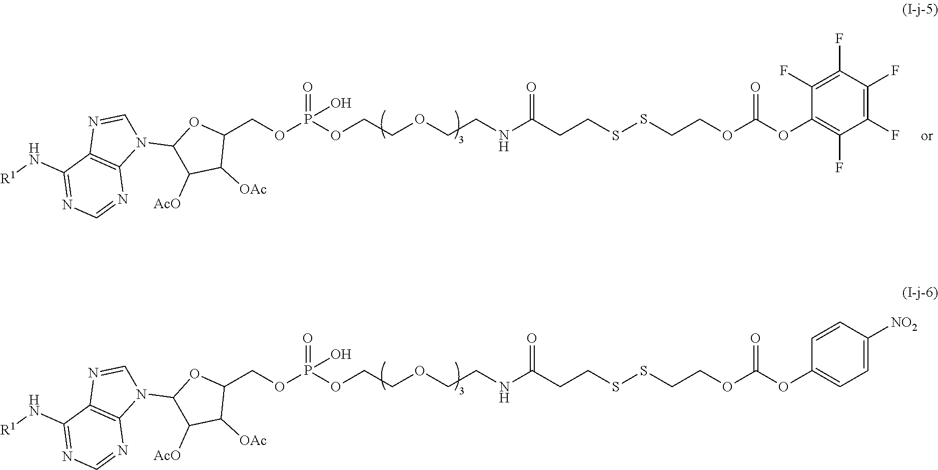

or a salt thereof.

In some embodiments, the compound of Formula (I-j) is of Formula (I-j-5), or a salt thereof. In some embodiments, the compound of Formula (I-j) is of Formula (I-j-6), or a salt thereof.

In some embodiments, the compound of Formula (I-j) is of Formula (I′-j):

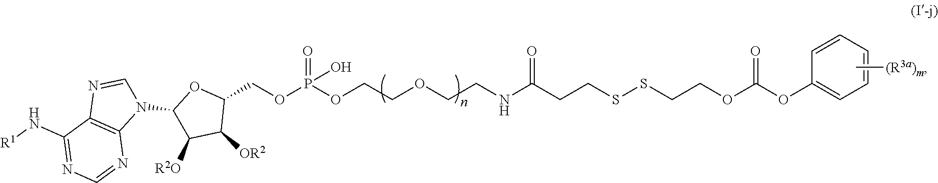

or a salt thereof.

In some embodiments of Formulae (I-j) and (I′-j), at least one instance of R^{3a} is halogen or —NO₂. In some embodiments of Formulae (I-j) and (I′-j), each instance of R^{3a} is independently halogen. In some embodiments of Formulae (I-j) and (I′-j), at least one instance of R^{3a} is fluoro. In some embodiments of Formulae (I-j) and (I′-j), at least one instance of R^{3a} is —NO₂.

In some embodiments, the compound of Formula (I′-j) is of Formulae (I′-j-1) or (I′-j-2):

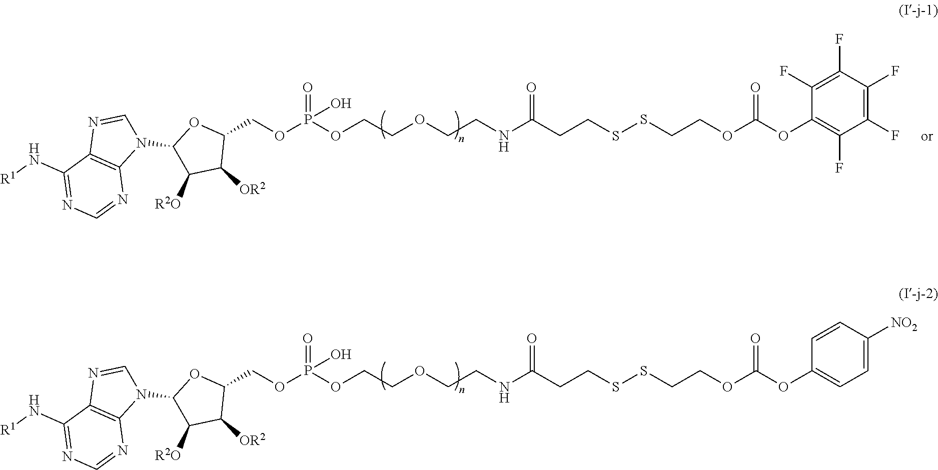

or a salt thereof.

In some embodiments, the compound of Formula (I′-j) is of Formula (I′-j-1), or a salt thereof. In some embodiments, the compound of Formula (I′-j) is of Formula (I′-j-2), or a salt thereof.

In some embodiments, the compound of Formula (I′-j) is of Formulae (I′-j-3) or (I′-j-4):

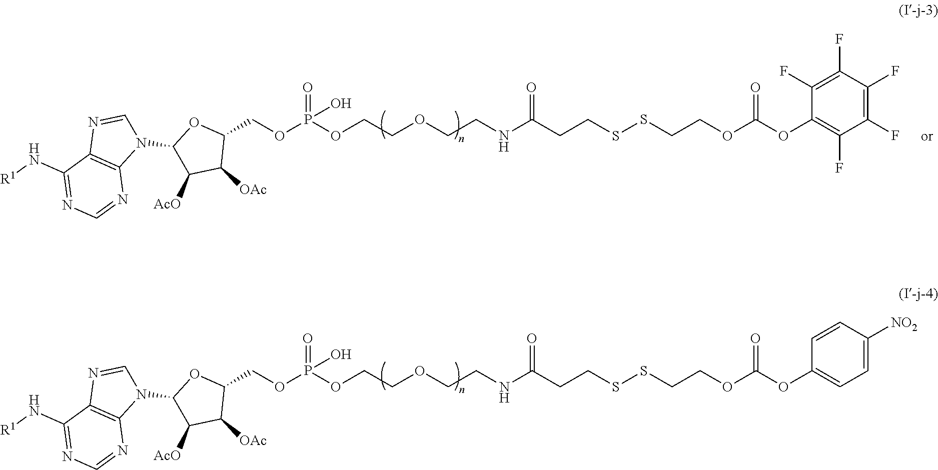

or a salt thereof.

In some embodiments, the compound of Formula (I′-j) is of Formula (I′-j-3), or a salt thereof. In some embodiments, the compound of Formula (I′-j) is of Formula (I′-j-4), or a salt thereof.

In some embodiments, the compound of Formula (I′) is of Formulae (I′-j-5) or (I′-j-6):

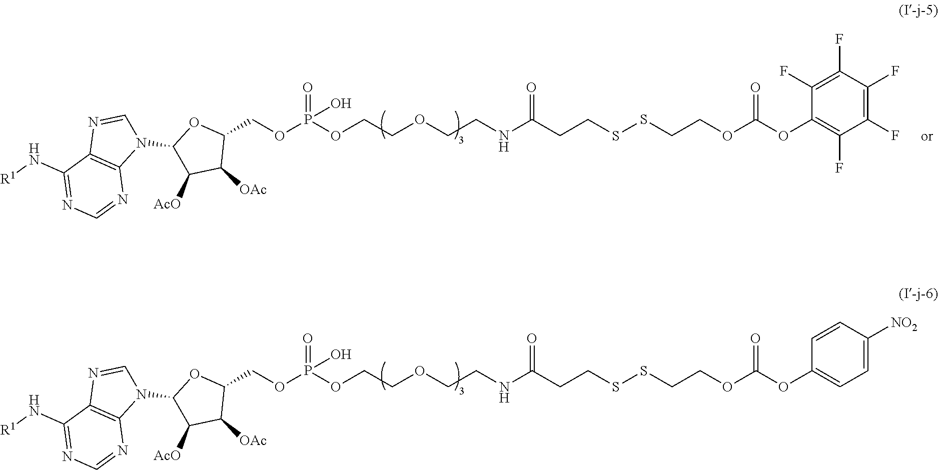

or a salt thereof.

In some embodiments, the compound of Formula (I′-j) is of Formula (I′-j-5), or a salt thereof. In some embodiments, the compound of Formula (I′-j) is of Formula (I′-j-6), or a salt thereof.

In some embodiments, the compound of Formula (I-jj) is of Formulae (I-jj-1) or (I-jj-2):

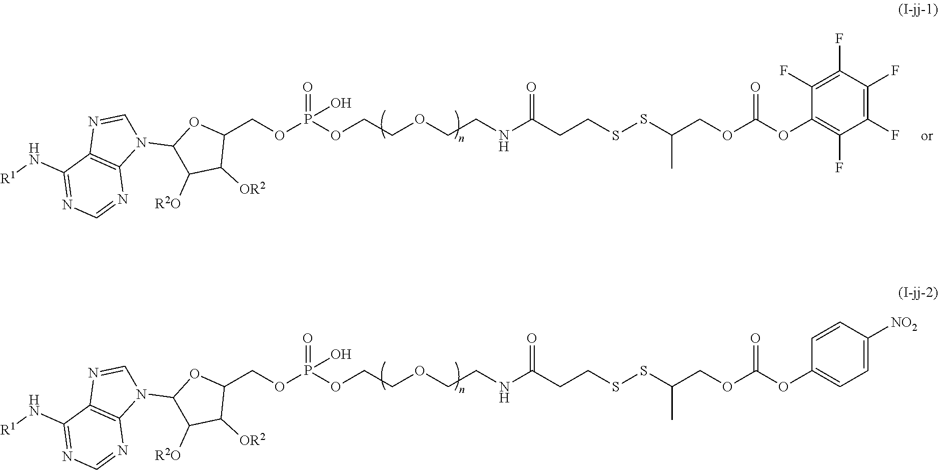

or a salt thereof.

In some embodiments, the compound of Formula (I-jj) is of Formula (I-jj-1), or a salt thereof. In some embodiments, the compound of Formula (I-jj) is of Formula (I-jj-2), or a salt thereof.

In some embodiments, the compound of Formula (I-jj) is of Formulae (I-jj-3) or (I-jj-4):

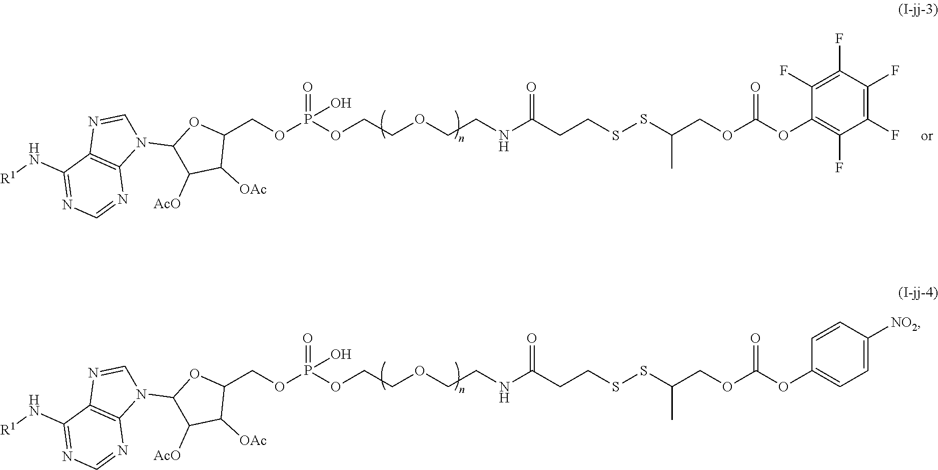

or a salt thereof.

In some embodiments, the compound of Formula (I-jj) is of Formula (I-jj-3), or a salt thereof. In some embodiments, the compound of Formula (I-jj) is of Formula (I-jj-4), or a salt thereof.

In some embodiments, the compound of Formula (I) is of Formulae (I-jj-5) or (I-jj-6):

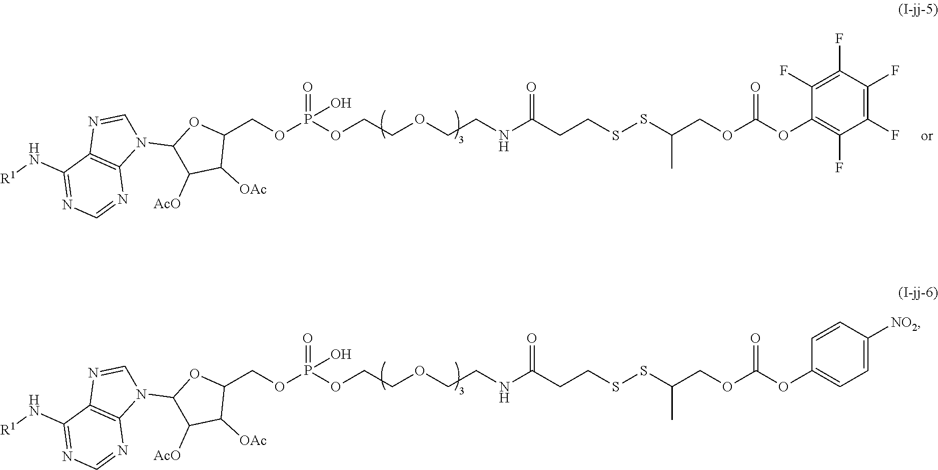

or a salt thereof.

In some embodiments, the compound of Formula (I-jj) is of Formula (I-jj-5), or a salt thereof. In some embodiments, the compound of Formula (I-jj) is of Formula (I-jj-6), or a salt thereof.

In some embodiments, the compound of Formula (I-jj) is of Formula (I′-jj):

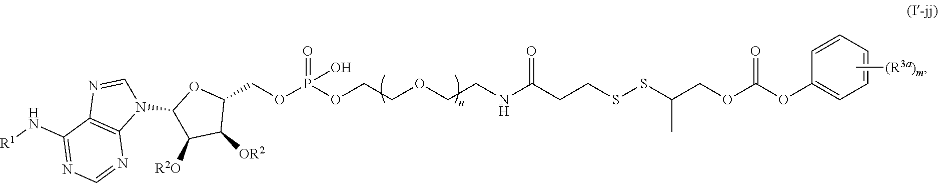

or a salt thereof.

In some embodiments of Formulae (I-jj) and (I′-jj), at least one instance of R^{3a} is halogen or —NO₂. In some embodiments of Formulae (I-jj) and (I′-jj), each instance of R^{3a} is independently halogen. In some embodiments of Formulae (I-jj) and (I′-jj), at least one instance of R^{3a} is fluoro. In some embodiments of Formulae (I-jj) and (I′-jj), at least one instance of R^{3a} is —NO₂.

In some embodiments, the compound of Formula (I′-jj) is of Formulae (I′-jj-1) or (I′-jj-2):

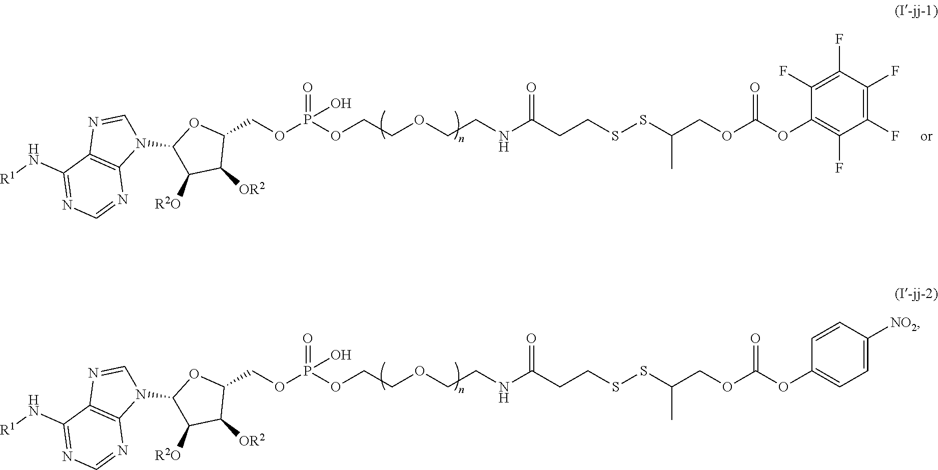

or a salt thereof.

In some embodiments, the compound of Formula (I′-jj) is of Formula (I′-jj-1), or a salt thereof. In some embodiments, the compound of Formula (I′-jj) is of Formula (I′-jj-2), or a salt thereof.

In some embodiments, the compound of Formula (I′-jj) is of Formulae (I′-jj-3) or (I′-jj-4):

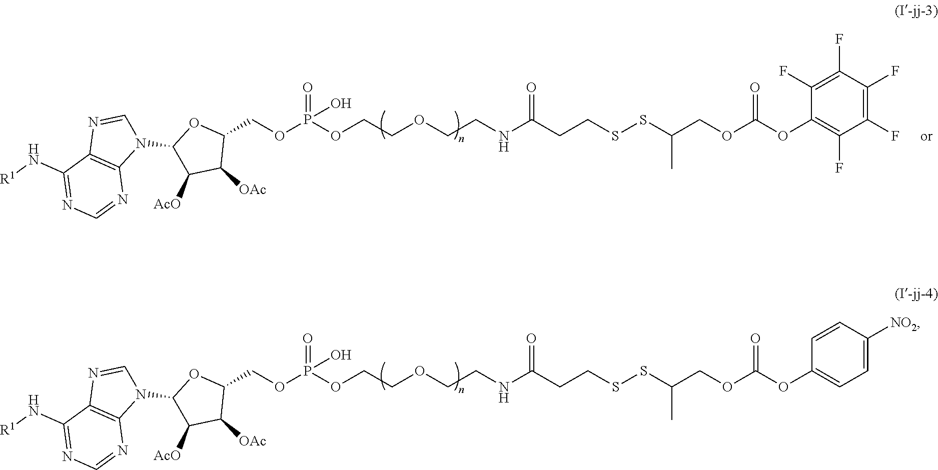

or a salt thereof.

In some embodiments, the compound of Formula (I′-jj) is of Formula (I′-jj-3), or a salt thereof. In some embodiments, the compound of Formula (I′-jj) is of Formula (I′-jj-4), or a salt thereof.

In some embodiments, the compound of Formula (I′) is of Formulae (I′-jj-5) or (I′-jj-6):

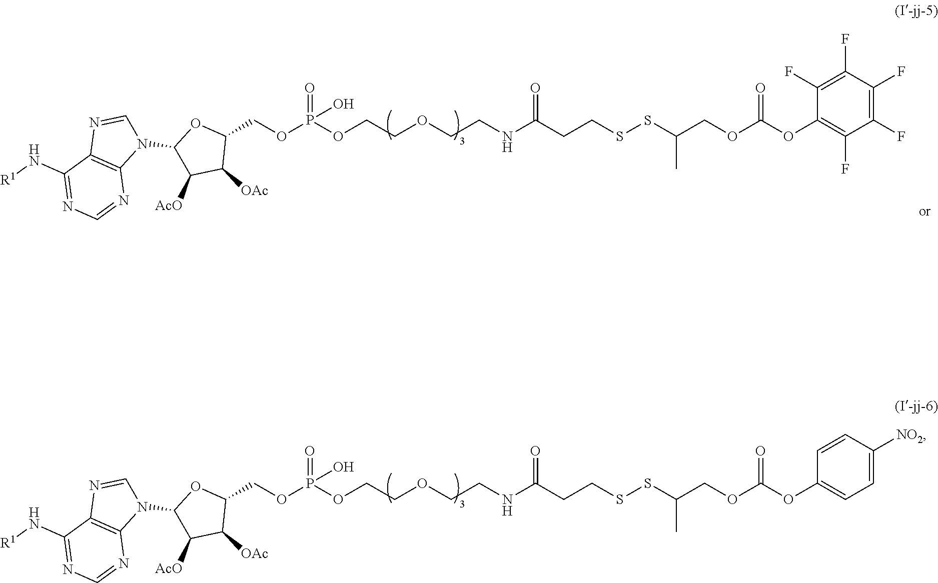

or a salt thereof.

In some embodiments, the compound of Formula (I′-jj) is of Formula (I′-jj-5), or a salt thereof. In some embodiments, the compound of Formula (I′-jj) is of Formula (I′-jj-6), or a salt thereof.

In some embodiments, the compound of Formula (I) is of Formula (I-k) or (I-kk):

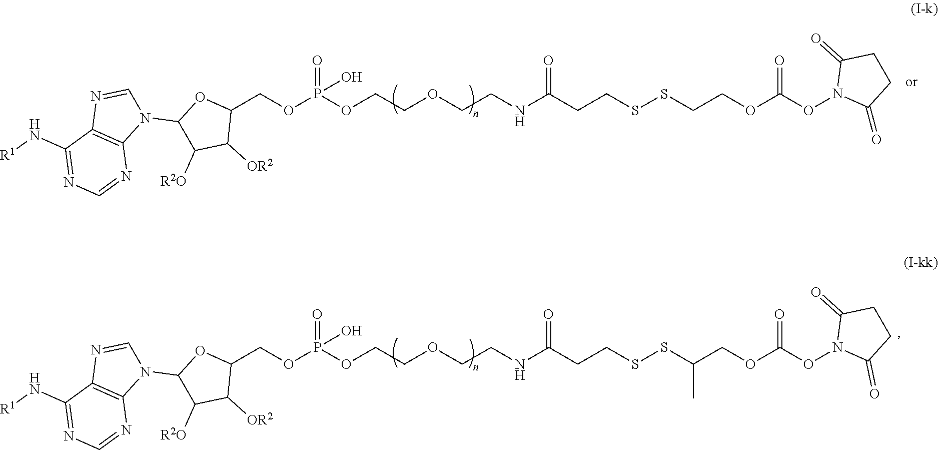

or a salt thereof, wherein n is an integer between 0 and 30, inclusive.

In some embodiments, the compound of Formula (I) is of Formula (I-k), or a salt thereof. In some embodiments, the compound of Formula (I) is of Formula (I-kk), or a salt thereof.

In some embodiments, the compound of Formula (I-k) or (I-kk) is of Formula (I-k-1) or (I-kk-1):

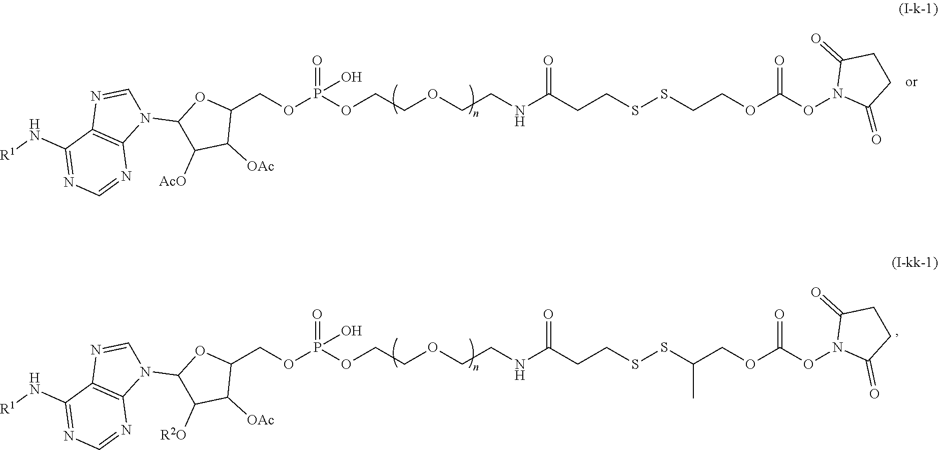

or a salt thereof.

In some embodiments, the compound of Formula (I-k) is of Formula (I-k-1), or a salt thereof. In some embodiments, the compound of Formula (I-kk) is of Formula (I-kk-1), or a salt thereof.

In some embodiments, the compound of Formula (I-k) or (I-kk) is of Formula (I-k-2) or (I-kk-2):

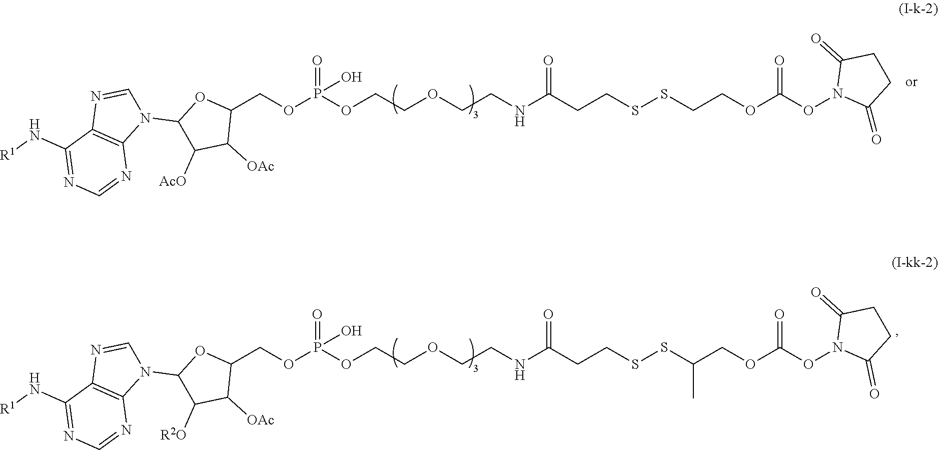

or a salt thereof.

In some embodiments, the compound of Formula (I-k) is of Formula (I-k-2), or a salt thereof. In some embodiments, the compound of Formula (I-kk) is of Formula (I-kk-2), or a salt thereof.

In some embodiments, the compound of Formula (I) is of Formula (I′-k) or (I′-kk):

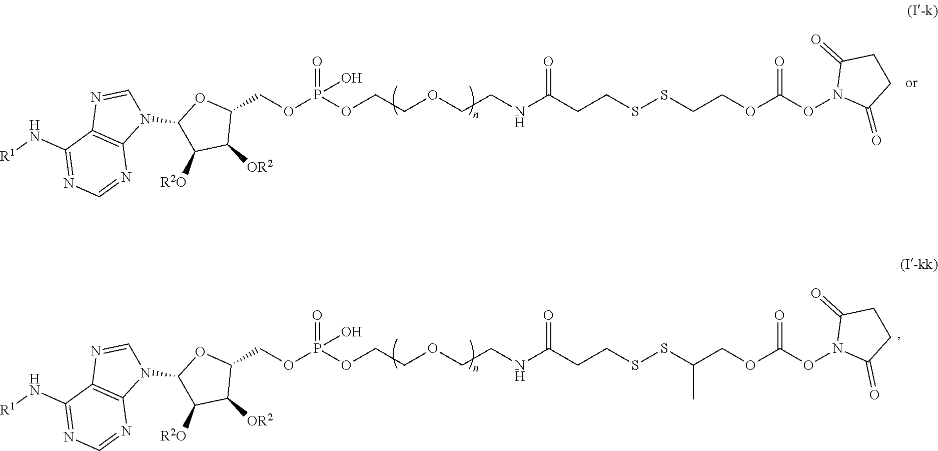

or a salt thereof, wherein n is an integer between 0 and 30, inclusive.

In some embodiments, the compound of Formula (I) is of Formula (I′-k), or a salt thereof. In some embodiments, the compound of Formula (I) is of Formula (I′-kk), or a salt thereof.

In some embodiments, the compound of Formula (I′-k) or (I′-kk) is of Formula (I′-k-1) or (I′-kk-1):

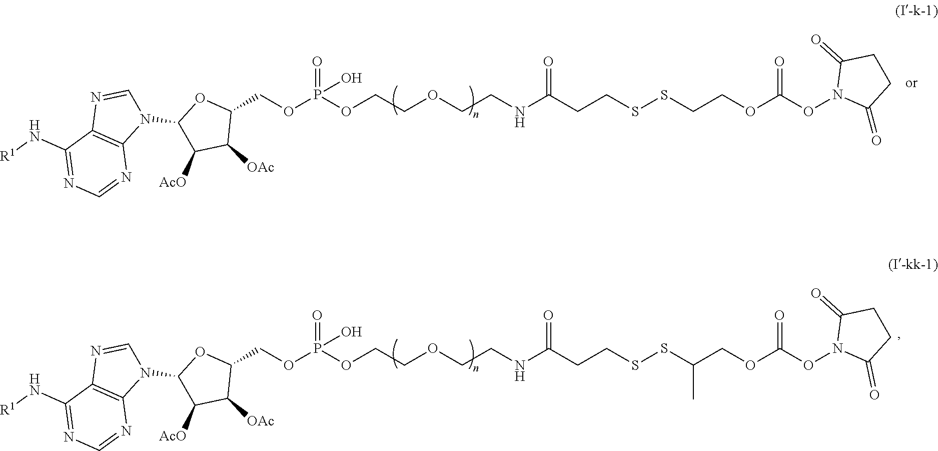

or a salt thereof.

In some embodiments, the compound of Formula (I′-k) is of Formula (I′-k-1), or a salt thereof. In some embodiments, the compound of Formula (I′-kk) is of Formula (I′-kk-1), or a salt thereof.

In some embodiments, the compound of Formula (I′-k) or (I′-kk) is of Formula (I′-k-2) or (I′-kk-2):

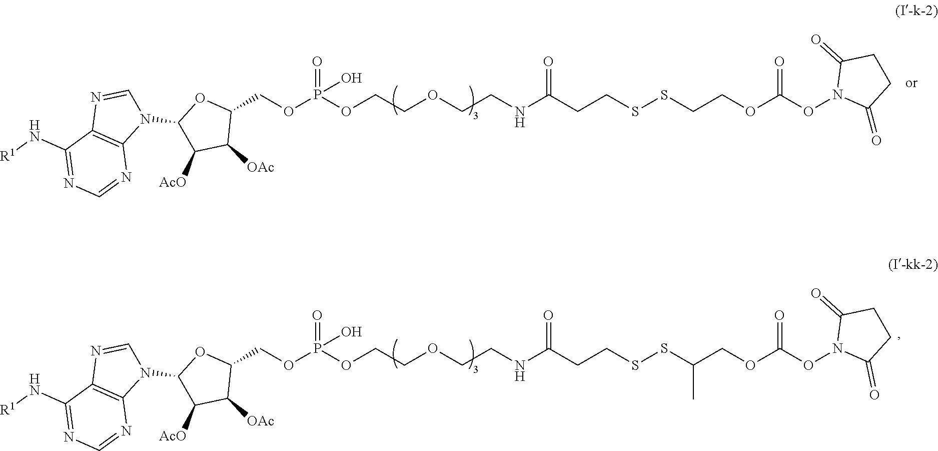

or a salt thereof.

In some embodiments, the compound of Formula (I′-k) is of Formula (I′-k-2), or a salt thereof. In some embodiments, the compound of Formula (I′-kk) is of Formula (I′-kk-2), or a salt thereof.

In some embodiments, the compound of Formula (II) is of Formula (II-a-1) or (II-a-2):

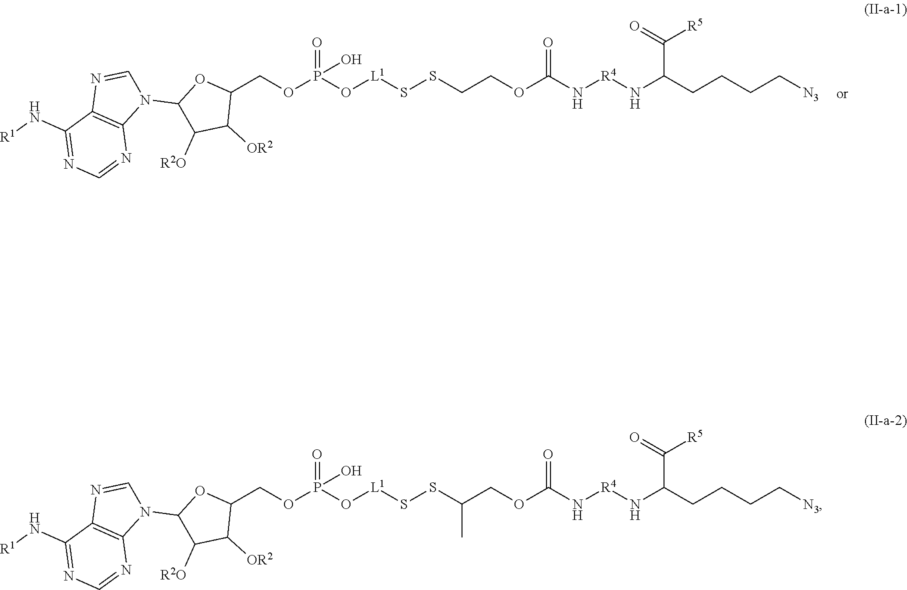

or a salt thereof.

In some embodiments, the compound of Formula (II) is of Formula (II-a-1), or a salt thereof. In some embodiments, the compound of Formula (II) is of Formula (II-a-2), or a salt thereof.

In some embodiments, the compound of Formula (II-a-1) or (II-a-2) is of Formula (II′-a-1) or (II′-a-2):

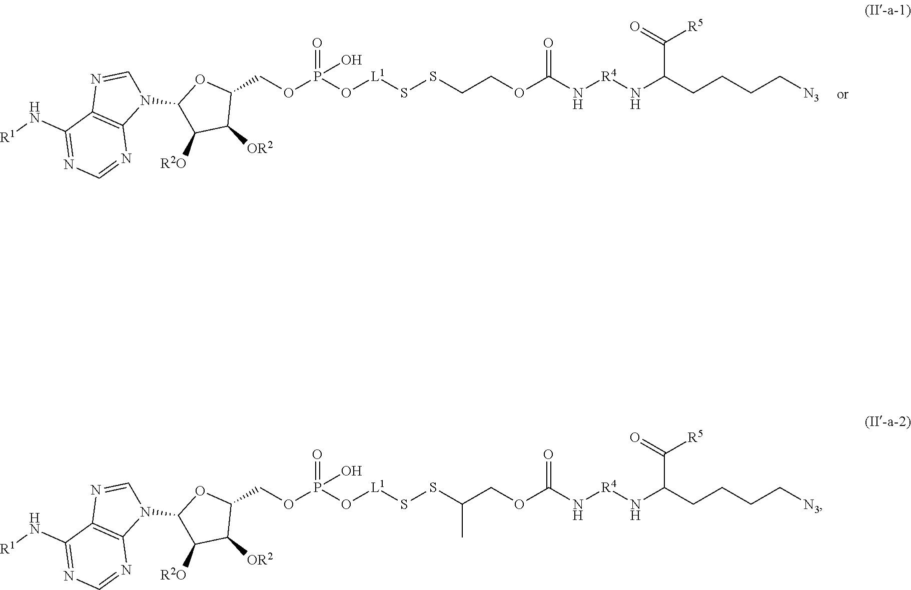

or a salt thereof.

In some embodiments, the compound of Formula (II-a-1) is of Formula (II′-a-1), or a salt thereof. In some embodiments, the compound of Formula (II-a-2) is of Formula (II′-a-2), or a salt thereof.

In some embodiments of Formulae (II-a-1) and (II′-a-1), R⁵ is —OH. In some embodiments of Formulae (II-a-1) and (II′-a-1), R⁵ is —NH₂. In some embodiments of Formulae (II-a-2) and (II′-a-2), R⁵ is —OH. In some embodiments of Formulae (II-a-2) and (II′-a-2), R⁵ is —NH₂.

In some embodiments, the compound of Formula (II) is of Formula (II-b):

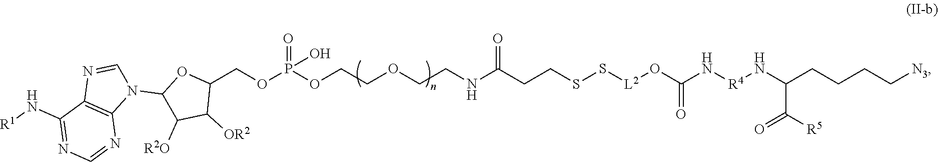

or a salt thereof.

In some embodiments, the compound of Formula (II-b) is of Formula (II′-b):

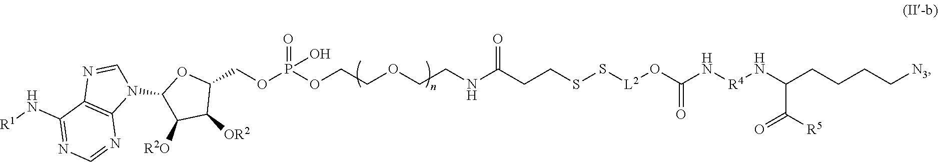

or a salt thereof.

In some embodiments of Formulae (II-b) and (II′-b), R⁵ is —OH. In some embodiments of Formulae (II-b) and (II′-b), R⁵ is —NH₂.

In some embodiments, the compound of Formula (II) is of Formula (II-c-1) or (II-c-2):

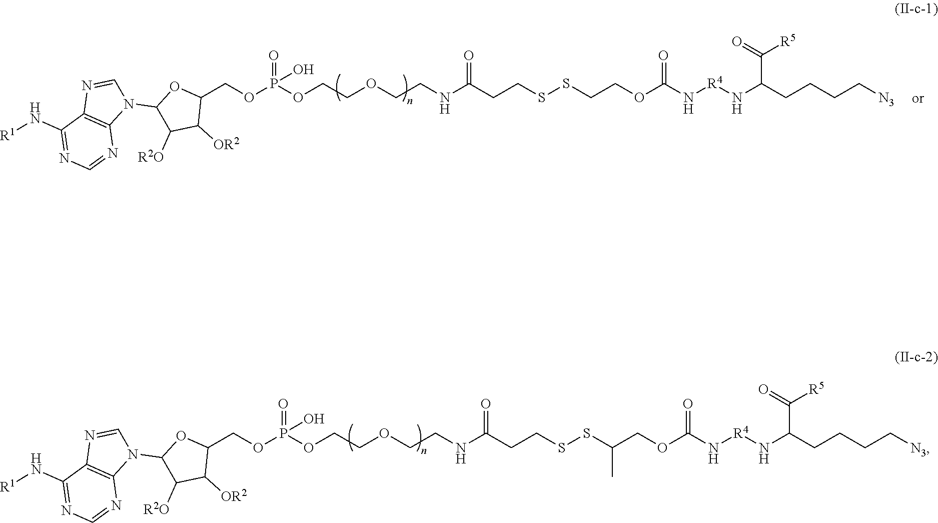

or a salt thereof.

In some embodiments, the compound of Formula (II) is of Formula (II-c-1), or a salt thereof. In some embodiments, the compound of Formula (II) is of Formula (II-c-2), or a salt thereof.

In some embodiments, the compound of Formula (II-c-1) or (II-c-2) is of Formula (II′-c-1) or (II′-c-2):

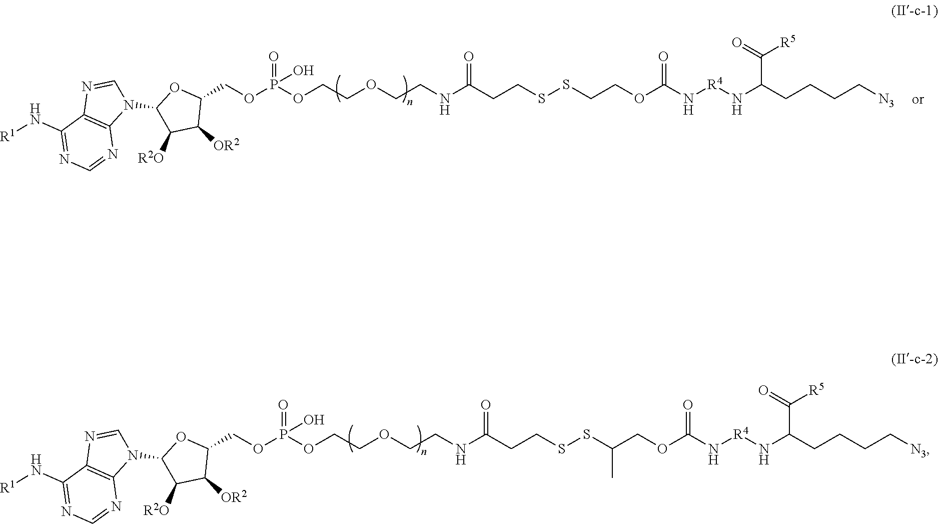

or a salt thereof.

In some embodiments, the compound of Formula (II-c-1) is of Formula (II′-c-1), or a salt thereof. In some embodiments, the compound of Formula (II-c-2) is of Formula (II′-c-2), or a salt thereof.

In some embodiments of Formulae (II-c-1) and (II′-c-1), R⁵ is —OH. In some embodiments of Formulae (II-c-1) and (II′-c-1), R⁵ is —NH₂. In some embodiments of Formulae (II-c-2) and (II′-c-2), R⁵ is —OH. In some embodiments of Formulae (II-c-2) and (II′-c-2), R⁵ is —NH₂.

In some embodiments, the compound of Formula (III) is of Formula (III-a-1) or (III-a-2):

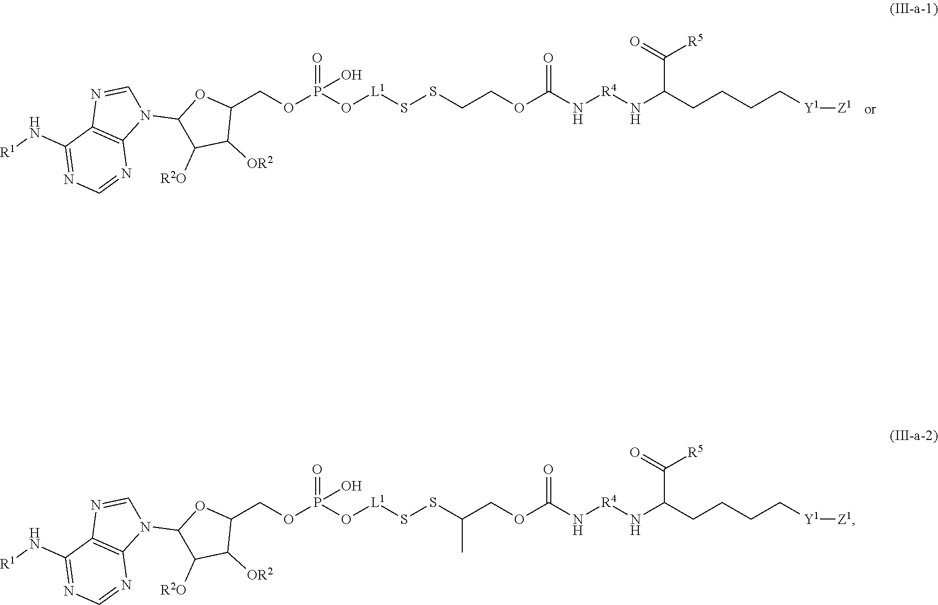

or a salt thereof.

In some embodiments, the compound of Formula (III) is of Formula (III-a-1), or a salt thereof. In some embodiments, the compound of Formula (III) is of Formula (III-a-2), or a salt thereof.

In some embodiments, the compound of Formula (III-a-1) or (III-a-2) is of Formula (III′-a-1) or (III′-a-2):

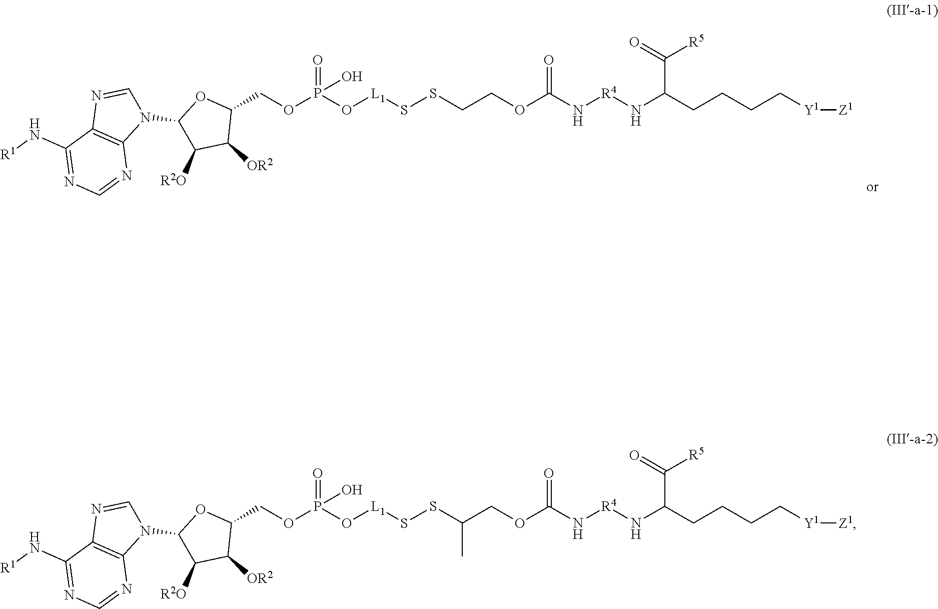

or a salt thereof.

In some embodiments, the compound of Formula (III-a-1) is of Formula (III-a-1), or a salt thereof. In some embodiments, the compound of Formula (III-a-2) is of Formula (III′-a-2), or a salt thereof.

In some embodiments of Formulae (III-a-1) and (III′-a-1), R⁵ is —OH. In some embodiments of Formulae (III-a-1) and (III′-a-1), R⁵ is —NH₂. In some embodiments of Formulae (III-a-2) and (III′-a-2), R⁵ is —OH. In some embodiments of Formulae (III-a-2) and (III′-a-2), R⁵ is —NH₂.

In some embodiments, the compound of Formula (III) is of Formula (III-b):

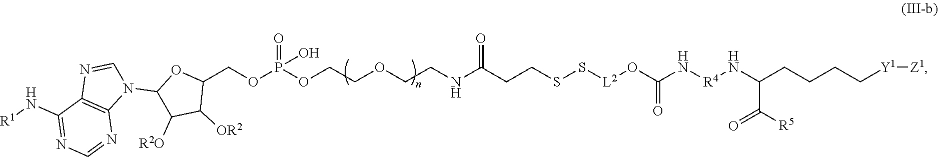

or a salt thereof.

In some embodiments, the compound of Formula (III-b) is of Formula (III′-b):

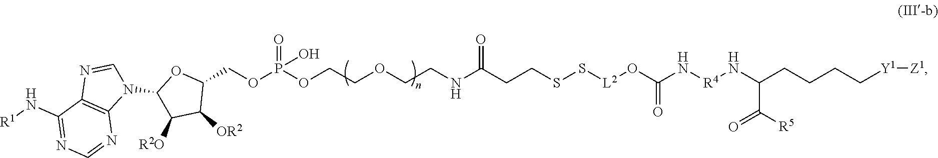

or a salt thereof.

In some embodiments of Formulae (III-b) and (III′-b), R⁵ is —OH. In some embodiments of Formulae (III-b) and (III′-b), R⁵ is —NH₂.

In some embodiments, the compound of Formula (III) is of Formula (III-c-1) or (III-c-2):

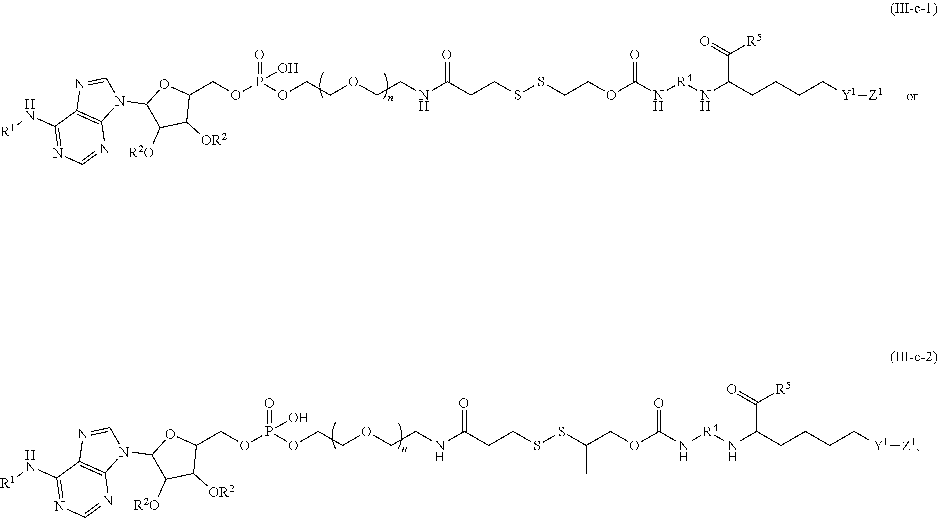

or a salt thereof.

In some embodiments, the compound of Formula (III) is of Formula (III-c-1), or a salt thereof. In some embodiments, the compound of Formula (III) is of Formula (III-c-2), or a salt thereof.

In some embodiments, the compound of Formula (III-c-1) or (III-c-2) is of Formula (III′-c) or (III′-c-2):

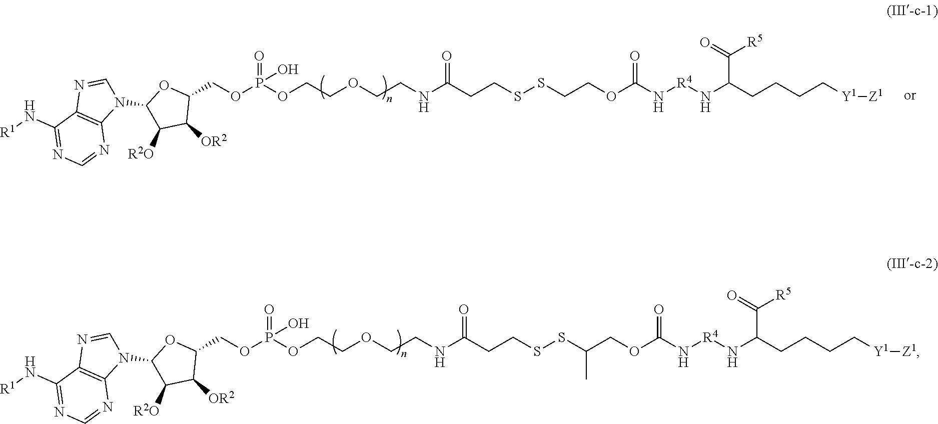

or a salt thereof.

In some embodiments, the compound of Formula (III-c-1) is of Formula (III′-c-1), or a salt thereof. In some embodiments, the compound of Formula (III-c-2) is of Formula (III′-c-2), or a salt thereof.

In some embodiments of Formulae (III-c-1) and (III′-c-1), R⁵ is —OH. In some embodiments of Formulae (III-c-1) and (III′-c-1), R⁵ is —NH₂. In some embodiments of Formulae (III-c-2) and (III′-c-2), R⁵ is —OH. In some embodiments of Formulae (III-c-2) and (III′-c-2), R⁵ is —NH₂.

In some embodiments, the compound of Formula (III) is of Formula (III-d):

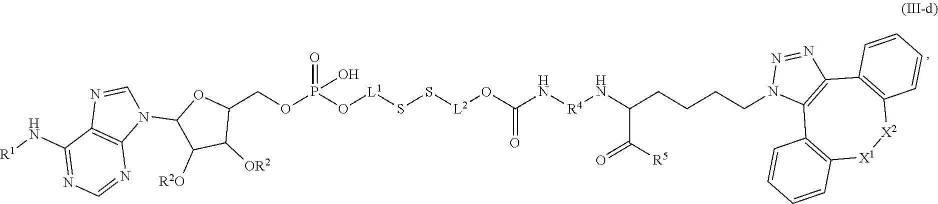

or a salt thereof, wherein one of X¹ and X² is CH and the other is N—Z¹.

In some embodiments, the compound of Formula (III-d) is of Formula (III-d-1):

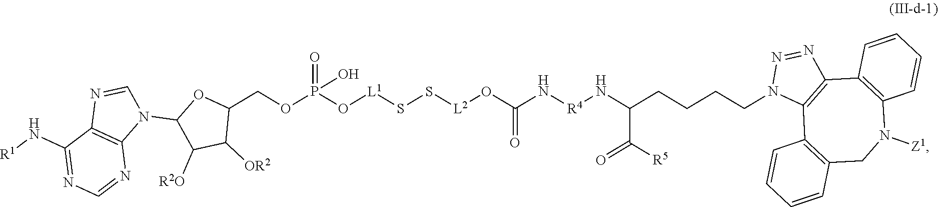

or a salt thereof.

In some embodiments, the compound of Formula (III-d) is of Formula (III-d-2):

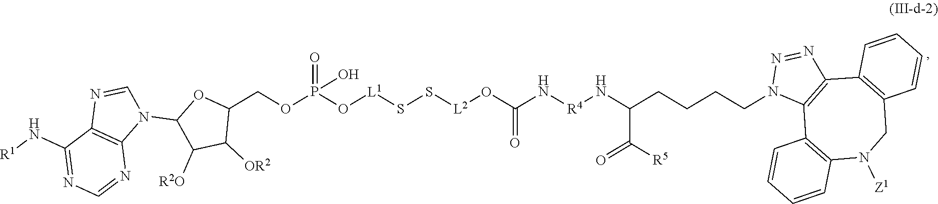

or a salt thereof.

In some embodiments, the compound of Formula (III-d) is of Formula (III′-d):

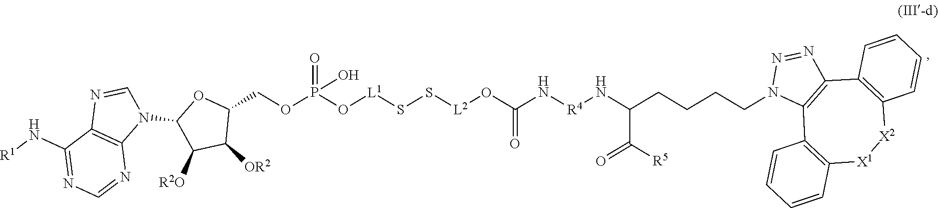

or a salt thereof.

In some embodiments, the compound of Formula (III′-d) is of Formula (III′-d-1):

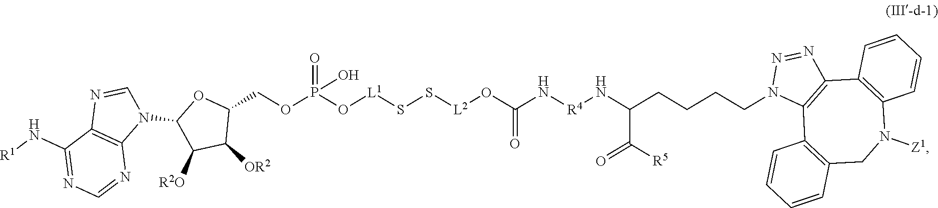

or a salt thereof.

In some embodiments, the compound of Formula (III′-d) is of Formula (III′-d-2):

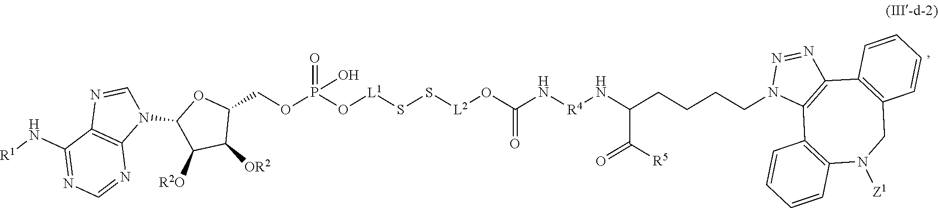

or a salt thereof.

In some embodiments of Formulae (III-d), (III-d-1), (III-d-2), (III′-d), (III′-d-1), and (III′-d-2), R⁵ is —OH. In some embodiments of Formulae (III-d), (III-d-1), (III-d-2), (III′-d), (III′-d-1), and (III′-d-2), R⁵ is —NH₂.

In some embodiments, the compound of Formula (III) is of Formula (III-e) or (III-ee):

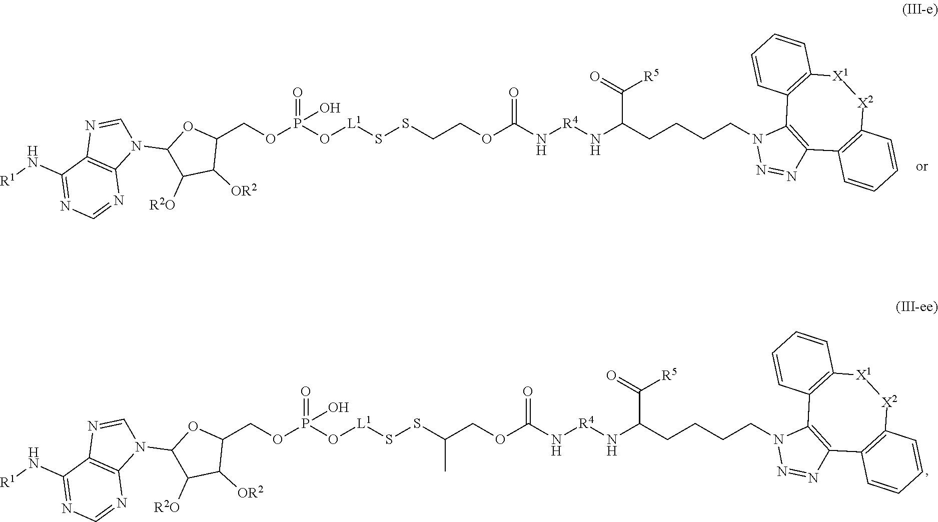

or a salt thereof, wherein one of X¹ and X² is CH and the other is N—Z¹.

In some embodiments, the compound of Formula (III) is of Formula (III-e), or a salt thereof. In some embodiments, the compound of Formula (III) is of Formula (III-ee), or a salt thereof.

In some embodiments, the compound of Formula (III-e) or (III-ee) is of Formula (III-e-1) or (III-ee-1):

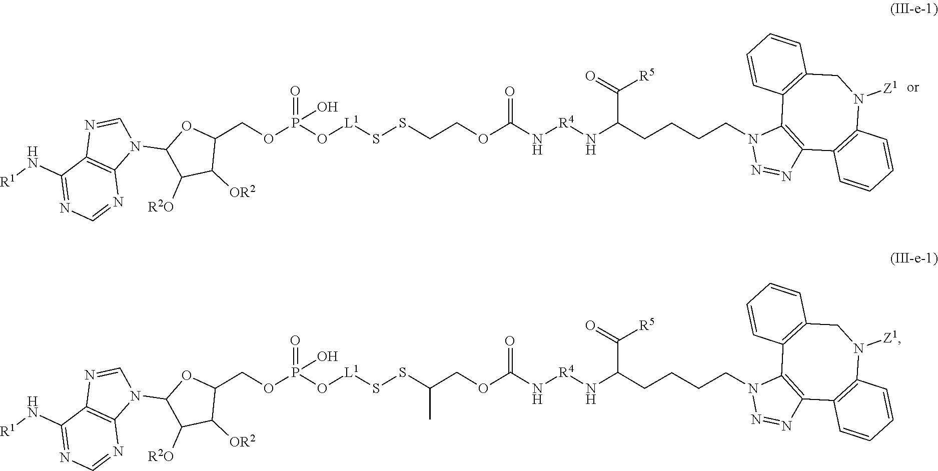

or a salt thereof.

In some embodiments, the compound of Formula (III-e) is of Formula (III-e-1), or a salt thereof. In some embodiments, the compound of Formula (III-ee) is of Formula (III-ee-1), or a salt thereof.

In some embodiments, the compound of Formula (III-e) or (III-ee) is of Formula (III-e-2) or (III-ee-2):

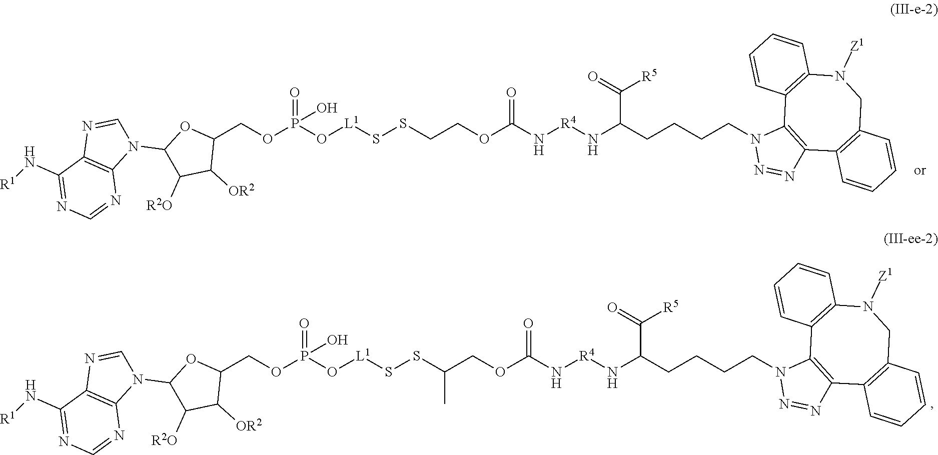

or a salt thereof.

In some embodiments, the compound of Formula (III-e) is of Formula (III-e-2), or a salt thereof. In some embodiments, the compound of Formula (III-ee) is of Formula (III-ee-2), or a salt thereof.

In some embodiments, the compound of Formula (III-e) or (III-ee) is of Formula (III′-e) or (III′-ee):

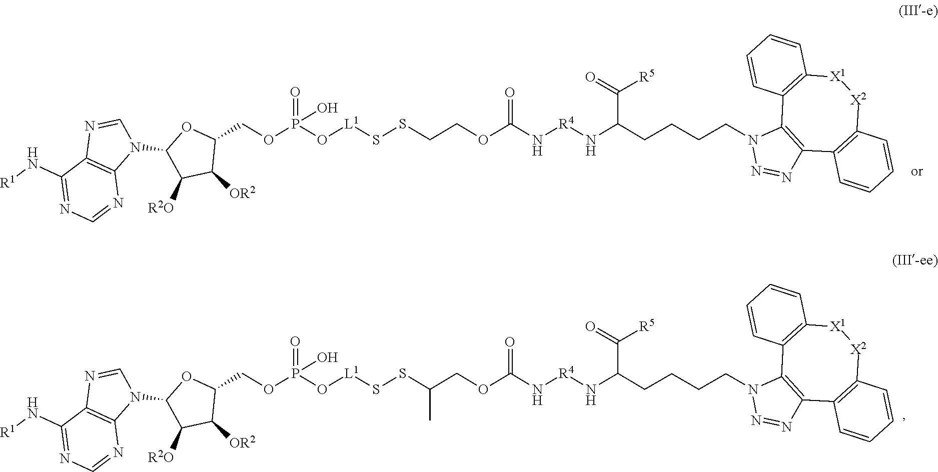

or a salt thereof.

In some embodiments, the compound of Formula (III-e) is of Formula (III′-e), or a salt thereof. In some embodiments, the compound of Formula (III-ee) is of Formula (III′-ee), or a salt thereof.

In some embodiments, the compound of Formula (III′-e) or (III′-ee) is of Formula (III′-e-1) or (III′-ee-1):

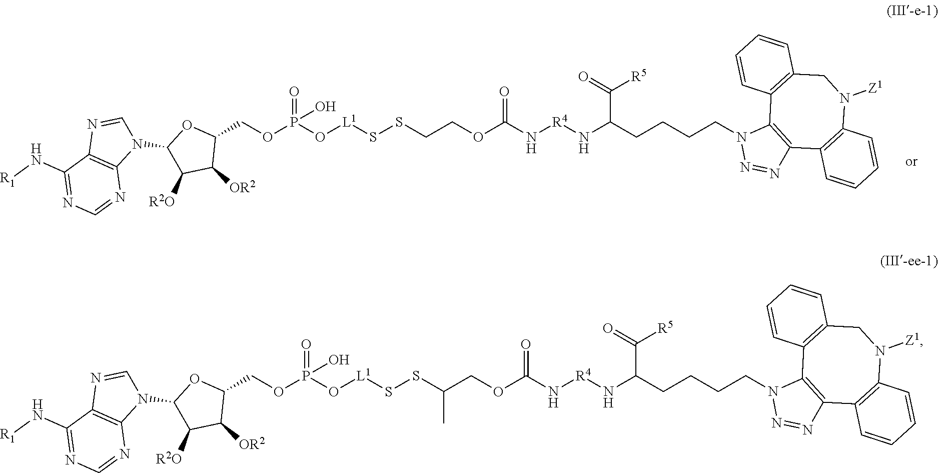

or a salt thereof.

In some embodiments, the compound of Formula (III′-e) is of Formula (III′-e-1), or a salt thereof. In some embodiments, the compound of Formula (III′-ee) is of Formula (III′-ee-1), or a salt thereof.

In some embodiments, the compound of Formula (III′-e) or (III′-ee) is of Formula (III′-e-2) or (III′-ee-2):

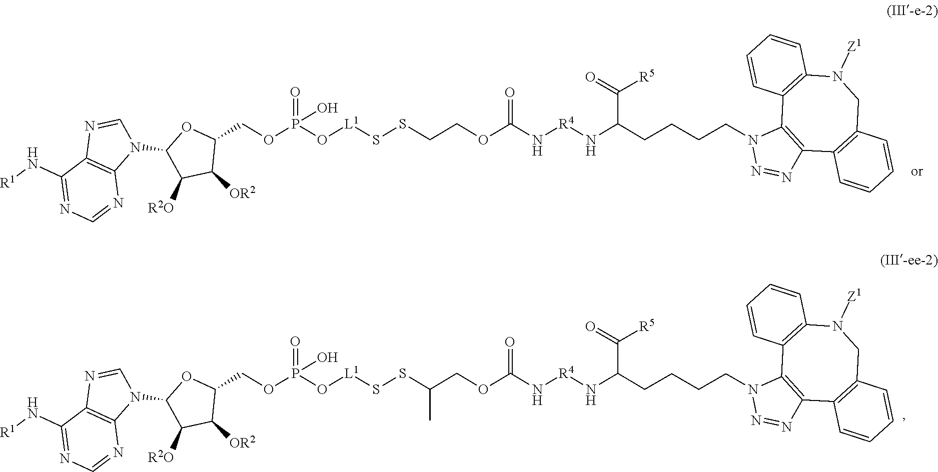

or a salt thereof.

In some embodiments, the compound of Formula (III′-e) is of Formula (III′-e-2), or a salt thereof. In some embodiments, the compound of Formula (III′-ee) is of Formula (III′-ee-2), or a salt thereof.

In some embodiments of Formulae (III-e), (III-e-1), (III-e-2), (III′-e), (III′-e-1), and (III′-e-2), R⁵ is —OH. In some embodiments of Formulae (III-e), (III-e-1), (III-e-2), (III′-e), (III′-e-1), and (III′-e-2), R⁵ is —NH₂. In some embodiments of Formulae (III-ee), (III-ee-1), (III-ee-2), (III′-ee), (III′-ee-1), and (III′-ee-2), R⁵ is —OH. In some embodiments of Formulae (III-ee), (III-ee-1), (III-ee-2), (III′-ee), (III′-ee-1), and (III′-ee-2), R⁵ is —NH₂.

In some embodiments, the compound of Formula (III) is of Formula (III-f):

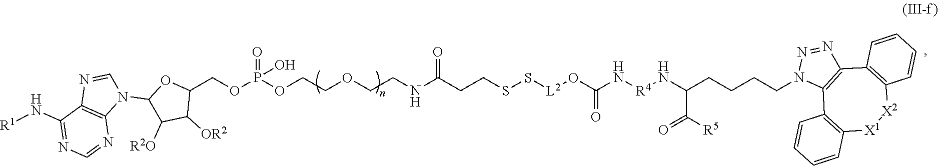

or a salt thereof, wherein one of X¹ and X² is CH and the other is N—Z¹.

In some embodiments, the compound of Formula (III-f) is of Formula (III-f-1):

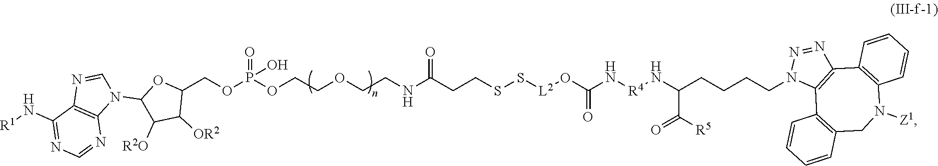

or a salt thereof.

In some embodiments, the compound of Formula (III-f) is of Formula (III-f-2):

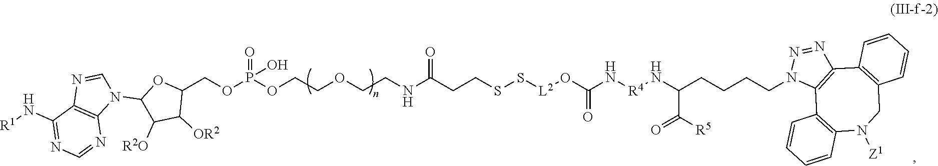

or a salt thereof.

In some embodiments, the compound of Formula (III-f) is of Formula (III′-f):

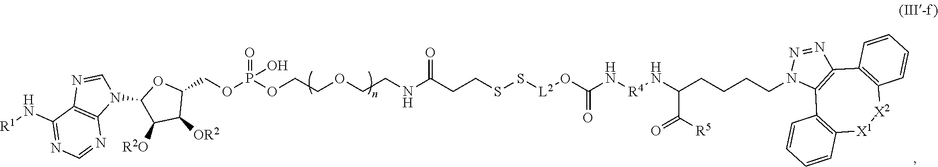

or a salt thereof.

In some embodiments, the compound of Formula (III′-f) is of Formula (III′-f-1):

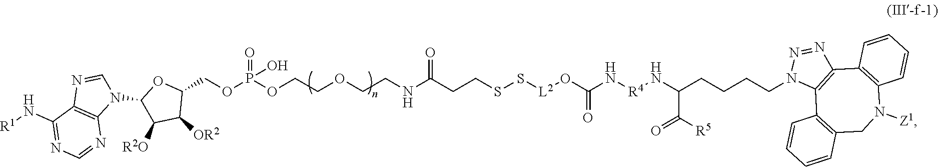

or a salt thereof.

In some embodiments, the compound of Formula (III′-f) is of Formula (III′-f-2):

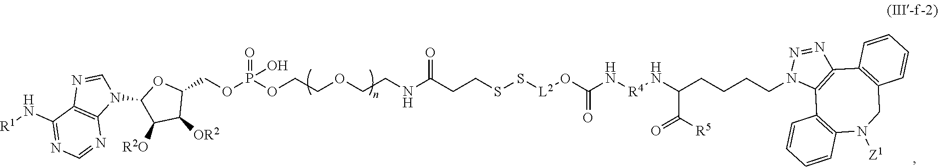

or a salt thereof.

In some embodiments of Formulae (III-f), (III-f-1), (II-f-2), (III′-f-1), and (III′-f-2), R⁵ is —OH. In some embodiments of Formulae (III-f), (ITT-f-1), (II-f-2), (III′-f-1), and (III′-f-2), R⁵ is —NH₂.

In some embodiments, the compound of Formula (III) is of Formulae (III-g) or (III-gg):

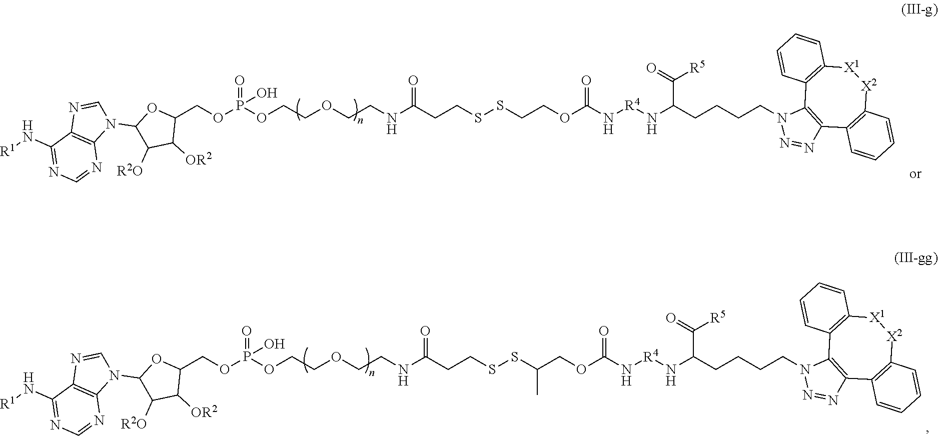

or a salt thereof, wherein one of X¹ and X² is CH and the other is N—Z¹.

In some embodiments, the compound of Formula (III) is of Formula (III-g), or a salt thereof. In some embodiments, the compound of Formula (III) is of Formula (III-gg), or a salt thereof.

In some embodiments, the compound of Formula (III-g) or (III-gg) is of Formula (III-g-1) or (III-gg-1):

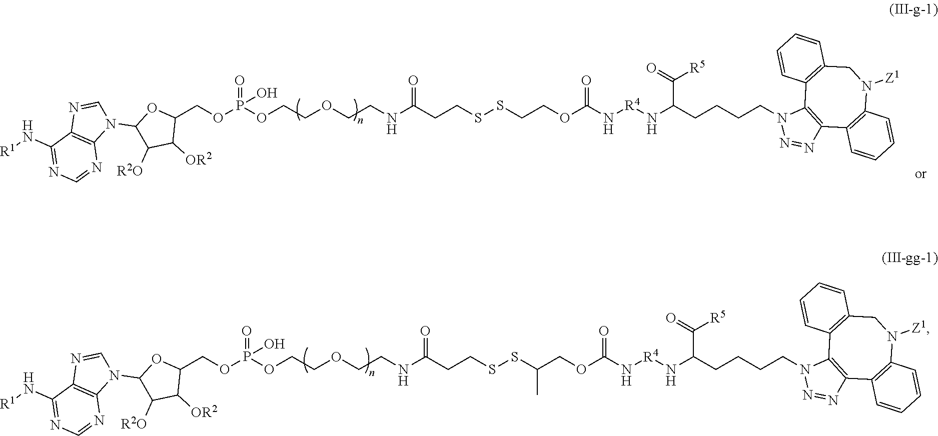

or a salt thereof.

In some embodiments, the compound of Formula (III-g) is of Formula (III-g-1), or a salt thereof. In some embodiments, the compound of Formula (III-gg) is of Formula (III-gg-1), or a salt thereof.

In some embodiments, the compound of Formula (III-g) or (III-gg) is of Formula (III-g-2) or (III-gg-2):

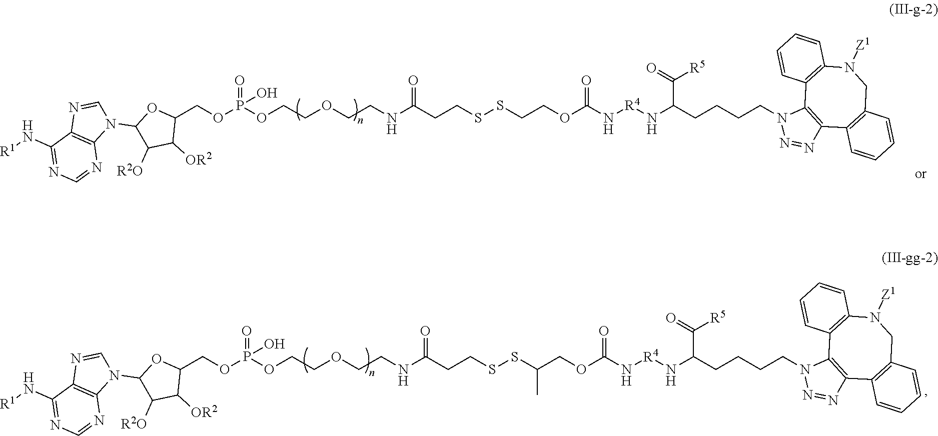

or a salt thereof.

In some embodiments, the compound of Formula (III-g) is of Formula (III-g-2), or a salt thereof. In some embodiments, the compound of Formula (III-gg) is of Formula (III-gg-2), or a salt thereof.

In some embodiments, the compound of Formula (III-g) or (III-gg) is of Formulae (III′-g) or (III′-gg):

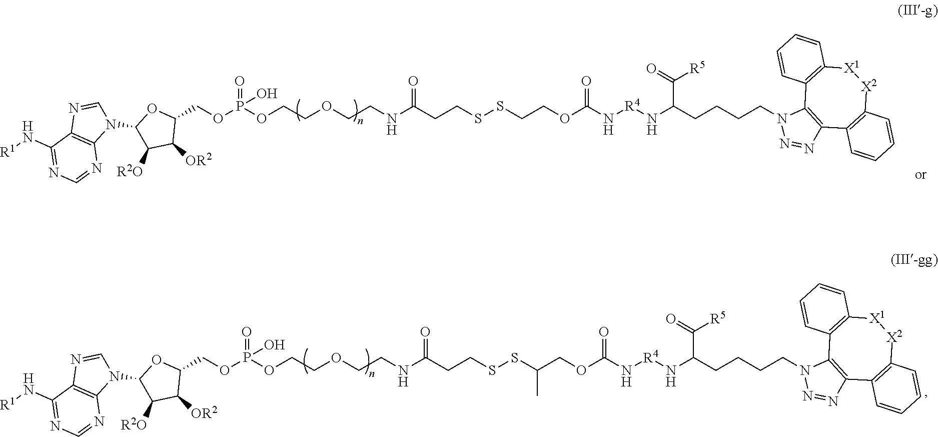

or a salt thereof.

In some embodiments, the compound of Formula (III-g) is of Formula (III′-g), or a salt thereof. In some embodiments, the compound of Formula (III-gg) is of Formula (III′-gg), or a salt thereof.

In some embodiments, the compound of Formula (III′-g) or (III′-gg) is of Formula (III′-g-1) or (III′-gg-1):

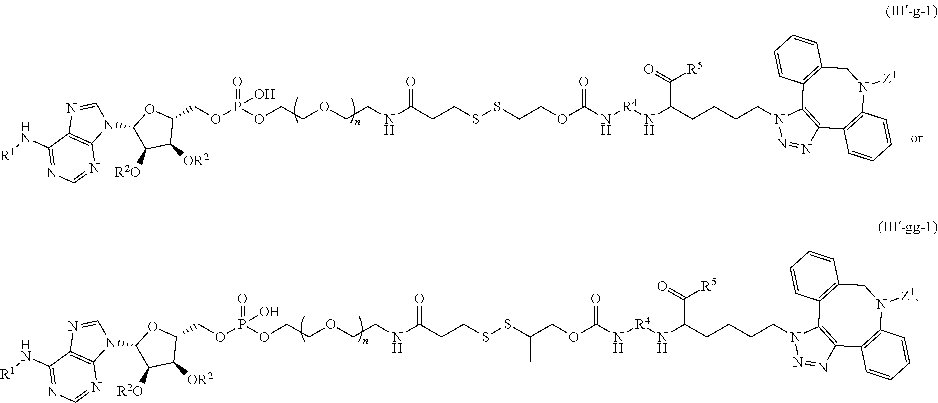

or a salt thereof.

In some embodiments, the compound of Formula (III′-g) is of Formula (III′-g-1), or a salt thereof. In some embodiments, the compound of Formula (III′-gg) is of Formula (III′-gg-1), or a salt thereof.

In some embodiments, the compound of Formula (III′-g) or (III′-gg) is of Formula (III′-g-2) or (III′-gg-2):

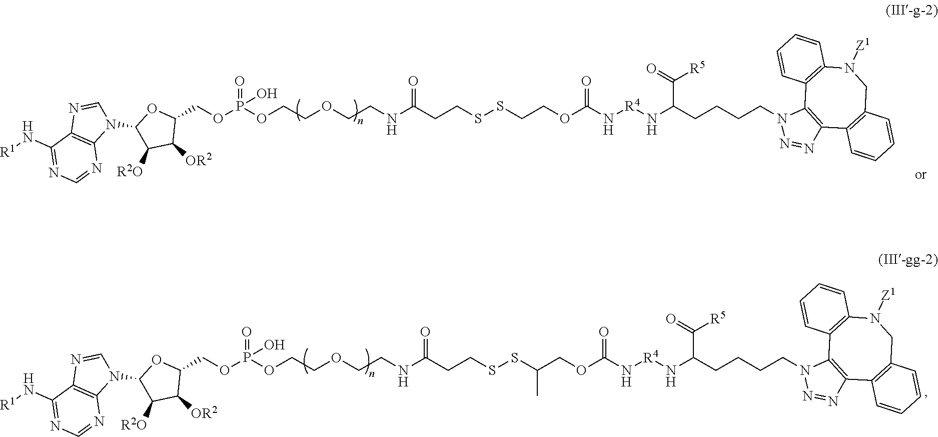

or a salt thereof.

In some embodiments, the compound of Formula (III′-g) is of Formula (III′-g-2), or a salt thereof. In some embodiments, the compound of Formula (III′-gg) is of Formula (III′-gg-2), or a salt thereof.

In some embodiments of Formulae (III-g), (III-g-1), (III-g-2), (III′-g), (III′-g-1), and (III′-g-2), R⁵ is —OH. In some embodiments of Formulae (III-g), (III-g-1), (III-g-2), (III′-g), (III′-g-1), and (III′-g-2), R⁵ is —NH₂. In some embodiments of Formulae (III-gg), (III-gg-1), (III-gg-2), (III′-gg), (III′-gg-1), and (III′-gg-2), R⁵ is —OH. In some embodiments of Formulae (III-gg), (III-gg-1), (III-gg-2), (III′-gg), (III′-gg-1), and (III′-gg-2), R⁵ is —NH₂.

### Methods of Preparation

In another aspect, the present disclosure provides a method of preparing a compound of Formula (I):

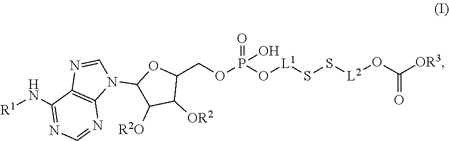

or a salt thereof, comprising coupling a compound of Formula (IV):

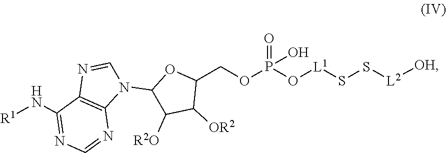

or a salt thereof, with a compound of Formula (V):

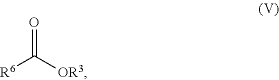

or a salt thereof, under suitable conditions to obtain the compound of Formula (I), or salt thereof, wherein:
R¹ is a solid support;
each instance of R² is independently hydrogen or an oxygen protecting group;
R³ is optionally substituted alkyl, optionally substituted heterocyclyl, optionally substituted aryl, or optionally substituted heteroaryl;
R⁶ is halogen or —OR³;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof; and
L² is optionally substituted C₂ alkylene.

As generally described herein, R⁶ is halogen or —OR³.

In some embodiments, R⁶ is halogen. In some embodiments, R⁶ is chloro.

In some embodiments, R⁶ is —OR³. In some embodiments, R⁶ is —OR³, wherein each instance of R³ in the compound of Formula (V), or salt thereof, is independently optionally substituted aryl or optionally substituted heteroaryl.

In some embodiments, R⁶ is R³ is

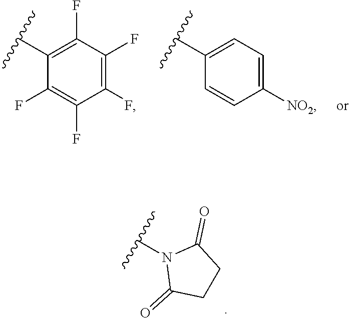

As generally described herein, R⁷ is an oxygen protecting group.

In another aspect, the present disclosure provides a method of preparing a compound of Formula (II):

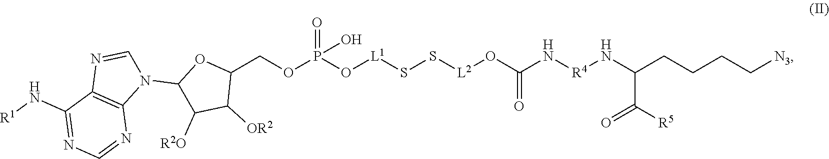

or a salt thereof, comprising coupling a compound of Formula (I):

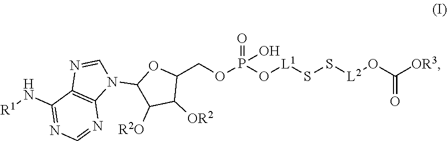

or a salt thereof, with a compound of Formula (XII):

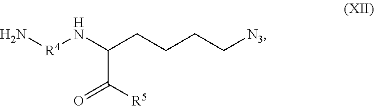

or a salt thereof, under suitable conditions to obtain the compound of Formula (II), or salt thereof, wherein:
R¹ is a solid support;
each instance of R² is independently hydrogen or an oxygen protecting group;
R³ is optionally substituted alkyl, optionally substituted heterocyclyl, optionally substituted aryl, or optionally substituted heteroaryl;
R⁴ is a peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof; and
L² is optionally substituted C₂ alkylene.

In another aspect, the present disclosure provides a method of preparing a compound of Formula (III-d):

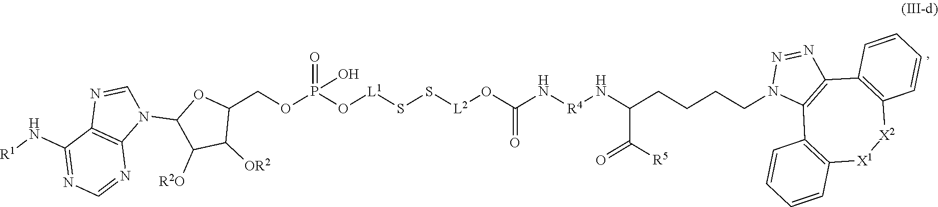

or a salt thereof, comprising coupling a compound of Formula (II):

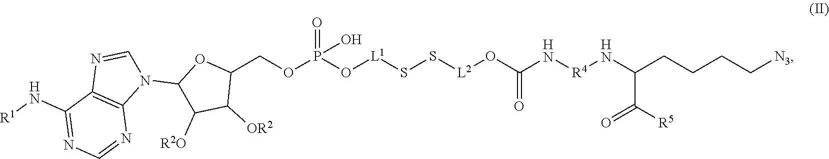

or a salt thereof, with a compound of Formula (XIII):

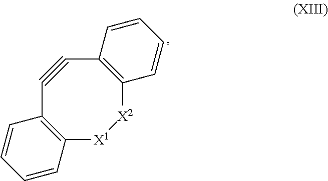

or a salt thereof, under suitable conditions to obtain the compound of Formula (III-d), or salt thereof, wherein:
R¹ is a solid support;
each instance of R² is independently hydrogen or an oxygen protecting group;
R⁴ is a peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof;
L² is optionally substituted C₂ alkylene; and
one of X¹ and X² is CH and the other is N—Z¹, wherein Z¹ comprises an oligonucleotide.

In certain embodiments, the method of preparing a compound of Formula (III) (e.g., a compound of Formula (III-d)), or salt thereof, comprises a “click chemistry” reaction (e.g., a Huisgen alkyne-azide cycloaddition).

Various conditions are suitable for the reaction of a compound of Formula (XIII), or a salt thereof, with a compound of Formula (II), or a salt thereof, to produce a compound of Formula (III) (e.g., a compound of Formula (III-d)), or a salt thereof, and one of ordinary skill in the art will readily understand that such conditions may be substituted and still be compatible using the methods disclosed herein. For example, such a reaction may be performed in the presence of a solvent. Suitable solvents for performing this reaction include, but are not limited to, water, aqueous NaHCO₃ (e.g., 0.1 M NaHCO₃), dimethylsulfoxide, dimethylformamide, acetonitrile, and combinations thereof. In some embodiments, the reaction of a compound of Formula (XIII), or a salt thereof, with a compound of Formula (II), or a salt thereof, is performed in water, aqueous NaHCO₃ (e.g., 0.1 M NaHCO₃), or a combination thereof.

The reaction of a compound of Formula (XIII), or a salt thereof, with a compound of Formula (II), or a salt thereof, to produce a compound of Formula (III) (e.g., a compound of Formula (III-d)), or a salt thereof, may be also performed for varying amounts of time. The reaction may comprise a reaction time of approximately 5 minutes, approximately 10 minutes, approximately 15 minutes, approximately 20 minutes, approximately 25 minutes, approximately 30 minutes, approximately 35 minutes, approximately 40 minutes, approximately 45 minutes, approximately 50 minutes, approximately 55 minutes, approximately 1 hour, approximately 2 hours, approximately 3 hours, approximately 4 hours, or approximately 5 hours. In some embodiments, the reaction of a compound of Formula (XIII), or a salt thereof, with a compound of Formula (II), or a salt thereof, is performed for a reaction time of approximately 20 minutes. In some embodiments, the reaction of a compound of Formula (XIII), or a salt thereof, with a compound of Formula (II), or a salt thereof, is performed for a reaction time of approximately 40 minutes.

The reaction of a compound of Formula (XIII), or a salt thereof, with a compound of Formula (II), or a salt thereof, to produce a compound of Formula (III) (e.g., a compound of Formula (III-d)), or a salt thereof, may be performed at various temperatures. For example, the reaction of a compound of Formula (XIII), or a salt thereof, with a compound of Formula (II), or a salt thereof, may comprise a reaction temperature of approximately 15° C., approximately 20° C., approximately 25° C., approximately 30° C., approximately 35° C., approximately 37° C., approximately 40° C., approximately 45° C., or approximately 50° C. In certain embodiments, the reaction temperature may be in a range of approximately 15° C. to approximately 50° C., approximately 15° C. to approximately 45° C., approximately 15° C. to approximately 40° C., approximately 15° C. to approximately 35° C., approximately 15° C. to approximately 30° C., approximately 15° C. to approximately 25° C., approximately 15° C. to approximately 20° C., approximately 35° C. to approximately 45° C., or approximately 35° C. to approximately 40° C. In certain embodiments, the reaction temperature is approximately 20° C. In certain embodiments, the reaction temperature is approximately 25° C. In certain embodiments, the reaction temperature is room temperature.

The reaction of a compound of Formula (XIII), or a salt thereof, with a compound of Formula (II), or a salt thereof, to produce a compound of Formula (III) (e.g., a compound of Formula (III-d)), or a salt thereof, may be performed with a reducing agent. Suitable reducing agents for performing this reaction include, but are not limited to, sodium ascorbate, hydroxylamine, triethylamine, diisopropylethylamine, and combinations thereof. In some embodiments, the reaction of a compound of Formula (XIII), or a salt thereof, with a compound of Formula (II), or a salt thereof, is performed with sodium ascorbate as the reducing agent. In some embodiments, the reaction of a compound of Formula (XIII), or a salt thereof, with a compound of Formula (II), or a salt thereof, is performed with sodium ascorbate as the reducing agent, wherein the sodium ascorbate is added in one portion. In some embodiments, the reaction of a compound of Formula (XIII), or a salt thereof, with a compound of Formula (II), or a salt thereof, is performed with sodium ascorbate as the reducing agent, wherein the sodium ascorbate is added in two or more portions. In some embodiments, the reaction of a compound of Formula (XIII), or a salt thereof, with a compound of Formula (II), or a salt thereof, is performed with sodium ascorbate as the reducing agent, wherein the sodium ascorbate is added in two portions.

The reaction of a compound of Formula (XIII), or a salt thereof, with a compound of Formula (II), or a salt thereof, to produce a compound of Formula (III) (e.g., a compound of Formula (III-d)), or a salt thereof, may be performed with a copper (II) compound. Suitable copper (II) compounds for performing this reaction include, but are not limited to, copper (II) tris(3-hydroxypropyltriazolylmethyl)amine (Cu(THPTA)), copper (II) sulfate, copper (II) acetate, and combinations thereof. In some embodiments, the reaction of a compound of Formula (XIII), or a salt thereof, with a compound of Formula (II), or a salt thereof, is performed with Cu(THPTA) as the copper (II) compound. In some embodiments, the reaction of a compound of Formula (XIII), or a salt thereof, with a compound of Formula (II), or a salt thereof, to produce a compound of Formula (III) (e.g., a compound of Formula (III-d)), or a salt thereof, may be performed with a copper (II) compound and a ligand. Suitable ligands for performing this reaction include, but are not limited to, tris(3-hydroxypropyltriazolylmethyl)amine, aminoguanidine, tris[(1-benzyl-1H-1,2,3-triazol-4-yl)methyl]amine, and combinations thereof. In some embodiments, the reaction of a compound of Formula (XIII), or a salt thereof, with a compound of Formula (II), or a salt thereof, is performed with tris(3-hydroxypropyltriazolylmethyl)amine as the ligand. The reaction of a compound of Formula (XIII), or a salt thereof, with a compound of Formula (II), or a salt thereof, to produce a compound of Formula (III) (e.g., a compound of Formula (III-d)), or a salt thereof, may be performed with a copper (I) compound. Suitable copper (I) compounds include, but are not limited to, copper (I) iodide, copper (I) bromide, copper (I) chloride, copper (I) thiophene-2-carboxylate (CuTC), tetrakis(acetonitrile)copper(I) hexafluorophosphate, tetrakis(acetonitrile)copper(I) tetrafluoroborate, and combinations thereof.

The reaction of a compound of Formula (XIII), or a salt thereof, with a compound of Formula (II), or a salt thereof, to produce a compound of Formula (III) (e.g., a compound of Formula (III-d)), or a salt thereof, may be performed with various molar ratios of the reagents to one another. For example, the ratio of the compound of Formula (XIII), or a salt thereof, to the compound of Formula (II), or a salt thereof, may be approximately 1:1, approximately 1:2, approximately 1:3, approximately 1:4, approximately 1:5, approximately 1:6, approximately 1:7, approximately 1:8, approximately 1:9, or approximately 1:10. In certain embodiments, a ratio greater than approximately 1:10 may be used. In certain embodiments, a ratio of the compound of Formula (XIII), or a salt thereof, to the compound of Formula (II), or a salt thereof, of approximately 1:4 is used. In certain embodiments, a ratio of the compound of Formula (XIII), or a salt thereof, to the compound of Formula (II), or a salt thereof, of approximately 1:3 is used. In certain embodiments, a ratio of the compound of Formula (XIII), or a salt thereof, to the compound of Formula (II), or a salt thereof, of approximately 1:3.3 is used. For example, the ratio of the compound of Formula (XIII), or a salt thereof, to the reducing agent may be approximately 1:1, approximately 1:10, approximately 1:20, approximately 1:30, approximately 1:40, approximately 1:50, approximately 1:60, approximately 1:70, approximately 1:80, approximately 1:90, approximately 1:100, approximately 1:120, or approximately 1:150. In certain embodiments, a ratio of the compound of Formula (XIII), or a salt thereof, to the reducing agent of approximately 1:40 is used. In certain embodiments, a ratio of the compound of Formula (XIII), or a salt thereof, to the reducing agent of approximately 1:80 is used. In certain embodiments, a ratio of the compound of Formula (XIII), or a salt thereof, to the reducing agent of approximately 1:40 is used, wherein the reducing agent is added in two or more portions. In certain embodiments, a ratio of the compound of Formula (XIII), or a salt thereof, to the reducing agent of approximately 1:80 is used, wherein the reducing agent is added in two or more portions. For example, the ratio of the compound of Formula (XIII), or a salt thereof, to the copper (I) compound may be approximately 1:1, approximately 1:0.9, approximately 1:0.8, approximately 1:0.7, approximately 1:0.6, approximately 1:0.5, approximately 1:0.4, approximately 1:0.3, approximately 1:0.0, or approximately 1:0.1. In certain embodiments, a ratio of the compound of Formula (XIII), or a salt thereof, to the copper (I) compound of greater than approximately 1:1 may be used. In certain embodiments, a ratio of the compound of Formula (XIII), or a salt thereof, to the copper (I) compound of approximately 1:0.8 is used.

Any reaction described herein may further comprise a work up, which can consist of a single step or multiple steps. Various steps are suitable for the work up, and one of ordinary skill in the art will readily understand that such steps may be substituted and still be compatible using the methods disclosed herein. In some embodiments, a reaction may be concentrated under reduced pressure using evaporation or lyophilization. In some embodiments, a reaction may be purified using silica gel chromatography. In some embodiments, a reaction may be subjected to liquid-liquid extraction. In some embodiments, a reaction may be quenched. In some embodiments, a reaction may be quenched with a base (e.g. EDTA).

In some embodiments, R¹ is a polymeric support. In some embodiments, R¹ is a polymeric support of Oligo-Affinity Support (PS) (5′-Dimethoxytrityl-Adenosine-2′,3′-diacetate-N-Linked-Polymeric Support), available from Glen Research (Catalog Number 26-4001).

In some embodiments, at least one instance of R² is hydrogen.

In some embodiments, at least one instance of R² is an oxygen protecting group. In some embodiments, at least one instance of R² is —C(═O)R^{2a}, wherein R^{2a} is optionally substituted alkyl or optionally substituted aryl.

In some embodiments, at least one instance of R² is —C(═O)R^{2a}, wherein R^{2a} is optionally substituted C₁₋₃ alkyl. In some embodiments, at least one instance of R² is —C(═O)R^{2a}, wherein R^{2a} is substituted C₁₋₃ alkyl. In some embodiments, at least one instance of R² is —C(═O)R^{2a}, wherein R^{2a} is unsubstituted C₁₋₃ alkyl.

In some embodiments, at least one instance of R² is —C(═O)CH₃.

In some embodiments, each instance of R² is independently hydrogen.

In some embodiments, each instance of R² is independently an oxygen protecting group. In some embodiments, each instance of R² is independently —C(═O)R^{2a}, wherein R^{2a} is optionally substituted alkyl or optionally substituted aryl.

In some embodiments, each instance of R² is independently —C(═O)R^{2a}, wherein R^{2a} is optionally substituted C₁₋₃ alkyl. In some embodiments, each instance of R² is independently —C(═O)R^{2a}, wherein R^{2a} is substituted C₁₋₃ alkyl. In some embodiments, each instance of R² is independently —C(═O)R^{2a}, wherein R^{2a} is unsubstituted C₁₋₃ alkyl.

In some embodiments, each instance of R² is independently —C(═O)CH₃.

In some embodiments, R³ is optionally substituted heterocyclyl or optionally substituted aryl. In some embodiments, R³ is substituted heterocyclyl or substituted aryl. In some embodiments, R³ is substituted 5-6 membered heterocyclyl or substituted phenyl. In some embodiments, R³ is

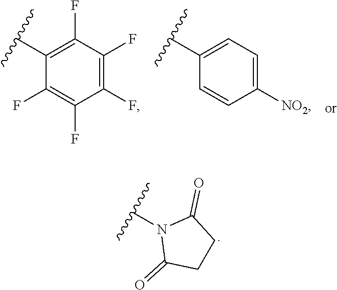

In some embodiments, R³ is optionally substituted heterocyclyl. In some embodiments, R³ is substituted heterocyclyl. In some embodiments, R³ is unsubstituted heterocyclyl.

In some embodiments, R³ is optionally substituted 5-6 membered heterocyclyl. In some embodiments, R³ is substituted 5-6 membered heterocyclyl. In some embodiments, R³ is unsubstituted 5-6 membered heterocyclyl. In some embodiments, R³ is substituted 5-6 membered heterocyclyl containing 1 ring N atom.

In some embodiments, R³ is optionally substituted 5 membered heterocyclyl. In some embodiments, R³ is substituted 5 membered heterocyclyl. In some embodiments, R³ is unsubstituted 5 membered heterocyclyl. In some embodiments, R³ is substituted 5 membered heterocyclyl containing 1 ring N atom.

In some embodiments, R³ is optionally substituted aryl. In some embodiments, R³ is substituted aryl. In some embodiments, R³ is unsubstituted aryl.

In some embodiments, R³ is optionally substituted phenyl. In some embodiments, R³ is substituted phenyl. In some embodiments, R³ is unsubstituted phenyl.

In some embodiments, R³ is phenyl substituted with one or more substituents selected from halogen, optionally substituted alkyl, optionally substituted heteroalkyl, —CN, —OR^{A}, —N(R^{A})₂, and —NO₂. In some embodiments, R³ is phenyl substituted with one or more substituents selected from halogen and —NO₂. In some embodiments, R³ is phenyl substituted with one or more substituents selected from fluoro, chloro, bromo, iodo, and —NO₂. In some embodiments, R³ is phenyl substituted with one or more substituents selected from fluoro, chloro, bromo, and —NO₂. In some embodiments, R³ is phenyl substituted with one or more substituents selected from fluoro, chloro, and —NO₂. In some embodiments, R³ is phenyl substituted with one or more substituents selected from fluoro and —NO₂. In some embodiments, R³ is phenyl substituted with at least one halogen or —NO₂. In some embodiments, R³ is phenyl substituted with at least one fluoro or —NO₂. In some embodiments, R³ is phenyl substituted with at least one fluoro. In some embodiments, R³ is phenyl substituted with at least one —NO₂.

In some embodiments, R⁴ comprises one or more amino acids selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, R⁴ comprises an amino acid comprising a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, R⁴ comprises an amino acid comprising an arginine post-translational modification. For example, as described herein, arginine modifications include symmetric dimethylarginine (SDMA), asymmetric dimethylarginine (ADMA), and citrullinated arginine.

In some embodiments, R⁴ comprises an amino acid comprising a phosphorylated side chain. In some embodiments, R⁴ comprises an amino acid comprising phosphorylated threonine (e.g., phospho-threonine). In some embodiments, R⁴ comprises an amino acid comprising phosphorylated tyrosine (e.g., phospho-tyrosine). In some embodiments, R⁴ comprises an amino acid comprising phosphorylated serine (e.g., phospho-serine).

In some embodiments, R⁴ comprises an amino acid comprising a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, R⁴ comprises an amino acid comprising an oxidative modification. In some embodiments, R⁴ comprises an amino acid comprising a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, R⁴ comprises an amino acid comprising a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, R⁵ is —OH. In some embodiments, R⁵ is —NH₂.

In some embodiments, L¹ comprises optionally substituted alkylene, optionally substituted heteroalkylene, or a combination thereof.

In some embodiments, L¹ comprises optionally substituted alkylene. In some embodiments, L¹ comprises optionally substituted C₁-C₁₀₀ alkylene. In some embodiments, L¹ comprises optionally substituted C₁-C₇₅ alkylene. In some embodiments, L¹ comprises optionally substituted C₁-C₅₀ alkylene. In some embodiments, L¹ comprises optionally substituted C₁-C₂₅ alkylene. In some embodiments, L¹ comprises optionally substituted C₁-C₁₂ alkylene. In some embodiments, L¹ comprises optionally substituted C₁-C₆ alkylene. In some embodiments, L¹ comprises optionally substituted C₁-C₃ alkylene. In some embodiments, L¹ comprises optionally substituted linear alkylene. In some embodiments, L¹ comprises optionally substituted linear C₁-C₁₂ alkylene. In some embodiments, L¹ comprises optionally substituted linear C₁-C₆ alkylene. In some embodiments, L¹ comprises optionally substituted linear C₁-C₃ alkylene.

In some embodiments, L¹ comprises substituted alkylene. In some embodiments, L¹ comprises substituted C₁-C₁₀₀ alkylene. In some embodiments, L¹ comprises substituted C₁-C₇₅ alkylene. In some embodiments, L¹ comprises substituted C₁-C₅₀ alkylene. In some embodiments, L¹ comprises substituted C₁-C₂₅ alkylene. In some embodiments, L¹ comprises substituted C₁-C₁₂ alkylene. In some embodiments, L¹ comprises substituted C₁-C₆ alkylene. In some embodiments, L¹ comprises substituted C₁-C₃ alkylene. In some embodiments, L¹ comprises substituted linear alkylene. In some embodiments, L¹ comprises substituted linear C₁-C₁₂ alkylene. In some embodiments, L¹ comprises substituted linear C₁-C₆ alkylene. In some embodiments, L¹ comprises substituted linear C₁-C₃ alkylene.

In some embodiments, L¹ comprises unsubstituted alkylene. In some embodiments, L¹ comprises unsubstituted C₁-C₁₀₀ alkylene. In some embodiments, L¹ comprises unsubstituted C₁-C₇₅ alkylene. In some embodiments, L¹ comprises unsubstituted C₁-C₅₀ alkylene. In some embodiments, L¹ comprises unsubstituted C₁-C₂₅ alkylene. In some embodiments, L¹ comprises unsubstituted C₁-C₁₂ alkylene. In some embodiments, L¹ comprises unsubstituted C₁-C₆ alkylene. In some embodiments, L¹ comprises unsubstituted C₁-C₃ alkylene. In some embodiments, L¹ comprises unsubstituted linear alkylene. In some embodiments, L¹ comprises unsubstituted linear C₁-C₁₂ alkylene. In some embodiments, L¹ comprises unsubstituted linear C₁-C₆ alkylene. In some embodiments, L¹ comprises unsubstituted linear C₁-C₃ alkylene.

In some embodiments, L¹ comprises methylene, ethylene, n-propylene, n-butylene, n-pentylene, or n-hexylene. In some embodiments, L¹ comprises methylene, ethylene, or n-propylene. In some embodiments, L¹ comprises ethylene.

In some embodiments, L¹ comprises optionally substituted heteroalkylene. In some embodiments, L¹ comprises optionally substituted C₁-C₁₀₀ heteroalkylene. In some embodiments, L¹ comprises optionally substituted C₁-C₇₅ heteroalkylene. In some embodiments, L¹ comprises optionally substituted C₁-C₅₀ heteroalkylene. In some embodiments, L¹ comprises optionally substituted C₁-C₂₅ heteroalkylene. In some embodiments, L¹ comprises optionally substituted C₁-C₁₂ heteroalkylene. In some embodiments, L¹ comprises optionally substituted C₁-C₆ heteroalkylene. In some embodiments, L¹ comprises optionally substituted C₁-C₃ heteroalkylene. In some embodiments, L¹ comprises optionally substituted linear heteroalkylene. In some embodiments, L¹ comprises optionally substituted linear C₁-C₁₂ heteroalkylene. In some embodiments, L¹ comprises optionally substituted linear C₁-C₆ heteroalkylene. In some embodiments, L¹ comprises optionally substituted linear C₁-C₃ heteroalkylene.

In some embodiments, L¹ comprises substituted heteroalkylene. In some embodiments, L¹ comprises substituted C₁-C₁₀₀ heteroalkylene. In some embodiments, L¹ comprises substituted C₁-C₇₅ heteroalkylene. In some embodiments, L¹ comprises substituted C₁-C₅₀ heteroalkylene. In some embodiments, L¹ comprises substituted C₁-C₂₅ heteroalkylene. In some embodiments, L¹ comprises substituted C₁-C₁₂ heteroalkylene. In some embodiments, L¹ comprises substituted C₁-C₆ heteroalkylene. In some embodiments, L¹ comprises substituted C₁-C₃ heteroalkylene. In some embodiments, L¹ comprises substituted linear heteroalkylene. In some embodiments, L¹ comprises substituted linear C₁-C₁₂ heteroalkylene. In some embodiments, L¹ comprises substituted linear C₁-C₆ heteroalkylene. In some embodiments, L¹ comprises substituted linear C₁-C₃ heteroalkylene.

In some embodiments, L¹ comprises unsubstituted heteroalkylene. In some embodiments, L¹ comprises unsubstituted C₁-C₁₀₀ heteroalkylene. In some embodiments, L¹ comprises unsubstituted C₁-C₇₅ heteroalkylene. In some embodiments, L¹ comprises unsubstituted C₁-C₅₀ heteroalkylene. In some embodiments, L¹ comprises unsubstituted C₁-C₂₅ heteroalkylene. In some embodiments, L¹ comprises unsubstituted C₁-C₁₂ heteroalkylene. In some embodiments, L¹ comprises unsubstituted C₁-C₆ heteroalkylene. In some embodiments, L¹ comprises unsubstituted C₁-C₃ heteroalkylene. In some embodiments, L¹ comprises unsubstituted linear heteroalkylene. In some embodiments, L¹ comprises unsubstituted linear C₁-C₁₂ heteroalkylene. In some embodiments, L¹ comprises unsubstituted linear C₁-C₆ heteroalkylene. In some embodiments, L¹ comprises unsubstituted linear C₁-C₃ heteroalkylene.

In some embodiments, L¹ comprises

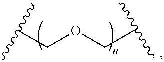

wherein n is an integer between 0 and 30, inclusive. In some embodiments, L¹ comprises —NHC(═O)— or —C(═O)NH—. In some embodiments, L¹ is

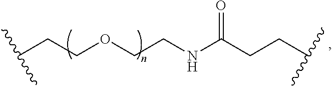

wherein n is an integer between 0 and 30, inclusive.

In some embodiments, L¹ comprises

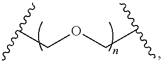

wherein n is an integer between 1 and 30, inclusive. In some embodiments, L¹ comprises

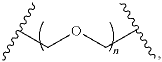

wherein n is an integer between 1 and 10, inclusive. In some embodiments, L¹ comprises

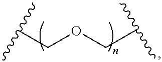

wherein n is an integer between 1 and 5, inclusive.

In some embodiments, L¹ comprises

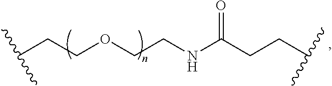

wherein n is an integer between 1 and 30, inclusive. In some embodiments, L¹ comprises

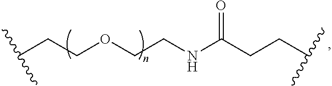

wherein n is an integer between 1 and 10, inclusive. In some embodiments, L¹ comprises

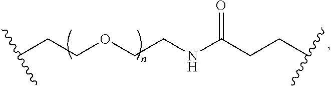

wherein n is an integer between 1 and 5, inclusive.

In some embodiments, L¹ is

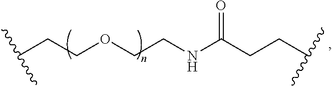

wherein n is an integer between 1 and 30, inclusive. In some embodiments, L¹ is

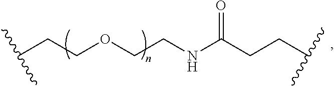

wherein n is an integer between 1 and 10, inclusive. In some embodiments, L¹ is

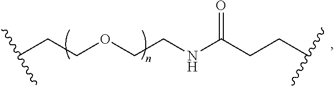

wherein n is an integer between 1 and 5, inclusive.

In some embodiments, L² is C₂ alkylene substituted with one or more substituents selected from halogen, optionally substituted alkyl, optionally substituted heteroalkyl, —OR^{A}, —N(R^{A})₂, and ═O. In some embodiments, L² is C₂ alkylene substituted with optionally substituted C₁₋₃ alkyl. In some embodiments, L² is C₂ alkylene substituted with unsubstituted C₁₋₃ alkyl. In some embodiments, L² is C₂ alkylene substituted with methyl, ethyl, n-propyl, or isopropyl. In some embodiments, L² is C₂ alkylene substituted with methyl.

In some embodiments, L² is unsubstituted C₂ alkylene (i.e., ethylene).

In some embodiments, L² is

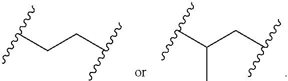

In some embodiments, the compound of Formula (I) is of Formula (I-a), or a salt thereof. In some embodiments, the compound of Formula (I-a) is of Formula (I-a-1), or a salt thereof. In some embodiments, the compound of Formula (I-a) is of Formula (I-a-2), or a salt thereof. In some embodiments, the compound of Formula (I-a) is of Formula (I′-a), or a salt thereof. In some embodiments, the compound of Formula (I′-a) is of Formula (I′-a-1), or a salt thereof. In some embodiments, the compound of Formula (I′-a) is of Formula (I′-a-2), or a salt thereof. In some embodiments of Formulae (I-a) and (I′-a), at least one instance of R^{3a} is halogen or —NO₂. In some embodiments of Formulae (I-a) and (I′-a), each instance of R^{3a} is independently halogen. In some embodiments of Formulae (I-a) and (I′-a), at least one instance of R^{3a} is fluoro. In some embodiments of Formulae (I-a) and (I′-a), at least one instance of R^{3a} is —NO₂.

In some embodiments, the compound of Formula (I) is of Formula (I-b), or a salt thereof. In some embodiments, the compound of Formula (I-b) is of Formula (I′-b), or a salt thereof.

In some embodiments, the compound of Formula (I) is of Formula (I-c), or a salt thereof. In some embodiments, the compound of Formula (I-c) is of Formula (I′-c), or a salt thereof. In some embodiments, the compound of Formula (I) is of Formula (I-cc), or a salt thereof. In some embodiments, the compound of Formula (I-c) is of Formula (I′-cc), or a salt thereof.

In some embodiments, the compound of Formula (I) is of Formula (I-d), or a salt thereof. In some embodiments, the compound of Formula (I-d) is of Formula (I-d-1), or a salt thereof. In some embodiments, the compound of Formula (I-d) is of Formula (I-d-2), or a salt thereof. In some embodiments, the compound of Formula (I-d) is of Formula (I′-d), or a salt thereof. In some embodiments of Formulae (I-d) and (I′-d), at least one instance of R^{3a} is halogen or —NO₂. In some embodiments of Formulae (I-d) and (I′-d), each instance of R^{3a} is independently halogen. In some embodiments of Formulae (I-d) and (I′-d), at least one instance of R^{3a} is fluoro. In some embodiments of Formulae (I-d) and (I′-d), at least one instance of R^{3a} is —NO₂. In some embodiments, the compound of Formula (I′-d) is of Formula (I′-d-1), or a salt thereof. In some embodiments, the compound of Formula (I′-d) is of Formula (I′-d-2), or a salt thereof.

In some embodiments, the compound of Formula (I) is of Formula (I-dd), or a salt thereof. In some embodiments, the compound of Formula (I-dd) is of Formula (I-dd-1), or a salt thereof. In some embodiments, the compound of Formula (I-dd) is of Formula (I-dd-2), or a salt thereof. In some embodiments, the compound of Formula (I-dd) is of Formula (I′-dd), or a salt thereof. In some embodiments of Formulae (I-dd) and (I′-dd), at least one instance of R^{3a} is halogen or —NO₂. In some embodiments of Formulae (I-dd) and (I′-dd), each instance of R^{3a} is independently halogen. In some embodiments of Formulae (I-dd) and (I′-dd), at least one instance of R^{3a} is fluoro. In some embodiments of Formulae (I-dd) and (I′-dd), at least one instance of R^{3a} is —NO₂. In some embodiments, the compound of Formula (I′-dd) is of Formula (I′-dd-1), or a salt thereof. In some embodiments, the compound of Formula (I′-dd) is of Formula (I′-dd-2), or a salt thereof.

In some embodiments, the compound of Formula (I) is of Formula (I-e), or a salt thereof. In some embodiments, the compound of Formula (I-e) is of Formula (I′-e), or a salt thereof. In some embodiments, the compound of Formula (I) is of Formula (I-ee), or a salt thereof. In some embodiments, the compound of Formula (I-e) is of Formula (I′-ee), or a salt thereof.

In some embodiments, the compound of Formula (I) is of Formula (I-f), or a salt thereof. In some embodiments, the compound of Formula (I-f) is of Formula (I′-f), or a salt thereof.

In some embodiments, the compound of Formula (I) is of Formula (I-g), or a salt thereof. In some embodiments, the compound of Formula (I-g) is of Formula (I-g-1), or a salt thereof. In some embodiments, the compound of Formula (I-g) is of Formula (I-g-2), or a salt thereof. In some embodiments, the compound of Formula (I-g) is of Formula (I′-g), or a salt thereof. In some embodiments of Formulae (I-g) and (I′-g), at least one instance of R^{3a} is halogen or —NO₂. In some embodiments of Formulae (I-g) and (I′-g), each instance of R^{3a} is independently halogen. In some embodiments of Formulae (I-g) and (I′-g), at least one instance of R^{3a} is fluoro. In some embodiments of Formulae (I-g) and (I′-g), at least one instance of R^{3a} is —NO₂. In some embodiments, the compound of Formula (I′-g) is of Formula (I′-g-1), or a salt thereof. In some embodiments, the compound of Formula (I′-g) is of Formula (I′-g-2), or a salt thereof.

In some embodiments, the compound of Formula (I) is of Formula (I-h), or a salt thereof. In some embodiments, the compound of Formula (I-h) is of Formula (I′-h), or a salt thereof.

In some embodiments, the compound of Formula (I) is of Formula (I-i), or a salt thereof. In some embodiments, the compound of Formula (I-i) is of Formula (I′-i), or a salt thereof. In some embodiments, the compound of Formula (I) is of Formula (I-ii), or a salt thereof. In some embodiments, the compound of Formula (I-i) is of Formula (I′-ii), or a salt thereof.

In some embodiments, the compound of Formula (I) is of Formula (I-j), or a salt thereof. In some embodiments, the compound of Formula (I-j) is of Formula (I-j-1), or a salt thereof. In some embodiments, the compound of Formula (I-j) is of Formula (I-j-2), or a salt thereof. In some embodiments, the compound of Formula (I-j) is of Formula (I-j-3), or a salt thereof. In some embodiments, the compound of Formula (I-j) is of Formula (I-j-4), or a salt thereof. In some embodiments, the compound of Formula (I-j) is of Formula (I-j-5), or a salt thereof. In some embodiments, the compound of Formula (I-j) is of Formula (I-j-6), or a salt thereof. In some embodiments, the compound of Formula (I-j) is of Formula (I′-j), or a salt thereof. In some embodiments of Formulae (I-j) and (I′-j), at least one instance of R^{3a} is halogen or —NO₂. In some embodiments of Formulae (I-j) and (I′-j), each instance of R^{3a} is independently halogen. In some embodiments of Formulae (I-j) and (I′-j), at least one instance of R^{3a} is fluoro. In some embodiments of Formulae (I-j) and (I′-j), at least one instance of R^{3a} is —NO₂. In some embodiments, the compound of Formula (I′-j) is of Formula (I′-j-1), or a salt thereof. In some embodiments, the compound of Formula (I′-j) is of Formula (I′-j-2), or a salt thereof. In some embodiments, the compound of Formula (I′-j) is of Formula (I′-j-3), or a salt thereof. In some embodiments, the compound of Formula (I′-j) is of Formula (I′-j-4), or a salt thereof. In some embodiments, the compound of Formula (I′-j) is of Formula (I′-j-5), or a salt thereof. In some embodiments, the compound of Formula (I′-j) is of Formula (I′-j-6), or a salt thereof. In some embodiments, the compound of Formula (I) is of Formula (I-jj), or a salt thereof. In some embodiments, the compound of Formula (I-jj) is of Formula (I-jj-1), or a salt thereof. In some embodiments, the compound of Formula (I-jj) is of Formula (I-jj-2), or a salt thereof. In some embodiments, the compound of Formula (I-jj) is of Formula (I-jj-3), or a salt thereof. In some embodiments, the compound of Formula (I-jj) is of Formula (I-jj-4), or a salt thereof. In some embodiments, the compound of Formula (I-jj) is of Formula (I-jj-5), or a salt thereof. In some embodiments, the compound of Formula (I-jj) is of Formula (I-jj-6), or a salt thereof. In some embodiments, the compound of Formula (I-jj) is of Formula (I′-jj), or a salt thereof. In some embodiments of Formulae (I-jj) and (I′-jj), at least one instance of R^{3a} is halogen or —NO₂. In some embodiments of Formulae (I-jj) and (I′-jj), each instance of R³ is independently halogen. In some embodiments of Formulae (I-jj) and (I′-jj), at least one instance of R^{3a} is fluoro. In some embodiments of Formulae (I-jj) and (I′-jj), at least one instance of R^{3a} is —NO₂. In some embodiments, the compound of Formula (I′-jj) is of Formula (I′-jj-1), or a salt thereof. In some embodiments, the compound of Formula (I′-jj) is of Formula (I′-jj-2), or a salt thereof. In some embodiments, the compound of Formula (I′-jj) is of Formula (I′-jj-3), or a salt thereof. In some embodiments, the compound of Formula (I′-jj) is of Formula (I′-jj-4), or a salt thereof. In some embodiments, the compound of Formula (I′-jj) is of Formula (I′-jj-5), or a salt thereof. In some embodiments, the compound of Formula (I′-jj) is of Formula (I′-jj-6), or a salt thereof.

In some embodiments, the compound of Formula (I) is of Formula (I-k), or a salt thereof. In some embodiments, the compound of Formula (I-k) is of Formula (I-k-1), or a salt thereof. In some embodiments, the compound of Formula (I-k) is of Formula (I-k-2), or a salt thereof. In some embodiments, the compound of Formula (I) is of Formula (I′-k), or a salt thereof. In some embodiments, the compound of Formula (I′-k) is of Formula (I′-k-1), or a salt thereof. In some embodiments, the compound of Formula (I′-k) is of Formula (I′-k-2), or a salt thereof. In some embodiments, the compound of Formula (I) is of Formula (I-kk), or a salt thereof. In some embodiments, the compound of Formula (I-kk) is of Formula (I-kk-1), or a salt thereof. In some embodiments, the compound of Formula (I-kk) is of Formula (I-kk-2), or a salt thereof. In some embodiments, the compound of Formula (I) is of Formula (I′-kk), or a salt thereof. In some embodiments, the compound of Formula (I′-kk) is of Formula (I′-kk-1), or a salt thereof. In some embodiments, the compound of Formula (I′-kk) is of Formula (I′-kk-2), or a salt thereof.

In some embodiments, the compound of Formula (II) is of Formula (II-a-1), or a salt thereof. In some embodiments, the compound of Formula (II-a-1) is of Formula (II′-a-1), or a salt thereof. In some embodiments of Formulae (II-a-1) and (II′-a-1), R⁵ is —OH. In some embodiments of Formulae (II-a-1) and (II′-a-1), R⁵ is —NH₂. In some embodiments, the compound of Formula (II) is of Formula (II-a-2), or a salt thereof. In some embodiments, the compound of Formula (II-a-2) is of Formula (II′-a-2), or a salt thereof. In some embodiments of Formulae (II-a-2) and (II′-a-2), R⁵ is —OH. In some embodiments of Formulae (II-a-2) and (II′-a-2), R⁵ is —NH₂.

In some embodiments, the compound of Formula (II) is of Formula (II-b), or a salt thereof. In some embodiments, the compound of Formula (II-b) is of Formula (II′-b), or a salt thereof. In some embodiments of Formulae (II-b) and (II′-b), R⁵ is —OH. In some embodiments of Formulae (II-b) and (II′-b), R⁵ is —NH₂.

In some embodiments, the compound of Formula (II) is of Formula (II-c-1), or a salt thereof. In some embodiments, the compound of Formula (II-c-1) is of Formula (II′-c-1), or a salt thereof. In some embodiments of Formulae (II-c-1) and (II′-c-1), R⁵ is —OH. In some embodiments of Formulae (II-c-1) and (II′-c-1), R⁵ is —NH₂. In some embodiments, the compound of Formula (II) is of Formula (II-c-2), or a salt thereof. In some embodiments, the compound of Formula (II-c-2) is of Formula (II′-c-2), or a salt thereof. In some embodiments of Formulae (II-c-2) and (II′-c-2), R⁵ is —OH. In some embodiments of Formulae (II-c-2) and (II′-c-2), R⁵ is —NH₂.

In some embodiments, the compound of Formula (III-d) is of Formula (III-d-1), or a salt thereof. In some embodiments, the compound of Formula (III-d) is of Formula (III-d-2), or a salt thereof. In some embodiments, the compound of Formula (III-d) is of Formula (III′-d), or a salt thereof. In some embodiments, the compound of Formula (III′-d) is of Formula (III′-d-1), or a salt thereof. In some embodiments, the compound of Formula (III′-d) is of Formula (III′-d-2), or a salt thereof. In some embodiments of Formulae (III-d), (III-d-1), (III-d-2), (III′-d), (III′-d-1), and (III′-d-2), R⁵ is —OH. In some embodiments of Formulae (III-d), (III-d-1), (III-d-2), (III′-d), (III′-d-1), and (III′-d-2), R⁵ is —NH₂.

In some embodiments, the compound of Formula (III-d) is of Formula (III-e), or a salt thereof. In some embodiments, the compound of Formula (III-e) is of Formula (III-e-1), or a salt thereof. In some embodiments, the compound of Formula (III-e) is of Formula (III-e-2), or a salt thereof. In some embodiments, the compound of Formula (III-e) is of Formula (III′-e), or a salt thereof. In some embodiments, the compound of Formula (III′-e) is of Formula (III′-e-1), or a salt thereof. In some embodiments, the compound of Formula (III′-e) is of Formula (III′-e-2), or a salt thereof. In some embodiments of Formulae (III-e), (III-e-1), (III-e-2), (III′-e), (III′-e-1), and (III′-e-2), R⁵ is —OH. In some embodiments of Formulae (III-e), (III-e-1), (III-e-2), (III′-e), (III′-e-1), and (III′-e-2), R⁵ is —NH₂. In some embodiments, the compound of Formula (III-d) is of Formula (III-ee), or a salt thereof. In some embodiments, the compound of Formula (III-ee) is of Formula (III-ee-1), or a salt thereof. In some embodiments, the compound of Formula (III-ee) is of Formula (III-ee-2), or a salt thereof. In some embodiments, the compound of Formula (III-ee) is of Formula (III′-ee), or a salt thereof. In some embodiments, the compound of Formula (III′-ee) is of Formula (III′-ee-1), or a salt thereof. In some embodiments, the compound of Formula (III′-ee) is of Formula (III′-ee-2), or a salt thereof. In some embodiments of Formulae (III-ee), (III-ee-1), (III-ee-2), (III′-ee), (III′-ee-1), and (III′-ee-2), R⁵ is —OH. In some embodiments of Formulae (III-ee), (III-ee-1), (III-ee-2), (III′-ee), (III′-ee-1), and (III′-ee-2), R⁵ is —NH₂.

In some embodiments, the compound of Formula (III-d) is of Formula (III-f), or a salt thereof. In some embodiments, the compound of Formula (III-f) is of Formula (ITT-f-1), or a salt thereof. In some embodiments, the compound of Formula (III-f) is of Formula (III-f-2), or a salt thereof. In some embodiments, the compound of Formula (III-f) is of Formula (III′-f), or a salt thereof. In some embodiments, the compound of Formula (III′-f) is of Formula (II-f-1), or a salt thereof. In some embodiments, the compound of Formula (III′-f) is of Formula (III′-f-2), or a salt thereof. In some embodiments of Formulae (III-f), (ITT-f-1), (III-f-2), (III′-f), (II-f-1), and (III′-f-2), R⁵ is —OH. In some embodiments of Formulae (III-f), (ITT-f-1), (III-f-2), (III′-f), (II-f-1), and (III′-f-2), R⁵ is —NH₂.

In some embodiments, the compound of Formula (III-d) is of Formulae (III-g), or a salt thereof. In some embodiments, the compound of Formula (III-g) is of Formula (III-g-1), or a salt thereof. In some embodiments, the compound of Formula (III-g) is of Formula (III-g-2), or a salt thereof. In some embodiments, the compound of Formula (III-g) is of Formulae (III′-g), or a salt thereof. In some embodiments, the compound of Formula (III′-g) is of Formula (III′-g-1), or a salt thereof. In some embodiments, the compound of Formula (III′-g) is of Formula (III′-g-2), or a salt thereof. In some embodiments of Formulae (III-g), (III-g-1), (III-g-2), (III′-g), (III′-g-1), and (III′-g-2), R⁵ is —OH. In some embodiments of Formulae (III-g), (III-g-1), (III-g-2), (III′-g), (III′-g-1), and (III′-g-2), R⁵ is —NH₂. In some embodiments, the compound of Formula (III-d) is of Formulae (III-gg), or a salt thereof. In some embodiments, the compound of Formula (III-gg) is of Formula (III-gg-1), or a salt thereof. In some embodiments, the compound of Formula (III-gg) is of Formula (III-gg-2), or a salt thereof. In some embodiments, the compound of Formula (III-gg) is of Formulae (III′-gg), or a salt thereof. In some embodiments, the compound of Formula (III′-gg) is of Formula (III′-gg-1), or a salt thereof. In some embodiments, the compound of Formula (III′-gg) is of Formula (III′-gg-2), or a salt thereof. In some embodiments of Formulae (III-gg), (III-gg-1), (III-gg-2), (III′-gg), (III′-gg-1), and (III′-gg-2), R⁵ is —OH. In some embodiments of Formulae (III-gg), (III-gg-1), (III-gg-2), (III′-gg), (III′-gg-1), and (III′-gg-2), R⁵ is —NH₂.

In some embodiments, the compound of Formula (IV) is of Formula (IV′):

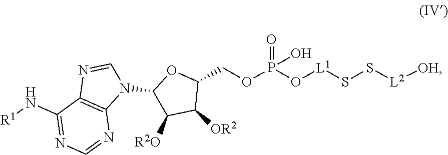

or a salt thereof.

In some embodiments, the compound of Formula (IV) is of Formula (IV-a-1) or (IV-a-2):

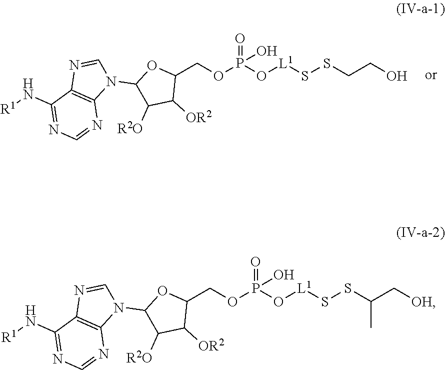

or a salt thereof.

In some embodiments, the compound of Formula (IV) is of Formula (IV-a-1), or a salt thereof. In some embodiments, the compound of Formula (IV) is of Formula (IV-a-2), or a salt thereof.

In some embodiments, the compound of Formula (IV-a-1) or (IV-a-2) is of Formula (IV′-a-1) or (IV′-a-2):

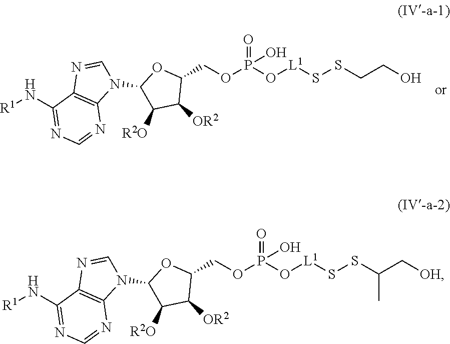

or a salt thereof.

In some embodiments, the compound of Formula (IV-a-1) is of Formula (IV′-a-1), or a salt thereof. In some embodiments, the compound of Formula (IV-a-2) is of Formula (IV′-a-2), or a salt thereof.

In some embodiments, the compound of Formula (IV) is of Formula (IV-b):

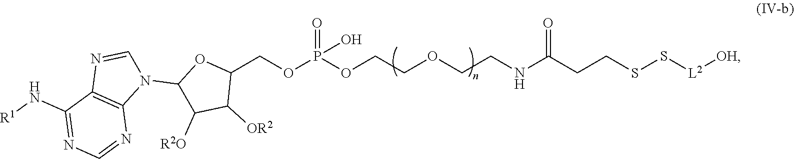

or a salt thereof, wherein n is an integer between 0 and 30, inclusive.

In some embodiments, the compound of Formula (IV-b) is of Formula (IV′-b):

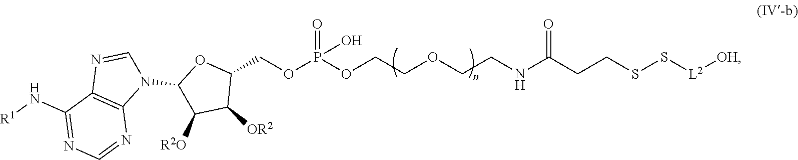

or a salt thereof.

In some embodiments, the compound of Formula (IV) is of Formula (IV-c) or (IV-cc):

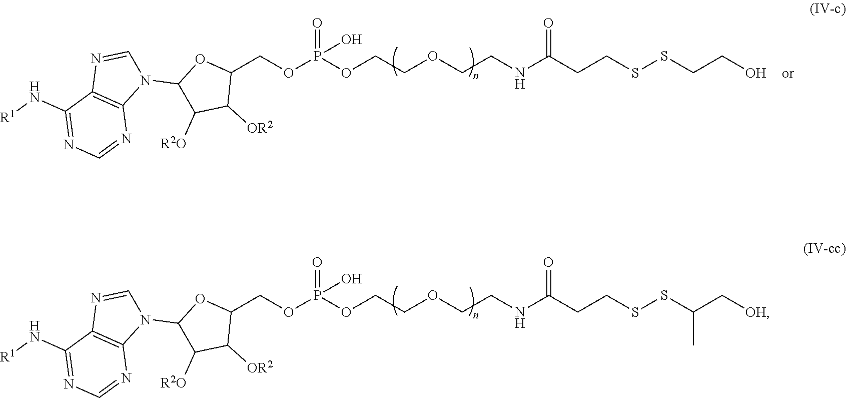

or a salt thereof, wherein n is an integer between 0 and 30, inclusive.

In some embodiments, the compound of Formula (IV) is of Formula (IV-c), or a salt thereof. In some embodiments, the compound of Formula (IV) is of Formula (IV-cc), or a salt thereof.

In some embodiments, the compound of Formula (IV-c) or (IV-cc) is of Formula (IV-c-1) or (IV-cc-1):

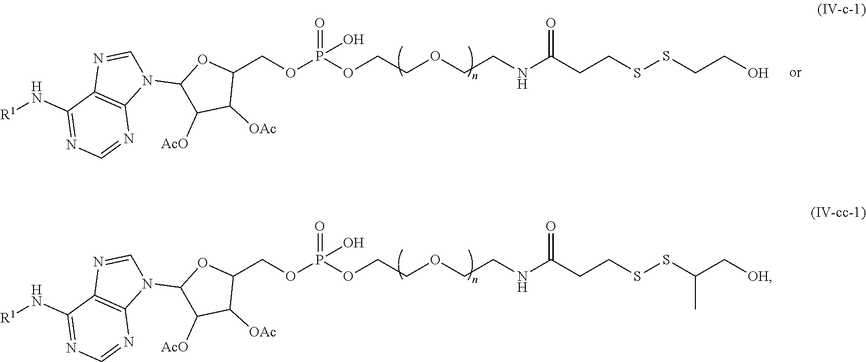

or a salt thereof.

In some embodiments, the compound of Formula (IV-c) is of Formula (IV-c-1), or a salt thereof. In some embodiments, the compound of Formula (IV-cc) is of Formula (IV-cc-1), or a salt thereof.

In some embodiments, the compound of Formula (IV-c) or (IV-cc) is of Formula (IV-c-2) or (IV-cc-2):

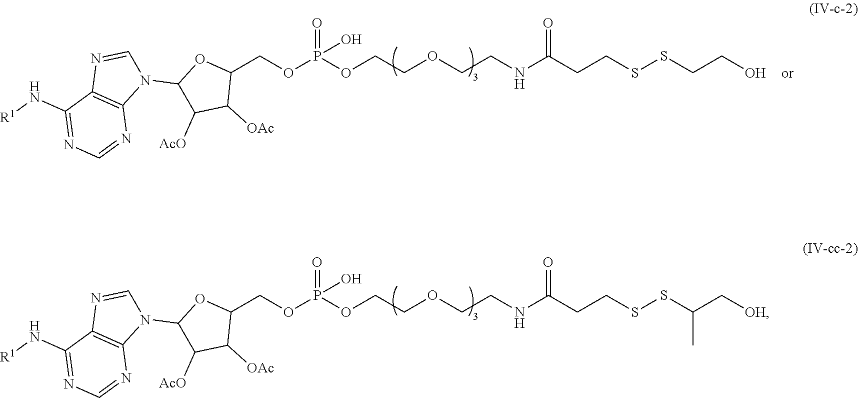

or a salt thereof.

In some embodiments, the compound of Formula (IV-c) is of Formula (IV-c-2), or a salt thereof. In some embodiments, the compound of Formula (IV-cc) is of Formula (IV-cc-2), or a salt thereof.

In some embodiments, the compound of Formula (IV-c) or (IV-cc) is of Formula (IV′-c) or (IV′-cc):

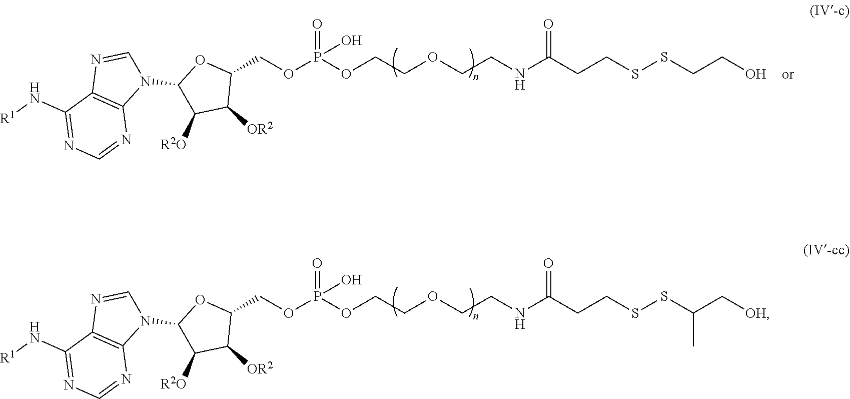

or a salt thereof.

In some embodiments, the compound of Formula (IV-c) is of Formula (IV′-c), or a salt thereof. In some embodiments, the compound of Formula (IV-cc) is of Formula (IV′-cc), or a salt thereof.

In some embodiments, the compound of Formula (IV′-c) or (IV′-cc) is of Formula (IV′-c-1) or (IV′-cc-1):

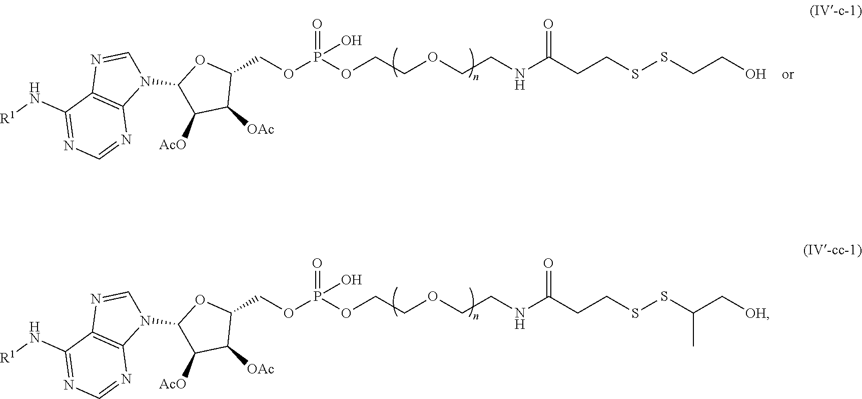

or a salt thereof.

In some embodiments, the compound of Formula (IV′-c) is of Formula (IV′-c-1), or a salt thereof. In some embodiments, the compound of Formula (IV′-cc) is of Formula (IV′-cc-1), or a salt thereof.

In some embodiments, the compound of Formula (IV′-c) or (IV′-cc) is of Formula (IV′-c-2) or (IV′-cc-2):

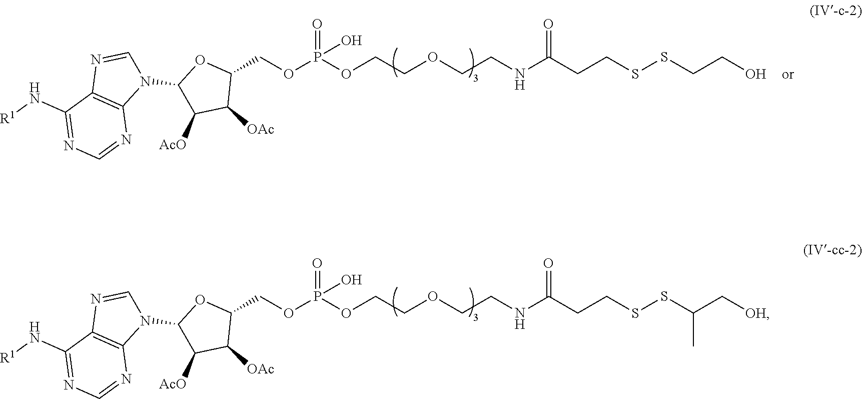

or a salt thereof.

In some embodiments, the compound of Formula (IV′-c) is of Formula (IV′-c-2), or a salt thereof. In some embodiments, the compound of Formula (IV′-cc) is of Formula (IV′-cc-2), or a salt thereof.

In some embodiments, the compound of Formula (V) is of Formula (V-a):

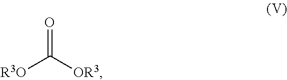

or a salt thereof, wherein each instance of R³ is independently optionally substituted aryl or optionally substituted heteroaryl.

In some embodiments, the compound of Formula (V) is of formula:

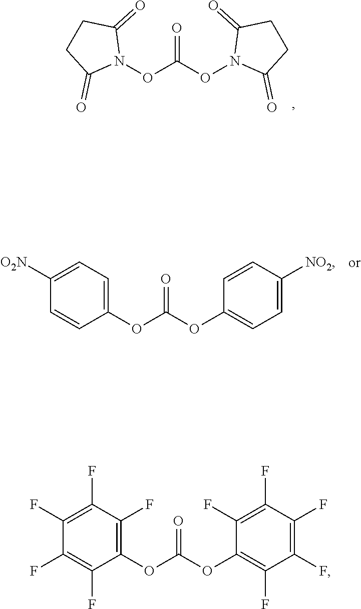 [SMILES: O=C(ON1C(=O)CCC1=O)ON1C(=O)CCC1=O ; O=C(Oc1ccc([N+](=O)[O-])cc1)Oc1ccc([N+](=O)[O-])cc1 ; O=C(Oc1c(F)c(F)c(F)c(F)c1F)Oc1c(F)c(F)c(F)c(F)c1F]

or a salt thereof.

In some embodiments, the compound of Formula (IV), or salt thereof, is prepared by reacting a compound of Formula (VI):

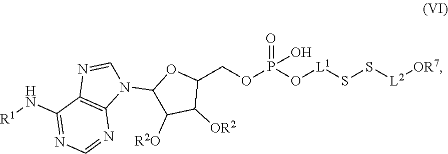

or a salt thereof, under suitable conditions to obtain the compound of Formula (IV), or salt thereof, wherein R¹ is an oxygen protecting group.

In some embodiments, the compound of Formula (VI) is of Formula (VI′):

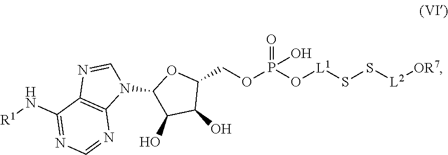

or a salt thereof.

In some embodiments, the compound of Formula (VI) is of Formula (VI-a) or (VI-aa):

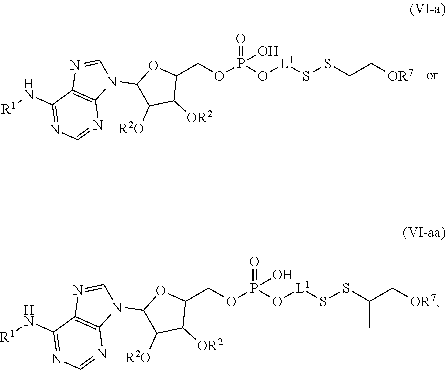

or a salt thereof.

In some embodiments, the compound of Formula (VI) is of Formula (VI-a), or a salt thereof. In some embodiments, the compound of Formula (VI) is of Formula (VI-aa), or a salt thereof.

In some embodiments, the compound of Formula (VI-a) or (VI-aa) is of Formula (VI′-a) or (VI′-aa):

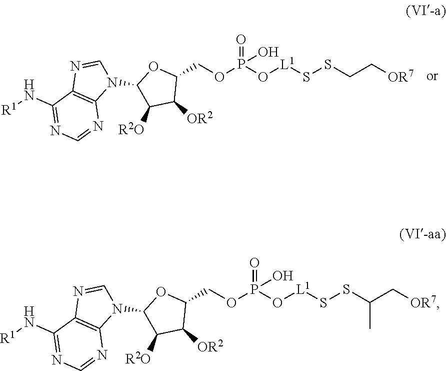

or a salt thereof.

In some embodiments, the compound of Formula (VI-a) is of Formula (VI′-a), or a salt thereof. In some embodiments, the compound of Formula (VI-aa) is of Formula (VI′-aa), or a salt thereof.

In some embodiments, the compound of Formula (VI) is of Formula (VI-b):

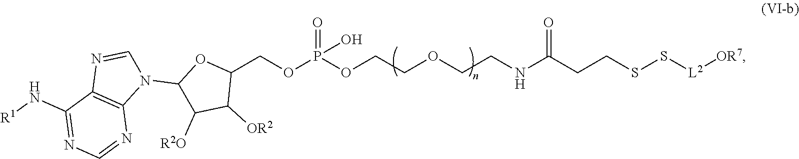

or a salt thereof, wherein n is an integer between 0 and 30, inclusive.

In some embodiments, the compound of Formula (VI-b) is of Formula (VI′-b):

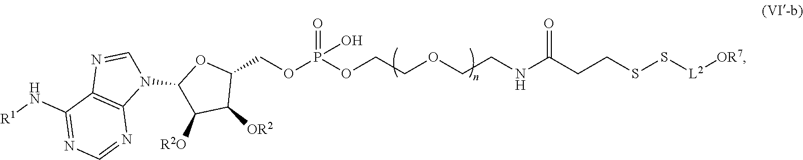

or a salt thereof.

In some embodiments, the compound of Formula (VI) is of Formula (VI-c) or (VI-cc):

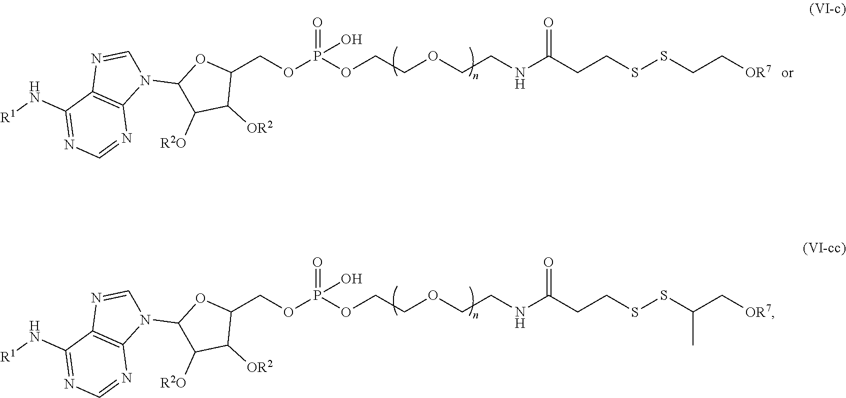

or a salt thereof, wherein n is an integer between 0 and 30, inclusive.

In some embodiments, the compound of Formula (VI) is of Formula (VI-c), or a salt thereof. In some embodiments, the compound of Formula (VI) is of Formula (VI-cc), or a salt thereof.

In some embodiments, the compound of Formula (VI-c) or (VI-cc) is of Formula (VI-c-1) or (VI-cc-1):

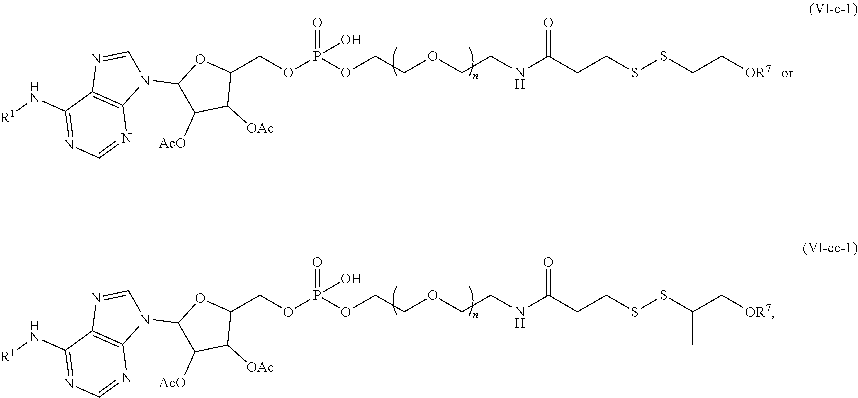

or a salt thereof.

In some embodiments, the compound of Formula (VI-c) is of Formula (VI-c-1), or a salt thereof. In some embodiments, the compound of Formula (VI-cc) is of Formula (VI-cc-1), or a salt thereof.

In some embodiments, the compound of Formula (VI-c) or (VI-cc) is of Formula (VI-c-2) or (VI-cc-2):

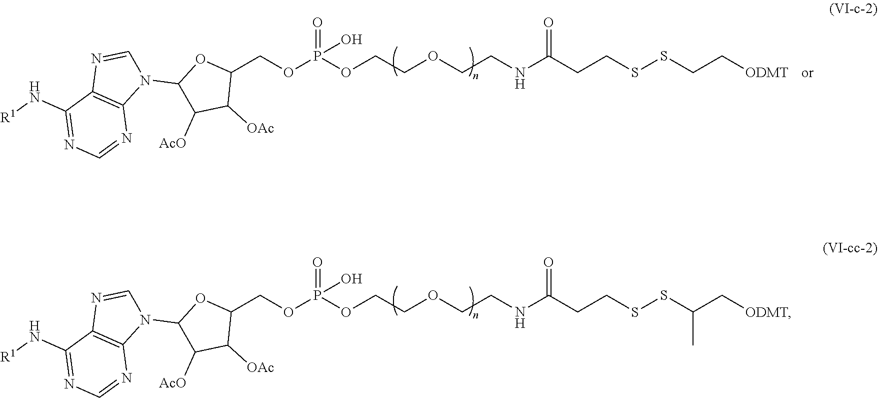

or a salt thereof.

In some embodiments, the compound of Formula (VI-c) is of Formula (VI-c-2), or a salt thereof. In some embodiments, the compound of Formula (VI-cc) is of Formula (VI-cc-2), or a salt thereof.

In some embodiments, the compound of Formula (VI-c) or (VI-cc) is of Formula (VI′-c) or (VI′-cc):

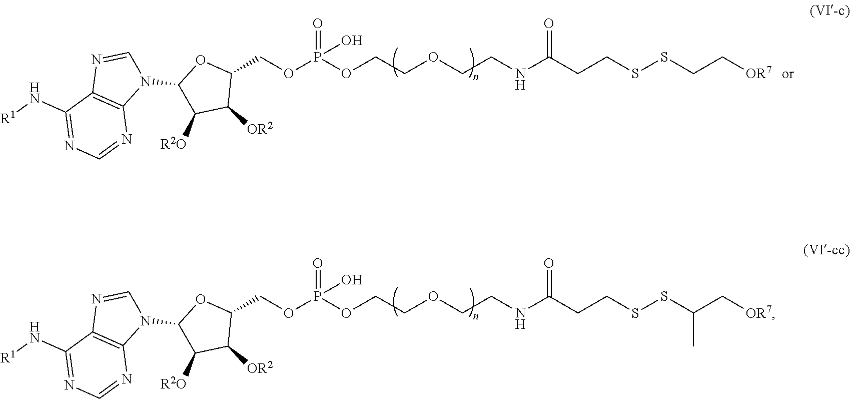

or a salt thereof.

In some embodiments, the compound of Formula (VI-c) is of Formula (VI′-c), or a salt thereof. In some embodiments, the compound of Formula (VI-cc) is of Formula (VI′-cc), or a salt thereof.

In some embodiments, the compound of Formula (VI′-c) or (VI′-cc) is of Formula (VI′-c-1) or (VI′-cc-1):

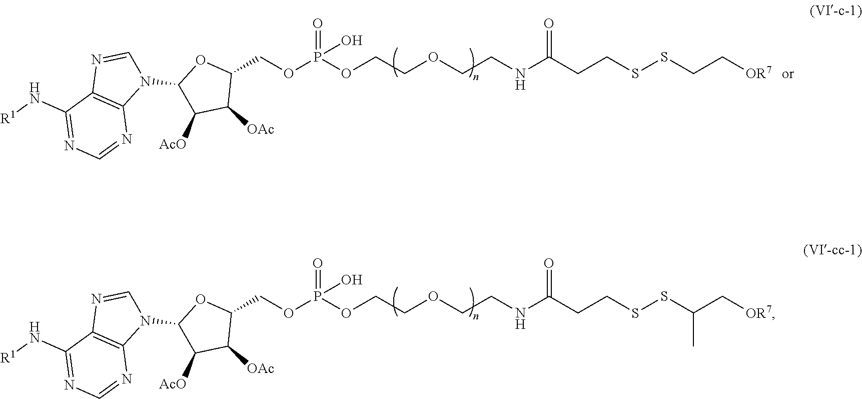

or a salt thereof.

In some embodiments, the compound of Formula (VI′-c) is of Formula (VI′-c-1), or a salt thereof. In some embodiments, the compound of Formula (VI′-cc) is of Formula (VI′-cc-1), or a salt thereof.

In some embodiments, the compound of Formula (VI′-c) or (VI′-cc) is of Formula (VI′-c-2) or (VI′-cc-2):

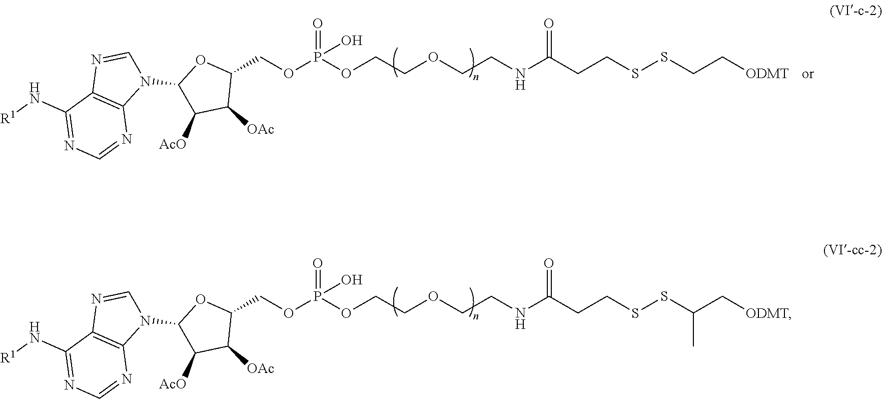

or a salt thereof.

In some embodiments, the compound of Formula (VI′-c) is of Formula (VI′-c-2), or a salt thereof. In some embodiments, the compound of Formula (VI′-cc) is of Formula (VI′-cc-2), or a salt thereof.

In some embodiments, the compound of Formula (VI), or salt thereof, is prepared by reacting a compound of Formula (VII):

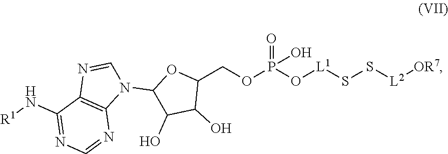

or a salt thereof, under suitable conditions to obtain the compound of Formula (VI), or salt thereof.

In some embodiments, the compound of Formula (VII) is of Formula (VII′):

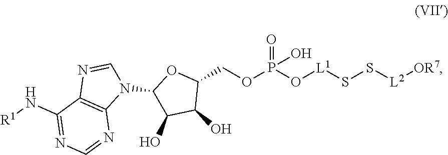

or a salt thereof.

In some embodiments, the compound of Formula (VII) is of Formula (VII-a) or (VII-aa):

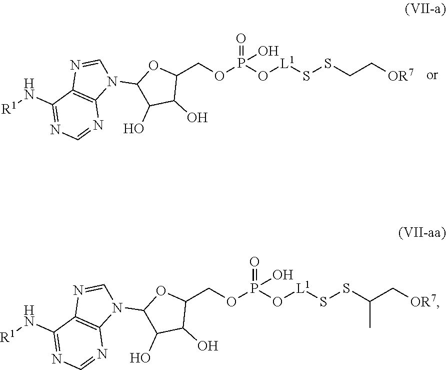

or a salt thereof.

In some embodiments, the compound of Formula (VII) is of Formula (VII-a), or a salt thereof. In some embodiments, the compound of Formula (VII) is of Formula (VII-aa), or a salt thereof.

In some embodiments, the compound of Formula (VII′) is of Formula (VII′-a) or (VII′-aa):

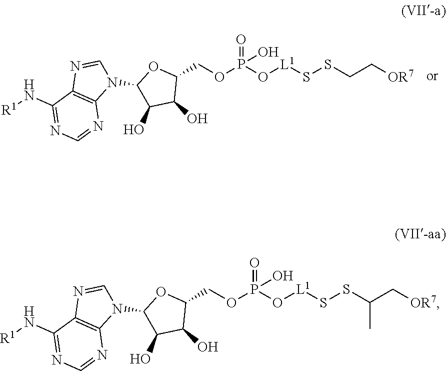

or a salt thereof.

In some embodiments, the compound of Formula (VII′) is of Formula (VII′-a), or a salt thereof. In some embodiments, the compound of Formula (VII′) is of Formula (VII′-aa), or a salt thereof.

In some embodiments, the compound of Formula (VII) is of Formula (VII-b):

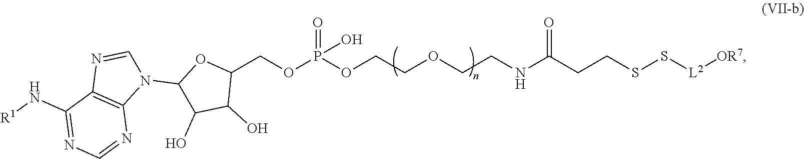

or a salt thereof, wherein n is an integer between 0 and 30, inclusive.

In some embodiments, the compound of Formula (VII-b) is of Formula (VII′-b):

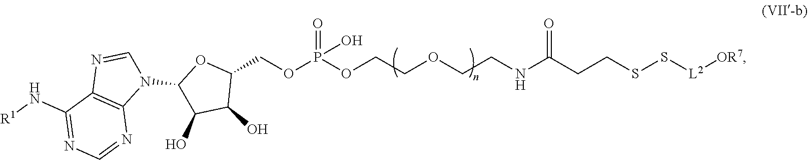

or a salt thereof.

In some embodiments, the compound of Formula (VII) is of Formula (VII-c) or (VII-cc):

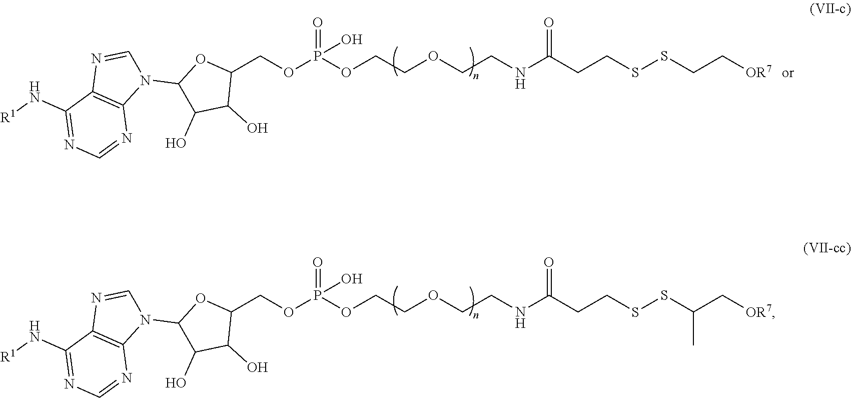

or a salt thereof, wherein n is an integer between 0 and 30, inclusive.

In some embodiments, the compound of Formula (VII) is of Formula (VII-c), or a salt thereof. In some embodiments, the compound of Formula (VII) is of Formula (VII-cc), or a salt thereof.

In some embodiments, the compound of Formula (VII-c) or (VII-cc) is of Formula (VII-c-1) or (VII-cc-1):

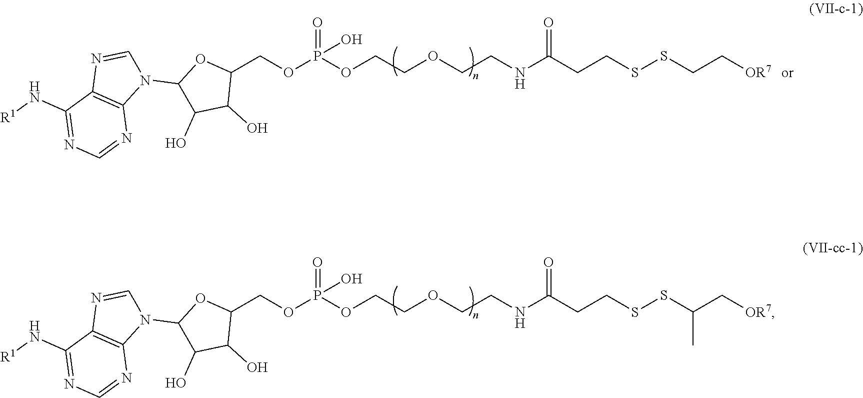

or a salt thereof.

In some embodiments, the compound of Formula (VII-c) is of Formula (VII-c-1), or a salt thereof. In some embodiments, the compound of Formula (VII-cc) is of Formula (VII-cc-1), or a salt thereof.

In some embodiments, the compound of Formula (VII-c) or (VII-cc) is of Formula (VII-c-2) or (VII-cc-2):

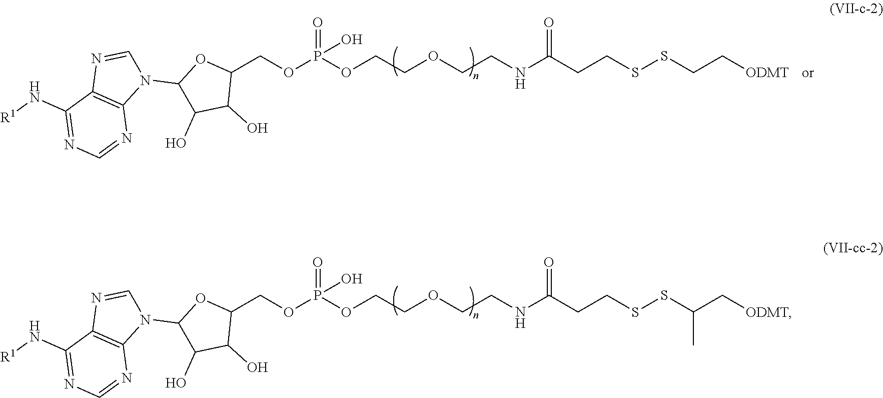

or a salt thereof.

In some embodiments, the compound of Formula (VII-c) is of Formula (VII-c-2), or a salt thereof. In some embodiments, the compound of Formula (VII-cc) is of Formula (VII-cc-2), or a salt thereof.

In some embodiments, the compound of Formula (VII-c) or (VII-cc) is of Formula (VII′-c) or (VII′-cc):

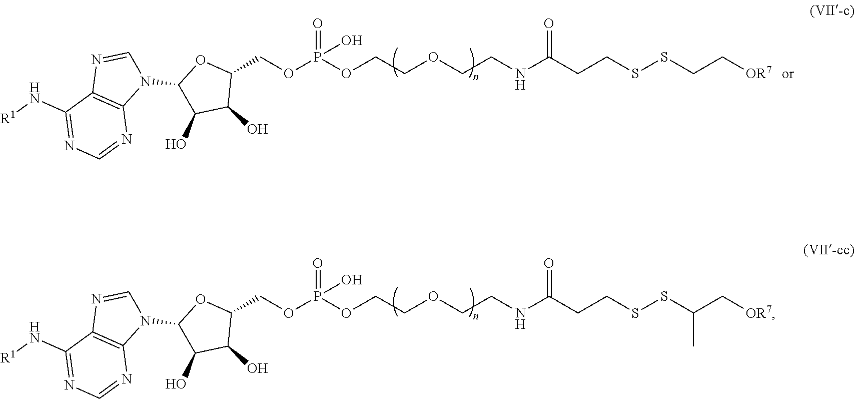

or a salt thereof.

In some embodiments, the compound of Formula (VII-c) is of Formula (VII′-c), or a salt thereof. In some embodiments, the compound of Formula (VII-cc) is of Formula (VII′-cc), or a salt thereof.

In some embodiments, the compound of Formula (VII′-c) or (VII′-cc) is of Formula (VII′-c-1) or (VII′-cc-1):

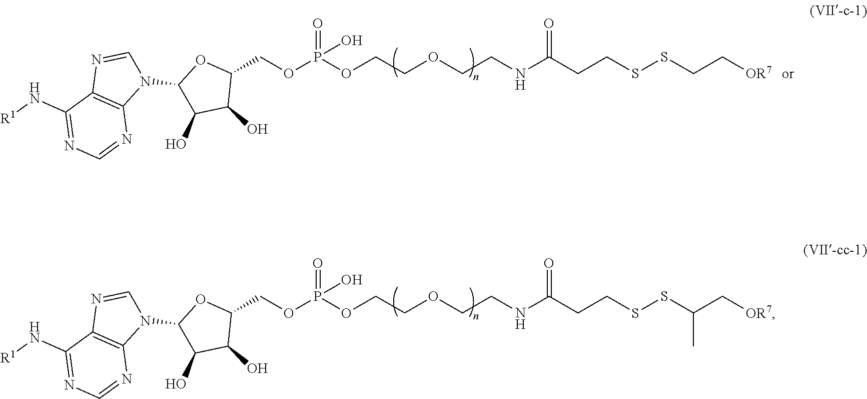

or a salt thereof.

In some embodiments, the compound of Formula (VII′-c) is of Formula (VII′-c-1), or a salt thereof. In some embodiments, the compound of Formula (VII′-cc) is of Formula (VII′-cc-1), or a salt thereof.

In some embodiments, the compound of Formula (VII′-c) or (VII′-cc) is of Formula (VII′-c-2) or (VII′-cc-2):

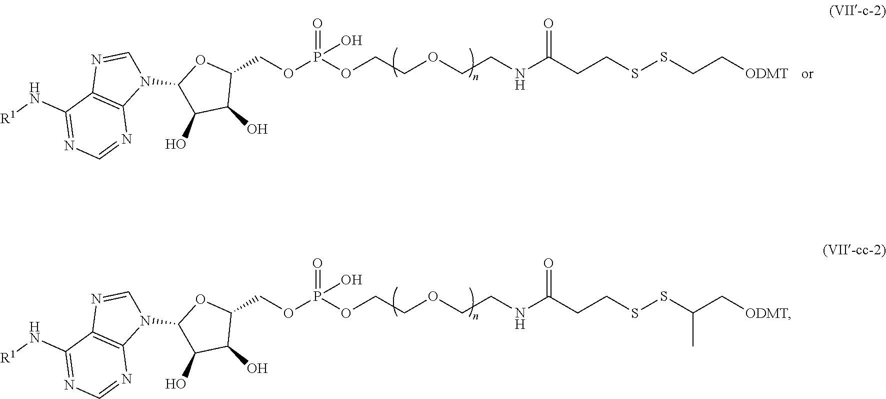

or a salt thereof.

In some embodiments, the compound of Formula (VII′-c) is of Formula (VII′-c-2), or a salt thereof. In some embodiments, the compound of Formula (VII′-cc) is of Formula (VII′-cc-2), or a salt thereof.

In some embodiments, the compound of Formula (VII-c), or salt thereof, is prepared by coupling a compound of Formula (VIII):

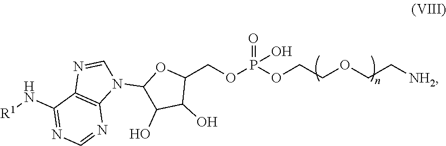

or a salt thereof, with a compound of Formula (IX-a):

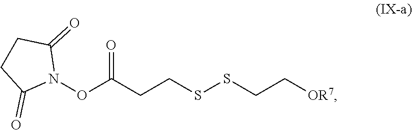

or a salt thereof, under suitable conditions to obtain the compound of Formula (VII-c), or salt thereof.

In some embodiments, the compound of Formula (VII-cc), or salt thereof, is prepared by coupling a compound of Formula (VIII), or a salt thereof, with a compound of Formula (IX-b):

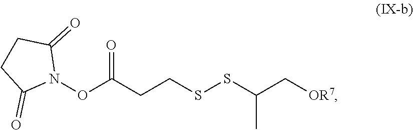

or a salt thereof, under suitable conditions to obtain the compound of Formula (VII-cc), or salt thereof.

In some embodiments, the compound of Formula (VIII) is of Formula (VIII′):

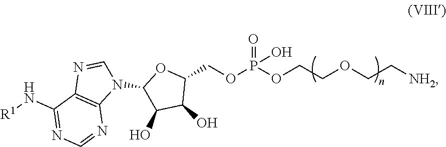

or a salt thereof.

In some embodiments, the compound of Formula (IX-a) is of Formula (IX-a-1):

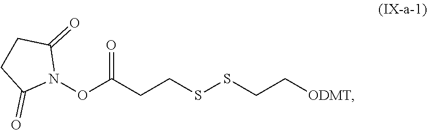

or a salt thereof.

In some embodiments, the compound of Formula (IX-a), or salt thereof, is prepared by reacting a compound of Formula (X-a):

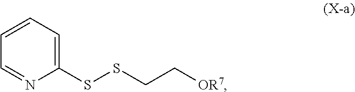

or a salt thereof, under suitable conditions to obtain the compound of Formula (IX-a), or salt thereof.

In some embodiments, the compound of Formula (X-a) is of Formula (X-a-1):

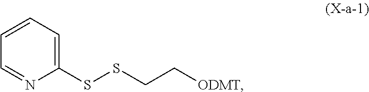

or a salt thereof.

In some embodiments, the compound of Formula (X), or salt thereof, is prepared by reacting a compound of Formula (XI-a):

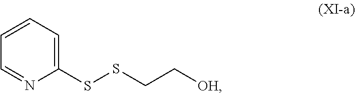 [SMILES: OCCSSc1ccccn1]

or a salt thereof, under suitable conditions to obtain the compound of Formula (X), or salt thereof.

In some embodiments, the compound of Formula (IX-b) is of Formula (IX-b):

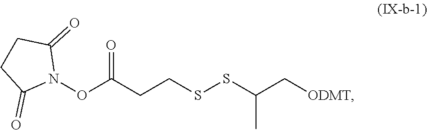

or a salt thereof.

In some embodiments, the compound of Formula (IX-b), or salt thereof, is prepared by reacting a compound of Formula (X-b):

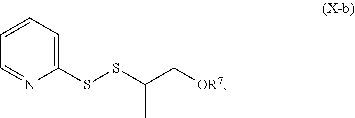

or a salt thereof, under suitable conditions to obtain the compound of Formula (IX-b), or salt thereof.

In some embodiments, the compound of Formula (X-b) is of Formula (X-b-1):

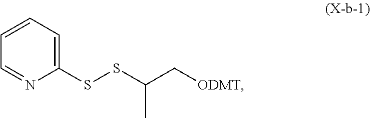

or a salt thereof.

In some embodiments, the compound of Formula (X-b), or salt thereof, is prepared by reacting a compound of Formula (XI-b):

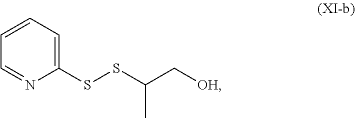

or a salt thereof, under suitable conditions to obtain the compound of Formula (X-b), or salt thereof.

In some embodiments, the compound of Formula (XII) is of Formula (XII-a):

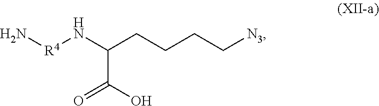

or a salt thereof.

In some embodiments, the compound of Formula (XII) is of Formula (XII-a):

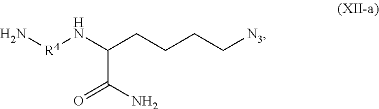

or a salt thereof.

In some embodiments, the compound of Formula (XIII) is of Formula (XIII-a):

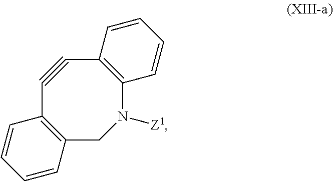

or a salt thereof.

In some embodiments, the compound of Formula (XIII) is of Formula (XIII-b):

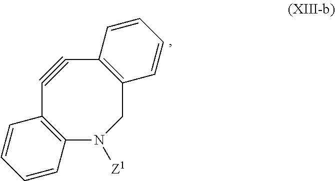

or a salt thereof.

As generally described herein, Z¹ comprises an oligonucleotide. In certain embodiments, the oligonucleotide is a single-stranded oligonucleotide. In certain embodiments, the oligonucleotide is a double-stranded oligonucleotide. In certain embodiments, the oligonucleotide has a length of at least 15, at least 20, at least 25, at least 30, at least 35, at least 40, at least 45, or at least 50 nucleotides. In certain embodiments, the oligonucleotide has a length in a range from 15 to 20, 15 to 25, 15 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, 25 to 50, 30 to 35, 30 to 40, 30 to 45, 30 to 50, 35 to 40, 35 to 45, 35 to 50, 40 to 45, 40 to 50, or 45 to 50 nucleotides. In some embodiments, the oligonucleotide has a length of at least 25 nucleotides.

In certain embodiments, at least one strand of the oligonucleotide has a sequence that is at least 50%, at least 55%, at least 60%, at least 65%, at least 70%, at least 75%, at least 80%, at least 85%, at least 90%, at least 95%, at least 99%, or 100% identical to 5′-CCACGCGTGGAACCCTTGGGATCCA-3′ (SEQ ID NO: 32). In some embodiments, at least one strand of the oligonucleotide has a sequence that is at least 80% identical to 5′-CCACGCGTGGAACCCTTGGGATCCA-3′ (SEQ ID NO: 32). In certain embodiments, at least one strand of the oligonucleotide has a sequence that is at least 50%, at least 55%, at least 60%, at least 65%, at least 70%, at least 75%, at least 80%, at least 85%, at least 90%, at least 95%, at least 99%, or 100% identical to 5′-TGG AGT CAA GGT CCT CTG ATG CCA T-3′ (SEQ ID NO: 33).

In some embodiments, Z¹ further comprises a linking group.

In some embodiments, the linking group comprises a polypeptidyl group. In certain embodiments, the polypeptidyl group comprises at least 5 amino acid residues, at least 10 amino acid residues, at least 15 amino acid residues, or at least 20 amino acid residues. In certain embodiments, the polypeptidyl group comprises between 5 and 10 amino acid residues, between 5 and 15 amino acid residues, between 5 and 20 amino acid residues, between 10 and 15 amino acid residues, between 10 and 20 amino acid residues, or between 15 and 20 amino acid residues. In some embodiments, the polypeptidyl group comprises between 5 and 15 amino acid residues.

In some embodiments, the polypeptidyl group has a length of at least about 20 Å, 25 Å, 30 Å, 35 Å, 40 Å, 45 Å, 50 Å, 55 Å, 60 Å, 65 Å, 70 Å, or 75 Å. In certain embodiments, the polypeptidyl group has a length in a range from 20 Å to 30 Å, 20 Å to 35 Å, 20 Å to 40 Å, 20 Å to 45 Å, 20 Å to 50 Å, 20 Å to 55 Å, 20 Å to 60 Å, 20 Å to 65 Å, 20 Å to 70 Å, 20 Å to 75 Å, 30 Å to 40 Å, 30 Å to 45 Å, 30 Å to 50 Å, 30 Å to 55 Å, 30 Å to 60 Å, 30 Å to 65 Å, 30 Å to 70 Å, 30 Å to 75 Å, 40 Å to 50 Å, 40 Å to 55 Å, 40 Å to 60 Å, 40 Å to 65 Å, 40 Å to 70 Å, 40 Å to 75 Å, 50 Å to 60 Å, 50 Å to 65 Å, 50 Å to 70 Å, 50 Å to 75 Å, 60 Å to 70 Å, or 60 Å to 75 Å.

In some embodiments, the polypeptidyl group comprises at least 1 negatively charged moiety at physiological pH. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 negatively charged moieties at physiological pH. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 negatively charged moieties at physiological pH. In some embodiments, the polypeptidyl group comprises between 1 and 10 negatively charged moieties at physiological pH.

In some embodiments, the polypeptidyl group comprises at least 1 aspartate residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 aspartate residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 aspartate residues. In some embodiments, the polypeptidyl group comprises between 1 and 10 aspartate residues.

In some embodiments, the polypeptidyl group comprises at least 1 phenylalanine residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 phenylalanine residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 phenylalanine residues.

In some embodiments, the polypeptidyl group comprises at least 1 glycine residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 glycine residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 glycine residues.

In some embodiments, the polypeptidyl group comprises at least 1 proline residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 proline residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 proline residues.

In some embodiments, the polypeptidyl group comprises at least 1 DD repeat, GG repeat, FF repeat, DDD repeat, GGG, and/or FFF repeat. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 DD repeats, GG repeats, FF repeats, DDD repeats, GGG, and/or FFF repeats. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 DD repeats, GG repeats, FF repeats, DDD repeats, GGG, and/or FFF repeats.

In some embodiments, the polypeptidyl group comprises a sequence selected from the group consisting of GPPPPPPPPG (SEQ ID NO: 34), isoEGWRW (SEQ ID NO: 35), DDGGGDDDFF (SEQ ID NO: 36), GGSSSGSGNDEEFQ (SEQ ID NO: 37), GGGGGDPDPDFF (SEQ ID NO: 38), GDGDGDGDGDFF (SEQ ID NO: 39), NNGGGNNNFF (SEQ ID NO: 40), and DDGGGCyCyCyFF (SEQ ID NO: 41), or a salt thereof, wherein Cy is a cysteic acid. In some embodiments, the polypeptidyl group comprises DDGGGDDDFF (SEQ ID NO: 36). In some embodiments, the oligonucleotide has a length of at least 25 nucleotides, and the polypeptidyl group comprises DDGGGDDDFF (SEQ ID NO: 36).

In some embodiments, the linking group further comprises at least one of optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof.

In some embodiments, Z¹ further comprises a binding group. In some embodiments, the binding group comprises a biotin moiety. In some embodiments, Z¹ further comprises a biotin moiety. In some embodiments, the biotin moiety is a bis-biotin moiety.

In some embodiments, the binding group comprises at least one tag sequence. In certain embodiments, the at least one tag sequence comprises at least one biotin ligase recognition sequence that permits biotinylation of the compound comprising Z¹ (e.g., incorporation of one or more biotin moieties, including biotin and bis-biotin moieties). In certain embodiments, the at least one tag sequence comprises at least one biotin ligase recognition sequence that permits biotinylation of Z¹. In certain embodiments, the at least one tag sequence comprises two biotin ligase recognition sequences oriented in tandem. In some cases, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem. In certain embodiments, the binding group comprises at least one biotin ligase recognition sequence having a biotin moiety attached thereto or at least two biotin ligase recognition sequences, each having a biotin moiety attached thereto.

In some embodiments, the binding group comprises or is conjugated to an avidin protein. In some embodiments, Z¹ further comprises an avidin protein. In some embodiments, the biotin moiety comprises an avidin protein. In some embodiments, the biotin moiety is conjugated to an avidin protein. The term “avidin protein” refers to a biotin-binding protein, generally having a biotin binding site at each of four subunits of the avidin protein. Non-limiting examples of avidin proteins include avidin, streptavidin, traptavidin, tamavidin, bradavidin, xenavidin, and homologs and variants thereof. In some cases, the avidin protein may have a monomeric, dimeric, or tetrameric form. In certain embodiments, the avidin protein is streptavidin in a tetrameric form (e.g., a homotetramer). In certain embodiments, the streptavidin in a tetrameric form may be bound to one component (e.g., a first component comprising a first mono-biotin moiety or a first bis-biotin moiety), two components (e.g., a first component comprising a first mono-biotin moiety or a first bis-biotin moiety and a second component comprising a second mono-biotin moiety or a second bis-biotin moiety), three components (e.g., a first component comprising a first bis-biotin moiety, a second component comprising a first mono-biotin moiety, and a third component comprising a second mono-biotin moiety), or four components (e.g., four components, each comprising a mono-biotin moiety).

In some embodiments, the compound of Formula (XIII), or salt thereof, is immobilized to a surface (e.g., a surface of a sample well). In some embodiments, the compound of Formula (XIII), or salt thereof, is immobilized to a surface of a sample well. In some embodiments, Z¹ is immobilized to a surface (e.g., a surface of a sample well). In some embodiments, Z¹ is immobilized to a surface of a sample well. In some embodiments, the avidin protein is immobilized to a surface (e.g., a surface of a sample well). In some embodiments, the avidin protein is immobilized to a surface of a sample well.

### Methods of Functionalizing a First Terminus of a Peptide and Sequencing a Peptide

In another aspect, the present disclosure provides a method of functionalizing a first terminus of a peptide comprising:
(a) conjugating a second terminus of the peptide to a solid support group; and
(b) conjugating the first terminus of the peptide to a linking group.

In another aspect, the present disclosure provides a method of sequencing a peptide, comprising:
(a) conjugating a second terminus of the peptide to a solid support group;
(b) conjugating a first terminus of the peptide to a linking group;
(c) exposing the peptide to a peptidase in a degradation process;
(d) obtaining data during the degradation process;
(e) analyzing the data to determine portions of the data corresponding to amino acids that are sequentially exposed at the second terminus of the peptide during the degradation process; and
(f) outputting an amino acid sequence representative of the peptide.

In some embodiments, the first terminus is a C-terminus. In some embodiments, the second terminus is an N-terminus.

In some embodiments, step (a) is performed before step (b). In some embodiments, modifying a second terminus of the peptide is performed before conjugating the second terminus of the peptide to a solid support group.

In some embodiments, modifying a second terminus of the peptide is performed before returning the second terminus to a pre-modified state. In some embodiments, modifying a second terminus of the peptide is performed before conjugating the second terminus of the peptide to a solid support group. In some embodiments, modifying a second terminus of the peptide is performed concurrently with conjugating the second terminus of the peptide to a solid support group.

In some embodiments, the method comprises the steps: modifying a second terminus of the peptide; conjugating the second terminus of the peptide to a solid support group; conjugating the first terminus of the peptide to a linking group; and returning the second terminus to a pre-modified state.

In some embodiments, the method comprises the steps: modifying a second terminus of the peptide; conjugating the second terminus of the peptide to a solid support group; conjugating a first terminus of the peptide to a linking group; returning the second terminus to a pre-modified state; exposing the peptide to a peptidase in a degradation process; obtaining data during the degradation process; analyzing the data to determine portions of the data corresponding to amino acids that are sequentially exposed at the second terminus of the peptide during the degradation process; and outputting an amino acid sequence representative of the peptide.

In some embodiments, the peptide remains in the solid phase while conjugated to the solid support group.

In some embodiments, the solid support group comprises a cleavable linker. In some embodiments, the method further comprises cleaving the cleavable linker. In some embodiments, the method further comprises cleaving the cleavable linker prior to exposing the peptide to a peptidase in a degradation process. In some embodiments, the method further comprises exposing the terminal amino acid to amino acid recognizers.

In some embodiments, the cleavable linker is cleaved upon exposure to a reducing agent.

In some embodiments, conjugating the second terminus of the peptide to the solid support group comprises modifying the second terminus. In some embodiments, cleaving the cleavable linker comprises returning the second terminus to a pre-modified state. In some embodiments, the pre-modified state is an N-terminus. In some embodiments, returning the second terminus to a pre-modified state comprises conversion of a metastable intermediate to an N-terminus.

In some embodiments, the peptide is in the solution phase after cleavage of the cleavable linker.

In some embodiments, the solid support group comprises a moiety of Formula (XIV):

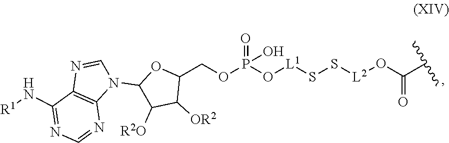

or a salt thereof, wherein:
R¹ is a solid support;
each instance of R² is independently hydrogen or an oxygen protecting group;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof; and
L² is optionally substituted C₂ alkylene.

In some embodiments, the moiety of Formula (XIV) is of Formula (XIV-a) or (XIV-aa):

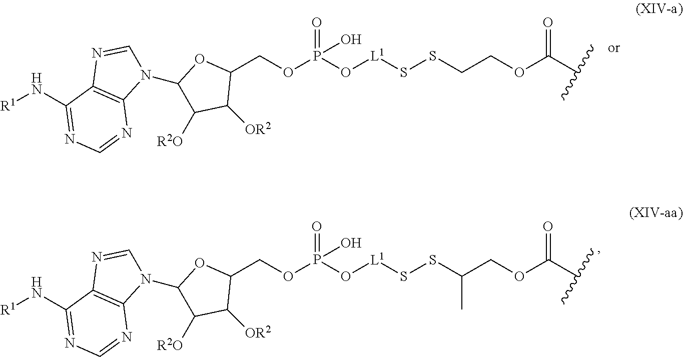

or a salt thereof.

In some embodiments, the moiety of Formula (XIV) is of Formula (XIV-a), or a salt thereof. In some embodiments, the moiety of Formula (XIV) is of Formula (XIV-aa), or a salt thereof.

In some embodiments, the moiety of Formula (XIV) is of Formula (XIV-b):

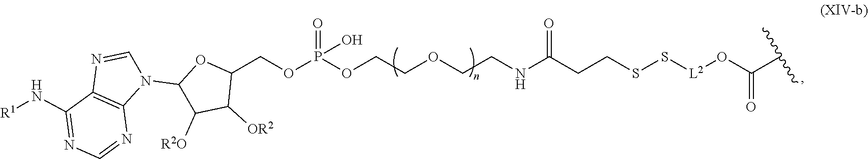

or a salt thereof, wherein n is an integer between 0 and 30, inclusive.

In some embodiments, the moiety of Formula (XIV) is of Formula (XIV-c) or (XIV-cc):

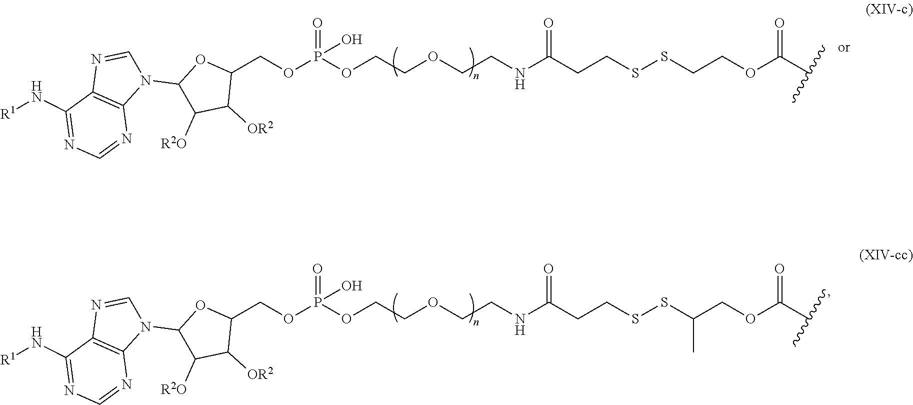

or a salt thereof, wherein n is an integer between 0 and 30, inclusive.

In some embodiments, the moiety of Formula (XIV) is of Formula (XIV-c), or a salt thereof. In some embodiments, the moiety of Formula (XIV) is of Formula (XIV-cc), or a salt thereof.

In some embodiments, the linking group comprises an oligonucleotide. In certain embodiments, the oligonucleotide is a single-stranded oligonucleotide. In certain embodiments, the oligonucleotide is a double-stranded oligonucleotide. In certain embodiments, the oligonucleotide has a length of at least 15, at least 20, at least 25, at least 30, at least 35, at least 40, at least 45, or at least 50 nucleotides. In certain embodiments, the oligonucleotide has a length in a range from 15 to 20, 15 to 25, 15 to 30, 15 to 35, 15 to 40, 15 to 45, 15 to 50, 20 to 25, 20 to 30, 20 to 35, 20 to 40, 20 to 45, 20 to 50, 25 to 30, 25 to 35, 25 to 40, 25 to 45, 25 to 50, 30 to 35, 30 to 40, 30 to 45, 30 to 50, 35 to 40, 35 to 45, 35 to 50, 40 to 45, 40 to 50, or 45 to 50 nucleotides. In some embodiments, the oligonucleotide has a length of at least 25 nucleotides.

In certain embodiments, at least one strand of the oligonucleotide has a sequence that is at least 50%, at least 55%, at least 60%, at least 65%, at least 70%, at least 75%, at least 80%, at least 85%, at least 90%, at least 95%, at least 99%, or 100% identical to 5′-CCACGCGTGGAACCCTTGGGATCCA-3′ (SEQ ID NO: 32). In some embodiments, at least one strand of the oligonucleotide has a sequence that is at least 80% identical to 5′-CCACGCGTGGAACCCTTGGGATCCA-3′ (SEQ ID NO: 32). In certain embodiments, at least one strand of the oligonucleotide has a sequence that is at least 50%, at least 55%, at least 60%, at least 65%, at least 70%, at least 75%, at least 80%, at least 85%, at least 90%, at least 95%, at least 99%, or 100% identical to 5′-TGG AGT CAA GGT CCT CTG ATG CCA T-3′ (SEQ ID NO: 33).

In some embodiments, the linking group comprises a polypeptidyl group. In certain embodiments, the polypeptidyl group comprises at least 5 amino acid residues, at least 10 amino acid residues, at least 15 amino acid residues, or at least 20 amino acid residues. In certain embodiments, the polypeptidyl group comprises between 5 and 10 amino acid residues, between 5 and 15 amino acid residues, between 5 and 20 amino acid residues, between 10 and 15 amino acid residues, between 10 and 20 amino acid residues, or between 15 and 20 amino acid residues. In some embodiments, the polypeptidyl group comprises between 5 and 15 amino acid residues.

In some embodiments, the polypeptidyl group has a length of at least about 20 Å, 25 Å, 30 Å, 35 Å, 40 Å, 45 Å, 50 Å, 55 Å, 60 Å, 65 Å, 70 Å, or 75 Å. In certain embodiments, the polypeptidyl group has a length in a range from 20 Å to 30 Å, 20 Å to 35 Å, 20 Å to 40 Å, 20 Å to 45 Å, 20 Å to 50 Å, 20 Å to 55 Å, 20 Å to 60 Å, 20 Å to 65 Å, 20 Å to 70 Å, 20 Å to 75 Å, 30 Å to 40 Å, 30 Å to 45 Å, 30 Å to 50 Å, 30 Å to 55 Å, 30 Å to 60 Å, 30 Å to 65 Å, 30 Å to 70 Å, 30 Å to 75 Å, 40 Å to 50 Å, 40 Å to 55 Å, 40 Å to 60 Å, 40 Å to 65 Å, 40 Å to 70 Å, 40 Å to 75 Å, 50 Å to 60 Å, 50 Å to 65 Å, 50 Å to 70 Å, 50 Å to 75 Å, 60 Å to 70 Å, or 60 Å to 75 Å.

In some embodiments, the polypeptidyl group comprises at least 1 negatively charged moiety at physiological pH. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 negatively charged moieties at physiological pH. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 negatively charged moieties at physiological pH. In some embodiments, the polypeptidyl group comprises between 1 and 10 negatively charged moieties at physiological pH.

In some embodiments, the polypeptidyl group comprises at least 1 aspartate residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 aspartate residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 aspartate residues. In some embodiments, the polypeptidyl group comprises between 1 and 10 aspartate residues.

In some embodiments, the polypeptidyl group comprises at least 1 phenylalanine residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 phenylalanine residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 phenylalanine residues.

In some embodiments, the polypeptidyl group comprises at least 1 glycine residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 glycine residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 glycine residues.

In some embodiments, the polypeptidyl group comprises at least 1 proline residue. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 proline residues. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 proline residues.

In some embodiments, the polypeptidyl group comprises at least 1 DD repeat, GG repeat, FF repeat, DDD repeat, GGG, and/or FFF repeat. In certain embodiments, the polypeptidyl group comprises at least 1, at least 2, at least 3, at least 4, at least 5, at least 6, at least 7, at least 8, at least 9, at least 10, at least 11, at least 12, at least 13, at least 14, or at least 15 DD repeats, GG repeats, FF repeats, DDD repeats, GGG, and/or FFF repeats. In certain embodiments, the polypeptidyl group comprises between 1 and 2, 1 and 3, 1 and 4, 1 and 5, 1 and 6, 1 and 7, 1 and 8, 1 and 9, 1 and 10, 1 and 11, 1 and 12, 1 and 13, 1 and 14, 1 and 15, 2 and 3, 2 and 4, 2 and 5, 2 and 6, 2 and 7, 2 and 8, 2 and 9, 2 and 10, 2 and 11, 2 and 12, 2 and 13, 2 and 14, 2 and 15, 3 and 4, 3 and 5, 3 and 6, 3 and 7, 3 and 8, 3 and 9, 3 and 10, 3 and 11, 3 and 12, 3 and 13, 3 and 14, 3 and 15, 4 and 5, 4 and 6, 4 and 7, 4 and 8, 4 and 9, 4 and 10, 4 and 11, 4 and 12, 4 and 13, 4 and 14, 4 and 15, 5 and 6, 5 and 7, 5 and 8, 5 and 9, 5 and 10, 5 and 11, 5 and 12, 5 and 13, 5 and 14, 5 and 15, 6 and 10, 6 and 15, 7 and 10, 7 and 15, 8 and 10, 8 and 15, 9 and 10, 9 and 15, or 10 and 15 DD repeats, GG repeats, FF repeats, DDD repeats, GGG, and/or FFF repeats.

In some embodiments, the polypeptidyl group comprises a sequence selected from the group consisting of GPPPPPPPPG (SEQ ID NO: 34), isoEGWRW (SEQ ID NO: 35), DDGGGDDDFF (SEQ ID NO: 36), GGSSSGSGNDEEFQ (SEQ ID NO: 37), GGGGGDPDPDFF (SEQ ID NO: 38), GDGDGDGDGDFF (SEQ ID NO: 39), NNGGGNNNFF (SEQ ID NO: 40), and DDGGGCyCyCyFF (SEQ ID NO: 41), or a salt thereof, wherein Cy is a cysteic acid. In some embodiments, the polypeptidyl group comprises DDGGGDDDFF (SEQ ID NO: 36). In some embodiments, the oligonucleotide has a length of at least 25 nucleotides, and the polypeptidyl group comprises DDGGGDDDFF (SEQ ID NO: 36).

In some embodiments, the linking group further comprises at least one of optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof.

In some embodiments, the linking group further comprises a binding group. In some embodiments, the binding group comprises a biotin moiety. In some embodiments, the linking group further comprises a biotin moiety. In some embodiments, the biotin moiety is a bis-biotin moiety.

In some embodiments, the binding group comprises at least one tag sequence. In certain embodiments, the at least one tag sequence comprises at least one biotin ligase recognition sequence that permits biotinylation of the compound comprising the linking group (e.g., incorporation of one or more biotin moieties, including biotin and bis-biotin moieties). In certain embodiments, the at least one tag sequence comprises at least one biotin ligase recognition sequence that permits biotinylation of the linking group. In certain embodiments, the at least one tag sequence comprises two biotin ligase recognition sequences oriented in tandem. In some cases, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem. In certain embodiments, the binding group comprises at least one biotin ligase recognition sequence having a biotin moiety attached thereto or at least two biotin ligase recognition sequences, each having a biotin moiety attached thereto.

In some embodiments, the binding group comprises or is conjugated to an avidin protein. In some embodiments, the linking group further comprises an avidin protein. In some embodiments, the biotin moiety comprises an avidin protein. In some embodiments, the biotin moiety is conjugated to an avidin protein. The term “avidin protein” refers to a biotin-binding protein, generally having a biotin binding site at each of four subunits of the avidin protein. Non-limiting examples of avidin proteins include avidin, streptavidin, traptavidin, tamavidin, bradavidin, xenavidin, and homologs and variants thereof. In some cases, the avidin protein may have a monomeric, dimeric, or tetrameric form. In certain embodiments, the avidin protein is streptavidin in a tetrameric form (e.g., a homotetramer). In certain embodiments, the streptavidin in a tetrameric form may be bound to one component (e.g., a first component comprising a first mono-biotin moiety or a first bis-biotin moiety), two components (e.g., a first component comprising a first mono-biotin moiety or a first bis-biotin moiety and a second component comprising a second mono-biotin moiety or a second bis-biotin moiety), three components (e.g., a first component comprising a first bis-biotin moiety, a second component comprising a first mono-biotin moiety, and a third component comprising a second mono-biotin moiety), or four components (e.g., four components, each comprising a mono-biotin moiety).

In some embodiments, the compound comprising the linking group is immobilized to a surface (e.g., a surface of a sample well). In some embodiments, the compound comprising the linking group is immobilized to a surface of a sample well. In some embodiments, the linking group is immobilized to a surface (e.g., a surface of a sample well). In some embodiments, the linking group is immobilized to a surface of a sample well. In some embodiments, the avidin protein is immobilized to a surface (e.g., a surface of a sample well). In some embodiments, the avidin protein is immobilized to a surface of a sample well.

In some embodiments, the linking group comprises a moiety of Formula (XV):

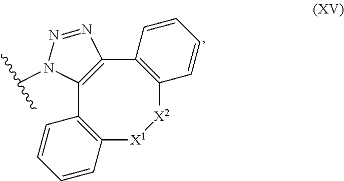

or a salt thereof, wherein:
one of X¹ and X² is CH and the other is N—Z¹; and
Z¹ comprises an oligonucleotide.

In some embodiments, the linking group comprises a moiety of Formula (XV-a):

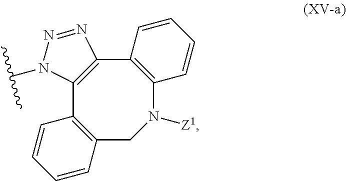

or a salt thereof.

In some embodiments, the linking group comprises a moiety of Formula (XV-b):

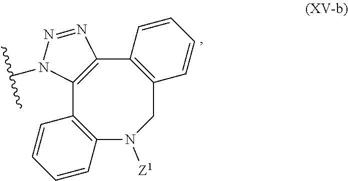

or a salt thereof.

In another aspect, the present disclosure provides a method of sequencing a peptide, comprising exposing a compound of Formula (III):

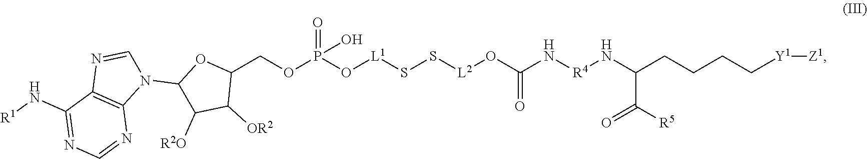

or a salt thereof, to a peptidase in a degradation process;
obtaining data during the degradation process;
analyzing the data to determine portions of the data corresponding to amino acids that are sequentially exposed at a terminus of the peptide during the degradation process; and
outputting an amino acid sequence representative of the peptide;
   wherein:
R¹ is a solid support;
each instance of R² is independently hydrogen or an oxygen protecting group;
R⁴ is the peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof;
L² is optionally substituted C₂ alkylene;
Y¹ comprises a click chemistry adduct; and
Z¹ comprises an oligonucleotide.

In some embodiments, the method further comprises coupling a compound of Formula (I):

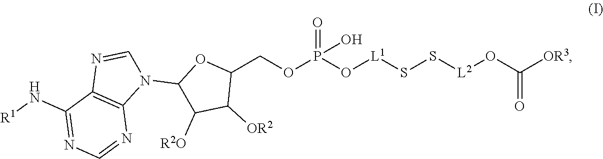

or a salt thereof, with a compound of Formula (XVI):

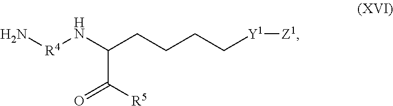

or a salt thereof, under suitable conditions to obtain the compound of Formula (III), or salt thereof, wherein R³ is optionally substituted alkyl, optionally substituted heterocyclyl, optionally substituted aryl, or optionally substituted heteroaryl.

In some embodiments, cleaving the compound of Formula (III), or salt thereof, from the solid support to provide the compound of Formula (XVI), or salt thereof, comprises conversion of a compound of Formula (XVII):

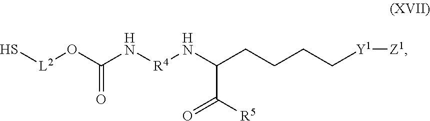

or a salt thereof, to the compound of Formula (XVI), or salt thereof. In some embodiments, the compound of Formula (XVII), or salt thereof, is of Formula (XVII-a):

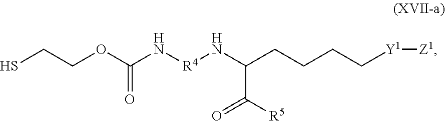

or a salt thereof.

In some embodiments, the compound of Formula (III) is of Formula (III-d):

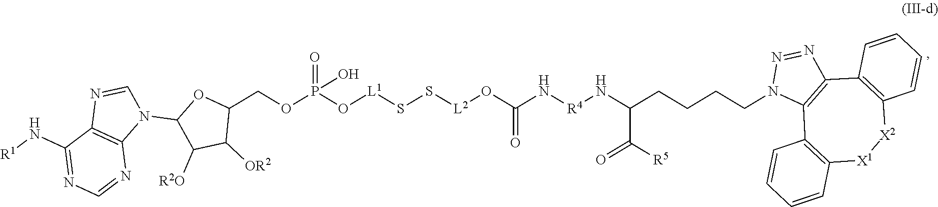

or a salt thereof, wherein one of X¹ and X² is CH and the other is N—Z¹.

In some embodiments, the method further comprises cleaving the compound of Formula (III), or salt thereof, from the solid support to provide a compound of Formula (XVI):

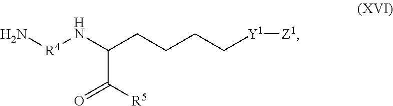

or a salt thereof.

In some embodiments, the method comprises exposing the compound of Formula (XVI), or salt thereof, to the peptidase in the degradation process.

In another aspect, the present disclosure provides a method of sequencing a peptide, comprising exposing a compound of Formula (III-d):

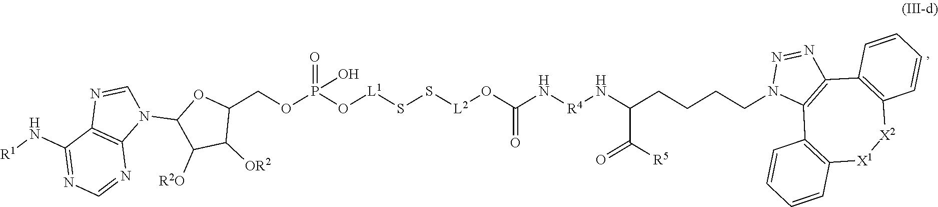

or a salt thereof, to a peptidase in a degradation process;
obtaining data during the degradation process;
analyzing the data to determine portions of the data corresponding to amino acids that are sequentially exposed at a terminus of the peptide during the degradation process; and
outputting an amino acid sequence representative of the peptide;
   wherein:
R¹ is a solid support;
each instance of R² is independently hydrogen or an oxygen protecting group;
R⁴ is the peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof;
L² is optionally substituted C₂ alkylene; and
one of X¹ and X² is CH and the other is N—Z¹, wherein Z¹ comprises an oligonucleotide.

In some embodiments, the method further comprises coupling a compound of Formula (II):

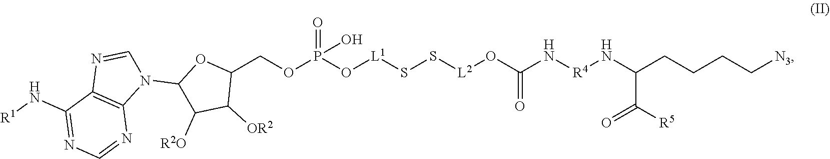

or a salt thereof, with a compound of Formula (XIII):

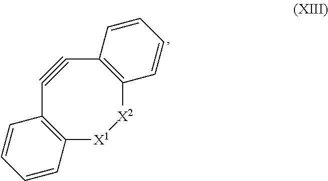

or a salt thereof, under suitable conditions to obtain the compound of Formula (III-d), or salt thereof.

In some embodiments, the method further comprises coupling a compound of Formula (I):

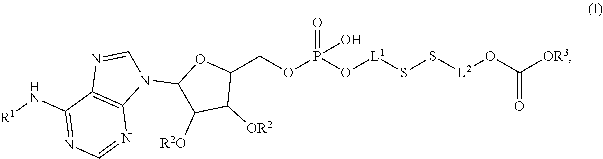

or a salt thereof, with a compound of Formula (XII):

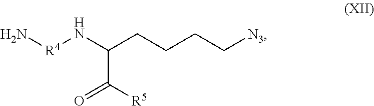

or a salt thereof, under suitable conditions to obtain the compound of Formula (II), or salt thereof, wherein R³ is optionally substituted alkyl, optionally substituted heterocyclyl, optionally substituted aryl, or optionally substituted heteroaryl.

In some embodiments, the method further comprises cleaving the compound of Formula (III-d), or salt thereof, from the solid support to provide a compound of Formula (XII):

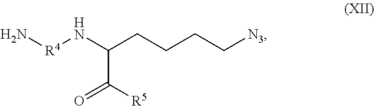

or a salt thereof.

In some embodiments, cleaving the compound of Formula (III-d), or salt thereof, from the solid support to provide the compound of Formula (XII), or salt thereof, comprises conversion of a compound of Formula (XVIII):

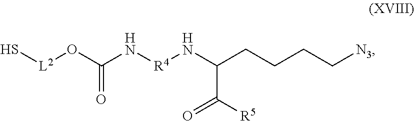

or a salt thereof, to the compound of Formula (XII), or salt thereof. In some embodiments, the compound of Formula (XVIII), or salt thereof, is of Formula (XVIII-a):

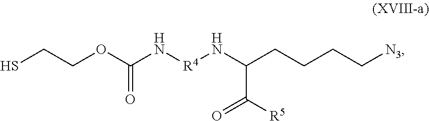

or a salt thereof.

In some embodiments, the method comprises exposing the compound of Formula (XII), or salt thereof, to the peptidase in the degradation process.

In some embodiments, R¹ is a polymeric support. In some embodiments, R¹ is a polymeric support of Oligo-Affinity Support (PS) (5′-Dimethoxytrityl-Adenosine-2′,3′-diacetate-N-Linked-Polymeric Support), available from Glen Research (Catalog Number 26-4001).

In some embodiments, at least one instance of R² is hydrogen.

In some embodiments, at least one instance of R² is an oxygen protecting group. In some embodiments, at least one instance of R² is —C(═O)R^{2a}, wherein R^{2a} is optionally substituted alkyl or optionally substituted aryl.

In some embodiments, at least one instance of R² is —C(═O)R^{2a}, wherein R^{2a} is optionally substituted C₁₋₃ alkyl. In some embodiments, at least one instance of R² is —C(═O)R^{2a}, wherein R^{2a} is substituted C₁₋₃ alkyl. In some embodiments, at least one instance of R² is —C(═O)R^{2a}, wherein R^{2a} is unsubstituted C₁₋₃ alkyl.

In some embodiments, at least one instance of R² is —C(═O)CH₃.

In some embodiments, each instance of R² is independently hydrogen.

In some embodiments, each instance of R² is independently an oxygen protecting group. In some embodiments, each instance of R² is independently —C(═O)R^{2a}, wherein R^{2a} is optionally substituted alkyl or optionally substituted aryl.

In some embodiments, each instance of R² is independently —C(═O)R^{2a}, wherein R^{2a} is optionally substituted C₁₋₃ alkyl. In some embodiments, each instance of R² is independently —C(═O)R^{2a}, wherein R^{2a} is substituted C₁₋₃ alkyl. In some embodiments, each instance of R² is independently —C(═O)R^{2a}, wherein R^{2a} is unsubstituted C₁₋₃ alkyl.

In some embodiments, each instance of R² is independently —C(═O)CH₃.

In some embodiments, R³ is optionally substituted heterocyclyl or optionally substituted aryl. In some embodiments, R³ is substituted heterocyclyl or substituted aryl. In some embodiments, R³ is substituted 5-6 membered heterocyclyl or substituted phenyl. In some embodiments, R³ is

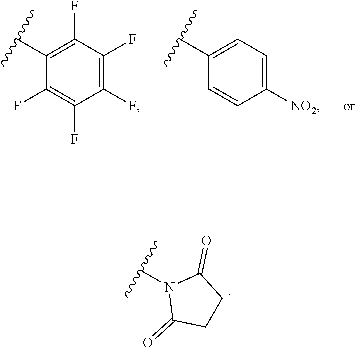 [SMILES: *c1c(F)c(F)c(F)c(F)c1F ; *c1ccc([N+](=O)[O-])cc1 ; *N1C(=O)CCC1=O]

In some embodiments, R³ is optionally substituted heterocyclyl. In some embodiments, R³ is substituted heterocyclyl. In some embodiments, R³ is unsubstituted heterocyclyl.

In some embodiments, R³ is optionally substituted 5-6 membered heterocyclyl. In some embodiments, R³ is substituted 5-6 membered heterocyclyl. In some embodiments, R³ is unsubstituted 5-6 membered heterocyclyl. In some embodiments, R³ is substituted 5-6 membered heterocyclyl containing 1 ring N atom.

In some embodiments, R³ is optionally substituted 5 membered heterocyclyl. In some embodiments, R³ is substituted 5 membered heterocyclyl. In some embodiments, R³ is unsubstituted 5 membered heterocyclyl. In some embodiments, R³ is substituted 5 membered heterocyclyl containing 1 ring N atom.

In some embodiments, R³ is optionally substituted aryl. In some embodiments, R³ is substituted aryl. In some embodiments, R³ is unsubstituted aryl.

In some embodiments, R³ is optionally substituted phenyl. In some embodiments, R³ is substituted phenyl. In some embodiments, R³ is unsubstituted phenyl.

In some embodiments, R³ is phenyl substituted with one or more substituents selected from halogen, optionally substituted alkyl, optionally substituted heteroalkyl, —CN, —OR^{A}, —N(R^{A})₂, and —NO₂. In some embodiments, R³ is phenyl substituted with one or more substituents selected from halogen and —NO₂. In some embodiments, R³ is phenyl substituted with one or more substituents selected from fluoro, chloro, bromo, iodo, and —NO₂. In some embodiments, R³ is phenyl substituted with one or more substituents selected from fluoro, chloro, bromo, and —NO₂. In some embodiments, R³ is phenyl substituted with one or more substituents selected from fluoro, chloro, and —NO₂. In some embodiments, R³ is phenyl substituted with one or more substituents selected from fluoro and —NO₂. In some embodiments, R³ is phenyl substituted with at least one halogen or —NO₂. In some embodiments, R³ is phenyl substituted with at least one fluoro or —NO₂. In some embodiments, R³ is phenyl substituted with at least one fluoro. In some embodiments, R³ is phenyl substituted with at least one —NO₂.

In some embodiments, R⁴ comprises one or more amino acids selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, R⁴ comprises an amino acid comprising a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, R⁴ comprises an amino acid comprising an arginine post-translational modification. For example, as described herein, arginine modifications include symmetric dimethylarginine (SDMA), asymmetric dimethylarginine (ADMA), and citrullinated arginine.

In some embodiments, R⁴ comprises an amino acid comprising a phosphorylated side chain. In some embodiments, R⁴ comprises an amino acid comprising phosphorylated threonine (e.g., phospho-threonine). In some embodiments, R⁴ comprises an amino acid comprising phosphorylated tyrosine (e.g., phospho-tyrosine). In some embodiments, R⁴ comprises an amino acid comprising phosphorylated serine (e.g., phospho-serine).

In some embodiments, R⁴ comprises an amino acid comprising a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, R⁴ comprises an amino acid comprising an oxidative modification. In some embodiments, R⁴ comprises an amino acid comprising a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, R⁴ comprises an amino acid comprising a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, R⁵ is —OH. In some embodiments, R⁵ is —NH₂.

In some embodiments, L¹ comprises optionally substituted alkylene, optionally substituted heteroalkylene, or a combination thereof.

In some embodiments, L¹ comprises optionally substituted alkylene. In some embodiments, L¹ comprises optionally substituted C₁-C₁₀₀ alkylene. In some embodiments, L¹ comprises optionally substituted C₁-C₇₅ alkylene. In some embodiments, L¹ comprises optionally substituted C₁-C₅₀ alkylene. In some embodiments, L¹ comprises optionally substituted C₁-C₂₅ alkylene. In some embodiments, L¹ comprises optionally substituted C₁-C₁₂ alkylene. In some embodiments, L¹ comprises optionally substituted C₁-C₆ alkylene. In some embodiments, L¹ comprises optionally substituted C₁-C₃ alkylene. In some embodiments, L¹ comprises optionally substituted linear alkylene. In some embodiments, L¹ comprises optionally substituted linear C₁-C₁₂ alkylene. In some embodiments, L¹ comprises optionally substituted linear C₁-C₆ alkylene. In some embodiments, L¹ comprises optionally substituted linear C₁-C₃ alkylene.

In some embodiments, L¹ comprises substituted alkylene. In some embodiments, L¹ comprises substituted C₁-C₁₀₀ alkylene. In some embodiments, L¹ comprises substituted C₁-C₇₅ alkylene. In some embodiments, L¹ comprises substituted C₁-C₅₀ alkylene. In some embodiments, L¹ comprises substituted C₁-C₂₅ alkylene. In some embodiments, L¹ comprises substituted C₁-C₁₂ alkylene. In some embodiments, L¹ comprises substituted C₁-C₆ alkylene. In some embodiments, L¹ comprises substituted C₁-C₃ alkylene. In some embodiments, L¹ comprises substituted linear alkylene. In some embodiments, L¹ comprises substituted linear C₁-C₁₂ alkylene. In some embodiments, L¹ comprises substituted linear C₁-C₆ alkylene. In some embodiments, L¹ comprises substituted linear C₁-C₃ alkylene.

In some embodiments, L¹ comprises unsubstituted alkylene. In some embodiments, L¹ comprises unsubstituted C₁-C₁₀₀ alkylene. In some embodiments, L¹ comprises unsubstituted C₁-C₇₅ alkylene. In some embodiments, L¹ comprises unsubstituted C₁-C₅₀ alkylene. In some embodiments, L¹ comprises unsubstituted C₁-C₂₅ alkylene. In some embodiments, L¹ comprises unsubstituted C₁-C₁₂ alkylene. In some embodiments, L¹ comprises unsubstituted C₁-C₆ alkylene. In some embodiments, L¹ comprises unsubstituted C₁-C₃ alkylene. In some embodiments, L¹ comprises unsubstituted linear alkylene. In some embodiments, L¹ comprises unsubstituted linear C₁-C₁₂ alkylene. In some embodiments, L¹ comprises unsubstituted linear C₁-C₆ alkylene. In some embodiments, L¹ comprises unsubstituted linear C₁-C₃ alkylene.

In some embodiments, L¹ comprises methylene, ethylene, n-propylene, n-butylene, n-pentylene, or n-hexylene. In some embodiments, L¹ comprises methylene, ethylene, or n-propylene. In some embodiments, L¹ comprises ethylene.

In some embodiments, L¹ comprises optionally substituted heteroalkylene. In some embodiments, L¹ comprises optionally substituted C₁-C₁₀₀ heteroalkylene. In some embodiments, L¹ comprises optionally substituted C₁-C₇₅ heteroalkylene. In some embodiments, L¹ comprises optionally substituted C₁-C₅₀ heteroalkylene. In some embodiments, L¹ comprises optionally substituted C₁-C₂₅ heteroalkylene. In some embodiments, L¹ comprises optionally substituted C₁-C₁₂ heteroalkylene. In some embodiments, L¹ comprises optionally substituted C₁-C₆ heteroalkylene. In some embodiments, L¹ comprises optionally substituted C₁-C₃ heteroalkylene. In some embodiments, L¹ comprises optionally substituted linear heteroalkylene. In some embodiments, L¹ comprises optionally substituted linear C₁-C₁₂ heteroalkylene. In some embodiments, L¹ comprises optionally substituted linear C₁-C₆ heteroalkylene. In some embodiments, L¹ comprises optionally substituted linear C₁-C₃ heteroalkylene.

In some embodiments, L¹ comprises substituted heteroalkylene. In some embodiments, L¹ comprises substituted C₁-C₁₀₀ heteroalkylene. In some embodiments, L¹ comprises substituted C₁-C₇₅ heteroalkylene. In some embodiments, L¹ comprises substituted C₁-C₅₀ heteroalkylene. In some embodiments, L¹ comprises substituted C₁-C₂₅ heteroalkylene. In some embodiments, L¹ comprises substituted C₁-C₁₂ heteroalkylene. In some embodiments, L¹ comprises substituted C₁-C₆ heteroalkylene. In some embodiments, L¹ comprises substituted C₁-C₃ heteroalkylene. In some embodiments, L¹ comprises substituted linear heteroalkylene. In some embodiments, L¹ comprises substituted linear C₁-C₁₂ heteroalkylene. In some embodiments, L¹ comprises substituted linear C₁-C₆ heteroalkylene. In some embodiments, L¹ comprises substituted linear C₁-C₃ heteroalkylene.

In some embodiments, L¹ comprises unsubstituted heteroalkylene. In some embodiments, L¹ comprises unsubstituted C₁-C₁₀₀ heteroalkylene. In some embodiments, L¹ comprises unsubstituted C₁-C₇₅ heteroalkylene. In some embodiments, L¹ comprises unsubstituted C₁-C₅₀ heteroalkylene. In some embodiments, L¹ comprises unsubstituted C₁-C₂₅ heteroalkylene. In some embodiments, L¹ comprises unsubstituted C₁-C₁₂ heteroalkylene. In some embodiments, L¹ comprises unsubstituted C₁-C₆ heteroalkylene. In some embodiments, L¹ comprises unsubstituted C₁-C₃ heteroalkylene. In some embodiments, L¹ comprises unsubstituted linear heteroalkylene. In some embodiments, L¹ comprises unsubstituted linear C₁-C₁₂ heteroalkylene. In some embodiments, L¹ comprises unsubstituted linear C₁-C₆ heteroalkylene. In some embodiments, L¹ comprises unsubstituted linear C₁-C₃ heteroalkylene.

In some embodiments, L¹ comprises

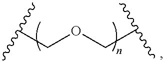

wherein n is an integer between 0 and 30, inclusive. In some embodiments, L¹ comprises —NHC(═O)— or —C(═O)NH—. In some embodiments, L¹ is

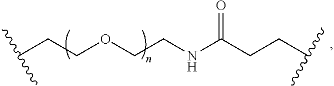

wherein n is an integer between 0 and 30, inclusive.

In some embodiments, L¹ comprises

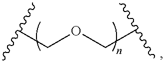

wherein n is an integer between 1 and 30, inclusive. In some embodiments, L¹ comprises

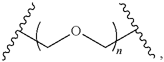

wherein n is an integer between 1 and 10, inclusive. In some embodiments, L¹ comprises

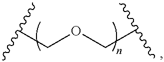

wherein n is an integer between 1 and 5, inclusive.

In some embodiments, L comprises

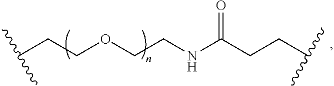

wherein n is an integer between 1 and 30, inclusive. In some embodiments, L¹ comprises

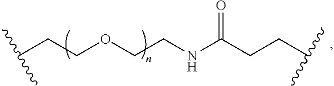

wherein n is an integer between 1 and 10, inclusive. In some embodiments, L¹ comprises

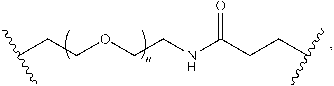

wherein n is an integer between 1 and 5, inclusive.

In some embodiments, L¹ is

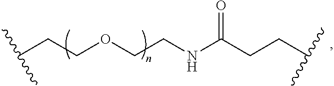

wherein n is an integer between 1 and 30, inclusive. In some embodiments, L¹ is

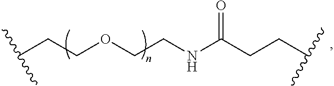

wherein n is an integer between 1 and 10, inclusive. In some embodiments, L¹ is

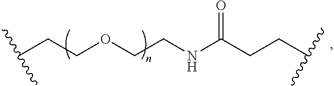

wherein n is an integer between 1 and 5, inclusive.

In some embodiments, L² is C₂ alkylene substituted with one or more substituents selected from halogen, optionally substituted alkyl, optionally substituted heteroalkyl, —OR^{A}, —N(R^{A})₂, and ═O. In some embodiments, L² is C₂ alkylene substituted with optionally substituted C₁₋₃ alkyl. In some embodiments, L² is C₂ alkylene substituted with unsubstituted C₁₋₃ alkyl. In some embodiments, L² is C₂ alkylene substituted with methyl, ethyl, n-propyl, or isopropyl. In some embodiments, L² is C₂ alkylene substituted with methyl.

In some embodiments, L² is unsubstituted C₂ alkylene (i.e., ethylene).

In some embodiments, L² is

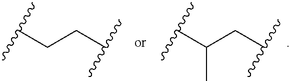

In some embodiments, the compound of Formula (I) is of Formula (I-a), or a salt thereof. In some embodiments, the compound of Formula (I-a) is of Formula (I-a-1), or a salt thereof. In some embodiments, the compound of Formula (I-a) is of Formula (I-a-2), or a salt thereof. In some embodiments, the compound of Formula (I-a) is of Formula (I′-a), or a salt thereof. In some embodiments, the compound of Formula (I′-a) is of Formula (I′-a-1), or a salt thereof. In some embodiments, the compound of Formula (I′-a) is of Formula (I′-a-2), or a salt thereof. In some embodiments of Formulae (I-a) and (I′-a), at least one instance of R^{3a} is halogen or —NO₂. In some embodiments of Formulae (I-a) and (I′-a), each instance of R^{3a} is independently halogen. In some embodiments of Formulae (I-a) and (I′-a), at least one instance of R^{3a} is fluoro. In some embodiments of Formulae (I-a) and (I′-a), at least one instance of R^{3a} is —NO₂.

In some embodiments, the compound of Formula (I) is of Formula (I-b), or a salt thereof. In some embodiments, the compound of Formula (I-b) is of Formula (I′-b), or a salt thereof.

In some embodiments, the compound of Formula (I) is of Formula (I-c), or a salt thereof. In some embodiments, the compound of Formula (I-c) is of Formula (I′-c), or a salt thereof. In some embodiments, the compound of Formula (I) is of Formula (I-cc), or a salt thereof. In some embodiments, the compound of Formula (I-c) is of Formula (I′-cc), or a salt thereof.

In some embodiments, the compound of Formula (I) is of Formula (I-d), or a salt thereof. In some embodiments, the compound of Formula (I-d) is of Formula (I-d-1), or a salt thereof. In some embodiments, the compound of Formula (I-d) is of Formula (I-d-2), or a salt thereof. In some embodiments, the compound of Formula (I-d) is of Formula (I′-d), or a salt thereof. In some embodiments of Formulae (I-d) and (I′-d), at least one instance of R^{3a} is halogen or —NO₂. In some embodiments of Formulae (I-d) and (I′-d), each instance of R^{3a} is independently halogen. In some embodiments of Formulae (I-d) and (I′-d), at least one instance of R^{3a} is fluoro. In some embodiments of Formulae (I-d) and (I′-d), at least one instance of R^{3a} is —NO₂. In some embodiments, the compound of Formula (I′-d) is of Formula (I′-d-1), or a salt thereof. In some embodiments, the compound of Formula (I′-d) is of Formula (I′-d-2), or a salt thereof.

In some embodiments, the compound of Formula (I) is of Formula (I-dd), or a salt thereof. In some embodiments, the compound of Formula (I-dd) is of Formula (I-dd-1), or a salt thereof. In some embodiments, the compound of Formula (I-dd) is of Formula (I-dd-2), or a salt thereof. In some embodiments, the compound of Formula (I-dd) is of Formula (I′-dd), or a salt thereof. In some embodiments of Formulae (I-dd) and (I′-dd), at least one instance of R^{3a} is halogen or —NO₂. In some embodiments of Formulae (I-dd) and (I′-dd), each instance of R^{3a} is independently halogen. In some embodiments of Formulae (I-dd) and (I′-dd), at least one instance of R^{3a} is fluoro. In some embodiments of Formulae (I-dd) and (I′-dd), at least one instance of R^{3a} is —NO₂. In some embodiments, the compound of Formula (I′-dd) is of Formula (I′-dd-1), or a salt thereof. In some embodiments, the compound of Formula (I′-dd) is of Formula (I′-dd-2), or a salt thereof.

In some embodiments, the compound of Formula (I) is of Formula (I-e), or a salt thereof. In some embodiments, the compound of Formula (I-e) is of Formula (I′-e), or a salt thereof. In some embodiments, the compound of Formula (I) is of Formula (I-ee), or a salt thereof. In some embodiments, the compound of Formula (I-e) is of Formula (I′-ee), or a salt thereof.

In some embodiments, the compound of Formula (I) is of Formula (I-f), or a salt thereof. In some embodiments, the compound of Formula (I-f) is of Formula (I′-f), or a salt thereof.

In some embodiments, the compound of Formula (I) is of Formula (I-g), or a salt thereof. In some embodiments, the compound of Formula (I-g) is of Formula (I-g-1), or a salt thereof. In some embodiments, the compound of Formula (I-g) is of Formula (I-g-2), or a salt thereof. In some embodiments, the compound of Formula (I-g) is of Formula (I′-g), or a salt thereof. In some embodiments of Formulae (I-g) and (I′-g), at least one instance of R^{3a} is halogen or —NO₂. In some embodiments of Formulae (I-g) and (I′-g), each instance of R^{3a} is independently halogen. In some embodiments of Formulae (I-g) and (I′-g), at least one instance of R^{3a} is fluoro. In some embodiments of Formulae (I-g) and (I′-g), at least one instance of R^{3a} is —NO₂. In some embodiments, the compound of Formula (I′-g) is of Formula (I′-g-1), or a salt thereof. In some embodiments, the compound of Formula (I′-g) is of Formula (I′-g-2), or a salt thereof.

In some embodiments, the compound of Formula (I) is of Formula (I-h), or a salt thereof. In some embodiments, the compound of Formula (I-h) is of Formula (I′-h), or a salt thereof.

In some embodiments, the compound of Formula (I) is of Formula (I-i), or a salt thereof. In some embodiments, the compound of Formula (I-i) is of Formula (I′-i), or a salt thereof. In some embodiments, the compound of Formula (I) is of Formula (I-ii), or a salt thereof. In some embodiments, the compound of Formula (I-i) is of Formula (I′-ii), or a salt thereof.

In some embodiments, the compound of Formula (I) is of Formula (I-j), or a salt thereof. In some embodiments, the compound of Formula (I-j) is of Formula (I-j-1), or a salt thereof. In some embodiments, the compound of Formula (I-j) is of Formula (I-j-2), or a salt thereof. In some embodiments, the compound of Formula (I-j) is of Formula (I-j-3), or a salt thereof. In some embodiments, the compound of Formula (I-j) is of Formula (I-j-4), or a salt thereof. In some embodiments, the compound of Formula (I-j) is of Formula (I-j-5), or a salt thereof. In some embodiments, the compound of Formula (I-j) is of Formula (I-j-6), or a salt thereof. In some embodiments, the compound of Formula (I-j) is of Formula (I′-j), or a salt thereof. In some embodiments of Formulae (I-j) and (I′-j), at least one instance of R^{3a} is halogen or —NO₂. In some embodiments of Formulae (I-j) and (I′-j), each instance of R^{3a} is independently halogen. In some embodiments of Formulae (I-j) and (I′-j), at least one instance of R^{3a} is fluoro. In some embodiments of Formulae (I-j) and (I′-j), at least one instance of R^{3a} is —NO₂. In some embodiments, the compound of Formula (I′-j) is of Formula (I′-j-1), or a salt thereof. In some embodiments, the compound of Formula (I′-j) is of Formula (I′-j-2), or a salt thereof. In some embodiments, the compound of Formula (I′-j) is of Formula (I′-j-3), or a salt thereof. In some embodiments, the compound of Formula (I′-j) is of Formula (I′-j-4), or a salt thereof. In some embodiments, the compound of Formula (I′-j) is of Formula (I′-j-5), or a salt thereof. In some embodiments, the compound of Formula (I′-j) is of Formula (I′-j-6), or a salt thereof. In some embodiments, the compound of Formula (I) is of Formula (I-jj), or a salt thereof. In some embodiments, the compound of Formula (I-jj) is of Formula (I-jj-1), or a salt thereof. In some embodiments, the compound of Formula (I-jj) is of Formula (I-jj-2), or a salt thereof. In some embodiments, the compound of Formula (I-jj) is of Formula (I-jj-3), or a salt thereof. In some embodiments, the compound of Formula (I-jj) is of Formula (I-jj-4), or a salt thereof. In some embodiments, the compound of Formula (I-jj) is of Formula (I-jj-5), or a salt thereof. In some embodiments, the compound of Formula (I-jj) is of Formula (I-jj-6), or a salt thereof. In some embodiments, the compound of Formula (I-jj) is of Formula (I′-jj), or a salt thereof. In some embodiments of Formulae (I-jj) and (I′-jj), at least one instance of R^{3a} is halogen or —NO₂. In some embodiments of Formulae (I-jj) and (I′-jj), each instance of R^{3a} is independently halogen. In some embodiments of Formulae (I-jj) and (I′-jj), at least one instance of R^{3a} is fluoro. In some embodiments of Formulae (I-jj) and (I′-jj), at least one instance of R^{3a} is —NO₂. In some embodiments, the compound of Formula (I′-jj) is of Formula (I′-jj-1), or a salt thereof. In some embodiments, the compound of Formula (I′-jj) is of Formula (I′-jj-2), or a salt thereof. In some embodiments, the compound of Formula (I′-jj) is of Formula (I′-jj-3), or a salt thereof. In some embodiments, the compound of Formula (I′-jj) is of Formula (I′-jj-4), or a salt thereof. In some embodiments, the compound of Formula (I′-jj) is of Formula (I′-jj-5), or a salt thereof. In some embodiments, the compound of Formula (I′-jj) is of Formula (I′-jj-6), or a salt thereof.

In some embodiments, the compound of Formula (I) is of Formula (I-k), or a salt thereof. In some embodiments, the compound of Formula (I-k) is of Formula (I-k-1), or a salt thereof. In some embodiments, the compound of Formula (I-k) is of Formula (I-k-2), or a salt thereof. In some embodiments, the compound of Formula (I) is of Formula (I′-k), or a salt thereof. In some embodiments, the compound of Formula (I′-k) is of Formula (I′-k-1), or a salt thereof. In some embodiments, the compound of Formula (I′-k) is of Formula (I′-k-2), or a salt thereof. In some embodiments, the compound of Formula (I) is of Formula (I-kk), or a salt thereof. In some embodiments, the compound of Formula (I-kk) is of Formula (I-kk-1), or a salt thereof. In some embodiments, the compound of Formula (I-kk) is of Formula (I-kk-2), or a salt thereof. In some embodiments, the compound of Formula (I) is of Formula (I′-kk), or a salt thereof. In some embodiments, the compound of Formula (I′-kk) is of Formula (I′-kk-1), or a salt thereof. In some embodiments, the compound of Formula (I′-kk) is of Formula (I′-kk-2), or a salt thereof.

In some embodiments, the compound of Formula (II) is of Formula (II-a-1), or a salt thereof. In some embodiments, the compound of Formula (II-a-1) is of Formula (II′-a-1), or a salt thereof. In some embodiments of Formulae (II-a-1) and (II′-a-1), R⁵ is —OH. In some embodiments of Formulae (II-a-1) and (II′-a-1), R⁵ is —NH₂. In some embodiments, the compound of Formula (II) is of Formula (II-a-2), or a salt thereof. In some embodiments, the compound of Formula (II-a-2) is of Formula (II′-a-2), or a salt thereof. In some embodiments of Formulae (II-a-2) and (II′-a-2), R⁵ is —OH. In some embodiments of Formulae (II-a-2) and (II′-a-2), R⁵ is —NH₂.

In some embodiments, the compound of Formula (II) is of Formula (II-b), or a salt thereof. In some embodiments, the compound of Formula (II-b) is of Formula (II′-b), or a salt thereof. In some embodiments of Formulae (II-b) and (II′-b), R⁵ is —OH. In some embodiments of Formulae (II-b) and (II′-b), R⁵ is —NH₂.

In some embodiments, the compound of Formula (II) is of Formula (II-c-1), or a salt thereof. In some embodiments, the compound of Formula (II-c-1) is of Formula (II′-c-1), or a salt thereof. In some embodiments of Formulae (II-c-1) and (II′-c-1), R⁵ is —OH. In some embodiments of Formulae (II-c-1) and (II′-c-1), R⁵ is —NH₂. In some embodiments, the compound of Formula (II) is of Formula (II-c-2), or a salt thereof. In some embodiments, the compound of Formula (II-c-2) is of Formula (II′-c-2), or a salt thereof. In some embodiments of Formulae (II-c-2) and (II′-c-2), R⁵ is —OH. In some embodiments of Formulae (II-c-2) and (II′-c-2), R⁵ is —NH₂.

In some embodiments, the compound of Formula (III-d) is of Formula (III-d-1), or a salt thereof. In some embodiments, the compound of Formula (III-d) is of Formula (III-d-2), or a salt thereof. In some embodiments, the compound of Formula (III-d) is of Formula (III′-d), or a salt thereof. In some embodiments, the compound of Formula (III′-d) is of Formula (III′-d-1), or a salt thereof. In some embodiments, the compound of Formula (III′-d) is of Formula (III′-d-2), or a salt thereof. In some embodiments of Formulae (III-d), (III-d-1), (III-d-2), (III′-d), (III′-d-1), and (III′-d-2), R⁵ is —OH. In some embodiments of Formulae (III-d), (III-d-1), (III-d-2), (III′-d), (III′-d-1), and (III′-d-2), R⁵ is —NH₂.

In some embodiments, the compound of Formula (III-d) is of Formula (III-e), or a salt thereof. In some embodiments, the compound of Formula (III-e) is of Formula (III-e-1), or a salt thereof. In some embodiments, the compound of Formula (III-e) is of Formula (III-e-2), or a salt thereof. In some embodiments, the compound of Formula (III-e) is of Formula (III′-e), or a salt thereof. In some embodiments, the compound of Formula (III′-e) is of Formula (III′-e-1), or a salt thereof. In some embodiments, the compound of Formula (III′-e) is of Formula (III′-e-2), or a salt thereof. In some embodiments of Formulae (III-e), (III-e-1), (III-e-2), (III′-e), (III′-e-1), and (III′-e-2), R⁵ is —OH. In some embodiments of Formulae (III-e), (III-e-1), (III-e-2), (III′-e), (III′-e-1), and (III′-e-2), R⁵ is —NH₂. In some embodiments, the compound of Formula (III-d) is of Formula (III-ee), or a salt thereof. In some embodiments, the compound of Formula (III-ee) is of Formula (III-ee-1), or a salt thereof. In some embodiments, the compound of Formula (III-ee) is of Formula (III-ee-2), or a salt thereof. In some embodiments, the compound of Formula (III-ee) is of Formula (III′-ee), or a salt thereof. In some embodiments, the compound of Formula (III′-ee) is of Formula (III′-ee-1), or a salt thereof. In some embodiments, the compound of Formula (III′-ee) is of Formula (III′-ee-2), or a salt thereof. In some embodiments of Formulae (III-ee), (III-ee-1), (III-ee-2), (III′-ee), (III′-ee-1), and (III′-ee-2), R⁵ is —OH. In some embodiments of Formulae (III-ee), (III-ee-1), (III-ee-2), (III′-ee), (III′-ee-1), and (III′-ee-2), R⁵ is —NH₂.

In some embodiments, the compound of Formula (III-d) is of Formula (III-f), or a salt thereof. In some embodiments, the compound of Formula (III-f) is of Formula (ITT-f-1), or a salt thereof. In some embodiments, the compound of Formula (III-f) is of Formula (III-f-2), or a salt thereof. In some embodiments, the compound of Formula (III-f) is of Formula (III′-f), or a salt thereof. In some embodiments, the compound of Formula (III′-f) is of Formula (II-f-1), or a salt thereof. In some embodiments, the compound of Formula (III′-f) is of Formula (III′-f-2), or a salt thereof. In some embodiments of Formulae (III-f), (ITT-f-1), (III-f-2), (III′-f), (II-f-1), and (III′-f-2), R⁵ is —OH. In some embodiments of Formulae (III-f), (ITT-f-1), (III-f-2), (III′-f), (II-f-1), and (III′-f-2), R⁵ is —NH₂.

In some embodiments, the compound of Formula (III-d) is of Formulae (III-g), or a salt thereof. In some embodiments, the compound of Formula (III-g) is of Formula (III-g-1), or a salt thereof. In some embodiments, the compound of Formula (III-g) is of Formula (III-g-2), or a salt thereof. In some embodiments, the compound of Formula (III-g) is of Formulae (III′-g), or a salt thereof. In some embodiments, the compound of Formula (III′-g) is of Formula (III′-g-1), or a salt thereof. In some embodiments, the compound of Formula (III′-g) is of Formula (III′-g-2), or a salt thereof. In some embodiments of Formulae (III-g), (III-g-1), (III-g-2), (III′-g), (III′-g-1), and (III′-g-2), R⁵ is —OH. In some embodiments of Formulae (III-g), (III-g-1), (III-g-2), (III′-g), (III′-g-1), and (III′-g-2), R⁵ is —NH₂. In some embodiments, the compound of Formula (III-d) is of Formulae (III-gg), or a salt thereof. In some embodiments, the compound of Formula (III-gg) is of Formula (III-gg-1), or a salt thereof. In some embodiments, the compound of Formula (III-gg) is of Formula (III-gg-2), or a salt thereof. In some embodiments, the compound of Formula (III-gg) is of Formulae (III′-gg), or a salt thereof. In some embodiments, the compound of Formula (III′-gg) is of Formula (III′-gg-1), or a salt thereof. In some embodiments, the compound of Formula (III′-gg) is of Formula (III′-gg-2), or a salt thereof. In some embodiments of Formulae (III-gg), (III-gg-1), (III-gg-2), (III′-gg), (III′-gg-1), and (III′-gg-2), R⁵ is —OH. In some embodiments of Formulae (III-gg), (III-gg-1), (III-gg-2), (III′-gg), (III′-gg-1), and (III′-gg-2), R⁵ is —NH₂.

In some embodiments, the peptide remains in the solid phase while conjugated to the solid support.

As generally described herein, Z¹ comprises an oligonucleotide. In some embodiments, Z¹ further comprises a linking group. In some embodiments, the linking group comprises a polypeptidyl group. In some embodiments, the linking group further comprises at least one of optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof.

In some embodiments, Z¹ further comprises a binding group. In some embodiments, the binding group comprises a biotin moiety. In some embodiments, Z¹ further comprises a biotin moiety. In some embodiments, the biotin moiety is a bis-biotin moiety.

In some embodiments, the binding group comprises or is conjugated to an avidin protein. In some embodiments, Z¹ further comprises an avidin protein. In some embodiments, the biotin moiety comprises an avidin protein. In some embodiments, the biotin moiety is conjugated to an avidin protein.

In some embodiments, the compound comprising Z¹ is immobilized to a surface (e.g., a surface of a sample well). In some embodiments, the compound comprising Z¹ is immobilized to a surface of a sample well. In some embodiments, Z¹ is immobilized to a surface (e.g., a surface of a sample well). In some embodiments, Z¹ is immobilized to a surface of a sample well. In some embodiments, the avidin protein is immobilized to a surface (e.g., a surface of a sample well). In some embodiments, the avidin protein is immobilized to a surface of a sample well.

In certain embodiments, the method comprises iterative detection and cleavage at a terminal end of a polypeptide.

In certain embodiments, the peptidase is an exopeptidase. An exopeptidase generally requires a polypeptide substrate to comprise at least one of a free amino group at its amino-terminus or a free carboxyl group at its carboxy-terminus. In some embodiments, an exopeptidase in accordance with the application hydrolyses a bond at or near a terminus of a polypeptide. In some embodiments, an exopeptidase hydrolyses a bond not more than three residues from a polypeptide terminus. For example, in some embodiments, a single hydrolysis reaction catalyzed by an exopeptidase cleaves a single amino acid, a dipeptide, or a tripeptide from a polypeptide terminal end.

In some embodiments, an exopeptidase in accordance with the application is an aminopeptidase or a carboxypeptidase, which cleaves a single amino acid from an amino- or a carboxy-terminus, respectively. In some embodiments, an exopeptidase in accordance with the application is a dipeptidyl-peptidase or a peptidyl-dipeptidase, which cleave a dipeptide from an amino- or a carboxy-terminus, respectively. In yet other embodiments, an exopeptidase in accordance with the application is a tripeptidyl-peptidase, which cleaves a tripeptide from an amino-terminus. Peptidase classification and activities of each class or subclass thereof is well known and described in the literature (see, e.g., Gurupriya, V. S. & Roy, S. C. Proteases and Protease Inhibitors in Male Reproduction. Proteases in Physiology and Pathology 195-216 (2017); and Brix, K. & Stöcker, W. Proteases: Structure and Function. Chapter 1). In some embodiments, a peptidase in accordance with the application removes more than three amino acids from a polypeptide terminus. Accordingly, in some embodiments, the peptidase is an endopeptidase, e.g., that cleaves preferentially at particular positions (e.g., before or after a particular amino acid). In some embodiments, the size of a polypeptide cleavage product of endopeptidase activity will depend on the distribution of cleavage sites (e.g., amino acids) within the polypeptide being analyzed.

An exopeptidase in accordance with the application may be selected or engineered based on the directionality of a sequencing reaction. For example, in embodiments of sequencing from an amino-terminus to a carboxy-terminus of a polypeptide, an exopeptidase comprises aminopeptidase activity. Conversely, in embodiments of sequencing from a carboxy-terminus to an amino-terminus of a polypeptide, an exopeptidase comprises carboxypeptidase activity. Examples of carboxypeptidases that recognize specific carboxy-terminal amino acids have been described in the literature (see, e.g., Garcia-Guerrero, M. C., et al. (2018) PNAS 115(17)).

In some embodiments, the peptidase is an aminopeptidase that selectively binds one or more types of amino acids. In some embodiments, an aminopeptidase is non-specific such that it cleaves most or all types of amino acids from a terminal end of a polypeptide. In some embodiments, an aminopeptidase is more efficient at cleaving one or more types of amino acids from a terminal end of a polypeptide as compared to other types of amino acids at the terminal end of the polypeptide. For example, an aminopeptidase in accordance with the application specifically cleaves alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and/or valine. In some embodiments, an aminopeptidase is a proline aminopeptidase. In some embodiments, an aminopeptidase is a proline iminopeptidase. In some embodiments, an aminopeptidase is a glutamate/aspartate-specific aminopeptidase. In some embodiments, an aminopeptidase is a methionine-specific aminopeptidase. In some embodiments, an aminopeptidase is a non-specific aminopeptidase. In some embodiments, a non-specific aminopeptidase is a zinc metalloprotease.

In some aspects, the disclosure provides an aminopeptidase having an amino acid sequence selected from Table 3. It should be appreciated that the example sequences in Table 3 and other examples described herein are meant to be non-limiting, and aminopeptidases in accordance with the disclosure can include any homologs, variants, or fragments thereof minimally containing domains or subdomains responsible for amino acid cleavage.

In some embodiments, an aminopeptidase has an amino acid sequence that is at least 80% identical to an amino acid sequence selected from Table 3. In some embodiments, an aminopeptidase has at least 40%, at least 50%, at least 60%, at least 70%, at least 80%, at least 85%, at least 90%, at least 92%, at least 94%, at least 95%, at least 96%, at least 98%, or higher, amino acid sequence identity to an amino acid sequence selected from Table 3. In some embodiments, an aminopeptidase has 25-50%, 50-60%, 60-70%, 70-80%, 80-90%, 90-95%, 92-99%, 94-99%, 95-99%, 40-100%, 50-100%, 60-100%, 70-100%, 80-100%, 90-100%, 92-100%, 94-100%, 95-100%, 96-100%, or 100% amino acid sequence identity to an amino acid sequence selected from Table 3.

In some embodiments, the aminopeptidase is a synthetic or recombinant aminopeptidase. In some embodiments, the aminopeptidase is a monomeric aminopeptidase. In some embodiments, the aminopeptidase is a multimeric aminopeptidase (e.g., a multimeric complex of monomeric subunits, which may be the same or different). In some embodiments, the aminopeptidase is a modified aminopeptidase and includes one or more amino acid mutations relative to a sequence set forth in Table 3.

In some embodiments, the aminopeptidase is an aminopeptidase obtained or derived from a particular source (e.g., organism). As described herein, in some embodiments, an aminopeptidase identified as being from a particular organism does not impart a requirement that the aminopeptidase have an amino acid sequence that is 100% identical to a naturally-occurring aminopeptidase from the organism, although it may in some embodiments.

TABLE 3
Non-limiting examples of aminopeptidases.

| Name | Sequence |
|---|---|
| Pyrococcus | MEVRNMVDYELLKKVVEAPGVSGYEFLGIRDVVIEEIKDYVDEVKVDKLGNVIAHKKGEGPKVMI |
| horikoshii TET II | AAHMDQIGLMVTHIEKNGFLRVAPIGGVDPKTLIAQRFKVWIDKGKFIYGVGASVPPHIQKPEDR |
| Aminopeptidase | KKAPDWDQIFIDIGAESKEEAEDMGVKIGTVITWDGRLERLGKHRFVSIAFDDRIAVYTILEVAK |
| (hTET II) | QLKDAKADVYFVATVQEEVGLRGARTSAFGIEPDYGFAIDVTIAADIPGTPEHKQVTHLGKGTAI |
| | KIMDRSVICHPTIVRWLEELAKKHEIPYQLEILLGGGTDAGAIHLTKAGVPTGALSVPARYIHSN |
| | TEVVDERDVDATVELMTKALENIHELKI (SEQ ID NO: 1) |
| AP30 | MEVRNMVDYELLKKVVEAPGVSGYEFLGIRDVVIEEIKDYVDEVKVDKLGNVIAHKKGEGPKVMI |
| | AAHMDQIGLMVTHIEKNGFLRVAPIGGVDPKTLIAQRFKVWIDKGKFIYGVGASVPPHIQKPEDR |
| | KKAPDWDQIFIDIGAESKEEAEDMGVKIGTVITWDGRLERLGKHRFVSIAFDDRIAVYTILEVAK |
| | QLKDAKADVYFVATVQEEVGLRGARTSAFGIEPDYGFAIDVTIAADIPGTPEHKQVTHLGKGTAI |
| | KIMDRSVICHPTIVRWLEELAKKHEIPYQLEILLGGGTDAGAIHLTKAGVPTGALSVPARYIHSN |
| | TEVVDERDVDATVELMTKALENIHELKIGGSHHHHHHHHHHGGGSGGGSGGGSGLNDFFEAQKIE |
| | WHEGGGSGGGSGGGSGLNDFFEAQKIEWHE (SEQ ID NO: 2) |
| Pyrococcus | MDLKGGESMVDWKLMQEIIEAPGVSGYEHLGIRDIVVDVLKEVADEVKVDKLGNVIAHFKGSSPR |
| horikoshii TET III | IMVAAHMDKIGVMVNHIDKDGYLHIVPIGGVLPETLVAQRIRFFTEKGERYGVVGVLPPHLRRGQ |
| Aminopeptidase | EDKGSKIDWDQIVVDVGASSKEEAEEMGFRVGTVGEFAPNFTRLNEHRFATPYLDDRICLYAMIE |
| (hTET III) | AARQLGDHEADIYIVGSVQEEVGLRGARVASYAINPEVGIAMDVTFAKQPHDKGKIVPELGKGPV |
| | MDVGPNINPKLRAFADEVAKKYEIPLQVEPSPRPTGTDANMQINREGVATAVLSIPIRYMHSQVE |
| | LADARDVDNTIKLAKALLEELKPMDFTP (SEQ ID NO: 3) |
| AP37 | MDLKGGESMVDWKLMQEIIEAPGVSGYEHLGIRDIVVDVLKEVADEVKVDKLGNVIAHFKGSSPR |
| | IMVAAHMDKIGVMVNHIDKDGYLHIVPIGGVLPETLVAQRIRFFTEKGERYGVVGVLPPHLRRGQ |
| | EDKGSKIDWDQIVVDVGASSKEEAEEMGFRVGTVGEFAPNFTRLNEHRFATPYLDDRICLYAMIE |
| | AARQLGDHEADIYIVGSVQEEVGLRGARVASYAINPEVGIAMDVTFAKQPHDKGKIVPELGKGPV |
| | MDVGPNINPKLRAFADEVAKKYEIPLQVEPSPRPTGTDANMQINREGVATAVLSIPIRYMHSQVE |
| | LADARDVDNTIKLAKALLEELKPMDFTPGHHHHHHHHHH (SEQ ID NO: 4) |
| Yersinia pestis Xaa- | MTQQEYQNRRQALLAKMAPGSAAIIFAAPEATRSADSEYPYRQNSDFSYLTGFNEPEAVLILVKS |
| Proly1 | DETHNHSVLFNRIRDLTAEIWFGRRLGQEAAPTKLAVDRALPFDEINEQLYLLLNRLDVIYHAQG |
| aminopeptidase | QYAYADNIVFAALEKLRHGFRKNLRAPATLTDWRPWLHEMRLFKSAEEIAVLRRAGEISALAHTR |
| (yPIP) | AMEKCRPGMFEYQLEGEILHEFTRHGARYPAYNTIVGGGENGCILHYTENECELRDGDLVLIDAG |
| | CEYRGYAGDITRTFPVNGKFTPAQRAVYDIVLAAINKSLTLFRPGTSIREVTEEVVRIMVVGLVE |
| | LGILKGDIEQLIAEQAHRPFFMHGLSHWLGMDVHDVGDYGSSDRGRILEPGMVLTVEPGLYIAPD |
| | ADVPPQYRGIGIRIEDDIVITATGNENLTASVVKDPDDIEALMALNHAGENLYFQLE (SEQ ID |
| | NO: 5) |
| yPIP-6x His | MTQQEYQNRRQALLAKMAPGSAAIIFAAPEATRSADSEYPYRQNSDFSYLTGFNEPEAVLILVKS |
| | DETHNHSVLFNRIRDLTAEIWFGRRLGQEAAPTKLAVDRALPFDEINEQLYLLLNRLDVIYHAQG |
| | QYAYADNIVFAALEKLRHGFRKNLRAPATLTDWRPWLHEMRLFKSAEEIAVLRRAGEISALAHTR |
| | AMEKCRPGMFEYQLEGEILHEFTRHGARYPAYNTIVGGGENGCILHYTENECELRDGDLVLIDAG |
| | CEYRGYAGDITRTFPVNGKFTPAQRAVYDIVLAAINKSLTLFRPGTSIREVTEEVVRIMVVGLVE |
| | LGILKGDIEQLIAEQAHRPFFMHGLSHWLGMDVHDVGDYGSSDRGRILEPGMVLTVEPGLYIAPD |
| | ADVPPQYRGIGIRIEDDIVITATGNENLTASVVKDPDDIEALMALNHAGENLYFQLEHHHHHH |
| | (SEQ ID NO: 6) |
| yPIP (truncated) | MTQQEYQNRRQALLAKMAPGSAAIIFAAPEATRSADSEYPYRQNSDFSYLTGFNEPEAVLILVKS |
| | DETHNHSVLFNRIRDLTAEIWFGRRLGQEAAPTKLAVDRALPFDEINEQLYLLLNRLDVIYHAQG |
| | QYAYADNIVFAALEKLRHGFRKNLRAPATLTDWRPWLHEMRLFKSAEEIAVLRRAGEISALAHTR |
| | AMEKCRPGMFEYQLEGEILHEFTRHGARYPAYNTIVGGGENGCILHYTENECELRDGDLVLIDAG |
| | CEYRGYAGDITRTFPVNGKFTPAQRAVYDIVLAAINKSLTLFRPGTSIREVTEEVVRIMVVGLVE |
| | LGILKGDIEQLIAEQAHRPFFMHGLSHWLGMDVHDVGDYGSSDRGRILEPGMVLTVEPGLYIAPD |
| | ADVPPQYRGIGIRIEDDIVITATGNENLTASVVKDPDDIEALMALNHAGENLYFQ (SEQ ID |
| | NO: 7) |
| AP70 | MTQQEYQNRRQALLAKMAPGSAAIIFAAPEATRSADSEYPYRQNSDFSYLTGFNEPEAVLILVKS |
| | DETHNHSVLFNRIRDLTAEIWFGRRLGQEAAPTKLAVDRALPFDEINEQLYLLLNRLDVIYHAQG |
| | QYAYADNIVFAALEKLRHGFRKNLRAPATLTDWRPWLHEMRLFKSAEEIAVLRRAGEISALAHTR |
| | AMEKCRPGMFEYQLEGEILHEFTRHGARYPAYNTIVGGGENGCILHYTENECELRDGDLVLIDAG |
| | CEYRGYAGDITRTFPVNGKFTPAQRAVYDIVLAAINKSLTLFRPGTSIREVTEEVVRIMVVGLVE |
| | LGILKGDIEQLIAEQAHRPFFMHGLSHWLGMDVHDVGDYGSSDRGRILEPGMVLTVEPGLYIAPD |
| | ADVPPQYRGIGIRIEDDIVITATGNENLTASVVKDPDDIEALMALNHAGENLYFQGGSHHHHHH |
| | (SEQ ID NO: 8) |
| L. pneumophila M1 | MMVKQGVFMKTDQSKVKKLSDYKSLDYFVIHVDLQIDLSKKPVESKARLTVVPNLNVDSHSNDLV |
| Aminopeptidase | LDGENMTLVSLQMNDNLLKENEYELTKDSLIIKNIPQNTPFTIEMTSLLGENTDLFGLYETEGVA |
| (Glu/Asp Specific) | LVKAESEGLRRVFYLPDRPDNLATYKTTIIANQEDYPVLLSNGVLIEKKELPLGLHSVTWLDDVP |
| | KPSYLFALVAGNLQRSVTYYQTKSGRELPIEFYVPPSATSKCDFAKEVLKEAMAWDERTFNLECA |
| | LRQHMVAGVDKYASGASEPTGLNLFNTENLFASPETKTDLGILRVLEVVAHEFFHYWSGDRVTIR |
| | DWFNLPLKEGLTTFRAAMFREELFGTDLIRLLDGKNLDERAPRQSAYTAVRSLYTAAAYEKSADI |
| | FRMMMLFIGKEPFIEAVAKFFKDNDGGAVTLEDFIESISNSSGKDLRSFLSWFTESGIPELIVTD |
| | ELNPDTKQYFLKIKTVNGRNRPIPILMGLLDSSGAEIVADKLLIVDQEEIEFQFENIQTRPIPSL |
| | LRSFSAPVHMKYEYSYQDLLLLMQFDTNLYNRCEAAKQLISALINDFCIGKKIELSPQFFAVYKA |
| | LLSDNSLNEWMLAELITLPSLEELIENQDKPDFEKLNEGRQLIQNALANELKTDFYNLLFRIQIS |
| | GDDDKQKLKGFDLKQAGLRRLKSVCFSYLLNVDFEKTKEKLILQFEDALGKNMTETALALSMLCE |
| | INCEEADVALEDYYHYWKNDPGAVNNWFSIQALAHSPDVIERVKKLMRHGDFDLSNPNKVYALLG |
| | SFIKNPFGFHSVTGEGYQLVADAIFDLDKINPTLAANLTEKFTYWDKYDVNRQAMMISTLKIIYS |
| | NATSSDVRTMAKKGLDKVKEDLPLPIHLTFHGGSTMQDRTAQLIADGNKENAYQLH (SEQ ID |
| | NO: 9) |
| E. coli methionine | MGTAISIKTPEDIEKMRVAGRLAAEVLEMIEPYVKPGVSTGELDRICNDYIVNEQHAVSACLGYH |
| aminopeptidase | GYPKSVCISINEVVCHGIPDDAKLLKDGDIVNIDVTVIKDGFHGDTSKMFIVGKPTIMGERLCRI |
| (Met specific) | TQESLYLALRMVKPGINLREIGAAIQKFVEAEGFSVVREYCGHGIGRGFHEEPQVLHYDSRETNV |
| | VLKPGMTFTIEPMVNAGKKEIRTMKDGWTVKTKDRSLSAQYEHTIVVTDNGCEILTLRKDDTIPA |
| | IISHD (SEQ ID NO: 10) |
| M. smegmatis | MGTLEANTNGPGSMLSRMPVSSRTVPFGDHETWVQVTTPENAQPHALPLIVLHGGPGMAHNYVAN |
| Proline | IAALADETGRTVIHYDQVGCGNSTHLPDAPADFWTPQLFVDEFHAVCTALGIERYHVLGQSWGGM |
| iminopeptidase | LGAEIAVRQPSGLVSLAICNSPASMRLWSEAAGDLRAQLPAETRAALDRHEAAGTITHPDYLQAA |
| (Pro specific) | AEFYRRHVCRVVPTPQDFADSVAQMEAEPTVYHTMNGPNEFHVVGTLGDWSVIDRLPDVTAPVLV |
| | IAGEHDEATPKTWQPFVDHIPDVRSHVFPGTSHCTHLEKPEEFRAVVAQFLHQHDLAADARV |
| | (SEQ ID NO: 11) |
| P. furiosus | MDTEKLMKAGEIAKKVREKAIKLARPGMLLLELAESIEKMIMELGGKPAFPVNLSINEIAAHYTP |
| methionine | YKGDTTVLKEGDYLKIDVGVHIDGFIADTAVTVRVGMEEDELMEAAKEALNAAISVARAGVEIKE |
| aminopeptidase | LGKAIENEIRKRGFKPIVNLSGHKIERYKLHAGISIPNIYRPHDNYVLKEGDVFAIEPFATIGAG |
| | QVIEVPPTLIYMYVRDVPVRVAQARFLLAKIKREYGTLPFAYRWLQNDMPEGQLKLALKTLEKAG |
| | AIYGYPVLKEIRNGIVAQFEHTIIVEKDSVIVTQDMINKSTLE (SEQ ID NO: 12) |
| Aeromonas sobria | HMSSPLHYVLDGIHCEPHFFTVPLDHQQPDDEETITLFGRTLCRKDRLDDELPWLLYLQGGPGFG |
| Proline | APRPSANGGWIKRALQEFRVLLLDQRGTGHSTPIHAELLAHLNPRQQADYLSHFRADSIVRDAEL |
| aminopeptidase | IREQLSPDHPWSLLGQSFGGFCSLTYLSLFPDSLHEVYLTGGVAPIGRSADEVYRATYQRVADKN |
| | RAFFARFPHAQAIANRLATHLQRHDVRLPNGQRLTVEQLQQQGLDLGASGAFEELYYLLEDAFIG |
| | EKLNPAFLYQVQAMQPFNTNPVFAILHELIYCEGAASHWAAERVRGEFPALAWAQGKDFAFTGEM |
| | IFPWMFEQFRELIPLKEAAHLLAEKADWGPLYDPVQLARNKVPVACAVYAEDMYVEFDYSRETLK |
| | GLSNSRAWITNEYEHNGLRVDGEQILDRLIRLNRDCLE (SEQ ID NO: 13) |
| Pyrococcus furiosus | MKERLEKLVKFMDENSIDRVFIAKPVNVYYFSGTSPLGGGYIIVDGDEATLYVPELEYEMAKEES |
| Proline | KLPVVKFKKFDEIYEILKNTETLGIEGTLSYSMVENFKEKSNVKEFKKIDDVIKDLRIIKTKEEI |
| Aminopeptidase (X- | EIIEKACEIADKAVMAAIEEITEGKREREVAAKVEYLMKMNGAEKPAFDTIIASGHRSALPHGVA |
| /-Pro) | SDKRIERGDLVVIDLGALYNHYNSDITRTIVVGSPNEKQREIYEIVLEAQKRAVEAAKPGMTAKE |
| | LDSIAREIIKEYGYGDYFIHSLGHGVGLEIHEWPRISQYDETVLKEGMVITIEPGIYIPKLGGVR |
| | IEDTVLITENGAKRLTKTERELL (SEQ ID NO: 14) |
| Elizabethkingia | MIPITTPVGNFKVWTKRFGTNPKIKVLLLHGGPAMTHEYMECFETFFQREGFEFYEYDQLGSYYS |
| meningoseptica | DQPTDEKLWNIDRFVDEVEQVRKAIHADKENFYVLGNSWGGILAMEYALKYQQNLKGLIVANMMA |
| Proline | SAPEYVKYAEVLSKQMKPEVLAEVRAIEAKKDYANPRYTELLFPNYYAQHICRLKEWPDALNRSL |
| aminopeptidase | KHVNSTVYTLMQGPSELGMSSDARLAKWDIKNRLHEIATPTLMIGARYDTMDPKAMEEQSKLVQK |
| | GRYLYCPNGSHLAMWDDQKVFMDGVIKFIKDVDTKSFN (SEQ ID NO: 15) |
| N. gonorrhoeae | MYEIKQPFHSGYLQVSEIHQIYWEESGNPDGVPVIFLHGGPGAGASPECRGFFNPDVFRIVIIDQ |
| Proline | RGCGRSHPYACAEDNTTWDLVADIEKVREMLGIGKWLVFGGSWGSTLSLAYAQTHPERVKGLVLR |
| Iminopeptidase | GIFLCRPSETAWLNEAGGVSRIYPEQWQKFVAPIAENRRNRLIEAYHGLLFHQDEEVCLSAAKAW |
| | ADWESYLIRFEPEGVDEDAYASLAIARLENHYFVNGGWLQGDKAILNNIGKIRHIPTVIVQGRYD |
| | LCTPMQSAWELSKAFPEAELRVVQAGHCAFDPPLADALVQAVEDILPRLL |
| | (SEQ ID NO: 16) |
| E. coli | MTQQPQAKYRHDYRAPDYQITDIDLTFDLDAQKTVVTAVSQAVRHGASDAPLRLNGEDLKLVSVH |
| Aminopeptidase N | INDEPWTAWKEEEGALVISNLPERFTLKIINEISPAANTALEGLYQSGDALCTQCEAEGFRHITY |
| (Zinc | YLDRPDVLARFTTKIIADKIKYPFLLSNGNRVAQGELENGRHWVQWQDPFPKPCYLFALVAGDFD |
| Metalloprotease) | VLRDTFTTRSGREVALELYVDRGNLDRAPWAMTSLKNSMKWDEERFGLEYDLDIYMIVAVDFFNM |
| | GAMENKGLNIFNSKYVLARTDTATDKDYLDIERVIGHEYFHNWTGNRVTCRDWFQLSLKEGLTVF |
| | RDQEFSSDLGSRAVNRINNVRTMRGLQFAEDASPMAHPIRPDMVIEMNNFYTLTVYEKGAEVIRM |
| | IHTLLGEENFQKGMQLYFERHDGSAATCDDFVQAMEDASNVDLSHFRRWYSQSGTPIVTVKDDYN |
| | PETEQYTLTISQRTPATPDQAEKQPLHIPFAIELYDNEGKVIPLQKGGHPVNSVLNVTQAEQTFV |
| | FDNVYFQPVPALLCEFSAPVKLEYKWSDQQLTFLMRHARNDFSRWDAAQSLLATYIKLNVARHQQ |
| | GQPLSLPVHVADAFRAVLLDEKIDPALAAEILTLPSVNEMAELFDIIDPIAIAEVREALTRTLAT |
| | ELADELLAIYNANYQSEYRVEHEDIAKRTLRNACLRFLAFGETHLADVLVSKQFHEANNMTDALA |
| | ALSAAVAAQLPCRDALMQEYDDKWHQNGLVMDKWFILQATSPAANVLETVRGLLQHRSFTMSNPN |
| | RIRSLIGAFAGSNPAAFHAEDGSGYLFLVEMLTDLNSRNPQVASRLIEPLIRLKRYDAKRQEKMR |
| | AALEQLKGLENLSGDLYEKITKALA (SEQ ID NO: 17) |
| P. falciparum M1 | PKIHYRKDYKPSGFIINQVTLNINIHDQETIVRSVLDMDISKHNVGEDLVFDGVGLKINEISINN |
| aminopeptidase | KKLVEGEEYTYDNEFLTIFSKFVPKSKFAFSSEVIIHPETNYALTGLYKSKNIIVSQCEATGFRR |
| | ITFFIDRPDMMAKYDVTVTADKEKYPVLLSNGDKVNEFEIPGGRHGARFNDPPLKPCYLFAVVAG |
| | DLKHLSATYITKYTKKKVELYVFSEEKYVSKLQWALECLKKSMAFDEDYFGLEYDLSRLNLVAVS |
| | DFNVGAMENKGLNIFNANSLLASKKNSIDFSYARILTVVGHEYFHQYTGNRVTLRDWFQLTLKEG |
| | LTVHRENLFSEEMTKTVTTRLSHVDLLRSVQFLEDSSPLSHPIRPESYVSMENFYTTTVYDKGSE |
| | VMRMYLTILGEEYYKKGFDIYIKKNDGNTATCEDFNYAMEQAYKMKKADNSANLNQYLLWFSQSG |
| | TPHVSFKYNYDAEKKQYSIHVNQYTKPDENQKEKKPLFIPISVGLINPENGKEMISQTTLELTKE |
| | SDTFVFNNIAVKPIPSLFRGFSAPVYIEDQLTDEERILLLKYDSDAFVRYNSCTNIYMKQILMNY |
| | NEFLKAKNEKLESFQLTPVNAQFIDAIKYLLEDPHADAGFKSYIVSLPQDRYIINFVSNLDTDVL |
| | ADTKEYIYKQIGDKLNDVYYKMFKSLEAKADDLTYFNDESHVDFDQMNMRTLRNTLLSLLSKAQY |
| | PNILNEIIEHSKSPYPSNWLTSLSVSAYFDKYFELYDKTYKLSKDDELLLQEWLKTVSRSDRKDI |
| | YEILKKLENEVLKDSKNPNDIRAVYLPFTNNLRRFHDISGKGYKLIAEVITKTDKFNPMVATQLC |
| | EPFKLWNKLDTKRQELMLNEMNTMLQEPQISNNLKEYLLRLTNK (SEQ ID NO: 18) |
| Puromycin-sensitive | MWLAAAAPSLARRLLFLGPPPPPLLLLVFSRSSRRRLHSLGLAAMPEKRPFERLPADVSPINYSL |
| aminopeptidase | CLKPDLLDFTFEGKLEAAAQVRQATNQIVMNCADIDIITASYAPEGDEEIHATGFNYQNEDEKVT |
| (NPEPPS) | LSFPSTLQTGTGTLKIDFVGELNDKMKGFYRSKYTTPSGEVRYAAVTQFEATDARRAFPCWDEPA |
| | IKATFDISLVVPKDRVALSNMNVIDRKPYPDDENLVEVKFARTPVMSTYLVAFVVGEYDFVETRS |
| | KDGVCVRVYTPVGKAEQGKFALEVAAKTLPFYKDYFNVPYPLPKIDLIAIADFAAGAMENWGLVT |
| | YRETALLIDPKNSCSSSRQWVALVVGHELAHQWFGNLVTMEWWTHLWLNEGFASWIEYLCVDHCF |
| | PEYDIWTQFVSADYTRAQELDALDNSHPIEVSVGHPSEVDEIFDAISYSKGASVIRMLHDYIGDK |
| | DFKKGMNMYLTKFQQKNAATEDLWESLENASGKPIAAVMNTWTKQMGFPLIYVEAEQVEDDRLLR |
| | LSQKKFCAGGSYVGEDCPQWMVPITISTSEDPNQAKLKILMDKPEMNVVLKNVKPDQWVKLNLGT |
| | VGFYRTQYSSAMLESLLPGIRDLSLPPVDRLGLQNDLFSLARAGIISTVEVLKVMEAFVNEPNYT |
| | VWSDLSCNLGILSTLLSHTDFYEEIQEFVKDVFSPIGERLGWDPKPGEGHLDALLRGLVLGKLGK |
| | AGHKATLEEARRRFKDHVEGKQILSADLRSPVYLTVLKHGDGTTLDIMLKLHKQADMQEEKNRIE |
| | RVLGATLLPDLIQKVLTFALSEEVRPQDTVSVIGGVAGGSKHGRKAAWKFIKDNWEELYNRYQGG |
| | FLISRLIKLSVEGFAVDKMAGEVKAFFESHPAPSAERTIQQCCENILLNAAWLKRDAESIHQYLL |
| | QRKASPPTV (SEQ ID NO: 19) |
| NPEPPS E366V | MWLAAAAPSLARRLLFLGPPPPPLLLLVFSRSSRRRLHSLGLAAMPEKRPFERLPADVSPINYSL |
| | CLKPDLLDFTFEGKLEAAAQVRQATNQIVMNCADIDIITASYAPEGDEEIHATGFNYQNEDEKVT |
| | LSFPSTLQTGTGTLKIDFVGELNDKMKGFYRSKYTTPSGEVRYAAVTQFEATDARRAFPCWDEPA |
| | IKATFDISLVVPKDRVALSNMNVIDRKPYPDDENLVEVKFARTPVMSTYLVAFVVGEYDFVETRS |
| | KDGVCVRVYTPVGKAEQGKFALEVAAKTLPFYKDYFNVPYPLPKIDLIAIADFAAGAMENWGLVT |
| | YRETALLIDPKNSCSSSRQWVALVVGHVLAHQWFGNLVTMEWWTHLWLNEGFASWIEYLCVDHCF |
| | PEYDIWTQFVSADYTRAQELDALDNSHPIEVSVGHPSEVDEIFDAISYSKGASVIRMLHDYIGDK |
| | DFKKGMNMYLTKFQQKNAATEDLWESLENASGKPIAAVMNTWTKQMGFPLIYVEAEQVEDDRLLR |
| | LSQKKFCAGGSYVGEDCPQWMVPITISTSEDPNQAKLKILMDKPEMNVVLKNVKPDQWVKLNLGT |
| | VGFYRTQYSSAMLESLLPGIRDLSLPPVDRLGLQNDLFSLARAGIISTVEVLKVMEAFVNEPNYT |
| | VWSDLSCNLGILSTLLSHTDFYEEIQEFVKDVFSPIGERLGWDPKPGEGHLDALLRGLVLGKLGK |
| | AGHKATLEEARRRFKDHVEGKQILSADLRSPVYLTVLKHGDGTTLDIMLKLHKQADMQEEKNRIE |
| | RVLGATLLPDLIQKVLTFALSEEVRPQDTVSVIGGVAGGSKHGRKAAWKFIKDNWEELYNRYQGG |
| | FLISRLIKLSVEGFAVDKMAGEVKAFFESHPAPSAERTIQQCCENILLNAAWLKRDAESIHQYLL |
| | QRKASPPTV (SEQ ID NO: 20) |
| Francisella | MIYEFVMTDPKIKYLKDYKPSNYLIDETHLIFELDESKTRVTANLYIVANRENRENNTLVLDGVE |
| tularensis | LKLLSIKLNNKHLSPAEFAVNENQLIINNVPEKFVLQTVVEINPSANTSLEGLYKSGDVFSTQCE |
| Aminopeptidase N | ATGFRKITYYLDRPDVMAAFTVKIIADKKKYPIILSNGDKIDSGDISDNQHFAVWKDPFKKPCYL |
| | FALVAGDLASIKDTYITKSQRKVSLEIYAFKQDIDKCHYAMQAVKDSMKWDEDRFGLEYDLDTFM |
| | IVAVPDFNAGAMENKGLNIFNTKYIMASNKTATDKDFELVQSVVGHEYFHNWTGDRVTCRDWFQL |
| | SLKEGLTVFRDQEFTSDLNSRDVKRIDDVRIIRSAQFAEDASPMSHPIRPESYIEMNNFYTVTVY |
| | NKGAEIIRMIHTLLGEEGFQKGMKLYFERHDGQAVTCDDFVNAMADANNRDFSLFKRWYAQSGTP |
| | NIKVSENYDASSQTYSLTLEQTTLPTADQKEKQALHIPVKMGLINPEGKNIAEQVIELKEQKQTY |
| | TFENIAAKPVASLFRDFSAPVKVEHKRSEKDLLHIVKYDNNAFNRWDSLQQIATNIILNNADLND |
| | EFLNAFKSILHDKDLDKALISNALLIPIESTIAEAMRVIMVDDIVLSRKNVVNQLADKLKDDWLA |
| | VYQQCNDNKPYSLSAEQIAKRKLKGVCLSYLMNASDQKVGTDLAQQLFDNADNMTDQQTAFTELL |
| | KSNDKQVRDNAINEFYNRWRHEDLVVNKWLLSQAQISHESALDIVKGLVNHPAYNPKNPNKVYSL |
| | IGGFGANFLQYHCKDGLGYAFMADTVLALDKFNHQVAARMARNLMSWKRYDSDRQAMMKNALEKI |
| | KASNPSKNVFEIVSKSLES (SEQ ID NO: 21) |
| T. aquaticus | MDAFTENLNKLAELAIRVGLNLEEGQEIVATAPIEAVDFVRLLAEKAYENGASLFTVLYGDNLIA |
| Aminopeptidase T | RKRLALVPEAHLDRAPAWLYEGMAKAFHEGAARLAVSGNDPKALEGLPPERVGRAQQAQSRAYRP |
| | TLSAITEFVTNWTIVPFAHPGWAKAVFPGLPEEEAVQRLWQAIFQATRVDQEDPVAAWEAHNRVL |
| | HAKVAFLNEKRFHALHFQGPGTDLTVGLAEGHLWQGGATPTKKGRLCNPNLPTEEVFTAPHRERV |
| | EGVVRASRPLALSGQLVEGLWARFEGGVAVEVGAEKGEEVLKKLLDTDEGARRLGEVALVPADNP |
| | IAKTGLVFFDTLFDENAASHIAFGQAYAENLEGRPSGEEFRRRGGNESMVHVDWMIGSEEVDVDG |
| | LLEDGTRVPLMRRGRWVI (SEQ ID NO: 22) |
| Bacillus | MAKLDETLTMLKALTDAKGVPGNEREARDVMKTYIAPYADEVTTDGLGSLIAKKEGKSGGPKVMI |
| stearothermophilus | AGHLDEVGFMVTQIDDKGFIRFQTLGGWWSQVMLAQRVTIVTKKGDITGVIGSKPPHILPSEARK |
| Peptidase M28 | KPVEIKDMFIDIGATSREEAMEWGVRPGDMIVPYFEFTVLNNEKMLLAKAWDNRIGCAVAIDVLK |
| | QLKGVDHPNTVYGVGTVQEEVGLRGARTAAQFIQPDIAFAVDVGIAGDTPGVSEKEAMGKLGAGP |
| | HIVLYDATMVSHRGLREFVIEVAEELNIPHHFDAMPGVGTDAGAIHLTGIGVPSLTIAIPTRYIH |
| | SHAAILHRDDYENTVKLLVEVIKRLDADKVKQLTFDE (SEQ ID NO: 23) |
| Vibrio cholera | MEDKVWISMGADAVGSLNPALSESLLPHSFASGSQVWIGEVAIDELAELSHTMHEQHNRCGGYMV |
| Aminopeptidase | HTSAQGAMAALMMPESIANFTIPAPSQQDLVNAWLPQVSADQITNTIRALSSFNNRFYTTTSGAQ |
| | ASDWLANEWRSLISSLPGSRIEQIKHSGYNQKSVVLTIQGSEKPDEWVIVGGHLDSTLGSHTNEQ |
| | SIAPGADDDASGIASLSEIIRVLRDNNFRPKRSVALMAYAAEEVGLRGSQDLANQYKAQGKKVVS |
| | VLQLDMTNYRGSAEDIVFITDYTDSNLTQFLTTLIDEYLPELTYGYDRCGYACSDHASWHKAGFS |
| | AAMPFESKFKDYNPKIHTSQDTLANSDPTGNHAVKFTKLGLAYVIEMANAGSSQVPDDSVLQDGT |
| | AKINLSGARGTQKRFTFELSQSKPLTIQTYGGSGDVDLYVKYGSAPSKSNWDCRPYQNGNRETCS |
| | FNNAQPGIYHVMLDGYTNYNDVALKASTQ (SEQ ID NO: 24) |
| Photobacterium | MEDKVWISIGSDASQTVKSVMQSNARSLLPESLASNGPVWVGQVDYSQLAELSHHMHEDHQRCGG |
| halotolerans | YMVHSSPESAIAASNMPQSLVAFSIPEISQQDTVNAWLPQVNSQAITGTITSLTSFINRFYTTTS |
| Aminopeptidase | GAQASDWLANEWRSLSASLPNASVRQVSHFGYNQKSVVLTITGSEKPDEWIVLGGHLDSTIGSHT |
| | NEQSVAPGADDDASGIASVTEIIRVLSENNFQPKRSIAFMAYAAEEVGLRGSQDLANQYKAEGKQ |
| | VISALQLDMTNYKGSVEDIVFITDYTDSNLTTFLSQLVDEYLPSLTYGFDTCGYACSDHASWHKA |
| | GFSAAMPFEAKFNDYNPMIHTPNDTLQNSDPTASHAVKFTKLGLAYAIEMASTTGGTPPPTGNVL |
| | KDGVPVNGLSGATGSQVHYSFELPAQKNLQISTAGGSGDVDLYVSFGSEATKQNWDCRPYRNGNN |
| | EVCTFAGATPGTYSIMLDGYRQFSGVTLKASTQ (SEQ ID NO: 25) |
| Yersinia pestis | MTQQPQAKYRHDYRAPDYTITDIDLDFALDAQKTTVTAVSKVKRQGTDVTPLILNGEDLTLISVS |
| Aminopeptidase N | VDGQAWPHYRQQDNTLVIEQLPADFTLTIVNDIHPATNSALEGLYLSGEALCTQCEAEGFRHITY |
| | YLDRPDVLARFTTRIVADKSRYPYLLSNGNRVGQGELDDGRHWVKWEDPFPKPSYLFALVAGDFD |
| | VLQDKFITRSGREVALEIFVDRGNLDRADWAMTSLKNSMKWDETRFGLEYDLDIYMIVAVDFFNM |
| | GAMENKGLNVFNSKYVLAKAETATDKDYLNIEAVIGHEYFHNWTGNRVTCRDWFQLSLKEGLTVF |
| | RDQEFSSDLGSRSVNRIENVRVMRAAQFAEDASPMAHAIRPDKVIEMNNFYTLTVYEKGSEVIRM |
| | MHTLLGEQQFQAGMRLYFERHDGSAATCDDFVQAMEDVSNVDLSLFRRWYSQSGTPLLTVHDDYD |
| | VEKQQYHLFVSQKTLPTADQPEKLPLHIPLDIELYDSKGNVIPLQHNGLPVHHVLNVTEAEQTFT |
| | FDNVAQKPIPSLLREFSAPVKLDYPYSDQQLTFLMQHARNEFSRWDAAQSLLATYIKLNVAKYQQ |
| | QQPLSLPAHVADAFRAILLDEHLDPALAAQILTLPSENEMAELFTTIDPQAISTVHEAITRCLAQ |
| | ELSDELLAVYVANMTPVYRIEHGDIAKRALRNTCLNYLAFGDEEFANKLVSLQYHQADNMTDSLA |
| | ALAAAVAAQLPCRDELLAAFDVRWNHDGLVMDKWFALQATSPAANVLVQVRTLLKHPAFSLSNPN |
| | RTRSLIGSFASGNPAAFHAADGSGYQFLVEILSDLNTRNPQVAARLIEPLIRLKRYDAGRQALMR |
| | KALEQLKTLDNLSGDLYEKITKALAA (SEQ ID NO: 26) |
| Vibrio anguillarum | MEEKVWISIGGDATQTALRSGAQSLLPENLINQTSVWVGQVPVSELATLSHEMHENHQRCGGYMV |
| Aminopeptidase | HPSAQSAMSVSAMPLNLNAFSAPEITQQTTVNAWLPSVSAQQITSTITTLTQFKNRFYTTSTGAQ |
| | ASNWIADHWRSLSASLPASKVEQITHSGYNQKSVMLTITGSEKPDEWVVIGGHLDSTLGSRTNES |
| | SIAPGADDDASGIAGVTEIIRLLSEQNFRPKRSIAFMAYAAEEVGLRGSQDLANRFKAEGKKVMS |
| | VMQLDMTNYQGSREDIVFITDYTDSNFTQYLTQLLDEYLPSLTYGFDTCGYACSDHASWHAVGYP |
| | AAMPFESKFNDYNPNIHSPQDTLQNSDPTGFHAVKFTKLGLAYVVEMGNASTPPTPSNQLKNGVP |
| | VNGLSASRNSKTWYQFELQEAGNLSIVLSGGSGDADLYVKYQTDADLQQYDCRPYRSGNNETCQF |
| | SNAQPGRYSILLHGYNNYSNASLVANAQ (SEQ ID NO: 27) |
| Salinivibrio | MEDKKVWISIGADAQQTALSSGAQPLLAQSVAHNGQAWIGEVSESELAALSHEMHENHHRCGGYI |
| spYCSC6 | VHSSAQSAMAASNMPLSRASFIAPAISQQALVTPWISQIDSALIVNTIDRLTDFPNRFYTTTSGA |
| Aminopeptidase | QASDWIKQRWQSLSAGLAGASVTQISHSGYNQASVMLTIEGSESPDEWVVVGGHLDSTIGSRTNE |
| | QSIAPGADDDASGIAAVTEVIRVLAQNNFQPKRSIAFVAYAAEEVGLRGSQDVANQFKQAGKDVR |
| | GVLQLDMTNYQGSAEDIVFITDYTDNQLTQYLTQLLDEYLPTLNYGFDTCGYACSDHASWHQVGY |
| | PAAMPFEAKFNDYNPNIHTPQDTLANSDSEGAHAAKFTKLGLAYTVELANADSSPNPGNELKLGE |
| | PINGLSGARGNEKYFNYRLDQSGELVIRTYGGSGDVDLYVKANGDVSTGNWDCRPYRSGNDEVCR |
| | FDNATPGNYAVMLRGYRTYDNVSLIVE (SEQ ID NO: 28) |
| Vibrio proteolyticus | MPPITQQATVTAWLPQVDASQITGTISSLESFTNRFYTTTSGAQASDWIASEWQALSASLPNASV |
| Aminopeptidase I | KQVSHSGYNQKSVVMTITGSEAPDEWIVIGGHLDSTIGSHTNEQSVAPGADDDASGIAAVTEVIR |
| | VLSENNFQPKRSIAFMAYAAEEVGLRGSQDLANQYKSEGKNVVSALQLDMTNYKGSAQDVVFITD |
| | YTDSNFTQYLTQLMDEYLPSLTYGFDTCGYACSDHASWHNAGYPAAMPFESKFNDYNPRIHTTQD |
| | TLANSDPTGSHAKKFTQLGLAYAIEMGSATGDTPTPGNQLE (SEQ ID NO: 29) |
| Vibrio proteolyticus | MPPITQQATVTAWLPQVDASQITGTISSLESFTNRFYTTTSGAQASDWIASEWQFLSASLPNASV |
| Aminopeptidase I | KQVSHSGYNQKSVVMTITGSEAPDEWIVIGGHLDSTIGSHTNEQSVAPGADDDASGIAAVTEVIR |
| (A55F) | VLSENNFQPKRSIAFMAYAAEEVGLRGSQDLANQYKSEGKNVVSALQLDMTNYKGSAQDVVFITD |
| | YTDSNFTQYLTQLMDEYLPSLTYGFDTCGYACSDHASWHNAGYPAAMPFESKFNDYNPRIHTTQD |
| | TLANSDPTGSHAKKFTQLGLAYAIEMGSATGDTPTPGNQLE (SEQ ID NO: 30) |
| P. furiosus | MVDWELMKKIIESPGVSGYEHLGIRDLVVDILKDVADEVKIDKLGNVIAHFKGSAPKVMVAAHMD |
| Aminopeptidase I | KIGLMVNHIDKDGYLRVVPIGGVLPETLIAQKIRFFTEKGERYGVVGVLPPHLRREAKDQGGKID |
| | WDSIIVDVGASSREEAEEMGFRIGTIGEFAPNFTRLSEHRFATPYLDDRICLYAMIEAARQLGEH |
| | EADIYIVASVQEEIGLRGARVASFAIDPEVGIAMDVTFAKQPNDKGKIVPELGKGPVMDVGPNIN |
| | PKLRQFADEVAKKYEIPLQVEPSPRPTGTDANVMQINREGVATAVLSIPIRYMHSQVELADARDV |
| | DNTIKLAKALLEELKPMDFTPLE (SEQ ID NO: 31) |

In certain embodiments, the peptidase is an exopeptidase. In certain embodiments, the peptidase is an aminopeptidase. In certain embodiments, the peptidase is proline aminopeptidase, a proline iminopeptidase, a glutamate/aspartate-specific aminopeptidase, a methionine-specific aminopeptidase, or a zinc metalloprotease. In certain embodiments, the peptidase is a TET aminopeptidase. In certain embodiments, the TET aminopeptidase is hTet. In certain embodiments, the TET aminopeptidase is pfuTet.

In some embodiments, reacting the peptide or the compound of Formula (III-d), or salt thereof, with a peptidase, in a degradation process comprises one or more amino acid recognizers (e.g., one or more amino acid binding proteins not having peptide cleavage activity). In some embodiments, an amino acid recognizer comprises an amino acid binding protein, such as a ClpS protein (e.g., Planctomycetia bacterium ClpS protein), a UBR protein (e.g., Kluyveromyces marxianus UBR protein), an Ntaq1 protein (e.g., Scleropages formosus Ntaq1 protein), or a variant or homolog thereof. In some embodiments, an amino acid recognizer comprises a label (e.g., a detectable label, such as a luminescent label). Examples of amino acid recognizers (e.g., recognition molecules) are described in detail in PCT International Publication No. WO2020/102741A1, filed Nov. 15, 2019, PCT International Publication No. WO2021/236983A2, filed May 20, 2021, and co-pending U.S. Ser. No. 63/395,328, filed Aug. 4, 2022, the relevant content of each of which is incorporated by reference in its entirety.

In some embodiments, reacting the peptide or the compound of Formula (III-d), or salt thereof, with a peptidase, in a degradation process (e.g., a reaction mixture) can be configured to achieve a time interval that allows for sufficient association events which provide a desired confidence level with a characteristic pattern. This can be achieved, for example, by configuring the reaction conditions based on various properties, including: linker identity, reagent concentration, molar ratio of one reagent to another (e.g., ratio of amino acid recognizer to cleaving reagent, ratio of one recognizer to another, ratio of one cleaving reagent to another), number of different reagent types (e.g., the number of different types of recognizers and/or cleaving reagents, the number of recognizer types relative to the number of cleaving reagent types), cleavage activity (e.g., aminopeptidase activity), binding properties (e.g., kinetic and/or thermodynamic binding parameters for recognition molecule binding), reagent modification (e.g., polyol and other recognizer modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkers with or without shielding element), surface modification (e.g., modification of sample well surface, including polypeptide immobilization), sample preparation (e.g., polypeptide fragment size, polypeptide modification for immobilization), and other aspects described herein.

In some embodiments, reacting the peptide or the compound of Formula (III-d), or salt thereof, with a peptidase, in a degradation process is performed under conditions in which recognition and cleavage of amino acids can occur simultaneously in a single reaction mixture. For example, in some embodiments, a peptide sequencing reaction is performed in a reaction mixture having a pH at which association events and cleavage events can occur. Accordingly, in some embodiments, a reaction mixture has a pH of between about 6.5 and about 9.0. In some embodiments, a reaction mixture has a pH of between about 7.0 and about 8.5 (e.g., between about 7.0 and about 8.0, between about 7.5 and about 8.5, between about 7.5 and about 8.0, or between about 8.0 and about 8.5).

In some embodiments, reacting the peptide or the compound of Formula (III-d), or salt thereof, with a peptidase, in a degradation process is performed in a reaction mixture comprising one or more buffering agents. In some embodiments, a reaction mixture comprises a buffering agent in a concentration of at least 10 mM (e.g., at least 20 mM and up to 250 mM, at least 50 mM, 10-250 mM, 10-100 mM, 20-100 mM, 50-100 mM, or 100-200 mM). In some embodiments, a reaction mixture comprises a buffering agent in a concentration of between about 10 mM and about 50 mM (e.g., between about 10 mM and about 25 mM, between about 25 mM and about 50 mM, or between about 20 mM and about 40 mM). Examples of buffering agents include, without limitation, HEPES (4-(2-hydroxyethyl)-1-piperazineethanesulfonic acid), Tris (tris(hydroxymethyl)aminomethane), and MOPS (3-(N-morpholino)propanesulfonic acid).

In some embodiments, reacting the peptide or the compound of Formula (III-d), or salt thereof, with a peptidase, in a degradation process is performed in a reaction mixture comprising salt in a concentration of at least 10 mM. In some embodiments, a reaction mixture comprises salt in a concentration of at least 10 mM (e.g., at least 20 mM, at least 50 mM, at least 100 mM, or more). In some embodiments, a reaction mixture comprises salt in a concentration of between about 10 mM and about 250 mM (e.g., between about 20 mM and about 200 mM, between about 50 mM and about 150 mM, between about 10 mM and about 50 mM, or between about 10 mM and about 100 mM). Examples of salts include, without limitation, sodium salts, potassium salts, and acetates, such as sodium chloride (NaCl), sodium acetate (NaOAc), and potassium acetate (KOAc).

Additional examples of components for use in reacting the peptide or the compound of Formula (III-d), or salt thereof, with a peptidase, in a degradation process (i.e., a reaction mixture) include divalent cations (e.g., Mg²⁺, Co²⁺) and surfactants (e.g., polysorbate 20). In some embodiments, a reaction mixture comprises a divalent cation in a concentration of between about 0.1 mM and about 50 mM (e.g., between about 10 mM and about 50 mM, between about 0.1 mM and about 10 mM, or between about 1 mM and about 20 mM). In some embodiments, a reaction mixture comprises a surfactant in a concentration of at least 0.01% (e.g., between about 0.01% and about 0.10%). In some embodiments, a reaction mixture comprises one or more components useful in single-molecule analysis, such as an oxygen-scavenging system (e.g., a PCA/PCD system or a Pyranose oxidase/Catalase/glucose system) and/or one or more triplet state quenchers (e.g., trolox, COT, and NBA).

In some embodiments, reacting the peptide or the compound of Formula (III-d), or salt thereof, with a peptidase, in a degradation process is performed at a temperature at which association events and cleavage events can occur. In some embodiments, a peptide sequencing reaction is performed at a temperature of at least 10° C. In some embodiments, a peptide sequencing reaction is performed at a temperature of between about 10° C. and about 50° C. (e.g., 15-45° C., 20-40° C., at or around 25° C., at or around 30° C., at or around 35° C., at or around 37° C.). In some embodiments, a peptide sequencing reaction is performed at or around room temperature.

In some embodiments, the relative occurrence of recognition and cleavage can be controlled by a concentration differential between one or more amino acid recognizers and at least one cleaving reagent. In some embodiments, the concentration differential can be optimized such that the number of signal pulses detected during recognition of an individual amino acid provides a desired confidence interval for identification. For example, if an initial sequencing reaction provides signal data with too few signal pulses between cleavage events to permit determination of characteristic patterns with a desired confidence interval, the sequencing reaction can be repeated using a decreased concentration of non-specific exopeptidase relative to recognition molecule.

In some embodiments, reacting the peptide or the compound of Formula (III-d), or salt thereof, with a peptidase, in a degradation process may be carried out by contacting a polypeptide with a reaction mixture comprising one or more amino acid recognizers and one or more cleaving reagents (e.g., peptidases). In some embodiments, a reaction mixture comprises an amino acid recognizer at a concentration of between about 10 nM and about 10 μM. In some embodiments, a reaction mixture comprises a cleaving reagent at a concentration of between about 500 nM and about 500 μM.

In some embodiments, reacting the peptide or the compound of Formula (III-d), or salt thereof, with a peptidase, in a degradation process (i.e., a reaction mixture) comprises an amino acid recognizer at a concentration of between about 100 nM and about 10 μM, between about 250 nM and about 10 μM, between about 100 nM and about 1 μM, between about 250 nM and about 1 μM, between about 250 nM and about 750 nM, or between about 500 nM and about 1 μM. In some embodiments, a reaction mixture comprises an amino acid recognizer at a concentration of about 100 nM, about 250 nM, about 500 nM, about 750 nM, or about 1 μM. In some embodiments, a reaction mixture comprises a cleaving reagent at a concentration of between about 500 nM and about 250 μM, between about 500 nM and about 100 μM, between about 1 μM and about 100 μM, between about 500 nM and about 50 μM, between about 1 μM and about 100 μM, between about 10 μM and about 200 μM, or between about 10 μM and about 100 μM. In some embodiments, a reaction mixture comprises a cleaving reagent at a concentration of about 1 μM, about 5 μM, about 10 μM, about 30 μM, about 50 μM, about 70 μM, or about 100 μM.

In some embodiments, reacting the peptide or the compound of Formula (III-d), or salt thereof, with a peptidase, in a degradation process (i.e., a reaction mixture) comprises an amino acid recognizer at a concentration of between about 10 nM and about 10 μM, and a cleaving reagent at a concentration of between about 500 nM and about 500 μM. In some embodiments, a reaction mixture comprises an amino acid recognizer at a concentration of between about 100 nM and about 1 μM, and a cleaving reagent at a concentration of between about 1 μM and about 100 μM. In some embodiments, a reaction mixture comprises an amino acid recognizer at a concentration of between about 250 nM and about 1 μM, and a cleaving reagent at a concentration of between about 10 μM and about 100 μM. In some embodiments, a reaction mixture comprises an amino acid recognizer at a concentration of about 500 nM, and a cleaving reagent at a concentration of between about 25 μM and about 75 μM. In some embodiments, the concentration of an amino acid recognizer and/or the concentration of a cleaving reagent in a reaction mixture is as described elsewhere herein.

In some embodiments, reacting the peptide or the compound of Formula (III-d), or salt thereof, with a peptidase, in a degradation process (i.e., a reaction mixture) comprises an amino acid recognizer and a cleaving reagent in a molar ratio of about 500:1, about 400:1, about 300:1, about 200:1, about 100:1, about 75:1, about 50:1, about 25:1, about 10:1, about 5:1, about 2:1, or about 1:1. In some embodiments, a reaction mixture comprises an amino acid recognizer and a cleaving reagent in a molar ratio of between about 10:1 and about 200:1. In some embodiments, a reaction mixture comprises an amino acid recognizer and a cleaving reagent in a molar ratio of between about 50:1 and about 150:1. In some embodiments, the molar ratio of an amino acid recognizer to a cleaving reagent in a reaction mixture is between about 1:1,000 and about 1:1 or between about 1:1 and about 100:1 (e.g., 1:1,000, about 1:500, about 1:200, about 1:100, about 1:10, about 1:5, about 1:2, about 1:1, about 5:1, about 10:1, about 50:1, about 100:1). In some embodiments, the molar ratio of an amino acid recognizer to a cleaving reagent in a reaction mixture is between about 1:100 and about 1:1 or between about 1:1 and about 10:1. In some embodiments, the molar ratio of an amino acid recognizer to a cleaving reagent in a reaction mixture is as described elsewhere herein.

In some embodiments, a reaction mixture comprises one or more amino acid recognizers and one or more cleaving reagents described herein. In some embodiments, a reaction mixture comprises at least three amino acid recognizers and at least one cleaving reagent. In some embodiments, the reaction mixture comprises two or more cleaving reagents. In some embodiments, the reaction mixture comprises at least one and up to ten cleaving reagents (e.g., 1-3 cleaving reagents, 2-10 cleaving reagents, 1-5 cleaving reagents, 3-10 cleaving reagents). In some embodiments, the reaction mixture comprises at least three and up to thirty amino acid recognizers (e.g., between 3 and 25, between 3 and 20, between 3 and 10, between 3 and 5, between 5 and 30, between 5 and 20, between 5 and 10, or between 10 and 20, amino acid recognizers).

In some embodiments, reacting the peptide or the compound of Formula (III-d), or salt thereof, with a peptidase, in a degradation process (i.e., a reaction mixture) comprises more than one amino acid recognizer and/or more than one cleaving reagent. In some embodiments, a reaction mixture described as comprising more than one amino acid recognizer or cleaving reagent refers to the mixture as having more than one type of amino acid recognizer or cleaving reagent. For example, in some embodiments, a reaction mixture comprises two or more cleaving reagents, where the two or more cleaving reagents refer to two or more types of aminopeptidases. In some embodiments, one type of aminopeptidase has an amino acid sequence that is different from another type of aminopeptidase in the reaction mixture. In some embodiments, one type of cleaving reagent cleaves an amino acid or subset of amino acids that is different from an amino acid or subset of amino acids cleaved by another type of cleaving reagent in the reaction mixture.

In some aspects, the application provides methods comprising obtaining data during a degradation process of a polypeptide. In some embodiments, the methods comprise analyzing the data to determine portions of the data corresponding to amino acids that are sequentially exposed at a terminus of the polypeptide during the degradation process. In some embodiments, the methods comprise outputting an amino acid sequence representative of the polypeptide. In some embodiments, the data is indicative of amino acid identity at the terminus of the polypeptide during the degradation process. In some embodiments, the data is indicative of a luminescent signal generated during the degradation process. In some embodiments, the data is indicative of an electrical signal generated during the degradation process.

In some embodiments, analyzing the data further comprises detecting a series of cleavage events and determining the portions of the data between successive cleavage events. In some embodiments, analyzing the data further comprises determining a type of amino acid for each of the individual portions. In some embodiments, each of the individual portions comprises a pulse pattern (e.g., a characteristic pattern), and analyzing the data further comprises determining a type of amino acid for one or more of the portions based on its respective pulse pattern. In some embodiments, determining the type of amino acid further comprises identifying an amount of time within a portion when the data is above a threshold value and comparing the amount of time to a duration of time for the portion. In some embodiments, determining the type of amino acid further comprises identifying at least one pulse duration for each of the one or more portions. In some embodiments, the pulse pattern comprises a mean pulse duration of between about 1 millisecond and about 10 seconds. In some embodiments, determining the type of amino acid further comprises identifying at least one interpulse duration for each of the one or more portions. In some embodiments, the amino acid sequence includes a series of amino acids corresponding to the portions. In some embodiments, the pulse pattern is produced by an amino acid recognizer associated with one or more reagents of a sequencing reaction. In some embodiments, the pulse pattern is produced by association and dissociation of an amino acid recognizer with one or more reagents of a sequencing reaction.

As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids). The association events between amino acid recognizers and different types of amino acids at the terminal end of the polypeptide produce distinctive changes in the signal, referred to herein as a characteristic pattern, which may be used to determine chemical characteristics of the polypeptide. In some embodiments, a characteristic pattern corresponding to one type of terminal amino acid can be used to determine structural information for the terminal amino acid and one or more amino acids contiguous to the terminal amino acid. Accordingly, in some embodiments, a characteristic pattern corresponding to one type of terminal amino acid can be used to determine structural information for at least two (e.g., at least three, at least four, at least five, two, three, four, or between two and five) amino acids of a polypeptide.

In some embodiments, a transition from one characteristic pattern to another is indicative of amino acid cleavage. As used herein, in some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

In some embodiments, signal data can be analyzed to extract signal pulse information by applying threshold levels to one or more parameters of the signal data. For example, in some embodiments, a threshold magnitude level may be applied to the signal data of a signal trace. In some embodiments, the threshold magnitude level is a minimum difference between a signal detected at a point in time and a baseline determined for a given set of data. In some embodiments, a signal pulse is assigned to each portion of the data that is indicative of a change in magnitude exceeding the threshold magnitude level and persisting for a duration of time. In some embodiments, a threshold time duration may be applied to a portion of the data that satisfies the threshold magnitude level to determine whether a signal pulse is assigned to that portion. For example, experimental artifacts may give rise to a change in magnitude exceeding the threshold magnitude level but that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a signal pulse is extracted from signal data based on a threshold magnitude level and a threshold time duration.

In some embodiments, a peak in magnitude of a signal pulse is determined by averaging the magnitude detected over a duration of time that persists above the threshold magnitude level. It should be appreciated that, in some embodiments, a “signal pulse” as used herein can refer to a change in signal data that persists for a duration of time above a baseline (e.g., raw signal data), or to signal pulse information extracted therefrom (e.g., processed signal data).

In some embodiments, signal pulse information can be analyzed to identify different types of amino acids in a polypeptide based on different characteristic patterns in a series of signal pulses. For example, the signal pulse information is indicative of different types of amino acids at a terminal end of a polypeptide (e.g., arginine, leucine, isoleucine, phenylalanine). By way of example, the signal pulses detected at the earliest time points provide information indicative of (at least) arginine at the terminus of the polypeptide based on a first characteristic pattern, and the signal pulses detected at the latest time points provide information indicative of at least phenylalanine at the terminus of the polypeptide based on a second characteristic pattern.

In some embodiments, each signal pulse of a characteristic pattern comprises a pulse duration corresponding to an association event between an amino acid recognizer and an amino acid ligand. In some embodiments, the pulse duration is characteristic of a dissociation rate of binding. In some embodiments, each signal pulse of a characteristic pattern is separated from another signal pulse of the characteristic pattern by an interpulse duration. In some embodiments, the interpulse duration is characteristic of an association rate of binding. In some embodiments, a change in magnitude in a signal can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

In some embodiments, polypeptide analysis is performed by detecting a series of signal pulses indicative of association of one or more amino acid recognizers with successive amino acids exposed at the terminus of a polypeptide in an ongoing degradation reaction. The series of signal pulses can be analyzed to determine characteristic patterns in the series of signal pulses, and the time course of characteristic patterns can be used to determine chemical characteristics throughout an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, the series of signal pulses comprises a series of changes in magnitude of an optical signal over time. In some embodiments, the series of changes in the optical signal comprises a series of changes in luminescence produced during association events. In some embodiments, luminescence is produced by a detectable label associated with one or more reagents of a sequencing reaction. For example, in some embodiments, each of the one or more amino acid recognizers comprises a luminescent label. In some embodiments, a cleaving reagent comprises a luminescent label. Examples of luminescent labels and their use in accordance with the application are provided herein.

In some embodiments, the series of signal pulses comprises a series of changes in magnitude of an electrical signal overtime. In some embodiments, the series of changes in the electrical signal comprises a series of changes in conductance produced during association events. In some embodiments, conductivity is produced by a detectable label associated with one or more reagents of a sequencing reaction. For example, in some embodiments, each of the one or more amino acid recognizers comprises a conductivity label. Examples of conductivity labels and their use in accordance with the application are provided elsewhere herein. Methods for identifying single molecules using conductivity labels have been described (see, e.g., U.S. Patent Publication No. 2017/0037462).

In some embodiments, the series of changes in conductance comprises a series of changes in conductance through a nanopore. For example, methods of evaluating receptor-ligand interactions using nanopores have been described (see, e.g., Thakur, A. K. & Movileanu, L. (2019) Nature Biotechnology 37(1)). The inventors have recognized and appreciated that such nanopores may be used to monitor peptide sequencing reactions in accordance with the application. Accordingly, in some embodiments, the disclosure provides methods of polypeptide analysis comprising contacting a single polypeptide molecule with one or more amino acid recognizers described herein, where the single polypeptide molecule is immobilized to a nanopore. In some embodiments, the methods further comprise detecting a series of changes in conductance through the nanopore indicative of association of the one or more amino acid recognizers with successive amino acids exposed at a terminus of the single polypeptide while the single polypeptide is being degraded.

As described herein, in some embodiments, amino acid recognizers of the disclosure may be used to determine at least one chemical characteristic of a polypeptide. In some embodiments, determining at least one chemical characteristic comprises determining the type of amino acid that is present at a terminal end of a polypeptide and/or the types of amino acids that are present at one or more positions contiguous to the amino acid at the terminal end. In some embodiments, determining the type of amino acid comprises determining the actual amino acid identity, for example by determining which of the naturally-occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid comprises a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, determining at least one chemical characteristic of a polypeptide comprises determining that an amino acid comprises an arginine post-translational modification. For example, as described herein, amino acid recognizers of the disclosure are capable of distinguishing between different arginine modifications, including symmetric dimethylarginine (SDMA), asymmetric dimethylarginine (ADMA), and citrullinated arginine.

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine).

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, as described herein, amino acid recognizers of the disclosure are capable of distinguishing between oxidized methionine and its unmodified variant. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, a protein or polypeptide can be digested into a plurality of smaller polypeptides and chemical characteristics can be determined for one or more of these smaller polypeptides. In some embodiments, a first terminus (e.g., N or C terminus) of a polypeptide is immobilized and the other terminus (e.g., the C or N terminus) is analyzed as described herein.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (e.g., and determining the relative position of one or more amino acid types without determining the identity of each amino acid in the polypeptide). However, in some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some embodiments, sequence information for a plurality of polypeptide products obtained from a longer polypeptide or protein (e.g., via enzymatic and/or chemical cleavage) can be analyzed to reconstruct or infer the sequence of the longer polypeptide or protein.

In some aspects, the polypeptide analysis described herein generates data indicating how a polypeptide interacts with a binding means while the polypeptide is being degraded by a cleaving means. As discussed above, the data can include a series of characteristic patterns corresponding to association events at a terminus of a polypeptide in between cleavage events at the terminus. In some embodiments, methods of polypeptide analysis described herein comprise contacting a single polypeptide molecule with a binding means and a cleaving means, where the binding means and the cleaving means are configured to achieve at least 10 association events prior to a cleavage event. In some embodiments, the means are configured to achieve the at least 10 association events between two cleavage events.

In some embodiments, a plurality of single-molecule sequencing reactions are performed in parallel in an array of sample wells. In some embodiments, an array comprises between about 10,000 and about 1,000,000 sample wells. The volume of a sample well may be between about 10⁻²¹ liters and about 10⁻¹⁵ liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells. In some embodiments, the binding means and the cleaving means are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

In some embodiments, a luminescent label refers to a fluorophore or a dye. Typically, a luminescent label comprises an aromatic or heteroaromatic compound and can be a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, fluoroscein, rhodamine, xanthene, or other like compound.

In some embodiments, a luminescent label comprises a dye selected from one or more of the following: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor® 660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY®493/501, BODIPY®530/550, BODIPY®558/568, BODIPY®564/570, BODIPY®576/589, BODIPY®581/591, BODIPY®630/650, BODIPY®650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488 Å, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™ 642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight®350, DyLight®405, DyLight®415-Col, DyLight®425Q, DyLight®485-LS, DyLight®488, DyLight®504Q, DyLight®510-LS, DyLight®515-LS, DyLight®521-LS, DyLight®530-R2, DyLight®543Q, DyLight®550, DyLight®554-R0, DyLight®554-R1, DyLight®590-R2, DyLight®594, DyLight®610-B1, DyLight®615-B2, DyLight®633, DyLight®633-B1, DyLight®633-B2, DyLight®650, DyLight®655-B1, DyLight®655-B2, DyLight®655-B3, DyLight®655-B4, DyLight®662Q, DyLight®675-B1, DyLight®675-B2, DyLight®675-B3, DyLight®675-B4, DyLight®679-C5, DyLight®680, DyLight®683Q, DyLight®690-B1, DyLight®690-B2, DyLight®696Q, DyLight®700-B1, DyLight®700-B1, DyLight®730-B1, DyLight®730-B2, DyLight®730-B3, DyLight®730-B4, DyLight®747, DyLight®747-B1, DyLight®747-B2, DyLight®747-B3, DyLight®747-B4, DyLight®755, DyLight®766Q, DyLight®775-B2, DyLight®775-B3, DyLight®775-B4, DyLight®780-B1, DyLight®780-B2, DyLight®780-B3, DyLight®800, DyLight®830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor®450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye®680LT, IRDye®750, IRDye®800CW, JOE, LightCycler®640R, LightCycler® Red 610, LightCycler® Red 640, LightCycler® Red 670, LightCycler® Red 705, Lissamine Rhodamine B, Napthofluorescein, Oregon Green®488, Oregon Green®514, Pacific Blue™ Pacific Green™, Pacific Orange™, PET, PF350, PF405, PF415, PF488, PF505, PF532, PF546, PF555P, PF568, PF594, PF610, PF633P, PF647P, Quasar®570, Quasar®670, Quasar®705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™ 633, Seta™ 650, Seta™ 660, Seta™ 670, Seta™ 680, Seta™ 700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1

Solid-phase library preparation (SPLP) shares many common features and advantages with solid-phase peptide synthesis, solid-phase oligonucleotide synthesis, combinatorial library, and DNA-encoded library (DEL). These advantages include that: (1) SPLP is clean (excess reagents and byproducts of each step can be drained after every step); (2) SPLP is flexible (solvent/buffer can easily be replaced at each step); (3) SPLP is fast (rate can be tuned by changing the concentration of reagents); (4) SPLP reactions are unbiased (equal opportunity for peptides to react if the yield can be maximized); (5) there are no concentration constraints (product released is directly loadable); (6) SPLP is compatible with automation; and (7) other peptide conjugation chemistry may be explored.

The inventors realized that performing library preparation for peptide sequencing in the solid phase is advantageous in multiple respects as compared to library preparation for peptide sequencing in solution. Solid-phase immobilization permits using a lower concentration of sample peptide while maximally loading an array of reaction chambers with sample, enabling greater sequencing and/or detection sensitivity. Solid-phase library preparation permits using optimal concentrations of reagents to increase stepwise yields without the requirement for purification. Additionally, solid-phase library preparation enables automated reagent addition, mixing, and cleanup using fluidic devices, and can shorten the reaction time and increase sample throughput.

### Example 2

This Example describes how various solid supports (beads) were screened for their use in SPLP. Click chemistry was performed at room temp. for 16 hrs at 50 uM K-linker concentration. Glen Research affinity beads exhibited the greatest click efficiency in several solvents (Table 4).

TABLE 4
Solid supports tested for use in SPLP

| | Click | Click | Click | Click | |
|---|---|---|---|---|---|
| | efficiency | efficiency | efficiency | efficiency | |
| | 100% water | 20% ACN | 20% DMF | 20% DMSO | Notes |
| Tentagel-S | x | x | x | not tested | Widely used in peptide |
| | | | | | synthesis. Currently |
| | | | | | used in the QSI library |
| | | | | | prep quench step |
| Tentagel-R | x | x | x | not tested | Used for DEL and long |
| | | | | | peptides synthesis |
| Tentagel-XV | x | x | 16% | not tested | Better swelling in water |
| | | | | | than most other |
| | | | | | Tentagel beads |
| Tentagel-N | x | x | x | not tested | Used for oligo synthesis |
| Glen | 48.5% | 70% | 57.6% | 65.2% | Previously used in the |
| Research | | | | | QSI library prep quench |
| affinity | | | | | and linker capturing |
| beads | | | | | |
| PEGA resin | x | 60% | 67.2% | 74.2% | Excellent swelling |
| | | | | | property in water, |
| | | | | | permeable for large |
| | | | | | molecules |
| NovaPEG | x | x | x | not tested | Discontinued |
| resin | | | | | |

### Example 3

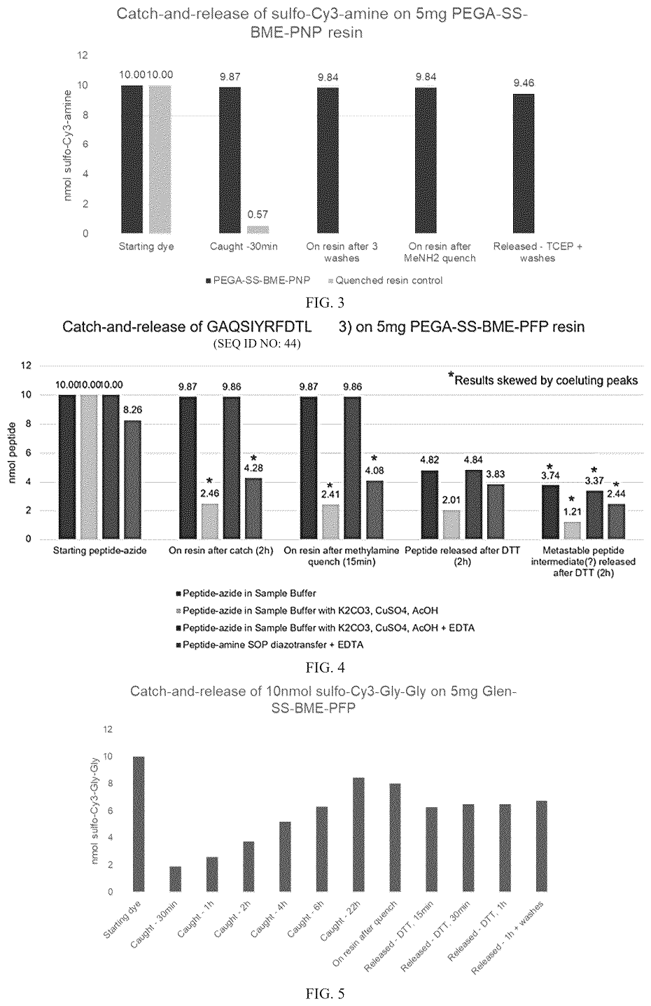

This Example describes chemical release from a PEGA solid support resin using Cy3-amine as a model. PEGA-amine was converted to PEGA-SS-BME-PNP (Scheme 1). To this PEGA-SS-BME-PNP resin (5 mg) was added Cy3-amine, which was “caught” by the resin (i.e., by reacting with the p-nitrophenyl carbonate) (FIG. 3). The resin was washed three times, causing no appreciable loss of caught Cy3-amine. The resin was quenched with methylamine and then treated with TCEP and washed, releasing the vast majority of Cy3 dye that had been caught by the resin.

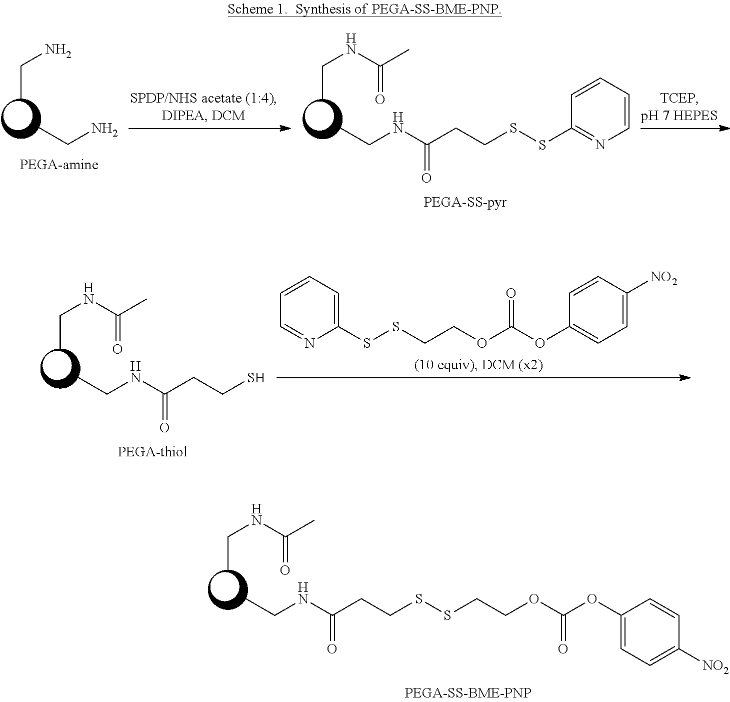

### Example 4

This Example describes peptide capture and release from a PEGA solid support resin. Because peptide N-termini react more slowly than Cy3-amine with the p-nitrophenyl (PNP) carbonate present in PEGA-SS-BME-PNP, a more active perfluorophenyl (PFP) carbonate was used instead (Scheme 2). The resultant compound, PEGA-SS-BME-PFP, showed increased effectiveness in the capture of input peptides compared to PEGA-SS-BME-PNP. Using a representative input peptide, greater than 60% efficiency in diazotransfer, capture, and release was observed (FIG. 4). After capture of the input peptide, treatment with TCEP causes disulfide cleavage, forming a metastable thiol intermediate (Scheme 3). Cyclization to release the oxathiolanone releases the free peptide.

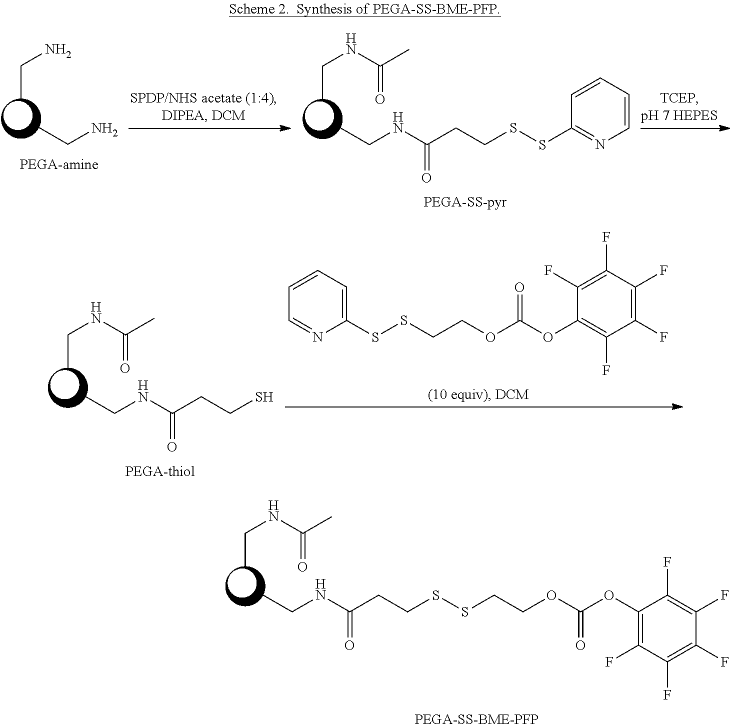

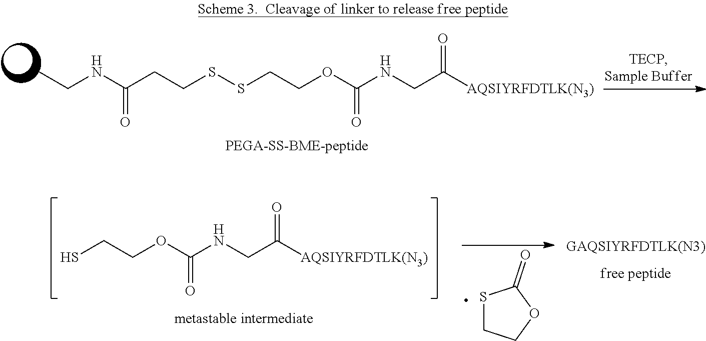

### Example 5

This Example describes the synthesis and use of the Glen-SS-BME-PFP resin:

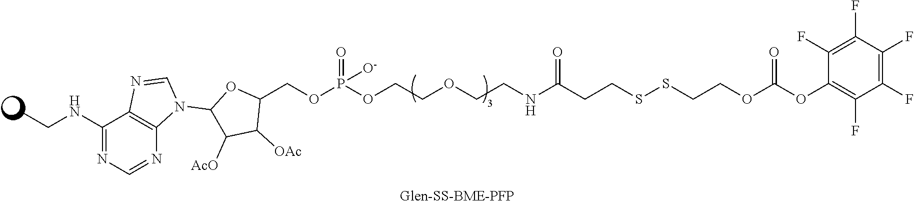

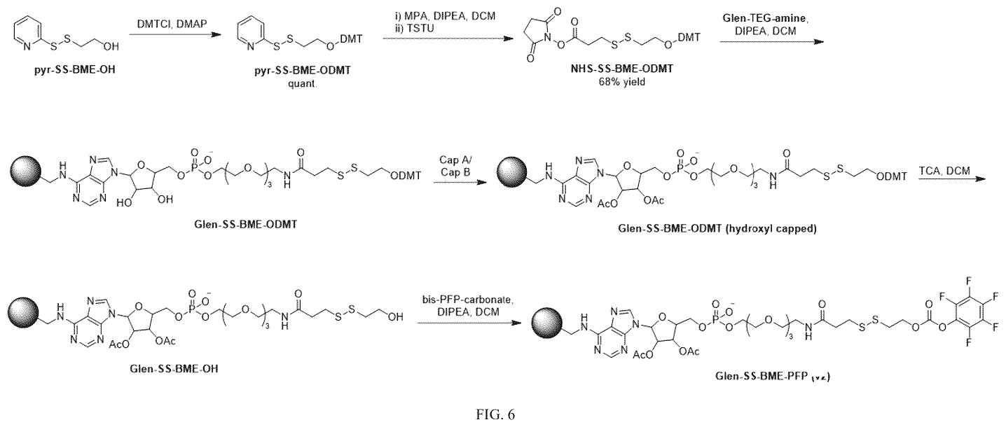

The PEGA-SS-BME-PFP resin demonstrated difficulty in forming a clicked complex with DBCO-dsDNA after peptide-azides are installed. Because the Glen resin showed reasonably efficient click chemistry to DBCO-dsDNA, it was used for the next resin. This workflow eliminated free OH groups that can potentially direct peptides to non-cleavable locations (FIG. 6). The starting material for this synthesis is Oligo-Affinity Support (PS) (5′-Dimethoxytrityl-Adenosine-2′,3′-diacetate-N-Linked-Polymeric Support), available from Glen Research (Catalog Number 26-4001).

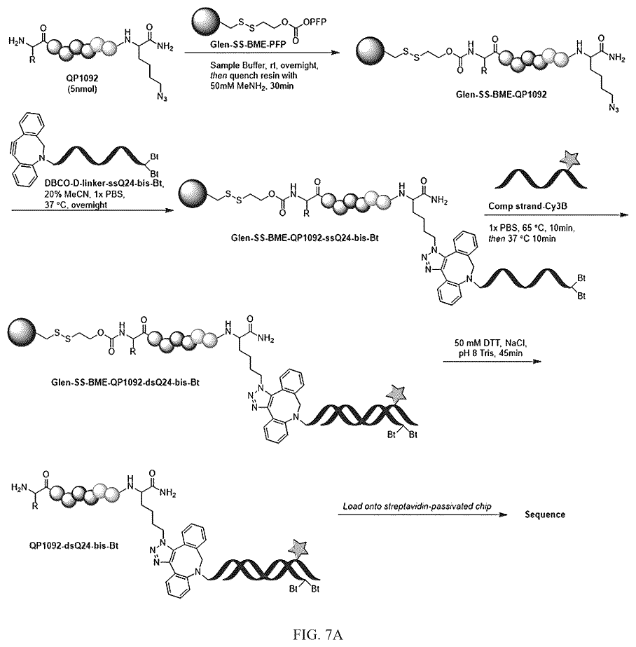

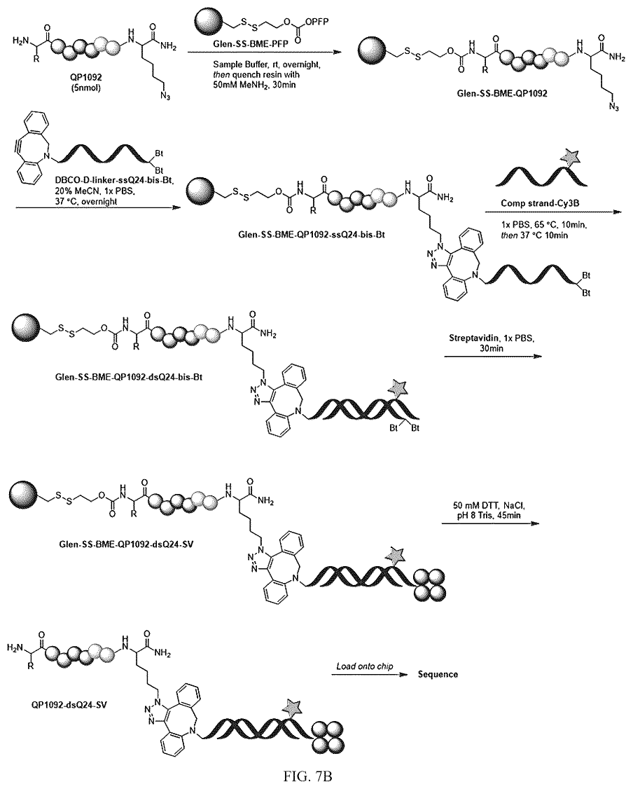

The Glen-SS-BME-PFP resin showed 85% catching efficiency for 10 nmol dye-labeled Gly-Gly after 22 hr and 84% release efficiency upon incubation with DTT for 15 min (FIG. 5). The Glen-SS-BME-PFP resin showed quantitative capture of model peptide azides of 0.75 nmol, quantitative click chemistry with excess DBCO-dsDNA, and 78% release efficiency. After capture of the input peptide, treatment with TCEP causes disulfide cleavage, forming a metastable thiol intermediate (Scheme 4). Cyclization to release the oxathiolanone releases the free peptide. Schematics of SPLP workflow are shown for on-chip streptavidinylation and on-resin streptavidinylation (FIGS. 7A-7B). Additional results are shown in Tables 5 and 6, and FIG. 8.

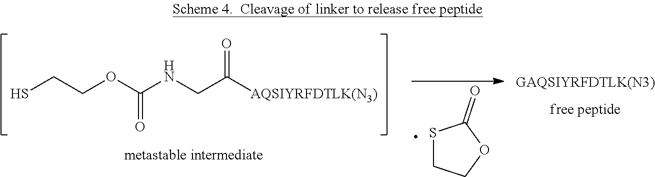

TABLE 5
Click chemistry of DNA on solid phase (Glen resin)

| | DNA | Overall yield (click |
|---|---|---|
| Input DBCO-DNA | structure | and release) (%)* |
| ds. DBCO-Q24 | B-duplex | 1.2 |
| ss. DBCO-Q24 | Hairpin | 10 |
| ss. DBCO-Q525 | Random coil | 0.3 |

*DNA (2.5 nmol) is the limiting reagent compared to peptides (5 nmol)

TABLE 6

| | | | |
|---|---|---|---|
| Peptide input | Overall yield (%) | Loading (%) | Alignment |

| | | | | |
|---|---|---|---|---|
| 5 | nmol | 10 | 89 | 40181 |
| 50 | pmol | N/A | 11* | 5500 |
| 50 | pmol | 81 | 60 | 11937 |
| 5 | pmol | >100** | 69 | 11454 |

*Tris in the loading solution
**Either caused by error in low concentration measurement or incomplete wash of dye

### Example 6

This Example describes further modifications to the SPLP sample preparation, including the preparation and use of additional Glen-SS resins.

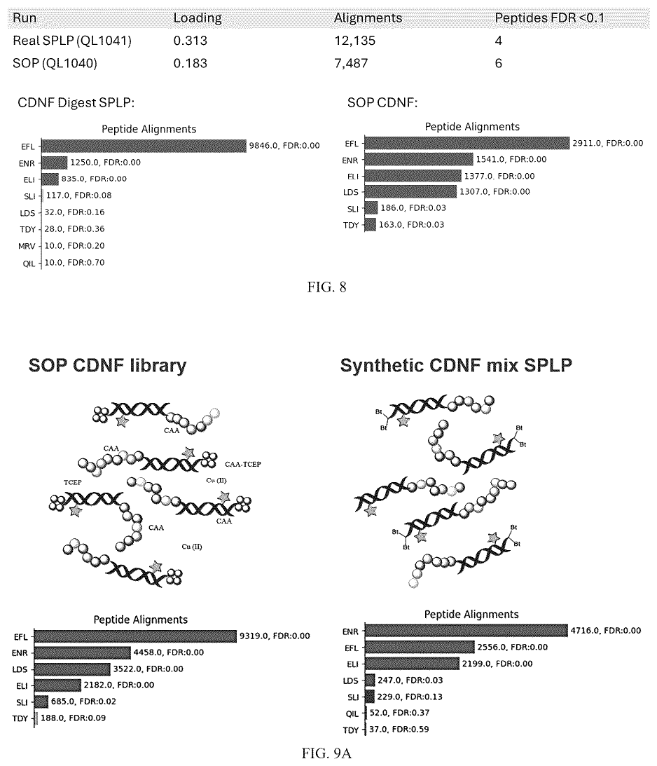

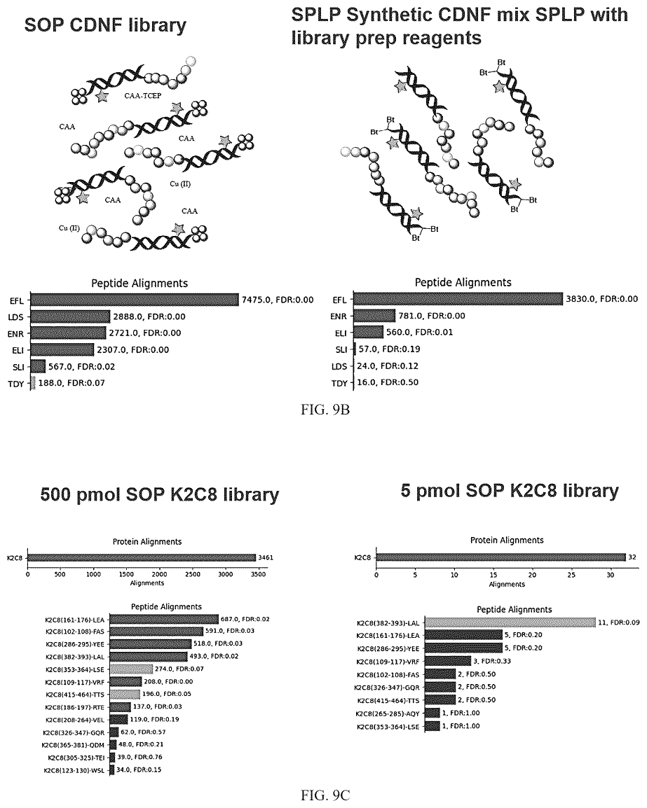

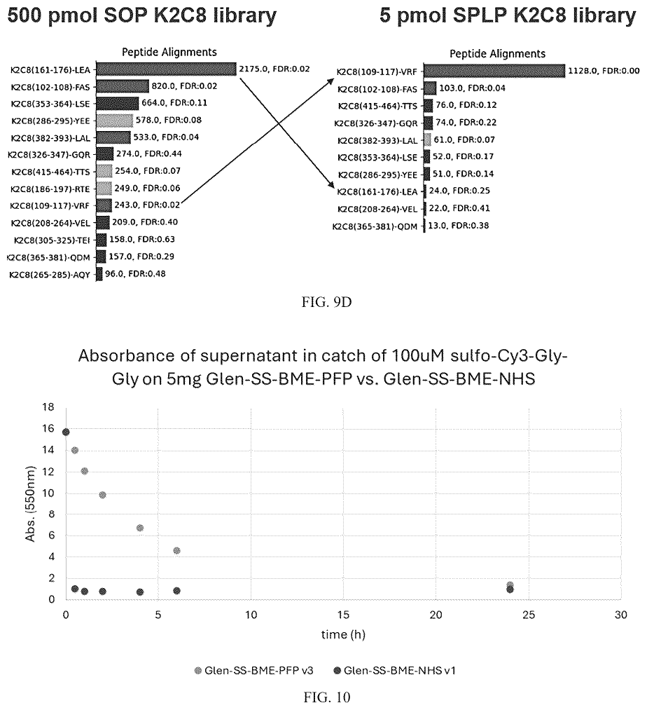

Peptide library CDNF was sequenced in the solution phase at 500 pmol (SOP) and 5 pmol protein concentration, as well as at 5 pmol in the presence of library preparation reagents (FIGS. 9A-9B). At low input, the presence of library preparation reagents may influence the sequencing alignment negatively. Additionally, libraries prepared through SPLP may show different peptide distributions from the solution phase preparations.

Peptide library K2C8 was sequenced in the solution phase at 500 pmol (SOP) and 5 pmol protein concentration (FIG. 9C). The 5 pmol solution phase sequencing produced significantly fewer peptide alignments (32 vs. 3461 for SOP) and peptide identifications (1 vs. 8 for SOP) than the 500 pmol solution phase sequencing. Peptide library K2C8 was sequenced in the solution phase at 500 pmol protein concentration (SOP), and at 5 pmol protein concentration using SPLP (FIG. 9D). The 5 pmol SPLP sequencing produced more peptide alignments (1632 vs. 6524 for SOP) and peptide identifications (3 vs. 7 for SOP) compared to the 5 pmol solution phase sequencing. SPLP also showed a peptide distribution change compared to the solution phase.

A modification of the Glen-SS-BME-PFP resin was prepared, Glen-SS-BME-NHS, containing an N-hydroxysuccinimide moiety instead of pentafluorophenoxy:

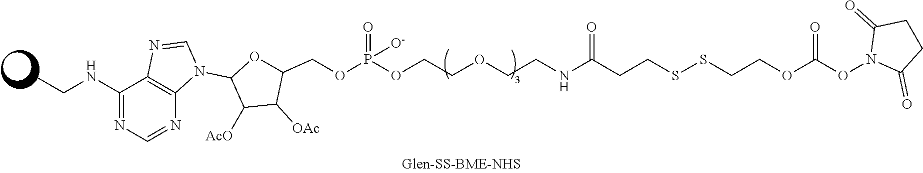

Both the Glen-SS-BME-PFP and Glen-SS-BME-NHS resins were reacted with a Cy3 peptide (Scheme 5). Complete catching of the Cy3 peptide (i.e., reaction of the resin with the Cy3 peptide) was observed with Glen-SS-BME-NHS resin within a 4 hr reaction time, whereas complete catching took longer with the Glen-SS-BME-PFP resin (FIG. 10). The duration of the catching reaction may be modified (e.g., to overnight) for slower reacting peptides.

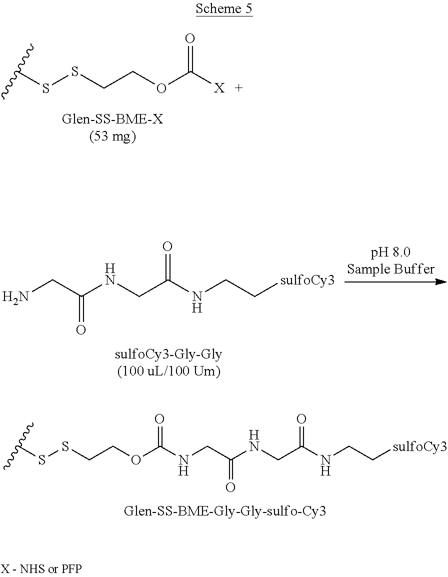

A modification of the Glen-SS-BME-NHS resin was prepared, Glen-SS-BMP-NHS, containing a methyl substituent on the ethylene group between the disulfide and carbamate moieties:

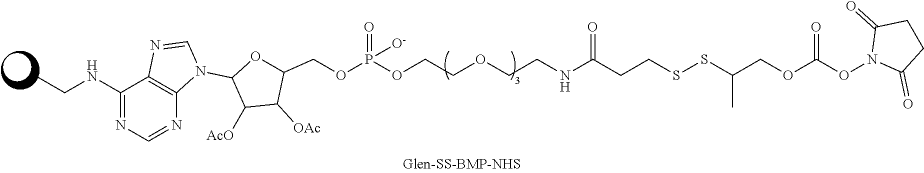

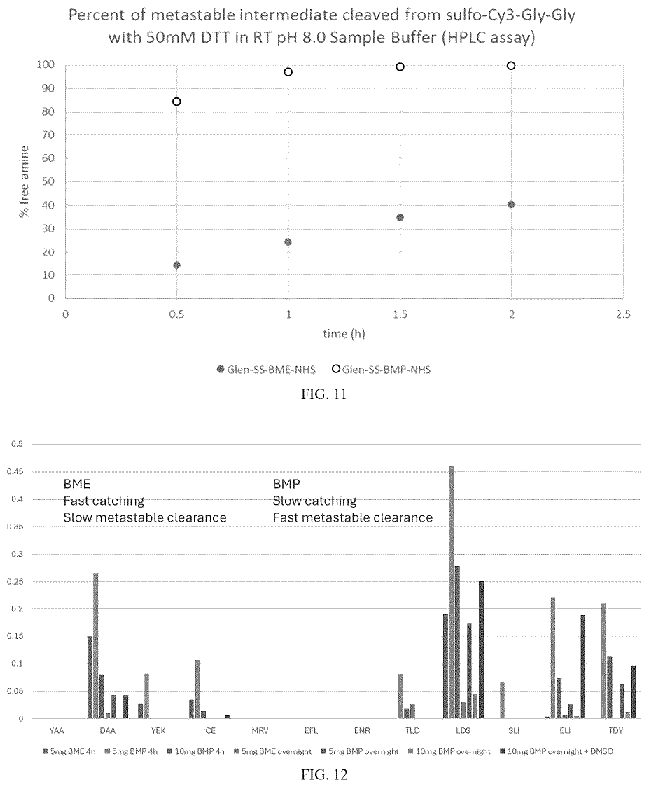

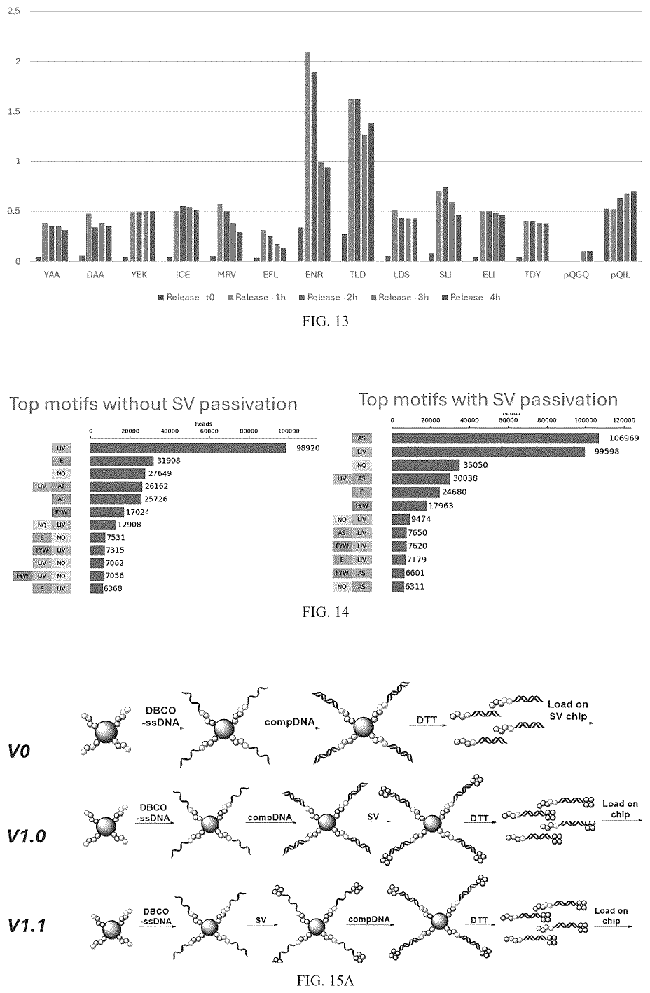

Scheme 6 shows the reaction of resin-peptide conjugates of the two resins (BME (where R=H) and BMP (where R=methyl)) to form a metastable intermediate followed by release of the free peptide. Release of the free peptide occurred more rapidly with conjugates derived from the BMP resin compared to the BME resin; metastable peptide-BMP adducts were cleared in 1.5 hrs at room temperature (FIG. 11). Additional studies using mass spectrometry revealed that the BME resin has faster catching but slower clearance of the metastable intermediate, while the BMP resin has slower catching but faster clearance of the metastable intermediate (FIG. 12). However, studies at elevated temperatures showed that metastable peptide-BME adducts were completely cleared within 1 hour of heating at 45° C. (FIG. 13).

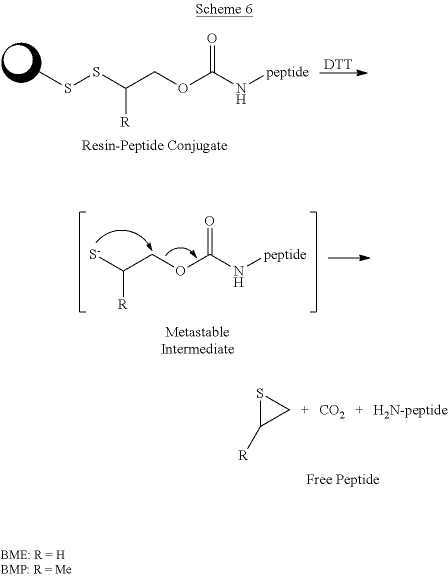

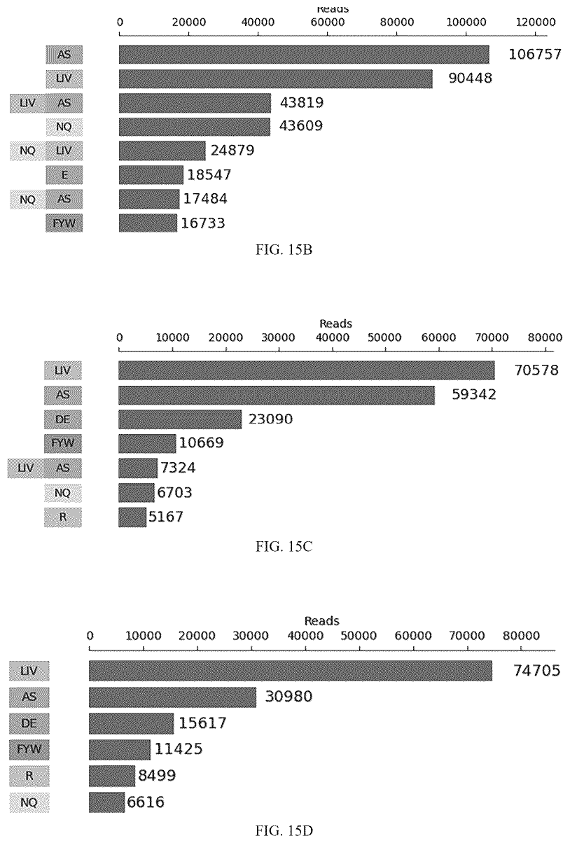

The initial SPLP workflow involved streptavidin (SV) passivation prior to the loading of peptide-DNA conjugates, but this produced “AS” artifacts related to SV and PS1220 (R binder). At low sample input, the “AS” artifacts are significant compared to sequencing signal, and negatively affect the alignment of synthetic CDNF control (FIG. 14). Modifications to the SPLP workflow (e.g., on-bead SV rather than on-chip SV) (FIG. 15A) led to less SV-binding sticking and reduction of the “AS” artifacts (FIGS. 15B-15D).

It was hypothesized that there were transient covalent interactions between DNA in the linker and the reactive groups on the resin. Different quenching reagents were tested to reduce the reactive groups on resin after peptide catching (i.e., to quench the resin). Unexpectedly, ethanolamine quenching allows reduction of non-specific linker sticking to the Glen-SS-BME-NHS resin (Table 8). The level of sticking after 4 hrs incubation is insignificant for libraries prepared at 5 pmol protein input.

TABLE 8
Quantification of non-specific sticking of DNA linker to resin

| Reagent (50 mM) used | | | | |
|---|---|---|---|---|
| to quench the reactive | | Quenching | | Non-specific |
| groups on resin | Resin | time | | sticking (nM) |
| Methylamine | Glen-SS-BME-NHS | 30 | mins | 157 |
| Methylamine | Glen-SS-BME-PFP | 30 | mins | 66 |
| Ethanolamine | Glen-SS-BME-NHS | 30 | mins | 108 ± 4 |
| Ethanolamine | Glen-SS-BME-NHS | 4 | hrs | 63 |

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the FIGURES, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A compound of Formula (I): 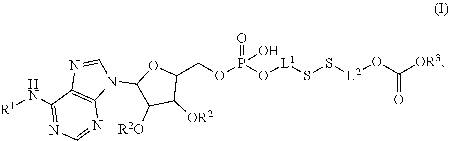
or a salt thereof, wherein:
R¹ is a solid support;
each instance of R² is independently hydrogen or an oxygen protecting group;
R³ is optionally substituted alkyl, optionally substituted heterocyclyl, optionally substituted aryl, or optionally substituted heteroaryl;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof; and
L² is optionally substituted C₂ alkylene.

2. The compound of claim 1 wherein the compound is of Formula (I′): 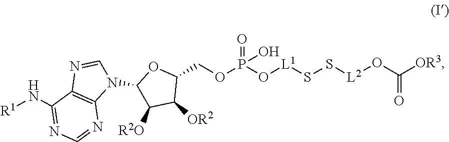
or a salt thereof.

3. The compound of claim 1 or claim 2, or salt thereof, wherein R³ is optionally substituted aryl.

4. The compound of any one of claims 1-3, or salt thereof, wherein R³ is phenyl substituted with at least one halogen or —NO₂.

5. The compound of any one of claims 1-4, or salt thereof, wherein R³ is phenyl substituted with at least one fluoro or —NO₂.

6. The compound of claim 1 or claim 2, or salt thereof, wherein R³ is optionally substituted heterocyclyl.

7. The compound of any one of claims 1-6, or salt thereof, wherein R³ is

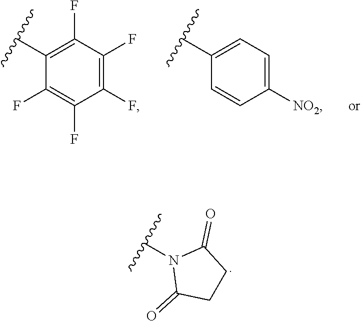 [SMILES: *c1c(F)c(F)c(F)c(F)c1F ; *c1ccc([N+](=O)[O-])cc1 ; *N1C(=O)CCC1=O]

8. A compound of Formula (II): 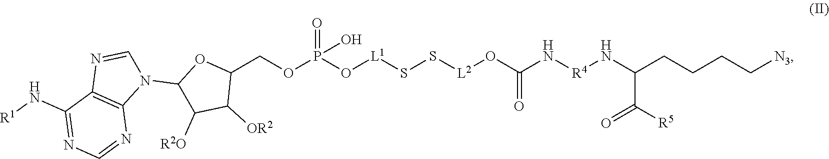
or a salt thereof, wherein:
R¹ is a solid support;
each instance of R² is independently hydrogen or an oxygen protecting group;
R⁴ is a peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof; and
L² is optionally substituted C₂ alkylene.

9. The compound of claim 8, wherein the compound is of Formula (II′): 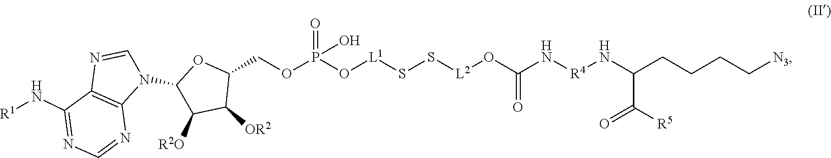
or a salt thereof.

10. A compound of Formula (III): 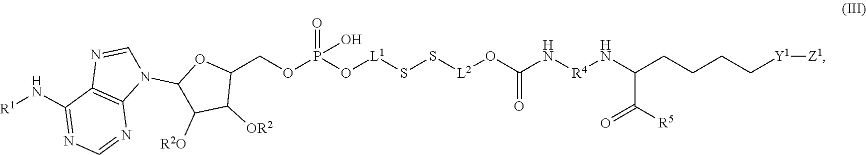
or a salt thereof, wherein:
R¹ is a solid support;
each instance of R² is independently hydrogen or an oxygen protecting group;
R⁴ is a peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof;
L² is optionally substituted C₂ alkylene;
Y¹ comprises a click chemistry adduct; and
Z¹ comprises an oligonucleotide.

11. The compound of claim 10, wherein the compound is of Formula (III′): 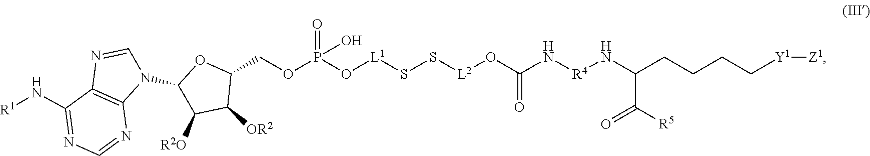
or a salt thereof.

12. The compound of any one of claims 1-11, or salt thereof, wherein R¹ is a polystyrene support.

13. The compound of any one of claims 1-12, or salt thereof, wherein at least one instance of R² is hydrogen.

14. The compound of any one of claims 1-13, or salt thereof, wherein at least one instance of R² is —C(═O)R^{2a}, wherein R^{2a} is optionally substituted alkyl or optionally substituted aryl.

15. The compound of any one of claims 1-14, or salt thereof, wherein at least one instance of R² is —C(═O)R^{2a}, wherein R^{2a} is optionally substituted C₁₋₆ alkyl.

16. The compound of any one of claims 1-15, or salt thereof, wherein at least one instance of R² is —C(═O)R^{2a}, wherein R^{2a} is unsubstituted C₁₋₃ alkyl.

17. The compound of any one of claims 1-16, or salt thereof, wherein at least one instance of R² is —C(═O)CH₃.

18. The compound of any one of claims 1-17, or salt thereof, wherein L¹ comprises optionally substituted alkylene, optionally substituted heteroalkylene, or a combination thereof.

19. The compound of any one of claims 1-18, or salt thereof, wherein L¹ comprises 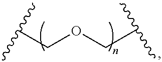
wherein n is an integer between 0 and 30, inclusive.

20. The compound of any one of claims 1-19, or salt thereof, wherein L¹ comprises —NHC(═O)— or —C(═O)NH—.

21. The compound of any one of claims 1-20, or salt thereof, wherein L¹ comprises ethylene.

22. The compound of any one of claims 1-21, or salt thereof, wherein L¹ is 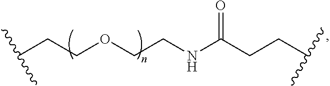
wherein n is an integer between 0 and 30, inclusive.

23. The compound of any one of claims 1-22, or salt thereof, wherein L² is

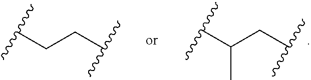

24. The compound of any one of claims 1-23, or salt thereof, wherein L² is ethylene.

25. The compound of any one of claims 1, 3-5, and 12-24, wherein the compound is of Formula (I-a): 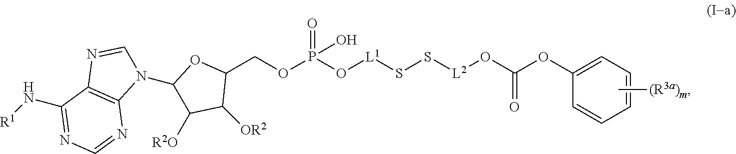
or a salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
m is 0, 1, 2, 3, 4, or 5.

26. The compound of claim 25, wherein the compound is of Formula (I′-a): 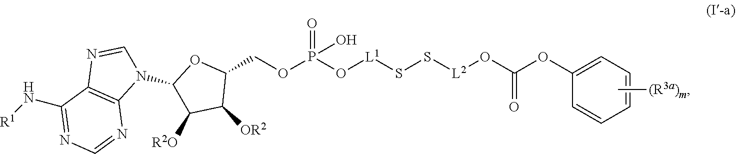
or a salt thereof.

27. The compound of any one of claims 1, 3-7, and 12-25, wherein the compound is of Formulae (I-a-1), (I-a-2), or (I-b): 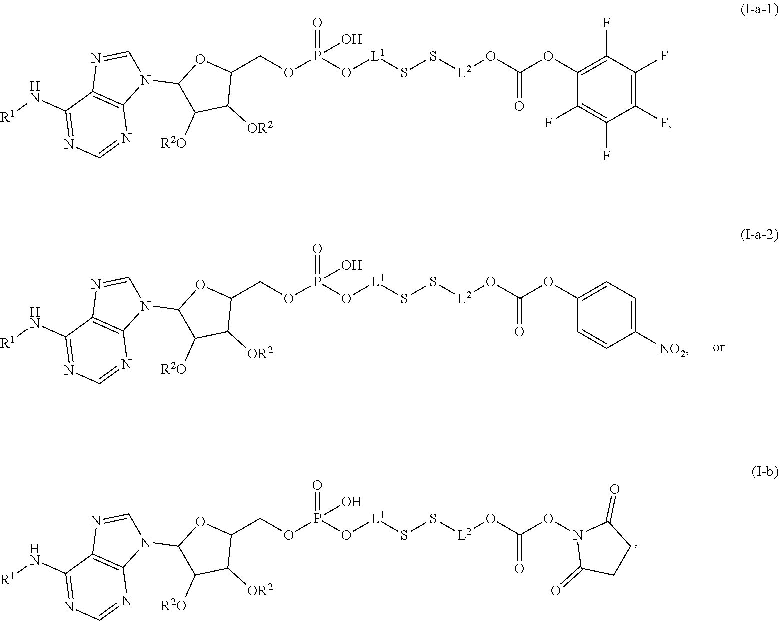
or a salt thereof.

28. The compound of any one of claims 1, 3-7, and 12-23, wherein the compound is of Formula (I-c) or (I-cc): 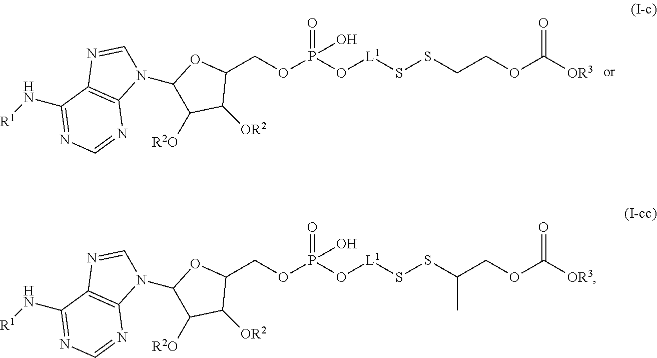
or a salt thereof.

29. The compound of any one of claims 1, 3-5, 12-25, and 28, wherein the compound is of Formula (I-d) or (I-dd): 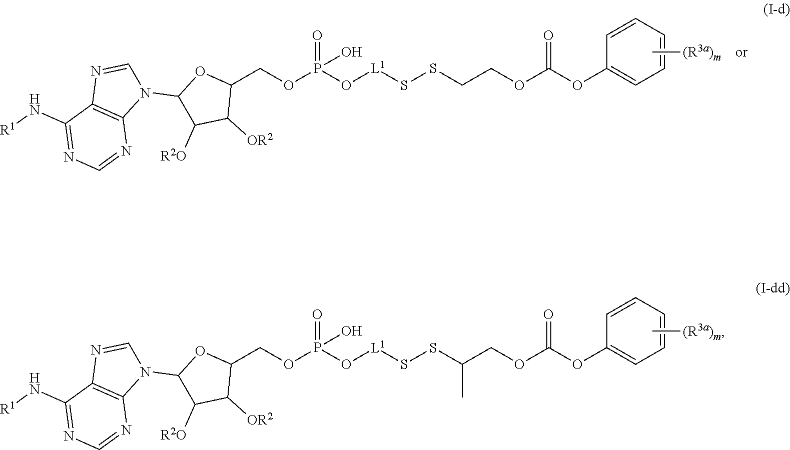
or a salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
m is 0, 1, 2, 3, 4, or 5.

30. The compound of any one of claims 1, 3-7, 12-25, and 27-29, wherein the compound is of Formulae (I-d-1), (I-d-2), (I-dd-1), (I-dd-2), (I-e), or (I-ee): 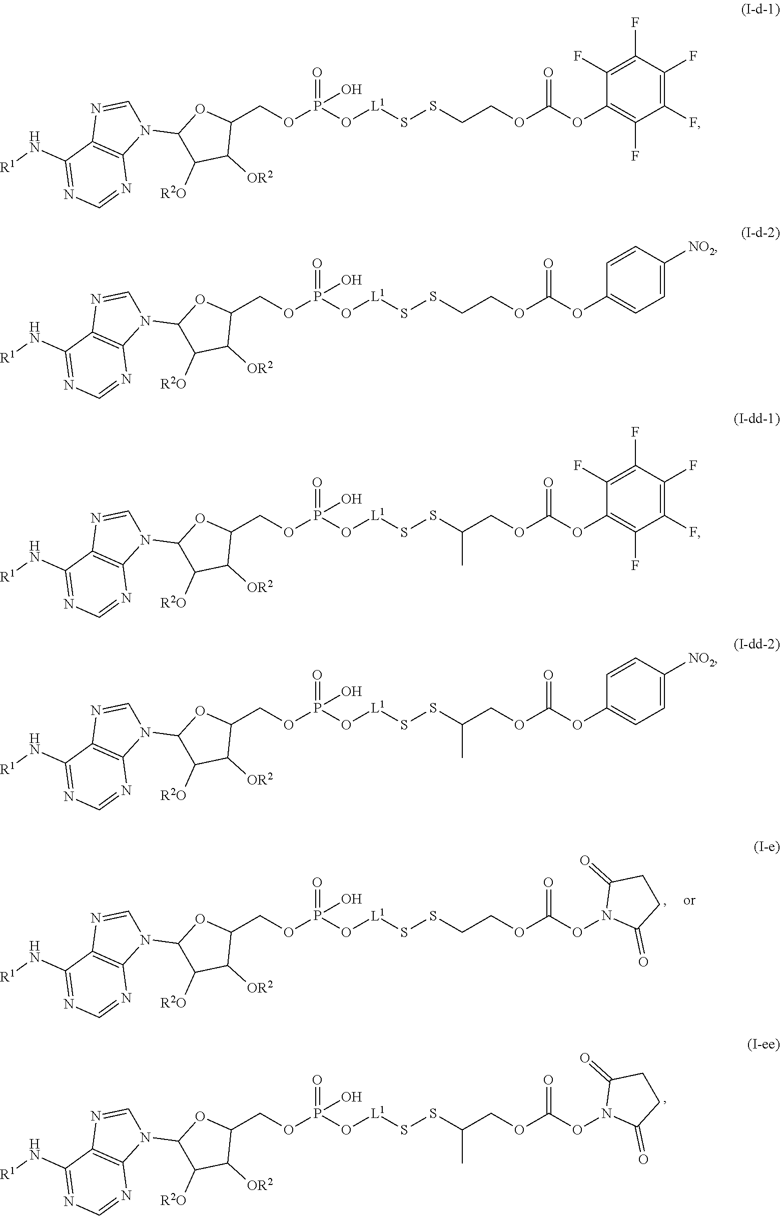
or a salt thereof.

31. The compound of any one of claims 1, 3-7, and 12-23, wherein the compound is of Formula (I-f): 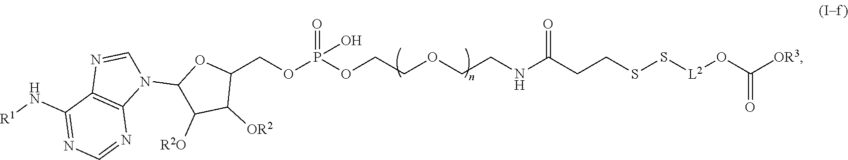
or a salt thereof, wherein n is an integer between 0 and 30, inclusive.

32. The compound of any one of claims 1, 3-5, 12-25, and 31, wherein the compound is of Formula (I-g): 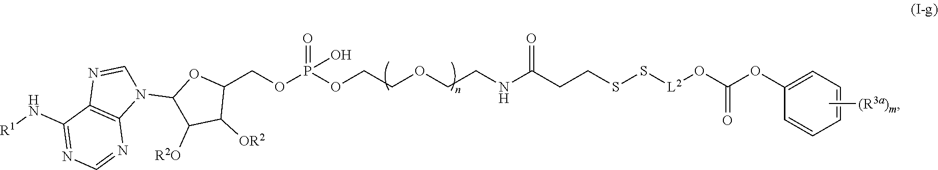
or a salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(RA)₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring;
m is 0, 1, 2, 3, 4, or 5; and
n is an integer between 0 and 30, inclusive.

33. The compound of any one of claims 1, 3-7, 12-25, 27, 31, and 32, wherein the compound is of Formulae (I-g-1), (I-g-2), or (I-h):

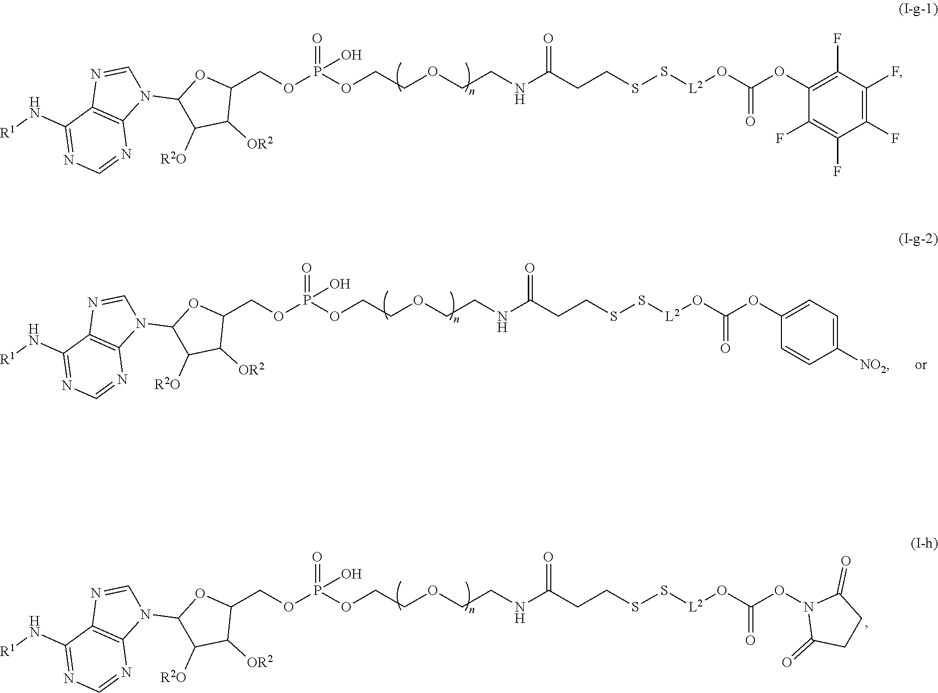

34. The compound of any one of claims 1, 3-7, 12-25, 28, and 31, wherein the compound is of Formulae (I-i) or (I-ii): 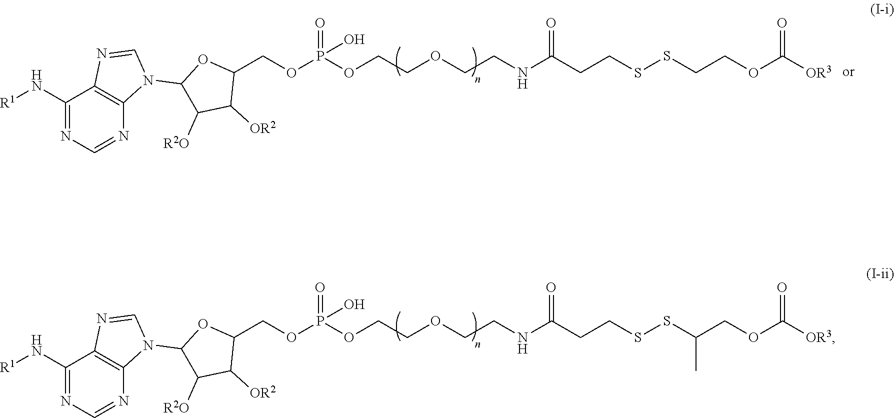
or a salt thereof, wherein n is an integer between 0 and 30, inclusive.

35. The compound of any one of claims 1, 3-5, 12-25, 28, 29, 31, and 32, wherein the compound is of Formula (I-j) or (I-jj): 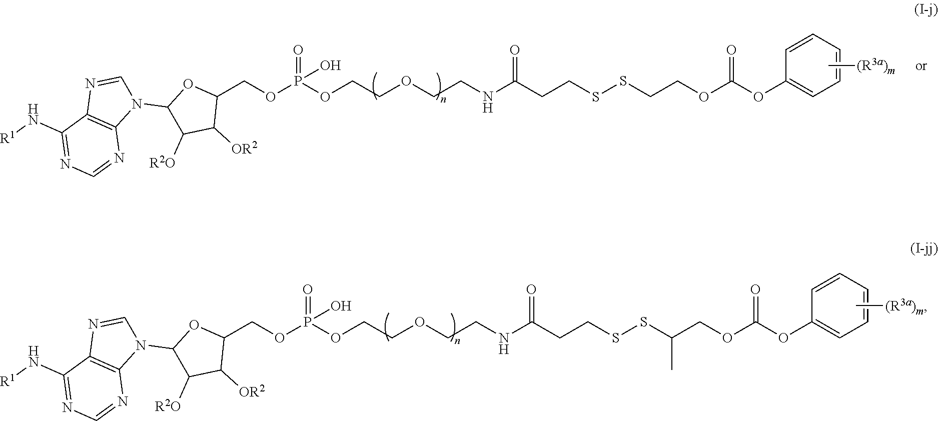
or a salt thereof, wherein:
each instance of R^{3a} is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring;
m is 0, 1, 2, 3, 4, or 5; and
n is an integer between 0 and 30, inclusive.

36. The compound of any one of claims 1, 3-7, 12-25, and 27-35, wherein the compound is of Formulae (I-j-1), (I-j-2), (I-jj-1), (I-jj-2), (I-k), or (I-kk): 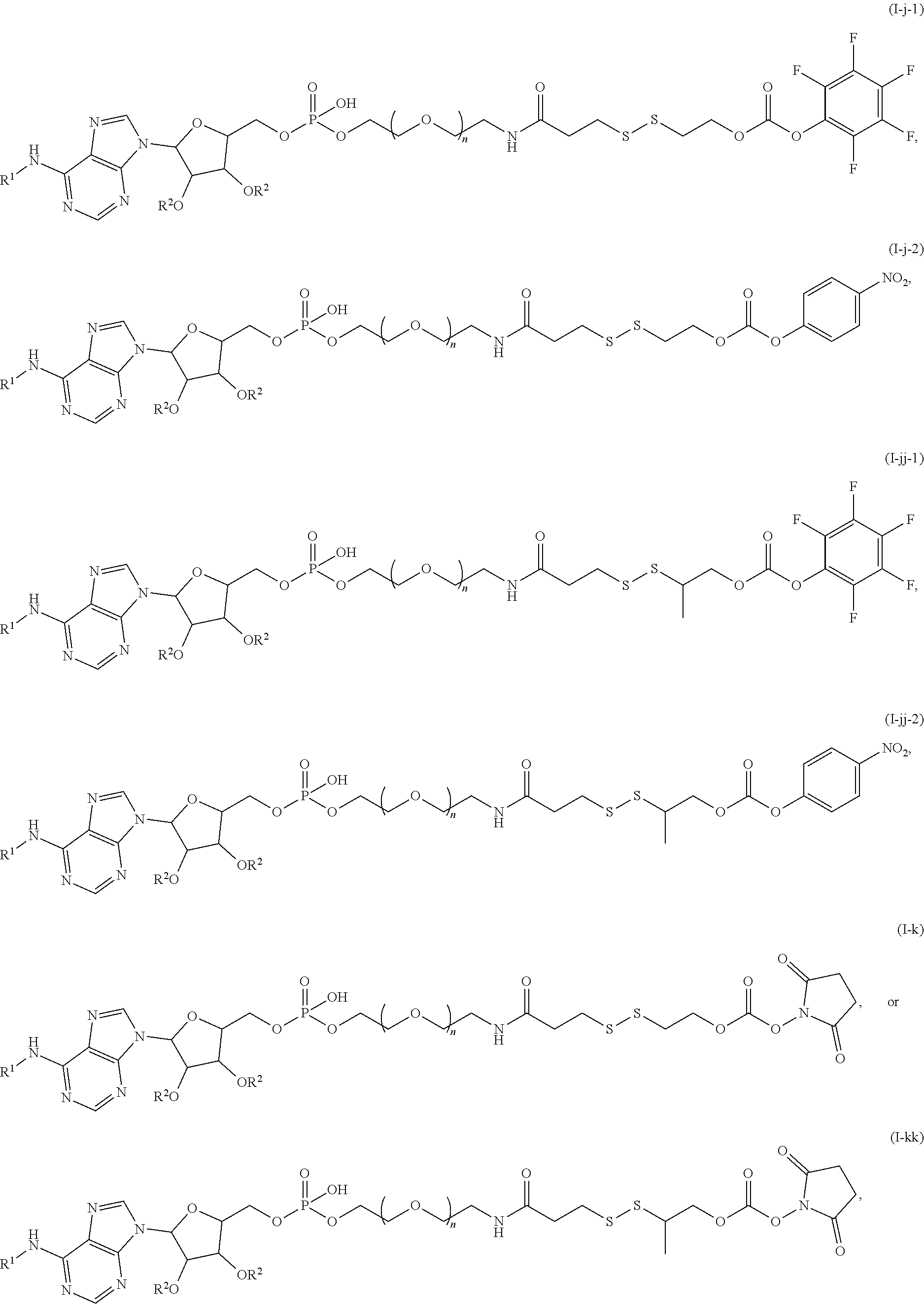
or a salt thereof, wherein n is an integer between 0 and 30, inclusive.

37. The compound of any one of claims 1, 3-7, 12-25, and 27-36, wherein the compound is of Formulae (I-j-3), (I-j-4), (I-jj-3), (I-jj-4), (I-k-1), or (I-kk-1): 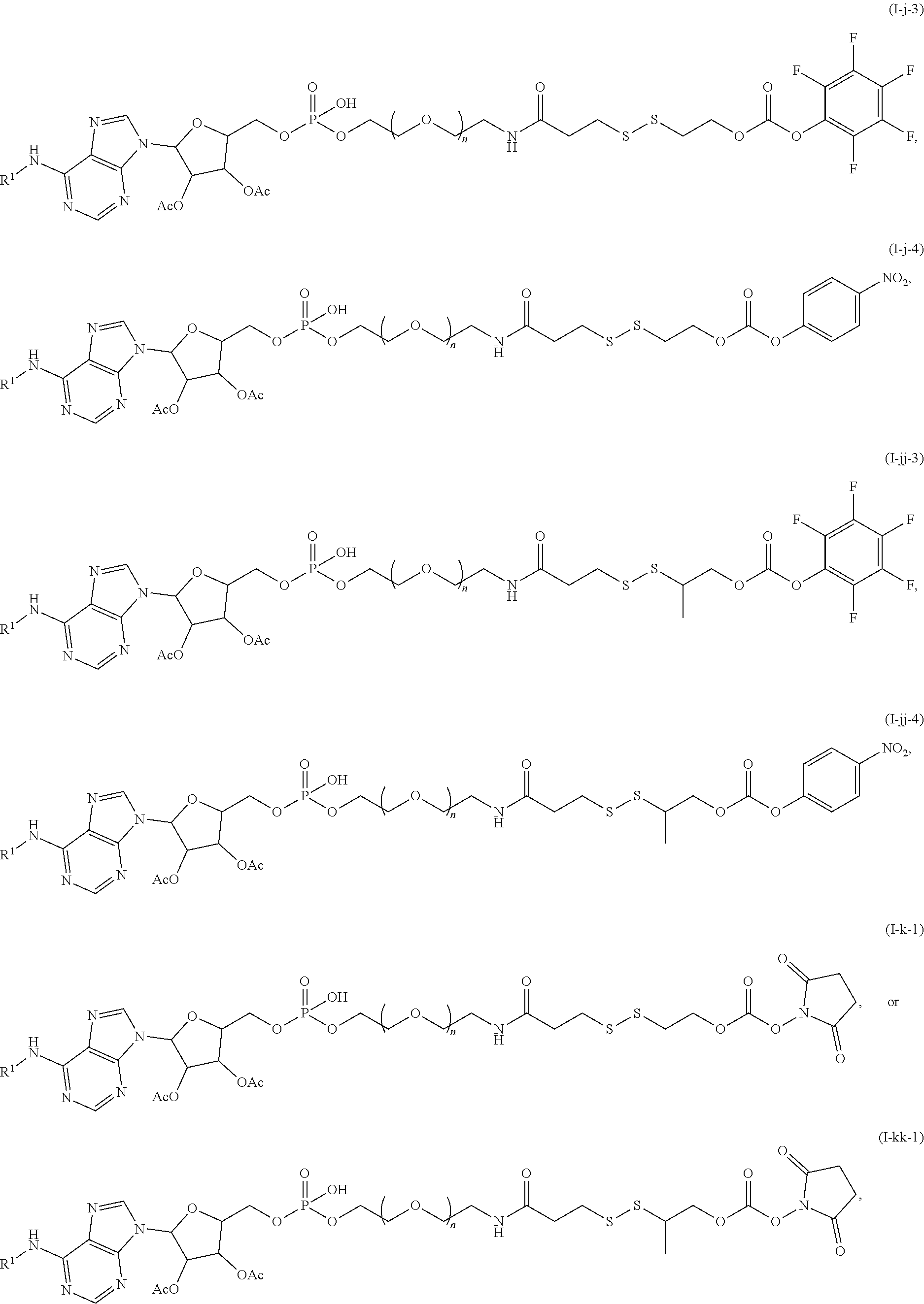
or a salt thereof, wherein n is an integer between 0 and 30, inclusive.

38. The compound of any one of claims 1, 3-7, 12-25, and 27-37, wherein the compound is of Formulae (I-j-5), (I-j-6), (I-jj-5), (I-jj-6), (I-k-2), or (I-kk-2): 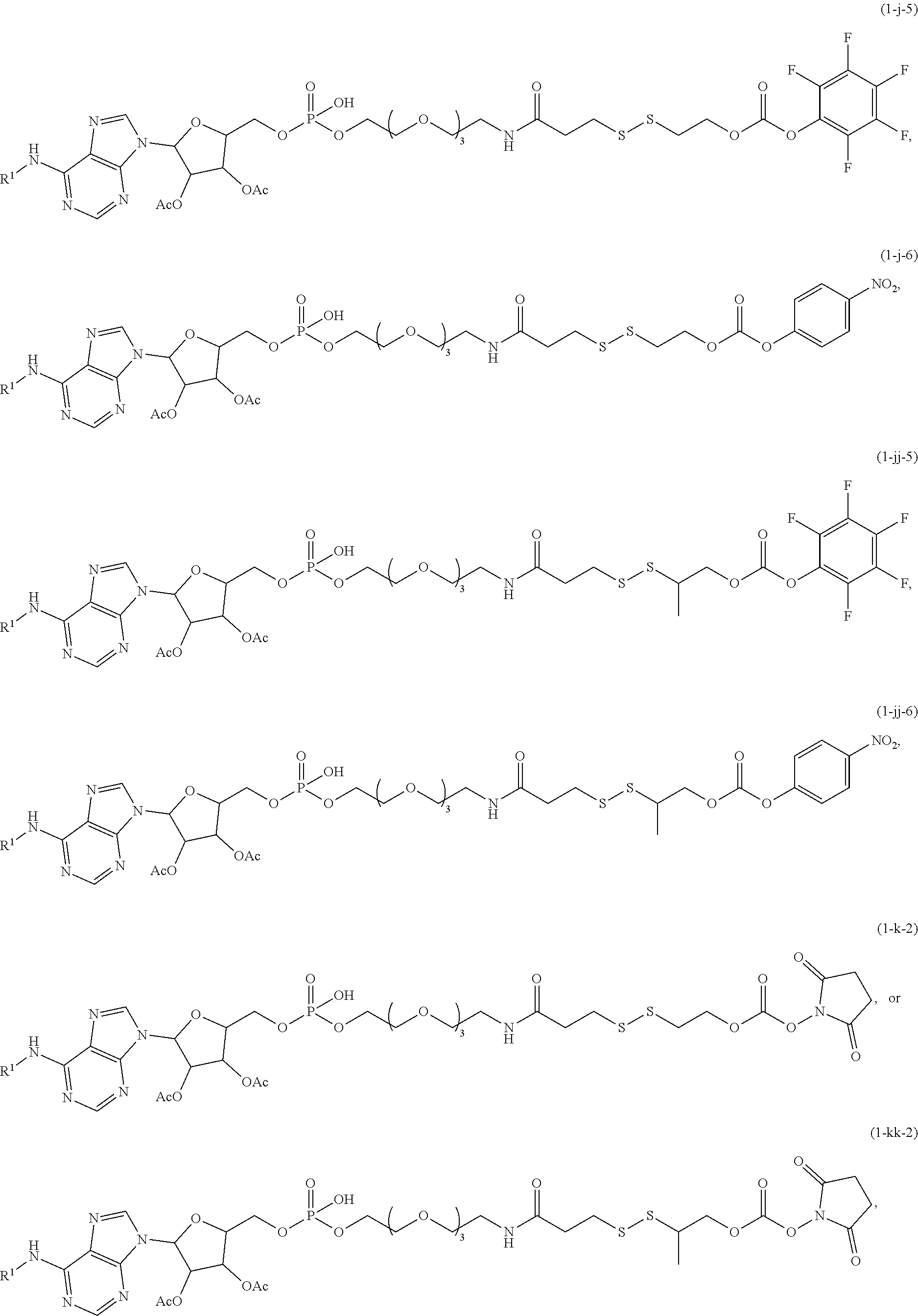
or a salt thereof.

39. The compound of any one of claims 1-7 and 12-38, wherein the compound is of Formula (I′-j-5) or (I′-jj-5): 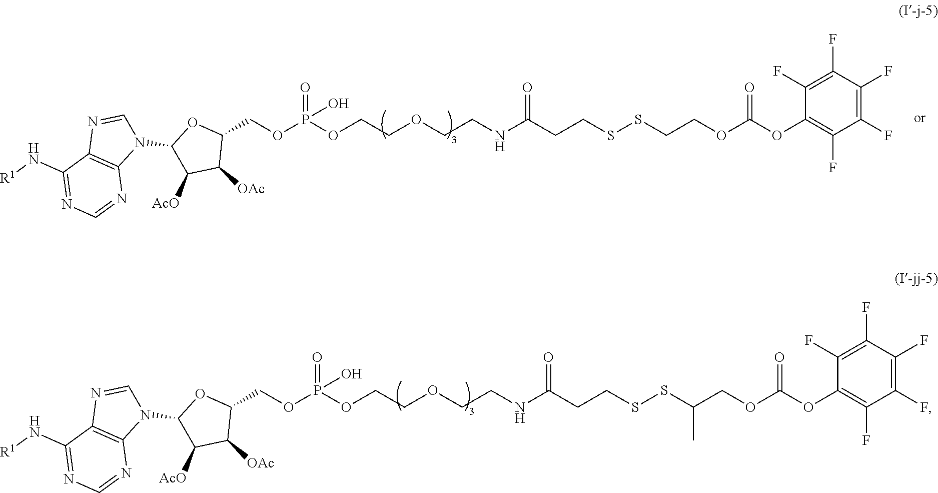
or a salt thereof.

40. The compound of any one of claims 25, 26, 29, 32, and 35, or salt thereof, wherein at least one instance of R^{3a} is halogen or —NO₂.

41. The compound of any one of claims 25, 26, 29, 32, 35, and 40, or salt thereof, wherein each instance of R^{3a} is independently halogen.

42. The compound of any one of claims 25, 26, 29, 32, 35, 40, and 41, or salt thereof, wherein at least one instance of R^{3a} is fluoro.

43. The compound of any one of claims 25, 26, 29, 32, 35, 40, and 42, or salt thereof, wherein at least one instance of R^{3a} is —NO₂.

44. The compound of any one of claims 8-23, or salt thereof, wherein R⁵ is —OH.

45. The compound of any one of claims 8-23, or salt thereof, wherein R⁵ is —NH₂.

46. The compound of any one of claims 8, 12-23, 44, and 45, wherein the compound is of Formulae (II-a-1), (II-a-2), or (II-b): 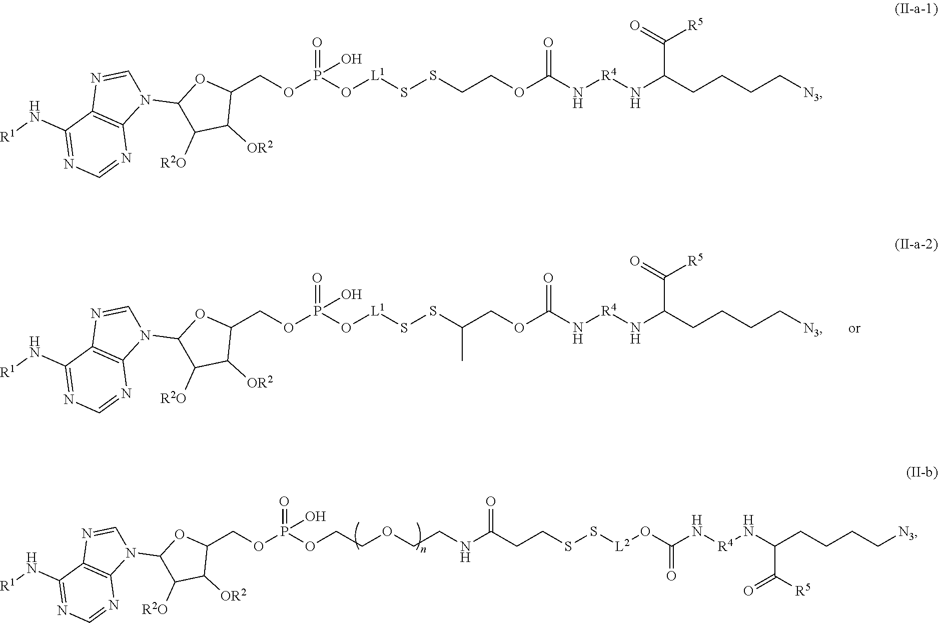
or a salt thereof, wherein n is an integer between 0 and 30, inclusive.

47. The compound of any one of claims 8, 12-23, 44, and 45, wherein the compound is of Formula (II-c-1) or (II-c-2): 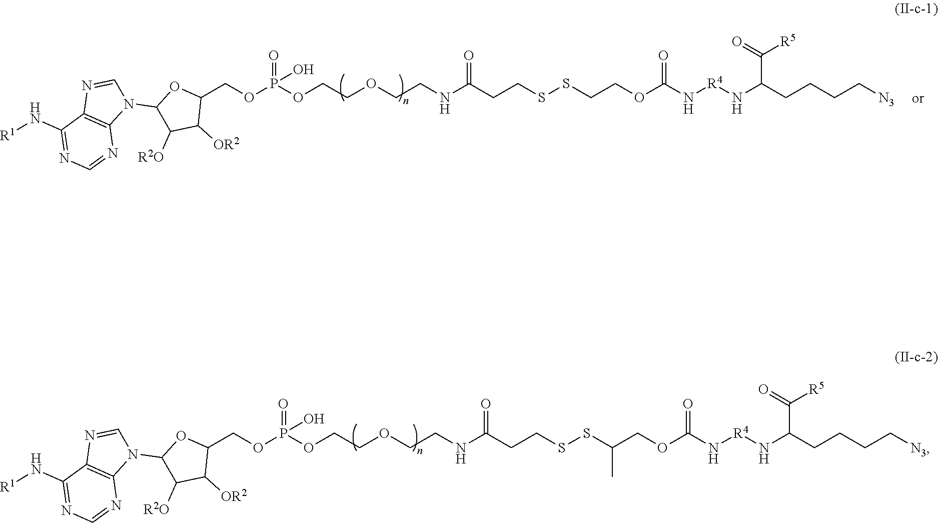
or a salt thereof, wherein n is an integer between 0 and 30, inclusive.

48. The compound of any one of claims 10, 12-23, 44, and 45, wherein the compound is of Formulae (III-a-1), (III-a-2), or (III-b): 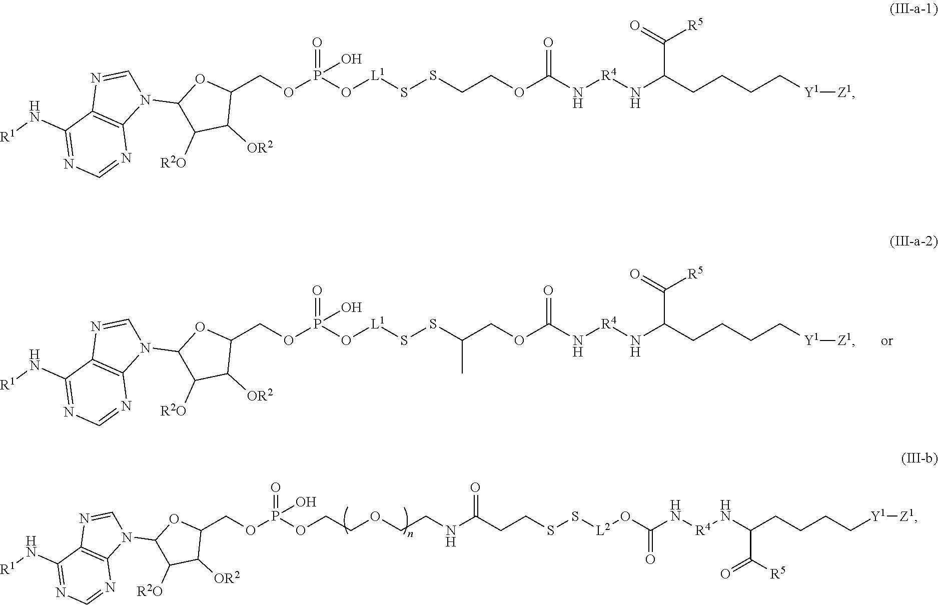
or a salt thereof, wherein n is an integer between 0 and 30, inclusive.

49. The compound of any one of claims 10, 12-23, 44, and 45, wherein the compound is of Formula (III-c-1) or (III-c-2): 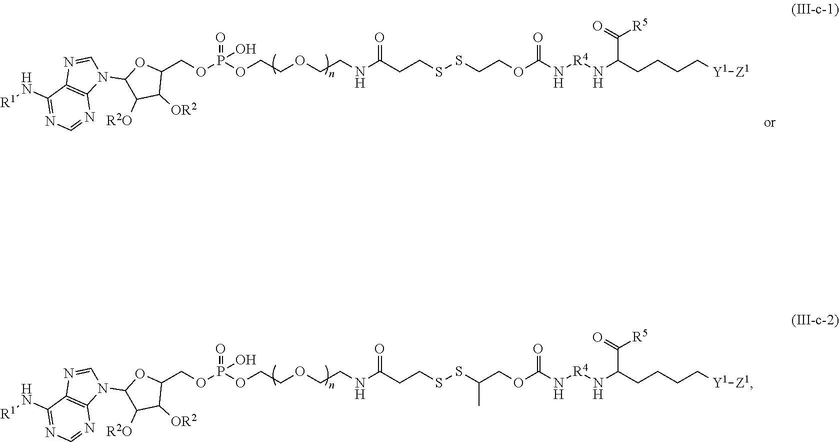
or a salt thereof, wherein n is an integer between 0 and 30, inclusive.

50. The compound of any one of claims 10-23, 44, 45, 48, and 49, or salt thereof, wherein Y¹ comprises a click chemistry adduct formed by a click reaction between a dibenzocyclooctyne (DBCO)-containing moiety and an azide group.

51. The compound of any one of claims 10, 12-23, 44, 45, and 50, wherein the compound is of Formula (III-d): 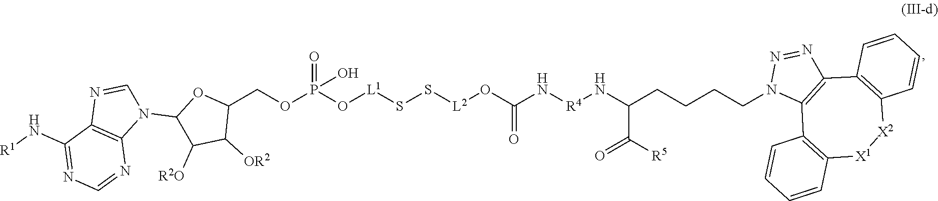
or a salt thereof, wherein one of X¹ and X² is CH and the other is N—Z¹.

52. The compound of claim 51, wherein the compound is of Formulae (III-d-1) or (III-d-2): 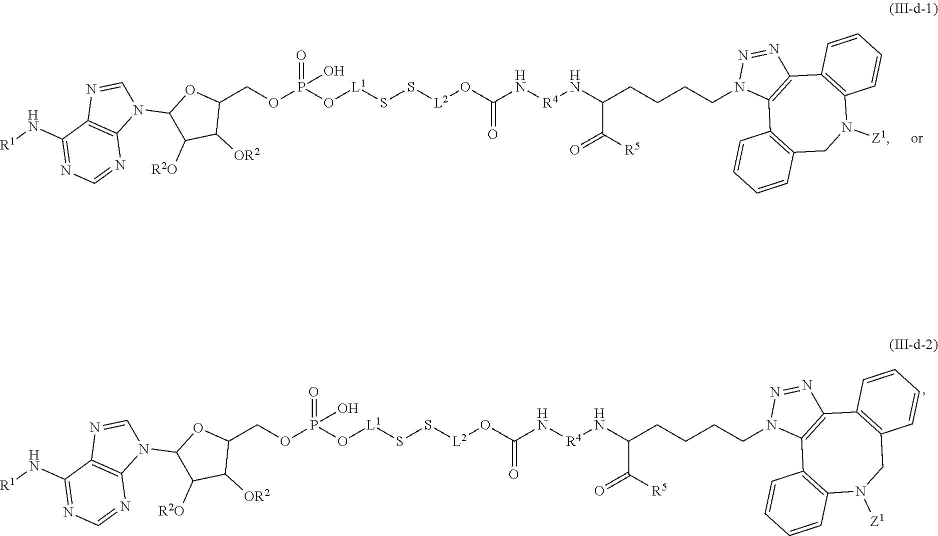
or a salt thereof.

53. The compound of any one of claims 10, 12-23, 44, 45, 48, 50, and 51, wherein the compound is of Formulae (III-e), (III-ee), or (III-f): 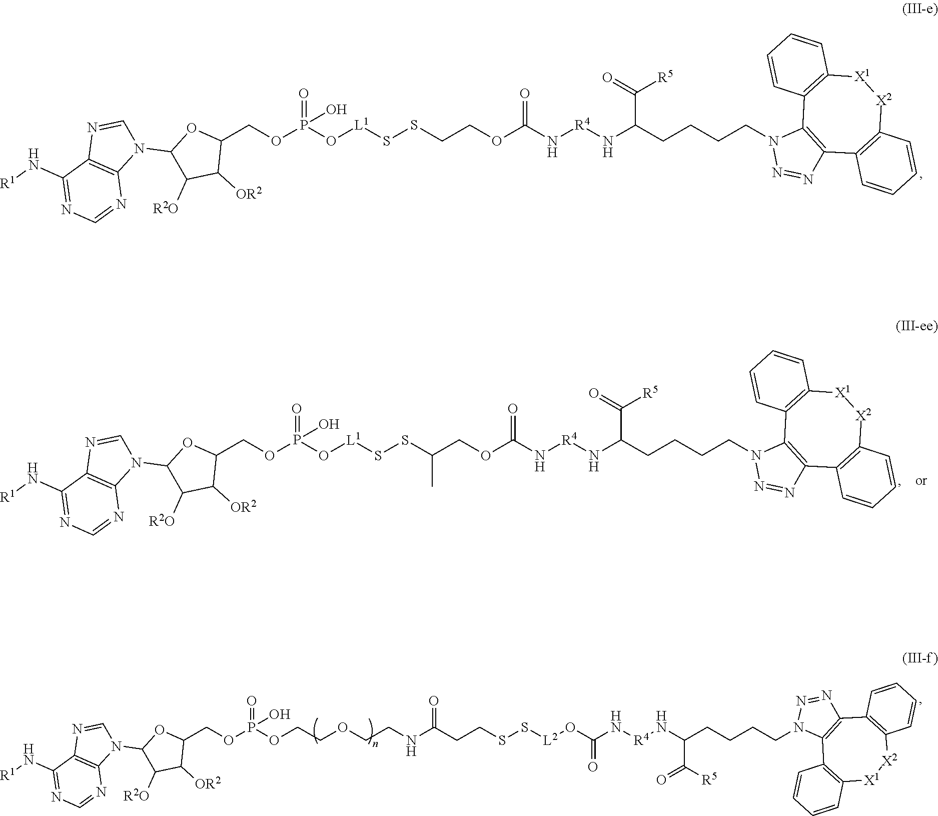
or a salt thereof, wherein one of X¹ and X² is CH and the other is N—Z¹; and n is an integer between 0 and 30, inclusive.

54. The compound of any one of claims 10, 12-23, 44, 45, 48-51, and 53, wherein the compound is of Formulae (III-g) or (III-gg): 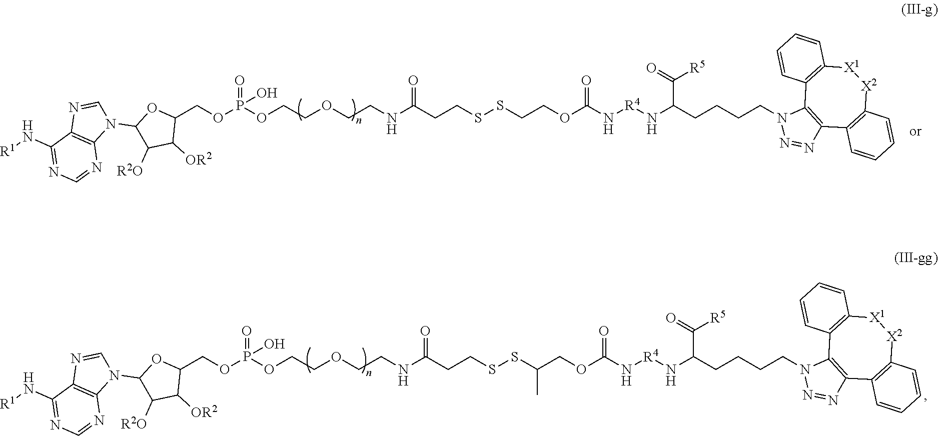
or a salt thereof, wherein one of X¹ and X² is CH and the other is N—Z¹; and n is an integer between 0 and 30, inclusive.

55. The compound of any one of claims 10-23, 44, 45, and 48-54, or salt thereof, wherein Z¹ comprises Q24.

56. The compound of any one of claims 10-23, 44, 45, and 48-55, or salt thereof, wherein Z¹ further comprises a biotin moiety.

57. The compound of claim 56, or salt thereof, wherein the biotin moiety is a bis-biotin moiety.

58. The compound of any one of claims 10-23, 44, 45, and 48-57, or salt thereof, wherein Z¹ further comprises an avidin protein.

59. The compound of claim 58, or salt thereof, wherein the avidin protein is streptavidin.

60. The compound of claim 58 or claim 59, or salt thereof, wherein the avidin protein is immobilized to a surface.

61. A method of preparing a compound of Formula (I): 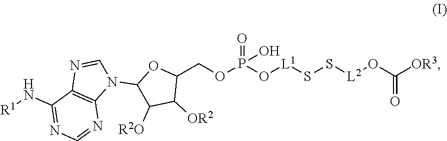
or a salt thereof, comprising coupling a compound of Formula (IV): 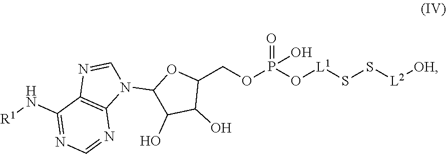
or a salt thereof, with a compound of Formula (V): 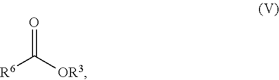
or a salt thereof, under suitable conditions to obtain the compound of Formula (I), or salt thereof, wherein:
R¹ is a solid support;
each instance of R² is independently hydrogen or an oxygen protecting group;
R³ is optionally substituted alkyl, optionally substituted heterocyclyl, optionally substituted aryl, or optionally substituted heteroaryl;
R⁶ is halogen or —OR³;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof.

62. The method of claim 61, wherein R³ is optionally substituted aryl.

63. The method of claim 61 or claim 62, wherein R³ is phenyl substituted with at least one fluoro or —NO₂.

64. The method of claim 61, wherein R³ is optionally substituted heterocyclyl.

65. The method of any one of claims 61-64, wherein R³ is

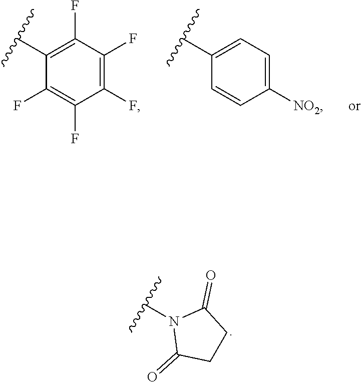 [SMILES: *c1c(F)c(F)c(F)c(F)c1F ; *c1ccc([N+](=O)[O-])cc1 ; *N1C(=O)CCC1=O]

66. The method of any one of claims 61-65, wherein the compound of Formula (IV) is of Formulae (IV-a-1), (IV-a-2), or (IV-b): 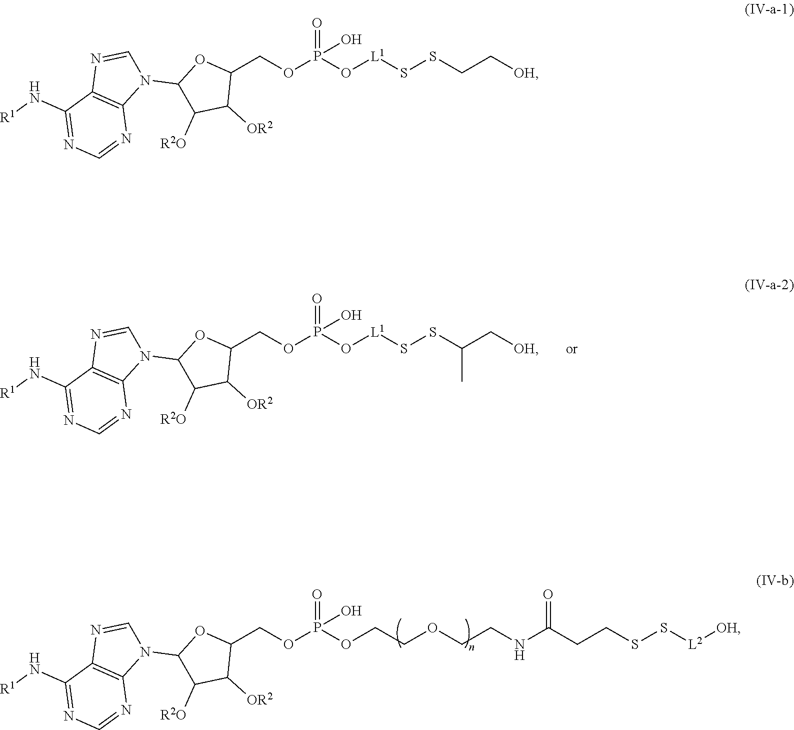
or a salt thereof, wherein n is an integer between 0 and 30, inclusive.

67. The method of any one of claims 61-66, wherein the compound of Formula (IV) is of Formula (IV-c) or (IV-cc): 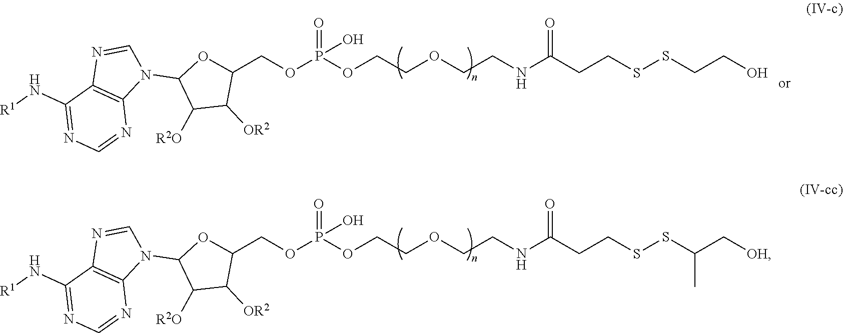
or a salt thereof, wherein n is an integer between 0 and 30, inclusive.

68. The method of claim 67, wherein the compound of Formula (IV) is of Formula (IV-c-1) or (IV-cc-1): 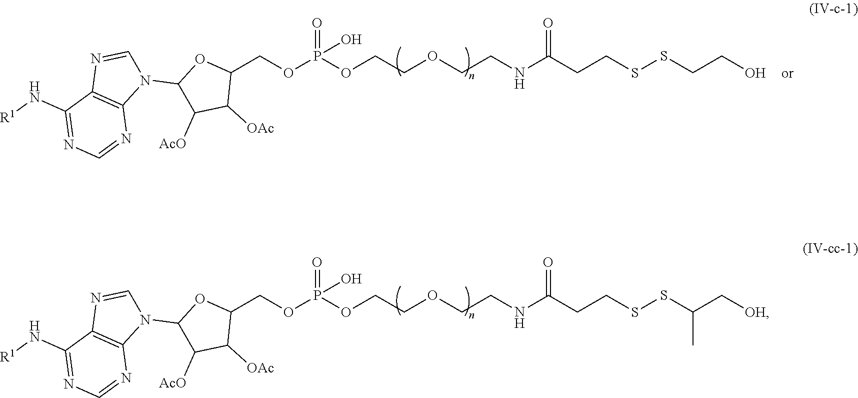
or a salt thereof.

69. The method of any one of claims 61-68, wherein the compound of Formula (IV), or salt thereof, is prepared by reacting a compound of Formula (VI): 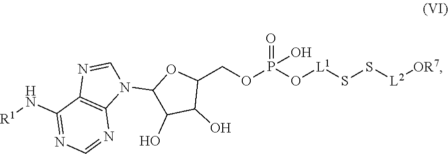
or a salt thereof, under suitable conditions to obtain the compound of Formula (IV), or salt thereof, wherein R⁷ is an oxygen protecting group.

70. The method of claim 69, wherein the compound of Formula (VI) is of Formulae (VI-a), (VI-aa), or (VI-b): 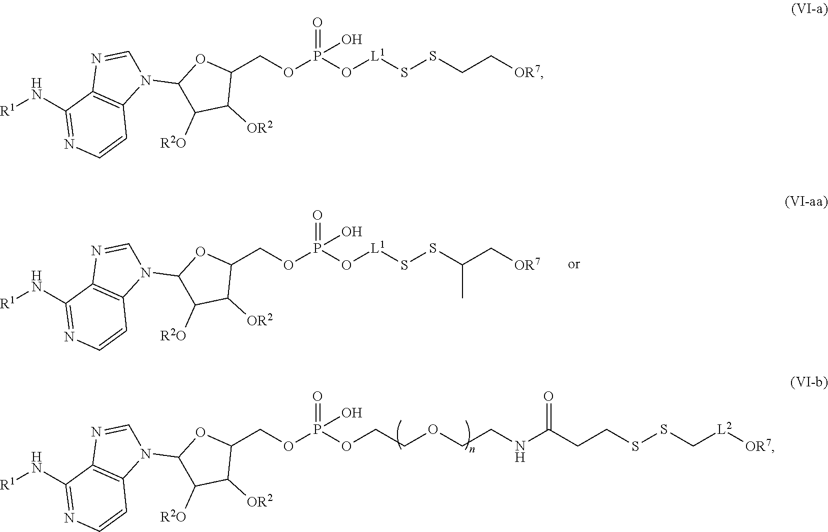
or a salt thereof, wherein n is an integer between 0 and 30, inclusive.

71. The method of claim 69 or claim 70, wherein the compound of Formula (VI) is of Formula (VI-c) or (VI-cc): 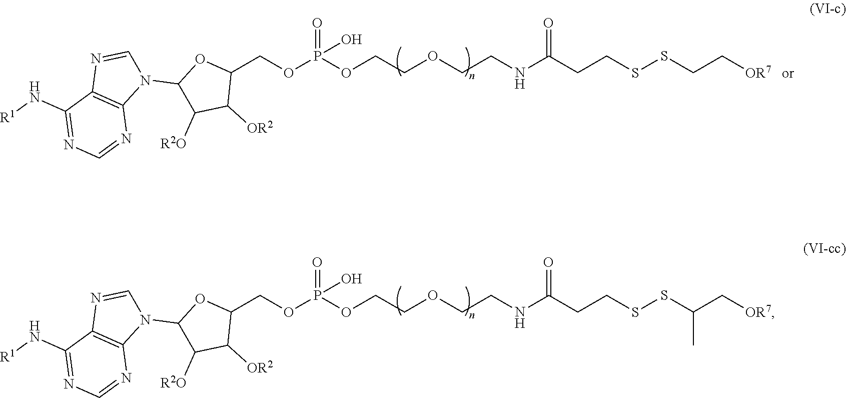
or a salt thereof, wherein n is an integer between 0 and 30, inclusive.

72. The method of claim 71, wherein the compound of Formula (VI-c) is of Formula (VI-c-2) or (VI-cc-2): 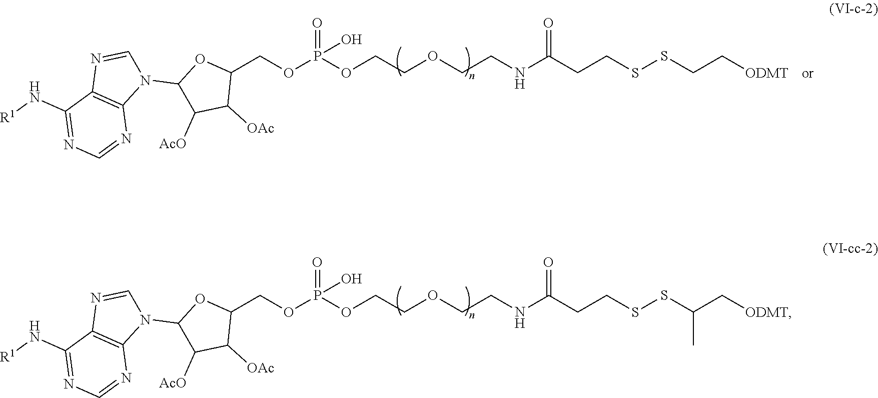
or a salt thereof.

73. The method of any one of claims 69-72, wherein the compound of Formula (VI), or salt thereof, is prepared by reacting a compound of Formula (VII): 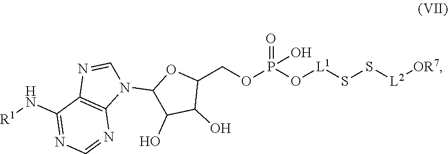
or a salt thereof, under suitable conditions to obtain the compound of Formula (VI), or salt thereof.

74. The method of claim 73, wherein the compound of Formula (VII) is of Formula (VII-c) or (VII-cc): 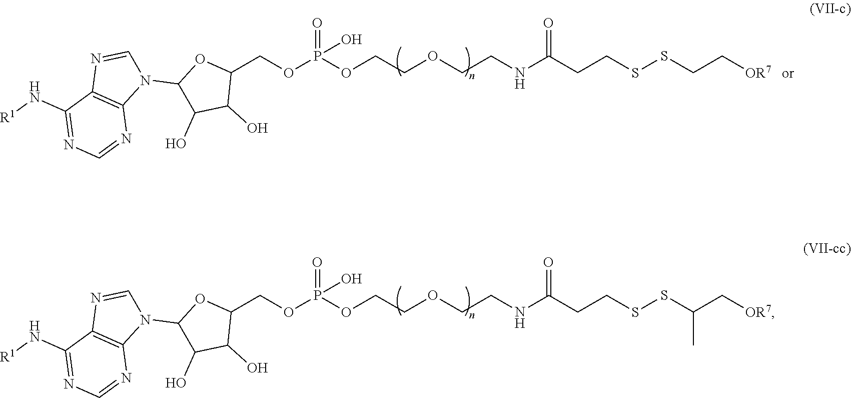
or a salt thereof, wherein n is an integer between 0 and 30, inclusive.

75. The method of claim 74, wherein the compound of Formula (VII-c), or salt thereof, is prepared by coupling a compound of Formula (VIII): 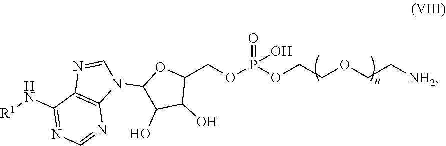
or a salt thereof, with a compound of Formula (IX-a): 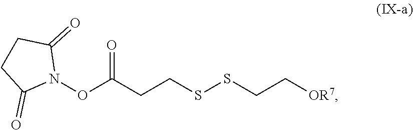
or a salt thereof, under suitable conditions to obtain the compound of Formula (VII-c), or salt thereof.

76. The method of claim 75, wherein the compound of Formula (IX-a), or salt thereof, is prepared by reacting a compound of Formula (X-a): 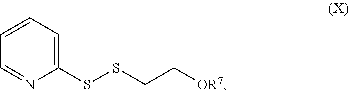
or a salt thereof, under suitable conditions to obtain the compound of Formula (IX), or salt thereof.

77. The method of claim 76, wherein the compound of Formula (X-a), or salt thereof, is prepared by reacting a compound of Formula (XI-a): 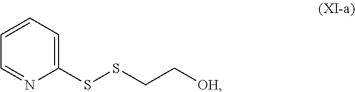 [SMILES: OCCSSc1ccccn1]
or a salt thereof, under suitable conditions to obtain the compound of Formula (X), or salt thereof.

78. The method of claim 74, wherein the compound of Formula (VII-cc), or salt thereof, is prepared by coupling a compound of Formula (VIII): 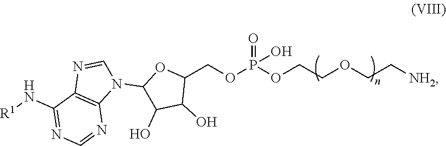
or a salt thereof, with a compound of Formula (IX-b): 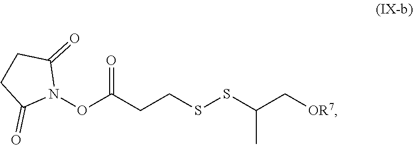
or a salt thereof, under suitable conditions to obtain the compound of Formula (VII-cc), or salt thereof.

79. The method of claim 78, wherein the compound of Formula (IX-b), or salt thereof, is prepared by reacting a compound of Formula (X-b): 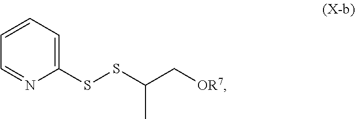
or a salt thereof, under suitable conditions to obtain the compound of Formula (IX-b), or salt thereof.

80. The method of claim 79, wherein the compound of Formula (X-b), or salt thereof, is prepared by reacting a compound of Formula (XI-b): 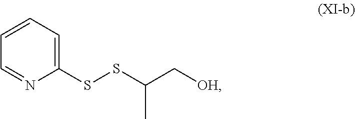
or a salt thereof, under suitable conditions to obtain the compound of Formula (X-b), or salt thereof.

81. A method of preparing a compound of Formula (II): 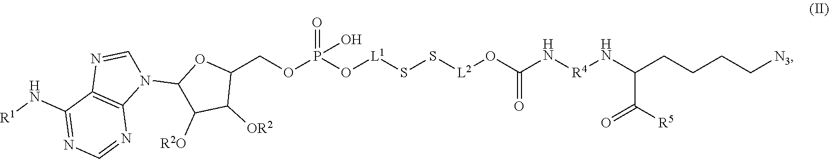
or a salt thereof, comprising coupling a compound of Formula (I): 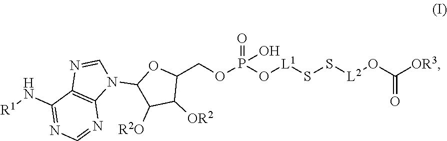
or a salt thereof, with a compound of Formula (XII): 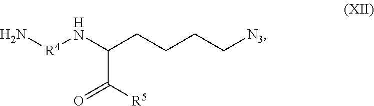
or a salt thereof, under suitable conditions to obtain the compound of Formula (II), or salt thereof, wherein:
R¹ is a solid support;
each instance of R² is independently hydrogen or an oxygen protecting group;
R³ is optionally substituted alkyl, optionally substituted heterocyclyl, optionally substituted aryl, or optionally substituted heteroaryl;
R⁴ is a peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof; and
L² is optionally substituted C₂ alkylene.

82. A method of preparing a compound of Formula (III-d): 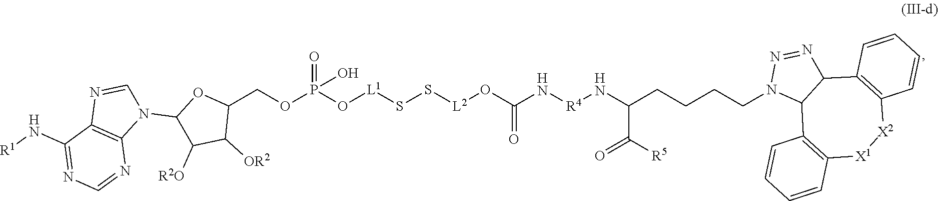
or a salt thereof, comprising coupling a compound of Formula (II): 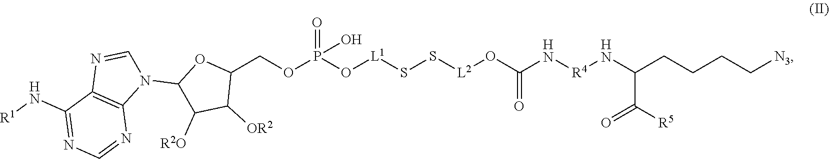
or a salt thereof, with a compound of Formula (XIII): 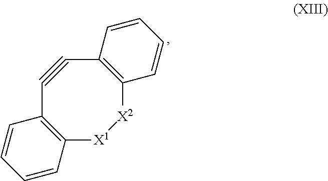
or a salt thereof, under suitable conditions to obtain the compound of Formula (III-d), or salt thereof, wherein:
R¹ is a solid support;
each instance of R² is independently hydrogen or an oxygen protecting group;
R⁴ is a peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof;
L² is optionally substituted C₂ alkylene; and
one of X¹ and X² is CH and the other is N—Z¹, wherein Z¹ comprises an oligonucleotide.

83. The method of any one of claims 61-82, wherein R¹ is a polymeric support.

84. The method of any one of claims 61-67, 69-71, and 73-83, wherein at least one instance of R² is hydrogen.

85. The method of any one of claims 61-72, and 78-83, wherein at least one instance of R² is —C(═O)R^{2a}, wherein R^{2a} is optionally substituted alkyl or optionally substituted aryl.

86. The method of any one of claims 61-72, 78-83, and 85, wherein at least one instance of R² is —C(═O)R^{2a}, wherein R^{2a} is optionally substituted C₁₋₆ alkyl.

87. The method of any one of claims 61-72, 78-83, 85, and 86, wherein at least one instance of R² is —C(═O)R^{2a}, wherein R^{2a} is unsubstituted C₁₋₃ alkyl.

88. The method of any one of claims 61-72, 78-83, and 85-87, wherein at least one instance of R² is —C(═O)CH₃.

89. The method of any one of claims 61-88, wherein L¹ comprises optionally substituted alkylene, optionally substituted heteroalkylene, or a combination thereof.

90. The method of any one of claims 61-89, wherein L¹ comprises 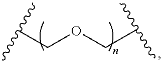
wherein n is an integer between 0 and 30, inclusive.

91. The method of any one of claims 61-90, wherein L¹ comprises —NHC(═O)— or —C(═O)NH—.

92. The method of any one of claims 61-91, wherein L¹ comprises ethylene.

93. The method of any one of claims 61-92, wherein L¹ is 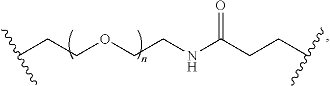
wherein n is an integer between 0 and 30, inclusive.

94. The method of any one of claims 61-93, wherein L² is or

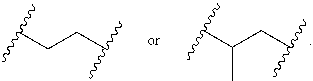

95. The method of any one of claims 61-94, wherein L² is ethylene.

96. The method of any one of claims 61-95, wherein R⁵ is —OH.

97. The method of any one of claims 61-95, wherein R⁵ is —NH₂.

98. The method of any one of claims 78-97, wherein the peptide remains in the solid phase while conjugated to the solid support.

99. The method of any one of claims 82-98, wherein Z¹ comprises Q24.

100. The method of any one of claims 82-99, wherein Z¹ further comprises a biotin moiety.

101. The method of claim 100, wherein the biotin moiety is a bis-biotin moiety.

102. The method of any one of claims 82-101, wherein Z¹ further comprises an avidin protein.

103. The method of claim 102, wherein the avidin protein is streptavidin.

104. The method of claim 102 or claim 103, wherein the avidin protein is immobilized to a surface.

105. A method of functionalizing a first terminus of a peptide comprising:
(a) conjugating a second terminus of the peptide to a solid support group; and
(b) conjugating the first terminus of the peptide to a linking group.

106. A method of sequencing a peptide, comprising:
(a) conjugating a second terminus of the peptide to a solid support group;
(b) conjugating a first terminus of the peptide to a linking group;
(c) exposing the peptide to a peptidase in a degradation process;
(d) obtaining data during the degradation process;
(e) analyzing the data to determine portions of the data corresponding to amino acids that are sequentially exposed at the second terminus of the peptide during the degradation process; and
(f) outputting an amino acid sequence representative of the peptide.

107. The method of claim 105 or 106, further comprising modifying the second terminus of the peptide.

108. The method of any one of claims 105-107, further comprising returning the second terminus of the peptide to a pre-modified state.

109. The method of any one of claims 105-108, wherein the first terminus is a C-terminus.

110. The method of any one of claims 105-109, wherein the second terminus is an N-terminus.

111. The method of any one of claims 105-110, wherein step (a) is performed before step (b).

112. The method of any one of claims 105-111, wherein the peptide remains in the solid phase while conjugated to the solid support group.

113. The method of any one of claims 105-112, wherein the solid support group comprises a cleavable linker.

114. The method of claim 113, further comprising cleaving the cleavable linker.

115. The method of claim 114, wherein the cleavable linker is cleaved upon exposure to a reducing agent.

116. The method of any one of claims 108-115, wherein the pre-modified state is an N-terminus.

117. The method of any one of claims 108-116, wherein returning the second terminus of the peptide to a pre-modified state comprises conversion of a metastable intermediate to an N-terminus.

118. The method of any one of claims 114-117, wherein the peptide is in the solution phase after cleavage of the cleavable linker.

119. The method of any one of claims 105-118, wherein the solid support group comprises a moiety of Formula (XIV): 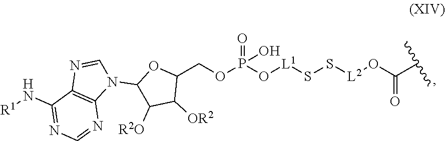
or a salt thereof, wherein:
R¹ is a solid support;
each instance of R² is independently hydrogen or an oxygen protecting group;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof; and
L² is optionally substituted C₂ alkylene.

120. The method of claim 119, wherein the moiety is of Formulae (XIV-a), (XIV-aa), or (XIV-b): 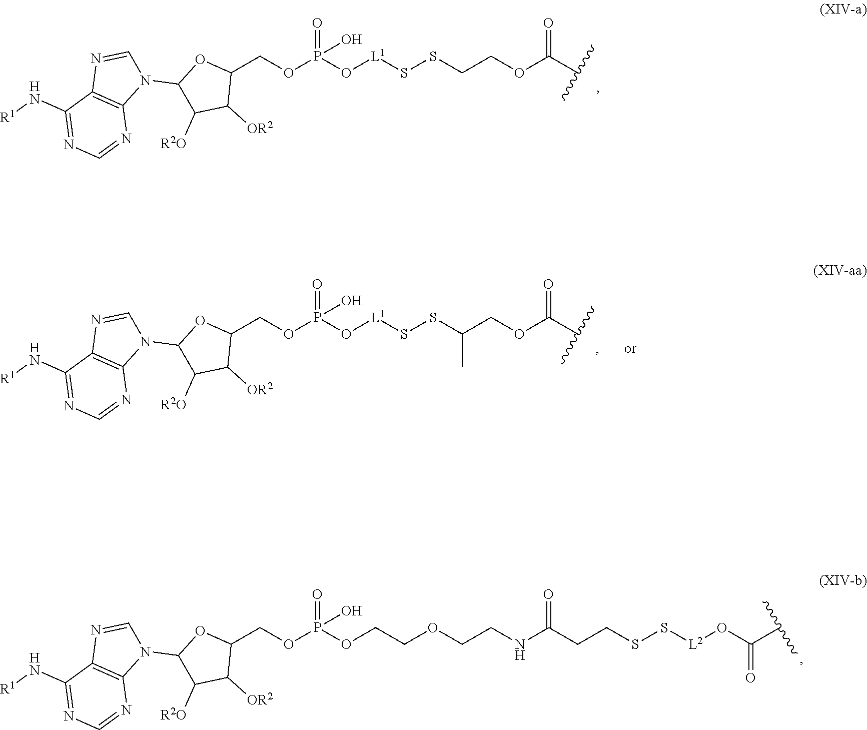
or a salt thereof, wherein n is an integer between 0 and 30, inclusive.

121. The method of claim 119 or claim 120, wherein the moiety is of Formula (XIV-c) or (XIV-cc): 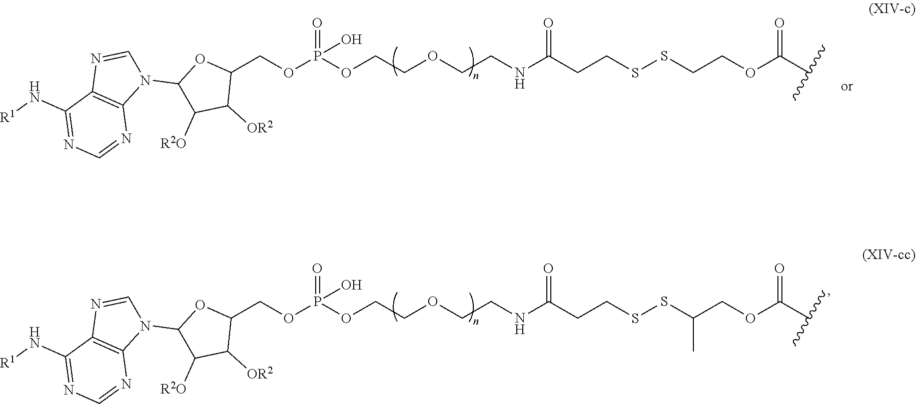
or a salt thereof, wherein n is an integer between 0 and 30, inclusive.

122. The method of any one of claims 105-121, wherein the linking group comprises an oligonucleotide.

123. The method of claim 122, wherein the oligonucleotide is Q24.

124. The method of any one of claims 105-123, wherein the linking group comprises a biotin moiety.

125. The method of claim 124, wherein the biotin moiety is a bis-biotin moiety.

126. The method of any one of claims 105-125, wherein the linking group comprises an avidin protein.

127. The method of claim 126, wherein the avidin protein is streptavidin.

128. The method of any one of claims 105-127, further comprising immobilizing the linking group to a surface

129. The method of any one of claims 126-128, wherein the avidin protein is immobilized to a surface.

130. The method of any one of claims 105-129, wherein the linking group comprises a moiety of Formula (XV): 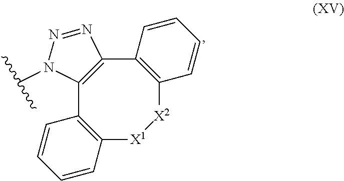
or a salt thereof, wherein:
one of X¹ and X² is CH and the other is N—Z¹; and
Z¹ comprises an oligonucleotide.

131. The method of claim 130, wherein the linking group comprises a moiety of Formulae (XV-a) or (XV-b): 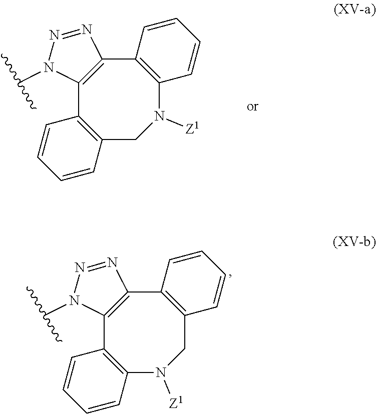
or a salt thereof.

132. A method of sequencing a peptide, comprising exposing a compound of Formula (III): 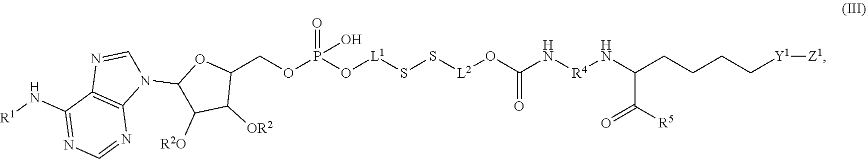
or a salt thereof, to a peptidase in a degradation process;
obtaining data during the degradation process;
analyzing the data to determine portions of the data corresponding to amino acids that are sequentially exposed at a terminus of the peptide during the degradation process; and
outputting an amino acid sequence representative of the peptide;
wherein:
R¹ is a solid support;
each instance of R² is independently hydrogen or an oxygen protecting group;
R⁴ is the peptide;
R⁵ is —OH or —NH₂;
L¹ is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, or a combination thereof;
L² is optionally substituted C₂ alkylene;
Y¹ comprises a click chemistry adduct; and
Z¹ comprises an oligonucleotide.

133. The method of claim 132, further comprising coupling a compound of Formula (I): 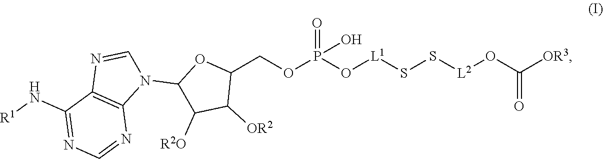
or a salt thereof, with a compound of Formula (XVI): 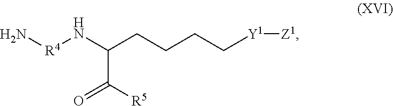
or a salt thereof, under suitable conditions to obtain the compound of Formula (III), or salt thereof, wherein R³ is optionally substituted alkyl, optionally substituted heterocyclyl, optionally substituted aryl, or optionally substituted heteroaryl.

134. The method of claim 132 or 133, wherein the compound of Formula (III) is of Formula (III-d): 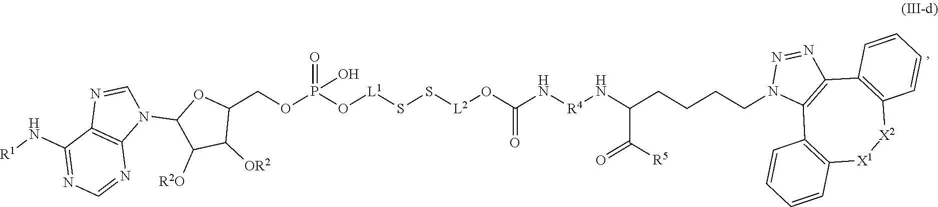
or a salt thereof, wherein one of X¹ and X² is CH and the other is N—Z¹.

135. The method of claim 134, further comprising coupling a compound of Formula (II): 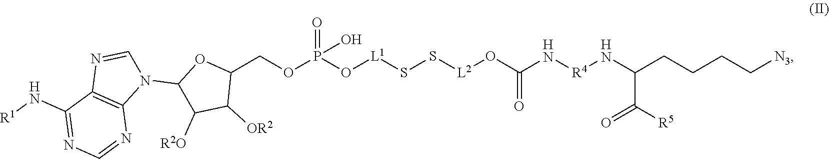
or a salt thereof, with a compound of Formula (XIII): 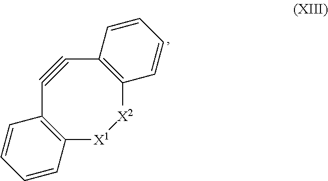
or a salt thereof, under suitable conditions to obtain the compound of Formula (III-d), or salt thereof.

136. The method of claim 135, further comprising coupling a compound of Formula (I): 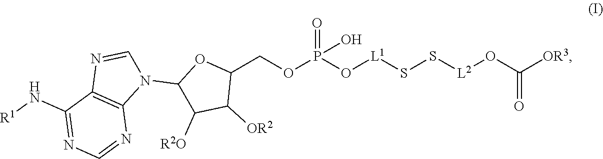
or a salt thereof, with a compound of Formula (XII): 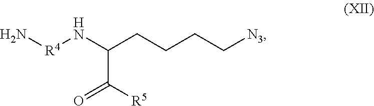
or a salt thereof, under suitable conditions to obtain the compound of Formula (II), or salt thereof, wherein R³ is optionally substituted alkyl, optionally substituted heterocyclyl, optionally substituted aryl, or optionally substituted heteroaryl.

137. The method of any one of claims 132-136, wherein the peptide remains in the solid phase while conjugated to the solid support.

138. The method of any one of claims 132, 133, or 137, further comprising cleaving the compound of Formula (III), or salt thereof, from the solid support to provide a compound of Formula (XVI): 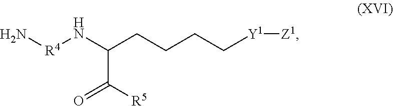
or a salt thereof.

139. The method of claim 138, wherein cleaving the compound of Formula (III), or salt thereof, from the solid support to provide the compound of Formula (XVI), or salt thereof, comprises conversion of a compound of Formula (XVII): 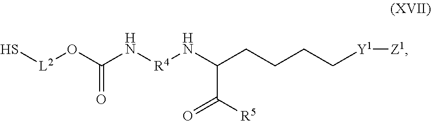
or a salt thereof, to the compound of Formula (XVI), or salt thereof.

140. The method of any one of claims 134-137, further comprising cleaving the compound of Formula (III-d), or salt thereof, from the solid support to provide a compound of Formula (XII): 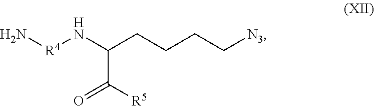
or a salt thereof.

141. The method of claim 140, wherein cleaving the compound of Formula (III-d), or salt thereof, from the solid support to provide the compound of Formula (XII), or salt thereof, comprises conversion of a compound of Formula (XVIII): 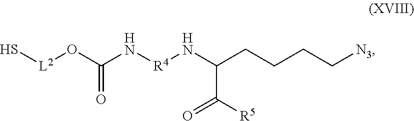
or a salt thereof, to the compound of Formula (XII), or salt thereof.

142. The method of any one of claims 138-141, comprising exposing the compound of Formula (XVI), or salt thereof, or the compound of Formula (XII), or salt thereof, to the peptidase in the degradation process.

143. The method of any one of claims 132-142, wherein Z¹ comprises Q24.

144. The method of any one of claims 132-143, wherein Z¹ further comprises a biotin moiety.

145. The method of claim 144, wherein the biotin moiety is a bis-biotin moiety.

146. The method of any one of claims 132-145, wherein Z¹ further comprises an avidin protein.

147. The method of claim 146, wherein the avidin protein is streptavidin.

148. The method of claim 146 or claim 147, wherein the avidin protein is immobilized to a surface.

149. The method of any one of claims 106-148, wherein the peptidase is an exopeptidase.

150. The method of any one of claims 106-149, wherein the peptidase is an aminopeptidase.

151. The method of any one of claims 106-150, wherein the peptidase is proline aminopeptidase, a proline iminopeptidase, a glutamate/aspartate-specific aminopeptidase, a methionine-specific aminopeptidase, or a zinc metalloprotease.

152. The method of any one of claims 106-151, wherein the peptidase is a TET aminopeptidase.

153. A kit comprising:
the compound of any one of claims 1-60, or a salt thereof; and
instructions for its use.


SEQUENCE LISTING (44 sequences)

SEQ ID NO: 1 - protein, 353 aa, Pyrococcus horikoshii
MEVRNMVDYELLKKVVEAPGVSGYEFLGIRDVVIEEIKDYVDEVKVDKLGNVIAHKKGEGPKVMIAAHMDQIGLMVTHIEKNGFLRVAPIGGVDPKTLIAQRFKVWIDKGKFIYGVGASVPPHIQKPEDRKKAPDWDQIFIDIGAESKEEAEDMGVKIGTVITWDGRLERLGKHRFVSIAFDDRIAVYTILEVAKQLKDAKADVYFVATVQEEVGLRGARTSAFGIEPDYGFAIDVTIAADIPGTPEHKQVTHLGKGTAIKIMDRSVICHPTIVRWLEELAKKHEIPYQLEILLGGGTDAGAIHLTKAGVPTGALSVPARYIHSNTEVVDERDVDATVELMTKALENIHELKI

SEQ ID NO: 2 - protein, 420 aa, synthetic construct
MEVRNMVDYELLKKVVEAPGVSGYEFLGIRDVVIEEIKDYVDEVKVDKLGNVIAHKKGEGPKVMIAAHMDQIGLMVTHIEKNGFLRVAPIGGVDPKTLIAQRFKVWIDKGKFIYGVGASVPPHIQKPEDRKKAPDWDQIFIDIGAESKEEAEDMGVKIGTVITWDGRLERLGKHRFVSIAFDDRIAVYTILEVAKQLKDAKADVYFVATVQEEVGLRGARTSAFGIEPDYGFAIDVTIAADIPGTPEHKQVTHLGKGTAIKIMDRSVICHPTIVRWLEELAKKHEIPYQLEILLGGGTDAGAIHLTKAGVPTGALSVPARYIHSNTEVVDERDVDATVELMTKALENIHELKIGGSHHHHHHHHHHGGGSGGGSGGGSGLNDFFEAQKIEWHEGGGSGGGSGGGSGLNDFFEAQKIEWHE

SEQ ID NO: 3 - protein, 353 aa, Pyrococcus horikoshii
MDLKGGESMVDWKLMQEIIEAPGVSGYEHLGIRDIVVDVLKEVADEVKVDKLGNVIAHFKGSSPRIMVAAHMDKIGVMVNHIDKDGYLHIVPIGGVLPETLVAQRIRFFTEKGERYGVVGVLPPHLRRGQEDKGSKIDWDQIVVDVGASSKEEAEEMGFRVGTVGEFAPNFTRLNEHRFATPYLDDRICLYAMIEAARQLGDHEADIYIVGSVQEEVGLRGARVASYAINPEVGIAMDVTFAKQPHDKGKIVPELGKGPVMDVGPNINPKLRAFADEVAKKYEIPLQVEPSPRPTGTDANMQINREGVATAVLSIPIRYMHSQVELADARDVDNTIKLAKALLEELKPMDFTP

SEQ ID NO: 4 - protein, 364 aa, synthetic construct
MDLKGGESMVDWKLMQEIIEAPGVSGYEHLGIRDIVVDVLKEVADEVKVDKLGNVIAHFKGSSPRIMVAAHMDKIGVMVNHIDKDGYLHIVPIGGVLPETLVAQRIRFFTEKGERYGVVGVLPPHLRRGQEDKGSKIDWDQIVVDVGASSKEEAEEMGFRVGTVGEFAPNFTRLNEHRFATPYLDDRICLYAMIEAARQLGDHEADIYIVGSVQEEVGLRGARVASYAINPEVGIAMDVTFAKQPHDKGKIVPELGKGPVMDVGPNINPKLRAFADEVAKKYEIPLQVEPSPRPTGTDANMQINREGVATAVLSIPIRYMHSQVELADARDVDNTIKLAKALLEELKPMDFTPGHHHHHHHHHH

SEQ ID NO: 5 - protein, 447 aa, Yersinia pestis
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQLE

SEQ ID NO: 6 - protein, 453 aa, synthetic construct
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQLEHHHHHH

SEQ ID NO: 7 - protein, 445 aa, synthetic construct
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQ

SEQ ID NO: 8 - protein, 454 aa, synthetic construct
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQGGSHHHHHH

SEQ ID NO: 9 - protein, 901 aa, unidentified
  source 1..901: note = L. pneumophila
MMVKQGVFMKTDQSKVKKLSDYKSLDYFVIHVDLQIDLSKKPVESKARLTVVPNLNVDSHSNDLVLDGENMTLVSLQMNDNLLKENEYELTKDSLIIKNIPQNTPFTIEMTSLLGENTDLFGLYETEGVALVKAESEGLRRVFYLPDRPDNLATYKTTIIANQEDYPVLLSNGVLIEKKELPLGLHSVTWLDDVPKPSYLFALVAGNLQRSVTYYQTKSGRELPIEFYVPPSATSKCDFAKEVLKEAMAWDERTFNLECALRQHMVAGVDKYASGASEPTGLNLFNTENLFASPETKTDLGILRVLEVVAHEFFHYWSGDRVTIRDWFNLPLKEGLTTFRAAMFREELFGTDLIRLLDGKNLDERAPRQSAYTAVRSLYTAAAYEKSADIFRMMMLFIGKEPFIEAVAKFFKDNDGGAVTLEDFIESISNSSGKDLRSFLSWFTESGIPELIVTDELNPDTKQYFLKIKTVNGRNRPIPILMGLLDSSGAEIVADKLLIVDQEEIEFQFENIQTRPIPSLLRSFSAPVHMKYEYSYQDLLLLMQFDTNLYNRCEAAKQLISALINDFCIGKKIELSPQFFAVYKALLSDNSLNEWMLAELITLPSLEELIENQDKPDFEKLNEGRQLIQNALANELKTDFYNLLFRIQISGDDDKQKLKGFDLKQAGLRRLKSVCFSYLLNVDFEKTKEKLILQFEDALGKNMTETALALSMLCEINCEEADVALEDYYHYWKNDPGAVNNWFSIQALAHSPDVIERVKKLMRHGDFDLSNPNKVYALLGSFIKNPFGFHSVTGEGYQLVADAIFDLDKINPTLAANLTEKFTYWDKYDVNRQAMMISTLKIIYSNATSSDVRTMAKKGLDKVKEDLPLPIHLTFHGGSTMQDRTAQLIADGNKENAYQLH

SEQ ID NO: 10 - protein, 265 aa, unidentified
  source 1..265: note = Escherichia coli methionine
MGTAISIKTPEDIEKMRVAGRLAAEVLEMIEPYVKPGVSTGELDRICNDYIVNEQHAVSACLGYHGYPKSVCISINEVVCHGIPDDAKLLKDGDIVNIDVTVIKDGFHGDTSKMFIVGKPTIMGERLCRITQESLYLALRMVKPGINLREIGAAIQKFVEAEGFSVVREYCGHGIGRGFHEEPQVLHYDSRETNVVLKPGMTFTIEPMVNAGKKEIRTMKDGWTVKTKDRSLSAQYEHTIVVTDNGCEILTLRKDDTIPAIISHD

SEQ ID NO: 11 - protein, 322 aa, unidentified
  source 1..322: note = M. smegmatis
MGTLEANTNGPGSMLSRMPVSSRTVPFGDHETWVQVTTPENAQPHALPLIVLHGGPGMAHNYVANIAALADETGRTVIHYDQVGCGNSTHLPDAPADFWTPQLFVDEFHAVCTALGIERYHVLGQSWGGMLGAEIAVRQPSGLVSLAICNSPASMRLWSEAAGDLRAQLPAETRAALDRHEAAGTITHPDYLQAAAEFYRRHVCRVVPTPQDFADSVAQMEAEPTVYHTMNGPNEFHVVGTLGDWSVIDRLPDVTAPVLVIAGEHDEATPKTWQPFVDHIPDVRSHVFPGTSHCTHLEKPEEFRAVVAQFLHQHDLAADARV

SEQ ID NO: 12 - protein, 303 aa, Pyrococcus furiosus
MDTEKLMKAGEIAKKVREKAIKLARPGMLLLELAESIEKMIMELGGKPAFPVNLSINEIAAHYTPYKGDTTVLKEGDYLKIDVGVHIDGFIADTAVTVRVGMEEDELMEAAKEALNAAISVARAGVEIKELGKAIENEIRKRGFKPIVNLSGHKIERYKLHAGISIPNIYRPHDNYVLKEGDVFAIEPFATIGAGQVIEVPPTLIYMYVRDVPVRVAQARFLLAKIKREYGTLPFAYRWLQNDMPEGQLKLALKTLEKAGAIYGYPVLKEIRNGIVAQFEHTIIVEKDSVIVTQDMINKSTLE

SEQ ID NO: 13 - protein, 428 aa, Aeromonas sobria
HMSSPLHYVLDGIHCEPHFFTVPLDHQQPDDEETITLFGRTLCRKDRLDDELPWLLYLQGGPGFGAPRPSANGGWIKRALQEFRVLLLDQRGTGHSTPIHAELLAHLNPRQQADYLSHFRADSIVRDAELIREQLSPDHPWSLLGQSFGGFCSLTYLSLFPDSLHEVYLTGGVAPIGRSADEVYRATYQRVADKNRAFFARFPHAQAIANRLATHLQRHDVRLPNGQRLTVEQLQQQGLDLGASGAFEELYYLLEDAFIGEKLNPAFLYQVQAMQPFNTNPVFAILHELIYCEGAASHWAAERVRGEFPALAWAQGKDFAFTGEMIFPWMFEQFRELIPLKEAAHLLAEKADWGPLYDPVQLARNKVPVACAVYAEDMYVEFDYSRETLKGLSNSRAWITNEYEHNGLRVDGEQILDRLIRLNRDCLE

SEQ ID NO: 14 - protein, 348 aa, Pyrococcus furiosus
MKERLEKLVKFMDENSIDRVFIAKPVNVYYFSGTSPLGGGYIIVDGDEATLYVPELEYEMAKEESKLPVVKFKKFDEIYEILKNTETLGIEGTLSYSMVENFKEKSNVKEFKKIDDVIKDLRIIKTKEEIEIIEKACEIADKAVMAAIEEITEGKREREVAAKVEYLMKMNGAEKPAFDTIIASGHRSALPHGVASDKRIERGDLVVIDLGALYNHYNSDITRTIVVGSPNEKQREIYEIVLEAQKRAVEAAKPGMTAKELDSIAREIIKEYGYGDYFIHSLGHGVGLEIHEWPRISQYDETVLKEGMVITIEPGIYIPKLGGVRIEDTVLITENGAKRLTKTERELL

SEQ ID NO: 15 - protein, 298 aa, Elizabethkingia meningoseptica
MIPITTPVGNFKVWTKRFGTNPKIKVLLLHGGPAMTHEYMECFETFFQREGFEFYEYDQLGSYYSDQPTDEKLWNIDRFVDEVEQVRKAIHADKENFYVLGNSWGGILAMEYALKYQQNLKGLIVANMMASAPEYVKYAEVLSKQMKPEVLAEVRAIEAKKDYANPRYTELLFPNYYAQHICRLKEWPDALNRSLKHVNSTVYTLMQGPSELGMSSDARLAKWDIKNRLHEIATPTLMIGARYDTMDPKAMEEQSKLVQKGRYLYCPNGSHLAMWDDQKVFMDGVIKFIKDVDTKSFN

SEQ ID NO: 16 - protein, 310 aa, unidentified
  source 1..310: note = N. gonorrhoeae
MYEIKQPFHSGYLQVSEIHQIYWEESGNPDGVPVIFLHGGPGAGASPECRGFFNPDVFRIVIIDQRGCGRSHPYACAEDNTTWDLVADIEKVREMLGIGKWLVFGGSWGSTLSLAYAQTHPERVKGLVLRGIFLCRPSETAWLNEAGGVSRIYPEQWQKFVAPIAENRRNRLIEAYHGLLFHQDEEVCLSAAKAWADWESYLIRFEPEGVDEDAYASLAIARLENHYFVNGGWLQGDKAILNNIGKIRHIPTVIVQGRYDLCTPMQSAWELSKAFPEAELRVVQAGHCAFDPPLADALVQAVEDILPRLL

SEQ ID NO: 17 - protein, 870 aa, Escherichia coli
MTQQPQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCEAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNMGAMENKGLNIFNSKYVLARTDTATDKDYLDIERVIGHEYFHNWTGNRVTCRDWFQLSLKEGLTVFRDQEFSSDLGSRAVNRINNVRTMRGLQFAEDASPMAHPIRPDMVIEMNNFYTLTVYEKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 18 - protein, 889 aa, unidentified
  source 1..889: note = P. falciparum
PKIHYRKDYKPSGFIINQVTLNINIHDQETIVRSVLDMDISKHNVGEDLVFDGVGLKINEISINNKKLVEGEEYTYDNEFLTIFSKFVPKSKFAFSSEVIIHPETNYALTGLYKSKNIIVSQCEATGFRRITFFIDRPDMMAKYDVTVTADKEKYPVLLSNGDKVNEFEIPGGRHGARFNDPPLKPCYLFAVVAGDLKHLSATYITKYTKKKVELYVFSEEKYVSKLQWALECLKKSMAFDEDYFGLEYDLSRLNLVAVSDFNVGAMENKGLNIFNANSLLASKKNSIDFSYARILTVVGHEYFHQYTGNRVTLRDWFQLTLKEGLTVHRENLFSEEMTKTVTTRLSHVDLLRSVQFLEDSSPLSHPIRPESYVSMENFYTTTVYDKGSEVMRMYLTILGEEYYKKGFDIYIKKNDGNTATCEDFNYAMEQAYKMKKADNSANLNQYLLWFSQSGTPHVSFKYNYDAEKKQYSIHVNQYTKPDENQKEKKPLFIPISVGLINPENGKEMISQTTLELTKESDTFVFNNIAVKPIPSLFRGFSAPVYIEDQLTDEERILLLKYDSDAFVRYNSCTNIYMKQILMNYNEFLKAKNEKLESFQLTPVNAQFIDAIKYLLEDPHADAGFKSYIVSLPQDRYIINFVSNLDTDVLADTKEYIYKQIGDKLNDVYYKMFKSLEAKADDLTYFNDESHVDFDQMNMRTLRNTLLSLLSKAQYPNILNEIIEHSKSPYPSNWLTSLSVSAYFDKYFELYDKTYKLSKDDELLLQEWLKTVSRSDRKDIYEILKKLENEVLKDSKNPNDIRAVYLPFTNNLRRFHDISGKGYKLIAEVITKTDKFNPMVATQLCEPFKLWNKLDTKRQELMLNEMNTMLQEPQISNNLKEYLLRLTNK

SEQ ID NO: 19 - protein, 919 aa, synthetic construct
MWLAAAAPSLARRLLFLGPPPPPLLLLVFSRSSRRRLHSLGLAAMPEKRPFERLPADVSPINYSLCLKPDLLDFTFEGKLEAAAQVRQATNQIVMNCADIDIITASYAPEGDEEIHATGFNYQNEDEKVTLSFPSTLQTGTGTLKIDFVGELNDKMKGFYRSKYTTPSGEVRYAAVTQFEATDARRAFPCWDEPAIKATFDISLVVPKDRVALSNMNVIDRKPYPDDENLVEVKFARTPVMSTYLVAFVVGEYDFVETRSKDGVCVRVYTPVGKAEQGKFALEVAAKTLPFYKDYFNVPYPLPKIDLIAIADFAAGAMENWGLVTYRETALLIDPKNSCSSSRQWVALVVGHELAHQWFGNLVTMEWWTHLWLNEGFASWIEYLCVDHCFPEYDIWTQFVSADYTRAQELDALDNSHPIEVSVGHPSEVDEIFDAISYSKGASVIRMLHDYIGDKDFKKGMNMYLTKFQQKNAATEDLWESLENASGKPIAAVMNTWTKQMGFPLIYVEAEQVEDDRLLRLSQKKFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQNDLFSLARAGIISTVEVLKVMEAFVNEPNYTVWSDLSCNLGILSTLLSHTDFYEEIQEFVKDVFSPIGERLGWDPKPGEGHLDALLRGLVLGKLGKAGHKATLEEARRRFKDHVEGKQILSADLRSPVYLTVLKHGDGTTLDIMLKLHKQADMQEEKNRIERVLGATLLPDLIQKVLTFALSEEVRPQDTVSVIGGVAGGSKHGRKAAWKFIKDNWEELYNRYQGGFLISRLIKLSVEGFAVDKMAGEVKAFFESHPAPSAERTIQQCCENILLNAAWLKRDAESIHQYLLQRKASPPTV

SEQ ID NO: 20 - protein, 919 aa, synthetic construct
MWLAAAAPSLARRLLFLGPPPPPLLLLVFSRSSRRRLHSLGLAAMPEKRPFERLPADVSPINYSLCLKPDLLDFTFEGKLEAAAQVRQATNQIVMNCADIDIITASYAPEGDEEIHATGFNYQNEDEKVTLSFPSTLQTGTGTLKIDFVGELNDKMKGFYRSKYTTPSGEVRYAAVTQFEATDARRAFPCWDEPAIKATFDISLVVPKDRVALSNMNVIDRKPYPDDENLVEVKFARTPVMSTYLVAFVVGEYDFVETRSKDGVCVRVYTPVGKAEQGKFALEVAAKTLPFYKDYFNVPYPLPKIDLIAIADFAAGAMENWGLVTYRETALLIDPKNSCSSSRQWVALVVGHVLAHQWFGNLVTMEWWTHLWLNEGFASWIEYLCVDHCFPEYDIWTQFVSADYTRAQELDALDNSHPIEVSVGHPSEVDEIFDAISYSKGASVIRMLHDYIGDKDFKKGMNMYLTKFQQKNAATEDLWESLENASGKPIAAVMNTWTKQMGFPLIYVEAEQVEDDRLLRLSQKKFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQNDLFSLARAGIISTVEVLKVMEAFVNEPNYTVWSDLSCNLGILSTLLSHTDFYEEIQEFVKDVFSPIGERLGWDPKPGEGHLDALLRGLVLGKLGKAGHKATLEEARRRFKDHVEGKQILSADLRSPVYLTVLKHGDGTTLDIMLKLHKQADMQEEKNRIERVLGATLLPDLIQKVLTFALSEEVRPQDTVSVIGGVAGGSKHGRKAAWKFIKDNWEELYNRYQGGFLISRLIKLSVEGFAVDKMAGEVKAFFESHPAPSAERTIQQCCENILLNAAWLKRDAESIHQYLLQRKASPPTV

SEQ ID NO: 21 - protein, 864 aa, Francisella tularensis
MIYEFVMTDPKIKYLKDYKPSNYLIDETHLIFELDESKTRVTANLYIVANRENRENNTLVLDGVELKLLSIKLNNKHLSPAEFAVNENQLIINNVPEKFVLQTVVEINPSANTSLEGLYKSGDVFSTQCEATGFRKITYYLDRPDVMAAFTVKIIADKKKYPIILSNGDKIDSGDISDNQHFAVWKDPFKKPCYLFALVAGDLASIKDTYITKSQRKVSLEIYAFKQDIDKCHYAMQAVKDSMKWDEDRFGLEYDLDTFMIVAVPDFNAGAMENKGLNIFNTKYIMASNKTATDKDFELVQSVVGHEYFHNWTGDRVTCRDWFQLSLKEGLTVFRDQEFTSDLNSRDVKRIDDVRIIRSAQFAEDASPMSHPIRPESYIEMNNFYTVTVYNKGAEIIRMIHTLLGEEGFQKGMKLYFERHDGQAVTCDDFVNAMADANNRDFSLFKRWYAQSGTPNIKVSENYDASSQTYSLTLEQTTLPTADQKEKQALHIPVKMGLINPEGKNIAEQVIELKEQKQTYTFENIAAKPVASLFRDFSAPVKVEHKRSEKDLLHIVKYDNNAFNRWDSLQQIATNIILNNADLNDEFLNAFKSILHDKDLDKALISNALLIPIESTIAEAMRVIMVDDIVLSRKNVVNQLADKLKDDWLAVYQQCNDNKPYSLSAEQIAKRKLKGVCLSYLMNASDQKVGTDLAQQLFDNADNMTDQQTAFTELLKSNDKQVRDNAINEFYNRWRHEDLVVNKWLLSQAQISHESALDIVKGLVNHPAYNPKNPNKVYSLIGGFGANFLQYHCKDGLGYAFMADTVLALDKFNHQVAARMARNLMSWKRYDSDRQAMMKNALEKIKASNPSKNVFEIVSKSLES

SEQ ID NO: 22 - protein, 408 aa, unidentified
  source 1..408: note = T. aquaticus
MDAFTENLNKLAELAIRVGLNLEEGQEIVATAPIEAVDFVRLLAEKAYENGASLFTVLYGDNLIARKRLALVPEAHLDRAPAWLYEGMAKAFHEGAARLAVSGNDPKALEGLPPERVGRAQQAQSRAYRPTLSAITEFVTNWTIVPFAHPGWAKAVFPGLPEEEAVQRLWQAIFQATRVDQEDPVAAWEAHNRVLHAKVAFLNEKRFHALHFQGPGTDLTVGLAEGHLWQGGATPTKKGRLCNPNLPTEEVFTAPHRERVEGVVRASRPLALSGQLVEGLWARFEGGVAVEVGAEKGEEVLKKLLDTDEGARRLGEVALVPADNPIAKTGLVFFDTLFDENAASHIAFGQAYAENLEGRPSGEEFRRRGGNESMVHVDWMIGSEEVDVDGLLEDGTRVPLMRRGRWVI

SEQ ID NO: 23 - protein, 362 aa, unidentified
  source 1..362: note = Bacillus stearothermophilus
MAKLDETLTMLKALTDAKGVPGNEREARDVMKTYIAPYADEVTTDGLGSLIAKKEGKSGGPKVMIAGHLDEVGFMVTQIDDKGFIRFQTLGGWWSQVMLAQRVTIVTKKGDITGVIGSKPPHILPSEARKKPVEIKDMFIDIGATSREEAMEWGVRPGDMIVPYFEFTVLNNEKMLLAKAWDNRIGCAVAIDVLKQLKGVDHPNTVYGVGTVQEEVGLRGARTAAQFIQPDIAFAVDVGIAGDTPGVSEKEAMGKLGAGPHIVLYDATMVSHRGLREFVIEVAEELNIPHHFDAMPGVGTDAGAIHLTGIGVPSLTIAIPTRYIHSHAAILHRDDYENTVKLLVEVIKRLDADKVKQLTFDE

SEQ ID NO: 24 - protein, 484 aa, unidentified
  source 1..484: note = Vibrio cholera
MEDKVWISMGADAVGSLNPALSESLLPHSFASGSQVWIGEVAIDELAELSHTMHEQHNRCGGYMVHTSAQGAMAALMMPESIANFTIPAPSQQDLVNAWLPQVSADQITNTIRALSSFNNRFYTTTSGAQASDWLANEWRSLISSLPGSRIEQIKHSGYNQKSVVLTIQGSEKPDEWVIVGGHLDSTLGSHTNEQSIAPGADDDASGIASLSEIIRVLRDNNFRPKRSVALMAYAAEEVGLRGSQDLANQYKAQGKKVVSVLQLDMTNYRGSAEDIVFITDYTDSNLTQFLTTLIDEYLPELTYGYDRCGYACSDHASWHKAGFSAAMPFESKFKDYNPKIHTSQDTLANSDPTGNHAVKFTKLGLAYVIEMANAGSSQVPDDSVLQDGTAKINLSGARGTQKRFTFELSQSKPLTIQTYGGSGDVDLYVKYGSAPSKSNWDCRPYQNGNRETCSFNNAQPGIYHVMLDGYTNYNDVALKASTQ

SEQ ID NO: 25 - protein, 488 aa, Photobacterium halotolerans
MEDKVWISIGSDASQTVKSVMQSNARSLLPESLASNGPVWVGQVDYSQLAELSHHMHEDHQRCGGYMVHSSPESAIAASNMPQSLVAFSIPEISQQDTVNAWLPQVNSQAITGTITSLTSFINRFYTTTSGAQASDWLANEWRSLSASLPNASVRQVSHFGYNQKSVVLTITGSEKPDEWIVLGGHLDSTIGSHTNEQSVAPGADDDASGIASVTEIIRVLSENNFQPKRSIAFMAYAAEEVGLRGSQDLANQYKAEGKQVISALQLDMTNYKGSVEDIVFITDYTDSNLTTFLSQLVDEYLPSLTYGFDTCGYACSDHASWHKAGFSAAMPFEAKFNDYNPMIHTPNDTLQNSDPTASHAVKFTKLGLAYAIEMASTTGGTPPPTGNVLKDGVPVNGLSGATGSQVHYSFELPAQKNLQISTAGGSGDVDLYVSFGSEATKQNWDCRPYRNGNNEVCTFAGATPGTYSIMLDGYRQFSGVTLKASTQ

SEQ ID NO: 26 - protein, 871 aa, Yersinia pestis
MTQQPQAKYRHDYRAPDYTITDIDLDFALDAQKTTVTAVSKVKRQGTDVTPLILNGEDLTLISVSVDGQAWPHYRQQDNTLVIEQLPADFTLTIVNDIHPATNSALEGLYLSGEALCTQCEAEGFRHITYYLDRPDVLARFTTRIVADKSRYPYLLSNGNRVGQGELDDGRHWVKWEDPFPKPSYLFALVAGDFDVLQDKFITRSGREVALEIFVDRGNLDRADWAMTSLKNSMKWDETRFGLEYDLDIYMIVAVDFFNMGAMENKGLNVFNSKYVLAKAETATDKDYLNIEAVIGHEYFHNWTGNRVTCRDWFQLSLKEGLTVFRDQEFSSDLGSRSVNRIENVRVMRAAQFAEDASPMAHAIRPDKVIEMNNFYTLTVYEKGSEVIRMMHTLLGEQQFQAGMRLYFERHDGSAATCDDFVQAMEDVSNVDLSLFRRWYSQSGTPLLTVHDDYDVEKQQYHLFVSQKTLPTADQPEKLPLHIPLDIELYDSKGNVIPLQHNGLPVHHVLNVTEAEQTFTFDNVAQKPIPSLLREFSAPVKLDYPYSDQQLTFLMQHARNEFSRWDAAQSLLATYIKLNVAKYQQQQPLSLPAHVADAFRAILLDEHLDPALAAQILTLPSENEMAELFTTIDPQAISTVHEAITRCLAQELSDELLAVYVANMTPVYRIEHGDIAKRALRNTCLNYLAFGDEEFANKLVSLQYHQADNMTDSLAALAAAVAAQLPCRDELLAAFDVRWNHDGLVMDKWFALQATSPAANVLVQVRTLLKHPAFSLSNPNRTRSLIGSFASGNPAAFHAADGSGYQFLVEILSDLNTRNPQVAARLIEPLIRLKRYDAGRQALMRKALEQLKTLDNLSGDLYEKITKALAA

SEQ ID NO: 27 - protein, 483 aa, Vibrio anguillarum
MEEKVWISIGGDATQTALRSGAQSLLPENLINQTSVWVGQVPVSELATLSHEMHENHQRCGGYMVHPSAQSAMSVSAMPLNLNAFSAPEITQQTTVNAWLPSVSAQQITSTITTLTQFKNRFYTTSTGAQASNWIADHWRSLSASLPASKVEQITHSGYNQKSVMLTITGSEKPDEWVVIGGHLDSTLGSRTNESSIAPGADDDASGIAGVTEIIRLLSEQNFRPKRSIAFMAYAAEEVGLRGSQDLANRFKAEGKKVMSVMQLDMTNYQGSREDIVFITDYTDSNFTQYLTQLLDEYLPSLTYGFDTCGYACSDHASWHAVGYPAAMPFESKFNDYNPNIHSPQDTLQNSDPTGFHAVKFTKLGLAYVVEMGNASTPPTPSNQLKNGVPVNGLSASRNSKTWYQFELQEAGNLSIVLSGGSGDADLYVKYQTDADLQQYDCRPYRSGNNETCQFSNAQPGRYSILLHGYNNYSNASLVANAQ

SEQ ID NO: 28 - protein, 482 aa, unidentified
  source 1..482: note = Salinivibrio sp
MEDKKVWISIGADAQQTALSSGAQPLLAQSVAHNGQAWIGEVSESELAALSHEMHENHHRCGGYIVHSSAQSAMAASNMPLSRASFIAPAISQQALVTPWISQIDSALIVNTIDRLTDFPNRFYTTTSGAQASDWIKQRWQSLSAGLAGASVTQISHSGYNQASVMLTIEGSESPDEWVVVGGHLDSTIGSRTNEQSIAPGADDDASGIAAVTEVIRVLAQNNFQPKRSIAFVAYAAEEVGLRGSQDVANQFKQAGKDVRGVLQLDMTNYQGSAEDIVFITDYTDNQLTQYLTQLLDEYLPTLNYGFDTCGYACSDHASWHQVGYPAAMPFEAKFNDYNPNIHTPQDTLANSDSEGAHAAKFTKLGLAYTVELANADSSPNPGNELKLGEPINGLSGARGNEKYFNYRLDQSGELVIRTYGGSGDVDLYVKANGDVSTGNWDCRPYRSGNDEVCRFDNATPGNYAVMLRGYRTYDNVSLIVE

SEQ ID NO: 29 - protein, 301 aa, Vibrio proteolyticus
MPPITQQATVTAWLPQVDASQITGTISSLESFTNRFYTTTSGAQASDWIASEWQALSASLPNASVKQVSHSGYNQKSVVMTITGSEAPDEWIVIGGHLDSTIGSHTNEQSVAPGADDDASGIAAVTEVIRVLSENNFQPKRSIAFMAYAAEEVGLRGSQDLANQYKSEGKNVVSALQLDMTNYKGSAQDVVFITDYTDSNFTQYLTQLMDEYLPSLTYGFDTCGYACSDHASWHNAGYPAAMPFESKFNDYNPRIHTTQDTLANSDPTGSHAKKFTQLGLAYAIEMGSATGDTPTPGNQLE

SEQ ID NO: 30 - protein, 301 aa, Vibrio proteolyticus
MPPITQQATVTAWLPQVDASQITGTISSLESFTNRFYTTTSGAQASDWIASEWQFLSASLPNASVKQVSHSGYNQKSVVMTITGSEAPDEWIVIGGHLDSTIGSHTNEQSVAPGADDDASGIAAVTEVIRVLSENNFQPKRSIAFMAYAAEEVGLRGSQDLANQYKSEGKNVVSALQLDMTNYKGSAQDVVFITDYTDSNFTQYLTQLMDEYLPSLTYGFDTCGYACSDHASWHNAGYPAAMPFESKFNDYNPRIHTTQDTLANSDPTGSHAKKFTQLGLAYAIEMGSATGDTPTPGNQLE

SEQ ID NO: 31 - protein, 348 aa, Pyrococcus furiosus
MVDWELMKKIIESPGVSGYEHLGIRDLVVDILKDVADEVKIDKLGNVIAHFKGSAPKVMVAAHMDKIGLMVNHIDKDGYLRVVPIGGVLPETLIAQKIRFFTEKGERYGVVGVLPPHLRREAKDQGGKIDWDSIIVDVGASSREEAEEMGFRIGTIGEFAPNFTRLSEHRFATPYLDDRICLYAMIEAARQLGEHEADIYIVASVQEEIGLRGARVASFAIDPEVGIAMDVTFAKQPNDKGKIVPELGKGPVMDVGPNINPKLRQFADEVAKKYEIPLQVEPSPRPTGTDANVMQINREGVATAVLSIPIRYMHSQVELADARDVDNTIKLAKALLEELKPMDFTPLE

SEQ ID NO: 32 - DNA, 25 bp, synthetic construct
  source 1..25: mol_type = other DNA
ccacgcgtggaacccttgggatcca

SEQ ID NO: 33 - DNA, 25 bp, synthetic construct
  source 1..25: mol_type = other DNA
tggagtcaaggtcctctgatgccat

SEQ ID NO: 34 - protein, 10 aa, synthetic construct
GPPPPPPPPG

SEQ ID NO: 35 - protein, 5 aa, synthetic construct
  MOD_RES 1: note = isoglutamate
XGWRW

SEQ ID NO: 36 - protein, 10 aa, synthetic construct
DDGGGDDDFF

SEQ ID NO: 37 - protein, 14 aa, synthetic construct
GGSSSGSGNDEEFQ

SEQ ID NO: 38 - protein, 12 aa, synthetic construct
GGGGGDPDPDFF

SEQ ID NO: 39 - protein, 12 aa, synthetic construct
GDGDGDGDGDFF

SEQ ID NO: 40 - protein, 10 aa, synthetic construct
NNGGGNNNFF

SEQ ID NO: 41 - protein, 10 aa, synthetic construct
  MOD_RES 6: note = cysteic acid
  MOD_RES 7: note = cysteic acid
  MOD_RES 8: note = cysteic acid
DDGGGCCCFF

SEQ ID NO: 42 - protein, 14 aa, synthetic construct
AQSIYRFDTLKNNN

SEQ ID NO: 43 - protein, 15 aa, synthetic construct
GAQSIYRFDTLKNNN

SEQ ID NO: 44 - protein, 11 aa, synthetic construct
GAQSIYRFDTL